(19) United States
(12) Patent Application Publication
(10) Pub. No.: US 2023/0092895 A1
(43) Pub. Date: Mar. 23, 2023

(54) TANDEM CD19 CAR-BASED COMPOSITIONS AND METHODS FOR IMMUNOTHERAPY

(71) Applicant: Obsidian Therapeutics, Inc., Cambridge, MA (US)

(72) Inventors: Vipin Suri, Belmont, MA (US); Michael Joseph Briskin, Lexington, MA (US); Brian Dolinski, Cambridge, MA (US); Kutlu Goksu Elpek, Arlington, MA (US); Tucker Ezell, Cambridge, MA (US); Scott Francis Heller, Stoughton, MA (US); Mara Christine Inniss, Beverly, MA (US); Tariq A. Kassum, Cambridge, MA (US); Nicole Kosmider, Cambridge, MA (US); Abhishek Kulkarni, Brooklin, MA (US); Meghan Langley, Stoughton, MA (US); Dan Jun Li, Cambridge, MA (US); Michelle Lynn Ols, Northborough, MA (US); Benjamin J. Primack, Brookline, MA (US); Celeste Richardson, Brookline, MA (US); Steven Mark Shamah, Acton, MA (US); James Storer, Medford, MA (US); Dexue Sun, Cambridge, MA (US); Vijaya Balakrishnan, Cambridge, MA (US); Andrew R.M. Bradbury, Santa Fe, NM (US); Michael Frank Erasmus, Santa Fe, NM (US); Fortunato Ferrara, Santa Fe, NM (US)

(21) Appl. No.: 17/753,320

(22) PCT Filed: Aug. 30, 2019
(86) PCT No.: PCT/US2019/049099

§ 371 Date: Feb. 28, 2022

(51) Int. Cl. C12N 9/06; C07K 16/28; C07K 14/54; C07K 14/715; C07K 14/725; A61K 35/17

(52) U.S. Cl. CPC C12N 9/003; C07K 16/2803; C07K 14/5434; C07K 14/5443; C07K 14/7155; C12Y 105/01003; C07K 14/7051; A61K 35/17; A61K 2039/505

(57) ABSTRACT

The present invention provides biocircuit systems, effector modules and compositions for cancer immunotherapy. Methods for inducing anti-cancer immune responses in a subject are also provided.

### REFERENCE TO THE SEQUENCE LISTING

The present application is being filed along with a Sequence Listing in electronic format. The Sequence Listing is provided as a file entitled 268052-454311_ST25.txt, created on Aug. 28, 2019, which is 1.83 Mbytes in size. The information in the electronic format of the sequence listing is incorporated herein by reference in its entirety.

### FIELD OF THE INVENTION

The present invention relates to compositions and methods for immunotherapy. Provided in the present invention include polypeptides of biocircuit systems, effector modules, stimulus response elements (SREs) and immunotherapeutic agents, polynucleotides encoding the same, vectors and cells containing the polypeptides and/or polynucleotides for use in cancer immunotherapy. In one embodiment, the compositions comprise destabilizing domains (DDs) which tune protein stability.

### BACKGROUND OF THE INVENTION

Cancer immunotherapy aims to eradicate cancer cells by rejuvenating the tumoricidal functions of tumor-reactive immune cells, predominantly T cells. Strategies of cancer immunotherapy including the recent development of checkpoint blockade, adoptive cell transfer (ACT) and cancer vaccines which can increase the anti-tumor immune effector cells have produced remarkable results in several tumors.

The impact of host anti-tumor immunity and cancer immunotherapy is impeded by three major hurdles: 1) low number of tumor antigen-specific T cells due to clonal deletion; 2) poor activation of innate immune cells and accumulation of tolerogenic antigen-presenting cells in the tumor microenvironment; and 3) formation of an immunosuppressive tumor microenvironment. Particularly, in solid tumors the therapeutic efficacy of immunotherapeutic regimens remains unsatisfactory due to lack of an effective an anti-tumor response in the immunosuppressive tumor microenvironment. Tumor cells often induce immune tolerance or suppression and such tolerance is acquired because even truly foreign tumor antigens will become tolerated. Such tolerance is also active and dominant because cancer vaccines and adoptive transfer of pre-activated immune effector cells (e.g., T cells), are subject to suppression by inhibitory factors in the tumor microenvironment (TME).

In addition, administration of engineered T cells could result in on/off target toxicities as well as a cytokine release syndrome (reviewed by Tey Clin. Transl. Immunol., 2014, 3: e17 10.1038).

Development of a tunable switch that can turn on or off the transgenic immunotherapeutic agent expression is needed in case of adverse events. For example, adoptive cell therapies may have a very long and an indefinite half-life. Since toxicity can be progressive, a safety switch is desired to eliminate the infused cells. Systems and methods that can tune the transgenic protein level and expression window with high flexibility can enhance therapeutic benefit and reduce potential side effects.

To develop regulatable therapeutic agents for disease therapy, in particular cancer immunotherapy, the present invention provides biocircuit systems to control the expression of immunotherapeutic agents. The biocircuit system comprises a stimulus and at least one effector module that responds to the stimulus. The effector module may include a stimulus response element (SRE) that binds and is responsive to a stimulus and an immunotherapeutic agent operably linked to the SRE. In one example, a SRE is a destabilizing domain (DD) which is destabilized in the absence of its specific ligand and can be stabilized by binding to its specific ligand.

### SUMMARY OF THE INVENTION

The present invention provides compositions and methods for immunotherapy. In some embodiments, the compositions relate to tunable systems and agents that induce anti-cancer immune responses in a cell or in a subject.

In some embodiments, the compositions may include but are not limited to (a) an effector module and (b) a chimeric antigen receptor (CAR). The chimeric antigen receptor may be operably linked to the effector module.

In one embodiment, the effector module may include a stimulus response element (SRE) operably linked to an immunotherapeutic agent. The immunotherapeutic agent may be a cytokine or a cytokine-cytokine receptor fusion protein. In some embodiments, the SRE may be a DD. The DD may be derived from a parent protein or a mutant protein having one, two, three or more amino acid mutations compared to the parent protein. The parent protein may be selected from (i′) human DHFR (hDHFR) (SEQ ID NO. 1); (ii′) E. coli DHFR (ecDHFR) (SEQ ID NO. 2); or (iii′) human protein FKBP (SEQ ID NO. 3; 1087).

In some aspects, the immunotherapeutic agent may be a cytokine. In one embodiment, the cytokine may be IL12. The IL12 may be a fusion protein that includes but is not limited to a p40 subunit, a linker, and a p35 subunit. In some aspects, the p40 subunit may be a p40 (23-328 of WT) (SEQ ID NO. 563), a p40 (WT) (SEQ ID NO.1091) or a p40 (23-328 of WT) (K217N) (SEQ ID NO. 578). In one embodiment, the p40 subunit may be p40 (23-328 of WT) (SEQ ID NO. 563). In some embodiments, the p35 subunit may be a p35 (57-253 of WT) (SEQ ID NO. 564) or a p35 (WT) (SEQ ID NO. 1093). In one aspect, the p35 subunit may be a p35 (57-253 of WT) (SEQ ID NO. 564).

The immunotherapeutic agent may also be a cytokine-cytokine receptor fusion protein. In some aspects, the cytokine-cytokine receptor fusion polypeptide may include the whole or a portion of SEQ. ID NO. 785, 803 fused to the whole or a portion of any of SEQ. ID NOs. 803; 1057, 1299 to produce a IL15-IL15 receptor fusion polypeptide.

In some embodiments, the SRE of the effector module is derived from a hDHFR parent protein. The DD may be a mutant protein having a single mutation selected from the group consisting of hDHFR (I17V), hDHFR (F59S), hDHFR (N65D), hDHFR (K81R), hDHFR (A107V), hDHFR (Y122I), hDHFR (N127Y), hDHFR (M140I), hDHFR (K185E), hDHFR (N186D), and hDHFR (M1400. In some aspects the DD may include a double mutation selected from the group consisting of hDHFR (M1del, I17A), hDHFR (M1del, N127Y), hDHFR (M1del, I17V), hDHFR (M1del, Y122I), hDHFR (M1del, K185E), hDHFR (C7R, Y163C), hDHFR (A10V, H88Y), hDHFR (Q36K, Y122I), hDHFR (M53T, R138I), hDHFR (T57A, I72A), hDHFR (E63G, I176F), hDHFR (G21T, Y122I), hDHFR (L74N, Y122I), hDHFR (V75F, Y122I), hDHFR (L94A, T147A), DHFR (V121A, Y22I), hDHFR (Y122I, A125F), hDHFR (H131R, E144G), hDHFR (T137R, F143L), hDHFR (Y178H, E18IG), hDHFR (Y183H, K185E), hDHFR (E162G, I176F), and hDHFR (M1del, M140I). In some embodiments, the DD may include a triple mutation selected from the group consisting of hDHFR (V9A, S93R, P150L), hDHFR (I8V, K133E, Y163C), hDHFR (L23S, V121A, Y157C), hDHFR (K19E, F89L, E181G), hDHFR (Q36F, N65F, Y122I), hDHFR (G54R, M140V, S168C), hDHFR (V110A, V136M, K177R), hDHFR (Q36F, Y122I, A125F), hDHFR (N49D, F59S, D153G), hDHFR (G21E, I72V, I176T), hDHFR (M1del, I17A, Y122I), hDHFR (M1del, I17V, Y122I), hDHFR (M1del, N127Y, Y122I), hDHFR (M1del, E162G, I176F), hDHFR (M1del, H131R, E144G), and hDHFR (M1del, Y122I, A125F). In some aspects, the DD may include a quadruple or higher mutation selected from the group consisting of hDHFR (M1del, Q36F, Y122I, A125F), hDHFR (M1del, Y122I, H131R, E144G), hDHFR (M1del, E31D, F32M, V116I), hDHFR (M1del, Q36F, N65F, Y122I), hDHFR (V2A, R33G, Q36R, L100P, K185R), hDHFR (M1del, D22S, F32M, R33S, Q36S, N65S), hDHFR (I17N, L98S, K99R, M112T, E151G, E162G, E172G), hDHFR (G16S, I17V, F89L, D96G, K123E, M140V, D146G, K156R), hDHFR (K81R, K99R, L100P, E102G, N108D, K123R, H128R, D142G, F180L, K185E), hDHFR (R138G, D142G, F143S, K156R, K158E, E162G, V166A, K177E, Y178C, K185E, N186S), hDHFR (N14S, P24S, F35L, M53T, K56E, R92G, S93G, N127S, H128Y, F135L, F143S, L159P, L160P, E173A, F180L), hDHFR (F35L, R37G, N65A, L68S, K69E, R71G, L80P, K99G, G117D, L132P, I139V, M140I, D142G, D146G, E173G, D187G), hDHFR (L28P, N30H, M38V, V44A, L68S, N73G, R78G, A97T, K99R, A107T, K109R, D111N, L134P, F135V, T147A, I152V, K158R, E172G, V182A, E184R), hDHFR (V2A, I17V, N30D, E31G, Q36R, F59S, K69E, I72T, H88Y, F89L, N108D, K109E, V110A, 1115V, Y122D, L132P, F135S, M140V, E144G, T147A, Y157C, V170A, K174R, N186S), hDHFR (L100P, E102G, Q103R, P104S, E105G, N108D, V113A, W114R, Y122C, M126I, N127R, H128Y, L132P, F135P, I139T, F148S, F149L, I152V, D153A, D169G, V170A, I176A, K177R, V182A, K185R, N186S), and hDHFR (A10T, Q13R, N14S, N20D, P24S, N30S, M38T, T40A, K47R, N49S, K56R, I61T, K64R, K69R, I72A, R78G, E82G, F89L, D96G, N108D, M112V, W114R, Y122D, K123E, I139V, Q141R, D142G, F148L, E151G, E155G, Y157R, Q171R, Y183C, E184G, K185del, D187N).

In one embodiment, the DD includes three mutations hDHFR (M1del, Y122I, N127Y). In one embodiment, the DD includes a mutant protein having three mutations hDHFR (M1del, I17V, Y122I). In one aspect, the mutant protein may include two mutations hDHFR (M1del, I17V).

The CAR described herein may include (a) an extracellular target moiety; (b) a transmembrane domain; (c) an intracellular signaling domain; and (d) optionally, one or more co-stimulatory domains. The extracellular target moiety may be selected from a single chain variable fragment (scFv), Ig NAR, Fab fragment, Fab′ fragment, F(ab)′2 fragment, F(ab)′3 fragment, Fv, bis-scFv, a (scFv)2, minibody, diabody, triabody, tetrabody, intrabody, disulfide stabilized Fv protein (dsFv), unibody, nanobody, and an antigen binding region derived from an antibody that specifically binds to any of a protein of interest, a ligand, a receptor, a receptor fragment or a peptide aptamer. In one embodiment, the extracellular target moiety may be an scFv derived from an antibody that specifically binds a CD19 antigen.

In some aspects, the scFv may be CD19 scFv is selected from the group consisting of: (a) an amino acid sequence selected from the group consisting of SEQ ID NOs: 465; 83-227; 1034-1036; or (b) a heavy chain variable region having an amino acid sequence independently selected from the group consisting of SEQ ID NO: 9-40, 1169, and a light chain variable region having an amino acid sequence independently selected from the group consisting of SEQ ID NOs: 41-82, 1170.

In some embodiments, the intracellular signaling domain of the CAR may be a signaling domain derived from T cell receptor CD3zeta or a cell surface molecule selected from the group consisting of FcR gamma, FcR beta, CD3 gamma, CD3 delta, CD3 epsilon, CD5, CD22, CD79a, CD79b, and CD66d. In some aspects, the co-stimulatory domain may be present and is selected from the group consisting of 4-1BB (CD137), 2B4, HVEM, ICOS, LAG3, DAP10, DAP12, CD27, CD28, OX40 (CD134), CD30, CD40, ICOS (CD278), glucocorticoid-induced tumor necrosis factor receptor (GITR), lymphocyte function-associated antigen-1 (LFA-1), CD2, CD7, LIGHT, NKG2C, and B7-H3. In one embodiment, the intracellular signaling domain of the CAR is a T cell receptor CD3zeta signaling domain may include the amino acid sequence of SEQ ID NO: 229. The intracellular signaling domain of the CAR may be a T cell receptor CD3zeta signaling domain comprising the amino acid sequence of SEQ ID NO: 467. In some aspects, the co-stimulatory domain may be present, said co-stimulatory domain may be selected from amino acid sequence of SEQ ID NOs: 233, 228-232, and 234-334.

In some embodiments, the transmembrane domain may be derived from (a) a molecule selected from the group consisting of CD8α, CD4, CD5, CD8, CD8α, CD9, CD16, CD22, CD33, CD28, CD37, CD45, CD64, CD80, CD86, CD148, DAP 10, EpoRI, GITR, LAG3, ICOS, Her2, OX40 (CD134), 4-1BB (CD137), CD152, CD154, PD-1, or CTLA-4 or (b) a transmembrane region of an alpha, beta or zeta chain of a T-cell receptor; or (c) the CD3 epsilon chain of a T-cell receptor; or a (d) an immunoglobulin selected from IgG1, IgD, IgG4, and an IgG4 Fc region. In one embodiment, the transmembrane domain may include the amino acid sequence selected from the group consisting of SEQ ID NOs. 369, 335-368, 370-385 and 697-707.

In some aspects, the CAR may further include a hinge region near the transmembrane domain. The hinge region may include an amino acid sequence selected from the group consisting of SEQ ID NOs. 400, 386-399, and 401-464.

The SRE described herein may be responsive to or interact with at least one stimulus. In one embodiment, the stimulus may be Trimethoprim or Methotrexate.

In some embodiments, the effector module may be selected from the group consisting of SEQ ID NO. 1121, 1123, 1129, 1131, 1339, 1135, 1137, 1139, and 1141. In some aspects, the CAR may be selected from the group consisting of SEQ ID NO. 1120, 1122, 1128, 1130, 1132, 1134, 1136, 1138, and 1140.

In one embodiment, the composition may include but is not limited to the amino acid sequence of SEQ ID NO. 1127, 1125, 1126, 1082, 1118, 1119, 1124, or 1127.

Also provided herein are polynucleotides, vectors and immune cells that include the compositions described herein. The present disclosure also provides a method of inducing an immune response in a subject. Such methods may include preparing an immune cell that includes the compositions of any of claims 1-27; and (b) administering an effective amount of the immune cells to the subject. In one embodiment, the composition may be capable of inducing an immune response. Also provided herein are the methods for inducing the expression of T cell activation markers in the subject. Such methods may include administering an effective amount of the compositions described herein.

### BRIEF DESCRIPTION OF THE DRAWINGS

The foregoing and other objects, features and advantages will be apparent from the following description of particular embodiments of the invention, as illustrated in the accompanying drawings. The drawings are not necessarily to scale; emphasis instead being placed upon illustrating the principles of various embodiments of the invention.

FIG. 1 shows the IL12 levels in tandem expression constructs.

FIG. 2 shows the regulation of IL12 with Shield-1 treatment.

FIG. 3 shows the effect of EF1a promoter on IL12 levels.

FIG. 4 shows the effect of different promoters on IL12 levels.

### DETAILED DESCRIPTION OF THE INVENTION

The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

### I. Introduction

Cancer immunotherapy aims at the induction or restoration of the reactivity of the immune system towards cancer. Significant advances in immunotherapy research have led to the development of various strategies which may broadly be classified into active immunotherapy and passive immunotherapy. In general, these strategies may be utilized to directly kill cancer cells or to counter the immunosuppressive tumor microenvironment. Active immunotherapy aims at induction of an endogenous, long-lasting tumor-antigen specific immune response. The response can further be enhanced by non-specific stimulation of immune response modifiers such as cytokines. In contrast, passive immunotherapy includes approaches where immune effector molecules such as tumor-antigen specific cytotoxic T cells or antibodies are administered to the host. This approach is short lived and requires multiple applications.

Despite significant advances, the efficacy of current immunotherapy strategies is limited by associated toxicities. These are often related to the narrow therapeutic window associated with immunotherapy, which in part, emerges from the need to push therapy dose to the edge of potentially fatal toxicity to get a clinically meaningful treatment effect. Further, dose expands in vivo since adoptively transferred immune cells continue to proliferate within the patient, often unpredictably.

A major risk involved in immunotherapy is the on-target but off tumor side effects resulting from T-cell activation in response to normal tissue expression of the tumor associated antigen (TAA). Clinical trials utilizing T cells expressing T-cell receptor against specific TAA reported skin rash, colitis and hearing loss in response to immunotherapy.

Immunotherapy may also produce on target, on-tumor toxicities that emerge when tumor cells are killed in response to the immunotherapy. The adverse effects include tumor lysis syndrome, cytokine release syndrome and the related macrophage activation syndrome. Importantly, these adverse effects may occur during the destruction of tumors, and thus even a successful on-tumor immunotherapy might result in toxicity. Approaches to regulatable control immunotherapy are thus highly desirable since they have the potential to reduce toxicity and maximize efficacy.

The present invention provides systems, compositions, immunotherapeutic agents and methods for cancer immunotherapy. These compositions provide tunable regulation of gene expression and function in immunotherapy. The present invention also provides biocircuit systems, effector modules, stimulus response elements (SREs) and payloads, as well as polynucleotides encoding any of the foregoing. In one aspect, the systems, compositions, immunotherapeutic agents and other components of the invention can be controlled by a separately added stimulus, which provides a significant flexibility to regulate cancer immunotherapy. Further, the systems, compositions and the methods of the present invention may also be combined with therapeutic agents such as chemotherapeutic agents, small molecules, gene therapy, and antibodies.

The tunable nature of the systems and compositions of the invention has the potential to improve the potency and duration of the efficacy of immunotherapies. Reversibly silencing the biological activity of adoptively transferred cells using compositions of the present invention allows maximizing the potential of cell therapy without irretrievably killing and terminating the therapy.

The present invention provides methods for fine tuning of immunotherapy after administration to patients. This in turn improves the safety and efficacy of immunotherapy and increases the subject population that may benefit from immunotherapy.

### II. Compositions of the Invention

According to the present invention, biocircuit systems are provided which comprise, at their core, at least one effector module. Such effector module comprise at least one effector module having associated, or integral therewith, one or more stimulus response elements (SREs). In general, a stimulus response element (SRE) may be operably linked to a payload which could be any protein of interest (POI) (e.g., an immunotherapeutic agent), to form an effector module. The SRE, when activated by a particular stimulus, e.g., a small molecule, can produce a signal or outcome, to regulate transcription and/or protein levels of the linked payload either up or down by perpetuating a stabilizing signal or destabilizing signal, or any other types of regulation. In accordance with the present invention, biocircuit systems, effector modules, SREs and components that tune expression levels and activities of any agents used for immunotherapy are provided.

As used herein, a “biocircuit” or “biocircuit system” is defined as a circuit within or useful in biologic systems comprising a stimulus and at least one effector module responsive to a stimulus, where the response to the stimulus produces at least one signal or outcome within, between, as an indicator of, or on a biologic system. Biologic systems are generally understood to be any cell, tissue, organ, organ system or organism, whether animal, plant, fungi, bacterial, or viral. It is also understood that biocircuits may be artificial circuits which employ the stimuli or effector modules taught by the present invention and effect signals or outcomes in acellular environments such as with diagnostic, reporter systems, devices, assays or kits. The artificial circuits may be associated with one or more electronic, magnetic, or radioactive components or parts.

In accordance with the present invention, a biocircuit system may be a destabilizing domain (DD) biocircuit system, a dimerization biocircuit system, a receptor biocircuit system, and a cell biocircuit system. Any of these systems may act as a signal to any other of these biocircuit systems.

### Effector Modules and SREs for Immunotherapy

In accordance with the present invention, biocircuit systems, effector modules, SREs, and components that tune expression levels and activities of any agents used for immunotherapy are provided. As non-limiting examples, an immunotherapeutic agent may be an antibody and fragments and variants thereof, a cancer specific T cell receptor (TCR) and variants thereof, an anti-tumor specific chimeric antigen receptor (CAR), a chimeric switch receptor, an inhibitor of a co-inhibitory receptor or ligand, an agonist of a co-stimulatory receptor and ligand, a cytokine, chemokine, a cytokine receptor, a chemokine receptor, a soluble growth factor, a metabolic factor, a suicide gene, a homing receptor, or any agent that induces an immune response in a cell and a subject.

As stated, the biocircuits of the invention include at least one effector module as a component of an effector module system. As used herein, an “effector module” is a single or multi-component construct or complex comprising at least (a) one or more stimulus response elements and (b) at least one payload (e.g. proteins of interest (POIs)). As used herein a “stimulus response element (SRE)” is a component of an effector module which is joined, attached, linked to or associated with one or more payloads of the effector module and in some instances, is responsible for the responsive nature of the effector module to one or more stimuli. As used herein, the “responsive” nature of an SRE to a stimulus may be characterized by a covalent or non-covalent interaction, a direct or indirect association or a structural or chemical reaction to the stimulus. Further, the response of any SRE to a stimulus may be a matter of degree or kind. The response may be a partial response. The response may be a reversible response. The response may ultimately lead to a regulated signal or output. Such output signal may be of a relative nature to the stimulus, e.g., producing a modulatory effect of between 1% and 100% or a factored increase or decrease such as 2-fold, 3-fold, 4-fold, 5-fold, 10-fold or more.

In some embodiments, the present invention provides methods for modulating protein expression, function or level. In some aspects, the modulation of protein expression, function or level refers to modulation of expression, function or level by at least about 20%, such as by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In some embodiments, the present invention provides methods for modulating protein, expression, function or level by measuring the stabilization ratio and destabilization ratio. As used herein, the stabilization ratio may be defined as the ratio of expression, function or level of a protein of interest in response to the stimulus to the expression, function or level of the protein of interest in the absence of the stimulus specific to the SRE. In some aspects, the stabilization ratio is at least 1, such as by at least 1-10, 1-20, 1-30, 1-40, 1-50, 1-60, 1-70, 1-80, 1-90, 1-100, 20-30, 20-40, 20-50, 20-60, 20-70, 20-80, 20-90, 20-95, 20-100, 30-40, 30-50, 30-60, 30-70, 30-80, 30-90, 30-95, 30-100, 40-50, 40-60, 40-70, 40-80, 40-90, 40-95, 40-100, 50-60, 50-70, 50-80, 50-90, 50-95, 50-100, 60-70, 60-80, 60-90, 60-95, 60-100, 70-80, 70-90, 70-95, 70-100, 80-90, 80-95, 80-100, 90-95, 90-100 or 95-100. As used herein, the destabilization ratio may be defined as the ratio of expression, function or level of a protein of interest in the absence of the stimulus specific to the effector module to the expression, function or level of the protein of interest, that is expressed constitutively and in the absence of the stimulus specific to the SRE. As used herein “constitutively” refers to the expression, function or level of a protein of interest that is not linked to an SRE and is therefore expressed both in the presence and absence of the stimulus. In some aspects, the destabilization ratio is at least 0, such as by at least 0.1, 0.2, 0.3, 0.4, 0.5, 0.6, 0.7, 0.8, 0.9, or at least, 0-0.1, 0-0.2, 0-0.3, 0-0.4, 0-0.5, 0-0.6, 0-0.7, 0-0.8, 0-0.9, 0.1-0.2, 0.1-0.3, 0.1-0.4, 0.1-0.5, 0.1-0.6, 0.1-0.7, 0.1-0.8, 0.1-0.9, 0.2-0.3, 0.2-0.4, 0.2-0.5, 0.2-0.6, 0.2-0.7, 0.2-0.8, 0.2-0.9, 0.3-0.4, 0.3-0.5, 0.3-0.6, 0.3-0.7, 0.3-0.8, 0.3-0.9, 0.4-0.5, 0.4-0.6, 0.4-0.7, 0.4-0.8, 0.4-0.9, 0.5-0.6, 0.5-0.7, 0.5-0.8, 0.5-0.9, 0.6-0.7, 0.6-0.8, 0.6-0.9, 0.7-0.8, 0.7-0.9 or 0.8-0.9.

In some embodiments, the first SRE of the effector module may stabilize the immunotherapeutic agent by a stabilization ratio of 1 or more, wherein the stabilization ratio may comprise the ratio of expression, function or level of the immunotherapeutic agent in the presence of the stimulus to the expression, function or level of the immunotherapeutic agent in the absence of the stimulus.

In some embodiments, the SRE may destabilize the immunotherapeutic agent by a destabilization ratio between 0, and 0.09, wherein the destabilization ratio may comprise the ratio of expression, function or level of the immunotherapeutic agent in the absence of the stimulus specific to the SRE to the expression, function or level of the immunotherapeutic agent that is expressed constitutively, and in the absence of the stimulus specific to the SRE.

The present invention also provides polynucleotides comprising the compositions of the invention.

The SRE of the effector module may be selected from, but is not limited to, a peptide, peptide complex, peptide-protein complex, protein, fusion protein, protein complex, protein-protein complex. The SRE may comprise one or more regions derived from any natural or mutated protein, or antibody. In this aspect, the SRE is an element, when responding to a stimulus, can tune intracellular localization, intramolecular activation, and/or degradation of payloads.

In some embodiments, effector modules of the present invention may comprise additional features that facilitate the expression and regulation of the effector module, such as one or more signal sequences (SSs), one or more cleavage and/or processing sites, one or more targeting and/or penetrating peptides, one or more tags, and/or one or more linkers. Additionally, effector modules of the present invention may further comprise other regulatory moieties such as inducible promoters, enhancer sequences, microRNA sites, and/or microRNA targeting sites. Each aspect or tuned modality may bring to the effector module or biocircuit a differentially tuned feature. For example, an SRE may represent a destabilizing domain, while mutations in the protein payload may alter its cleavage sites or dimerization properties or half-life and the inclusion of one or more microRNA or microRNA binding site may impart cellular detargeting or trafficking features. Consequently, the present invention embraces biocircuits which are multifactorial in their tenability. Such biocircuits may be engineered to contain one, two, three, four or more tuned features.

In some embodiments, effector modules of the present invention may include one or more degrons to tune expression. As used herein, a “degron” refers to a minimal sequence within a protein that is sufficient for the recognition and the degradation by the proteolytic system. An important property of degrons is that they are transferrable, that is, appending a degron to a sequence confers degradation upon the sequence. In some embodiments, the degron may be appended to the destabilizing domains, the payload or both. Incorporation of the degron within the effector module of the invention, confers additional protein instability to the effector module and may be used to minimize basal expression. In some embodiments, the degron may be an N degron, a phospho degron, a heat inducible degron, a photosensitive degron, an oxygen dependent degron. As a non-limiting example, the degron may be an Ornithine decarboxylase degron as described by Takeuchi et al. (Takeuchi J et al. (2008). Biochem J. 2008 Mar. 1; 410(2):401-7; the contents of which are incorporated by reference in their entirety).

In some aspects, the two or more immunotherapeutic agents may be the same type such as two antibodies, or different types such as a CAR construct and a cytokine IL12. Biocircuits and components utilizing such effector molecules are given in FIGS. 7-12 in International Publication No. WO2017/180587, the contents of which are herein incorporated by reference in their entirety.

In some embodiments, the composition for inducing an immune response may comprise an effector module. In some embodiments, the effector module may comprise a stimulus response element (SRE) operably linked to at least one payload. In one aspect, the payload may be an immunotherapeutic agent.

In some embodiments, the effector modules of the present invention may include molecular switches. As used herein, the term “molecular switches” refers to any molecule that may be reversibly shifted between two or more stable states in response to a stimulus (e.g., a ligand).

In some embodiments, molecular switches may be RNA-based switches. As a non-limiting example, the effector module may be provided in an RNA molecule that comprises a coding sequence for a destabilization domain (e.g., FKBP12, ecDHFR) fused to a translational repressor L7Ae, a kink-turn (K-turn) motif in the 5′UTR of a payload mRNA, and the coding sequence for the payload (e.g., peptide or protein). L7Ae is an archaeal protein that binds K-turn and K-loop motifs with high affinity. In the absence of a ligand that stabilizes the destabilization domain, fusion L7Ae protein is degraded and the payload peptide or protein is expressed (ON state), whereas in the presence of the ligand, the fusion L7Ae protein binds to the K-turn motif and represses expression of the payload peptide or protein (OFF state). Other translational regulation systems may also be used, such as, but not limited to, MS2-tethered repressors, Tet repressors, and microRNAs.

In some embodiments, molecular switches may be switched on/off via dimerization. For example, a first effector module may comprise an SRE which is a first member of a dimerization pair and optionally a first payload, and a second effector module may comprise an SRE which is second member of a dimerization pair and optionally a second payload, wherein the two members of the dimerization pair dimerizes upon addition of a dimerization ligand. In some embodiments, dimerization may restore stability of the members of the dimerization pair and/or the attached payloads. Dimerization induces interaction, whether direct or indirect, of the two payloads to create a desired effect. In some embodiments, dimerization may induce degradation of the dimerization pair and/or the attached payloads. For example, bivalent small-molecule can dimerize two molecules of an E3 ubiquitin ligase to induce self-degradation (Maniaci et al., Nat Commun. 2017 Oct. 10; 8(1):830). Dimerization pair may comprise, or be derived from, for example, an antibody and its antigen, two fragments of an antibody, a ligand-binding domain and a cognate receptor (e.g., any of those described in International Patent Publication NO: WO2017120546, the contents of each of which are incorporated herein by reference in their entirety), and an E3 ubiquitin ligase and a substrate (e.g., as described in Maniaci et al.).

In some embodiments, molecular switches may be conditionally active in specific cell types or under specific cellular conditions. For example, the stimulus required to stimulate the SREs of the effector modules may only be present in a particular cell type or under a particular cellular condition. This allows various applications, such as cell-type specific delivery of a payload, or detection of a particular cellular target. Based on this property, biocircuits or effector modules of the present invention may be developed as biosensors. For example, the SRE may be fused to a reporter protein (e.g., GFP). The SRE contains mutations such that the SRE is conditionally stable only in the presence of a stabilizing ligand which is also the target to be detected. When the target is present in the cell, the fusion protein is stabilized and the reporter activity can be detected.

In some embodiments, the immunotherapeutic agent may be selected from, but is not limited to a chimeric antigen receptor (CAR) and an antibody.

In some embodiments, biocircuits of the invention may be modified to reduce their immunogenicity. Immunogenicity is the result of a complex series of responses to a substance that is perceived as foreign and may include the production of neutralizing and non-neutralizing antibodies, formation of immune complexes, complement activation, mast cell activation, inflammation, hypersensitivity responses, and anaphylaxis. Several factors can contribute to protein immunogenicity, including, but not limited to protein sequence, route and frequency of administration and patient population. In a preferred embodiment, protein engineering may be used to reduce the immunogenicity of the compositions of the invention. In some embodiments, modifications to reduce immunogenicity may include modifications that reduce binding of the processed peptides derived from the parent sequence to MHC proteins. For example, amino acid modifications may be engineered such that there are no or a minimal of number of immune epitopes that are predicted to bind with high affinity, to any prevalent MHC alleles. Several methods of identifying MHC binding epitopes of known protein sequences are known in the art and may be used to score epitopes in the compositions of the present invention.

Epitope identification and subsequent sequence modification may be applied to reduce immunogenicity. The identification of immunogenic epitopes may be achieved either physically or computationally. Physical methods of epitope identification may include, for example, mass spectrometry and tissue culture/cellular techniques. Computational approaches that utilize information obtained on antigen processing, loading and display, structural and/or proteomic data toward identifying non-self-peptides that may result from antigen processing, and that are likely to have good binding characteristics in the groove of the MHC may also be utilized. One or more mutations may be introduced into the biocircuits of the invention directing the expression of the protein, to maintain its functionality while simultaneously rendering the identified epitope less or non-immunogenic.

In some embodiments, protein modifications engineered into the structure of the compositions of the invention to interfere with antigen processing and peptide loading such as glycosylation and PEGylation, may also be useful in the present invention. Compositions of the invention may also be engineered to include non-classical amino acid sidechains to design less immunogenic compositions.

In one embodiment, patients may also be stratified according to the immunogenic peptides presented by their immune cells and may be utilized as a parameter to determine suitable patient cohorts that may therapeutically benefit for the compositions of the invention.

In some embodiments, reduced immunogenicity may be achieved by limiting immunoproteasome processing. The proteasome is an important cellular protease that is found in two forms: the constitutive proteasome, which is expressed in all cell types and which contains active e.g. catalytic subunits and the immunoproteasome that is expressed in cell of the hematopoietic lineage, and which contains different active subunits termed low molecular weight proteins (LMP) namely LMP-2, LMP-7 and LMP-10. Immunoproteasomes exhibit altered peptidase activities and cleavage site preferences that result in more efficient liberation of many MHC class I epitopes. A well described function of the immunoproteasome is to generate peptides with hydrophobic C terminus that can be processed to fit in the groove of MHC class I molecules. Deol P et al. have shown that immunoproteasomes may lead to a frequent cleavage of specific peptide bonds and thereby to a faster appearance of a certain peptide on the surface of the antigen presenting cells; and enhanced peptide quantities (Deol P et al. (2007) J Immunol 178 (12) 7557-7562; the contents of which are incorporated herein reference in its entirety). This study indicates that reduced immunoproteasome processing may be accompanied by reduced immunogenicity. In some embodiments, immunogenicity of the compositions of the invention may be reduced by modifying the sequence encoding the compositions of the invention to prevent immunoproteasome processing. Biocircuits of the present invention may also be combined with immunoproteasome-selective inhibitors to achieve the same effects. Examples of inhibitors useful in the present invention include UK-101 (B1i selective compound), IPSI-001, ONX 0914 (PR-957), and PR-924 (IPSI).

### 1. Destabilizing Domains (DDs)

In some embodiments, biocircuit systems, effector modules, and compositions of the present invention relate to post-translational regulation of protein (payload) function anti-tumor immune responses of immunotherapeutic agents. In one embodiment, the SRE is a stabilizing/destabilizing domain (DD). The presence, absence or an amount of a small molecule ligand that binds to or interacts with the DD, can, upon such binding or interaction modulate the stability of the payload(s) and consequently the function of the payload. Depending on the degree of binding and/or interaction the altered function of the payload may vary, hence providing a “tuning” of the payload function. In one embodiment, the destabilizing domain may be referred to as the degradation domain.

In some embodiments, destabilizing domains described herein or known in the art may be used as SREs in the biocircuit systems of the present invention in association with any of the immunotherapeutic agents (payloads) taught herein. Destabilizing domains (DDs) are small protein domains that can be appended to a target protein of interest. DDs render the attached protein of interest unstable in the absence of a DD-binding ligand such that the protein is rapidly degraded by the ubiquitin-proteasome system of the cell (Stankunas, K., et al., Mol. Cell, 2003, 12: 1615-1624; Banaszynski, et al., Cell; 2006, 126(5): 995-1004; reviewed in Banaszynski, L. A., and Wandless, T. J. Chem. Biol.; 2006, 13:11-21 and Rakhit R et al., Chem Biol. 2014; 21(9):1238-1252). However, when a specific small molecule ligand binds its intended DD as a ligand binding partner, the instability is reversed, and protein function is restored. The conditional nature of DD stability allows a rapid and non-perturbing switch from stable protein to unstable substrate for degradation. Moreover, its dependency on the concentration of its ligand further provides tunable control of degradation rates.

In some embodiments, the desired characteristics of the DDs may include, but are not limited to, low protein levels in the absence of a ligand of the DD (i.e. low basal stability), large dynamic range, robust and predictable dose-response behavior, and rapid kinetics of degradation. DDs that bind to a desired ligand, but not endogenous molecules may be preferred.

Several protein domains with destabilizing properties and their paired small molecules have been identified and used to control protein expression, including FKBP/shield-1 system (Egeler et al., J Biol. Chem. 2011, 286(36): 32328-31336; the contents of which are incorporated herein by reference in their entirety), and ecDHFR and its ligand trimethoprim (TMP).

In some embodiments, the DDs of the present invention may be derived from some known sequences that have been approved to be capable of post-translational regulation of proteins.

In some embodiments, the DDs of the present invention may be developed from known proteins. Regions or portions or domains of wild type proteins may be utilized as SREs/DDs in whole or in part. They may be combined or rearranged to create new peptides, proteins, regions or domains of which any may be used as SREs/DDs or the starting point for the design of further SREs and/or DDs.

Ligands such as small molecules that are well known to bind candidate proteins can be tested for their regulation in protein responses. The small molecules may be clinically approved to be safe and have appropriate pharmaceutical kinetics and distribution. In some embodiments, the stimulus is a ligand of a destabilizing domain (DD), for example, a small molecule that binds a destabilizing domain and stabilizes the POI fused to the destabilizing domain.

In some embodiments, the SRE may comprise a destabilizing domain (DD). The DD may be derived from a parent protein or from a mutant protein having one, two, there, or more amino acid mutations compared to the parent protein. In some embodiments, the parent protein may be selected from, but is not limited to, human protein FKBP comprising the amino acid sequence of SEQ ID NO. 3 or 1087; human DHFR (hDHFR) comprising the amino acid sequence of SEQ ID NO. 2; or E. Coli DHFR (ecDHFR) comprising the amino acid sequence of SEQ ID NO. 1.

Some examples of the proteins that may be used to develop DDs and their ligands are listed in Table 1.

TABLE 1
Proteins and their binding ligands

| | | Protein | |
|---|---|---|---|
| | Protein | SEQ ID | |
| Protein | Sequence | NO. | Ligands |
| E. coli | MISLIAALAV | 1 | Methotrexate |
| Dihydrofolate | DRVIGMENAM | | (MTX) |
| reductase | PWNLPADLAW | | Trimethoprim |
| (ecDHFR) | FKRNTLNKPV | | (TMP) |
| (Uniprot | IMGRHTWESI | | |
| ID: | GRPLPGRKNI | | |
| P0ABQ4) | ILSSQPGTDD | | |
| | RVTWVKSVDE | | |
| | AIAACGDVPE | | |
| | IMVIGGGRVY | | |
| | EQFLPKAQKL | | |
| | YLTHIDAEVE | | |
| | GDTHFPDYEP | | |
| | DDWESVFSEF | | |
| | HDADAQNSHS | | |
| | YCFEILERR | | |
| Human | MVGSLNCIVA | 2 | Methotrexate |
| Dihydrofolate | VSQNMGIGKN | | (MTX) |
| reductase | GDLPWPPLRN | | Trimethoprim |
| (hDHFR) | EFRYFQRMTT | | (TMP) |
| (Uniprot | TSSVEGKQNL | | |
| ID: | VIMGKKTWFS | | |
| P00374) | IPEKNRPLKG | | |
| | RINLVLSREL | | |
| | KEPPQGAHFL | | |
| | SRSLDDALKL | | |
| | TEQPELANKV | | |
| | DMVWIVGGSS | | |
| | VYKEAMNHPG | | |
| | HLKLFVTRIM | | |
| | QDFESDTFFP | | |
| | EIDLEKYKLL | | |
| | PEYPGVLSDV | | |
| | QEEKGIKYKF | | |
| | EVYEKND | | |
| FK506 | MGVQVETISP | 1087 | Shield-1 |
| binding | GDGRTFPKRG | | |
| protein | QTCVVHYTGM | | |
| (FKBP) | LEDGKKFDSS | | |
| (Uniprot | RDRNKPFKFM | | |
| ID: | LGKQEVIRGW | | |
| P62942) | EEGVAQMSVG | | |
| | QRAKLTISPD | | |
| | YAYGATGHPG | | |
| | IIPPHATLVF | | |
| | DVELLKLE | | |
| FK506 | GVQVETISPG | 3 | Shield-1 |
| binding | DGRTFPKRGQ | | |
| protein | TCVVHYTGML | | |
| (FKBP) | EDGKKFDSSR | | |
| (Uniprot | DRNKPFKFML | | |
| ID: | GKQEVIRGWE | | |
| P62942; | EGVAQMSVGQ | | |
| M1del) | RAKLTISPDY | | |
| | AYGATGHPGI | | |
| | IPPHATLVFD | | |
| | VELLKLE | | |

In some embodiments, DDs of the invention may be derived from human dihydrofolate reductase (hDHFR). hDHFR is a small (18 kDa) enzyme that catalyzes the reduction of dihydrofolate and plays a vital role in variety of anabolic pathway. Dihydrofolate reductase (DHFR) is an essential enzyme that converts 7,8-dihydrofolate (DHF) to 5,6,7,8, tetrahydrofolate (THF) in the presence of nicotinamide adenine dihydrogen phosphate (NADPH). Anti-folate drugs such as methotrexate (MTX), a structural analogue of folic acid, which bind to DHFR more strongly than the natural substrate DHF, interferes with folate metabolism, mainly by inhibition of dihydrofolate reductase, resulting in the suppression of purine and pyrimidine precursor synthesis. Other inhibitors of hDHFR such as folate, TQD, Trimethoprim (TMP), epigallocatechin gallate (EGCG) and ECG (epicatechin gallate) can also bind to hDHFR mutants and regulates its stability. In one aspect of the invention, the DDs of the invention may be hDHFR mutants including the single mutation hDHFR (Y122I), hDHFR (K81R), hDHFR (F59S), hDHFR (I17V), hDHFR (N65D), hDHFR (A107V), hDHFR (N127Y), hDHFR (K185E), hDHFR (N186D), and hDHFR (M140I); double mutations: hDHFR (M53T, R138I), hDHFR (V75F, Y122I), hDHFR (Y122I, A125F), hDHFR (L74N, Y122I), hDHFR (L94A, T147A), hDHFR (G21T, Y122I), hDHFR (V121A, Y122I), hDHFR (Q36K, Y122I), hDHFR (C7R, Y163C), hDHFR (Y178H, E18IG), hDHFR (A10V, H88Y), hDHFR (T137R, F143L), hDHFR (E63G, I176F), hDHFR (T57A, I72A), hDHFR (H131R, E144G), and hDHFR (Y183H, K185E); and triple mutations: hDHFR (Q36F, N65F, Y122I), hDHFR (G21E, I72V, I176T), hDHFR (I8V, K133E, Y163C), hDHFR (V9A, S93R, P150L), hDHFR (K19E, F89L, E181G), hDHFR (G54R, M140V, S168C), hDHFR (L23S, V121A, Y157C), hDHFR (V110A, V136M, K177R), and hDHFR (N49D, F59S, D153G).

In one aspect, the parent protein is hDHFR and the DD comprises a mutant protein. The mutant protein may comprise a single mutation and may be selected from, but not limited to hDHFR (I17V), hDHFR (F59S), hDHFR (N65D), hDHFR (K81R), hDHFR (A107V), hDHFR (Y122I), hDHFR (N127Y), hDHFR (M1400, hDHFR (K185E), hDHFR (N186D), and hDHFR (M140I), hDHFR (M1del, N127Y), hDHFR (M1del, I17V), hDHFR (M1del, Y122I), and hDHFR (M1del, K185E). In some embodiments, the mutant protein may comprise two mutations and may be selected from, but not limited to, hDHFR (C7R, Y163C), hDHFR (A10V, H88Y), hDHFR (Q36K, Y122I), hDHFR (M53T, R138I), hDHFR (T57A, I72A), hDHFR (E63G, I176F), hDHFR (G21T, Y122I), hDHFR (L74N, Y122I), hDHFR (V75F, Y122I), hDHFR (L94A, T147A), DHFR (V121A, Y22I), hDHFR (Y122I, A125F), hDHFR (H131R, E144G), hDHFR (T137R, F143L), hDHFR (Y178H, E18IG), and hDHFR (Y183H, K185E), hDHFR (E162G, I176F) hDHFR (M1del, I17V, Y122I), hDHFR (M1del, Y122I, M1400, hDHFR (M1del, N127Y, Y122I), hDHFR (M1del, E162G, I176F), and hDHFR (M1del, H131R, E144G), and hDHFR (M1del, Y122I, A125F). In some embodiments, the mutant may comprise three mutations and the mutant may be selected from hDHFR (V9A, S93R, P150L), hDHFR (I8V, K133E, Y163C), hDHFR (L23S, V121A, Y157C), hDHFR (K19E, F89L, E181G), hDHFR (Q36F, N65F, Y122I), hDHFR (G54R, M140V, S168C), hDHFR (V110A, V136M, K177R), hDHFR (Q36F, Y122I, A125F), hDHFR (N49D, F59S, D153G), and hDHFR (G21E, I72V, I176T), hDHFR (M1del, Q36F, Y122I, A125F), hDHFR (M1del, Y122I, H131R, E144G), hDHFR (M1del, E31D, F32M, V116I), and hDHFR (M1del, Q36F, N65F, Y122I). In some embodiments, the mutant may comprise four or more mutations and the mutant may be selected from hDHFR (V2A, R33G, Q36R, L100P, K185R), hDHFR (M1del, D22S, F32M, R33S, Q36S, N65S), hDHFR (I17N, L98S, K99R, M112T, E151G, E162G, E172G), hDHFR (G16S, I17V, F89L, D96G, K123E, M140V, D146G, K156R), hDHFR (K81R, K99R, L100P, E102G, N108D, K123R, H128R, D142G, F180L, K185E), hDHFR (R138G, D142G, F143S, K156R, K158E, E162G, V166A, K177E, Y178C, K185E, N186S), hDHFR (N14S, P24S, F35L, M53T, K56E, R92G, S93G, N127S, H128Y, F135L, F143S, L159P, L160P, E173A, F180L), hDHFR (F35L, R37G, N65A, L68S, K69E, R71G, L80P, K99G, G117D, L132P, I139V, M140I, D142G, D146G, E173G, D187G), hDHFR (L28P, N30H, M38V, V44A, L68S, N73G, R78G, A97T, K99R, A107T, K109R, D111N, L134P, F135V, T147A, I152V, K158R, E172G, V182A, E184R), hDHFR (V2A, I17V, N30D, E31G, Q36R, F59S, K69E, I72T, H88Y, F89L, N108D, K109E, V110A, 1115V, Y122D, L132P, F135S, M140V, E144G, T147A, Y157C, V170A, K174R, N186S), hDHFR (L100P, E102G, Q103R, P104S, E105G, N108D, V113A, W114R, Y122C, M126I, N127R, H128Y, L132P, F135P, I139T, F148S, F149L, I152V, D153A, D169G, V170A, I176A, K177R, V182A, K185R, N186S), and hDHFR (A10T, Q13R, N14S, N20D, P24S, N30S, M38T, T40A, K47R, N49S, K56R, I61T, K64R, K69R, I72A, R78G, E82G, F89L, D96G, N108D, M112V, W114R, Y122D, K123E, I139V, Q141R, D142G, F148L, E151G, E155G, Y157R, Q171R, Y183C, E184G, K185del, D187N).

In one embodiment, the stimulus is a small molecule that binds to a SRE to post-translationally regulate protein levels. In one aspect, DHFR ligands: trimethoprim (TMP) and methotrexate (MTX) are used to stabilize hDHFR mutants.

In some embodiments, DD mutations that do not inhibit ligand binding may be preferentially selected. In some embodiments, ligand binding may be improved by mutation of residues in DHFR.

In some embodiments, the SREs of the present invention may be derived from oxidoreductases, transferases, polymerases, hydrolases, lyases, isomerases, ligases, direct ligand-gated ion channel receptors, G-protein-coupled receptors, Cytokine receptors, lntegrin receptors, Receptors associated with a tyrosine kinase, Nuclear receptors (steroid hormone receptors), Voltage-gated Calcium channels, Na+ channels Ryanodine-inositol 1,4,5-triphosphate receptor Calcium channel (RIR—CaC) family, Transient receptor potential CaL+ channel (TRP—CC) family, and nucleic acids.

### 2. Stimuli

Biocircuits of the invention are triggered by one or more stimuli. Stimuli may be selected from a ligand, an externally added or endogenous metabolite, the presence or absence of a defined ligand, pH, temperature, light, ionic strength, radioactivity, cellular location, subject site, microenvironment, the presence or the concentration of one or more metal ions.

In some embodiments, the stimulus is a ligand. Ligands may be nucleic acid-based, protein-based, lipid based, organic, inorganic or any combination of the foregoing. In some embodiments, the ligand is selected from the group consisting of a protein, peptide, nucleic acid, lipid, lipid derivative, sterol, steroid, metabolite derivative and a small molecule. In some embodiments, the stimulus is a small molecule. In some embodiments, the small molecules are cell permeable.

In some embodiments, any of the ligands in Table 1 may be useful in the present invention.

In some aspects, the ligand binds to FKBP. The ligand may be rapamycin, shield-1, Aquashield, and SLF.

In some embodiments, the ligand binds to dihydrofolate reductase. In some embodiments, the ligand binds to and inhibits dihydrofolate reductase function and is herein referred to as a dihydrofolate inhibitor.

In some embodiments, the ligand may be a selective inhibitor of human DHFR. Ligands of the invention may also be selective inhibitors of dihydrofolate reductases of bacteria and parasitic organisms such as Pneumocystis spp., Toxoplasma spp., Trypanosoma spp., Mycobacterium spp., and Streptococcus spp. Ligands specific to other DHFR may be modified to improve binding to human dihydrofolate reductase.

Examples of dihydrofolate inhibitors include, but are not limited to, Trimethoprim (TMP), Methotrexate (MTX), Pralatrexate, Piritrexim Pyrimethamine, Talotrexin, Chloroguanide, Pentamidine, Trimetrexate, aminopterin, C1 898 trihydrochloride, Pemetrexed Disodium, Raltitrexed, Sulfaguanidine, Folotyn, Iclaprim and Diaveridine.

In some embodiments, ligands include TMP-derived ligands containing portions of the ligand known to mediate binding to DHFR. Ligands may also be modified to reduce off-target binding to other folate metabolism enzymes and increase specific binding to DHFR.

### 3. Payloads: Immunotherapeutic Agents

In some embodiments, payloads of the present invention may be immunotherapeutic agents that induce immune responses in an organism. The immunotherapeutic agent may be, but is not limited to, an antibody and fragments and variants thereof, a chimeric antigen receptor (CAR), a chimeric switch receptor, a cytokine, chemokine, a cytokine receptor, a chemokine receptor, a cytokine-cytokine receptor fusion polypeptide, or any agent that induces an immune response. In one embodiment, the immunotherapeutic agent induces an anti-cancer immune response in a cell, or in a subject.

The biocircuits of the present invention may be monocistronic or multicistronic meaning one (monocistronic) or more than one (multicistronic) message (e.g. payload of interest) is produced. If two messages are produced, the biocircuit or effector module is considered bicistronic.

### Antibodies

In some embodiments, antibodies, fragments and variants thereof are payloads of the present invention.

In some embodiments, stability of the antibodies, or antibody fragments described herein can be evaluated for their stability. e.g. thermal stability. In some aspects, the antibodies described herein may be engineered to have increased thermal stability than a control binding molecule (e.g. a conventional scFv). Enhanced thermal stability has been associated with improved therapeutic properties of the antibody, antibody fragments, and/or the chimeric antigen receptors that contain the antibody fragments. Stability of the antibodies may be altered by mutagenesis of select amino acids within the antibody and stability i.e. thermal stability may be measured by method known in the art. Binding affinity and aggregation properties of the antibodies may be evaluated after engineering mutations, to ensure that these properties are not altered.

### Antibody Fragments and Variants

In some embodiments, antibody fragments and variants may comprise antigen binding regions from intact antibodies. Examples of antibody fragments and variants may include, but are not limited to Fab, Fab′, F(ab′)2, and Fv fragments; diabodies; linear antibodies; single-chain antibody molecules such as single chain variable fragment (scFv); and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)2 fragment that has two antigen-binding sites and is still capable of cross-linking with the antigen. Pharmaceutical compositions, biocircuits, biocircuit components, effector modules including their SREs or payloads of the present invention may comprise one or more of these fragments.

For the purposes herein, an “antibody” may comprise a heavy and light variable domain as well as an Fc region. As used herein, the term “native antibody” usually refers to a heterotetrameric glycoprotein of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain.

As used herein, the term “variable domain” refers to specific antibody domains found on both the antibody heavy and light chains that differ extensively in sequence among antibodies and are used in the binding and specificity of each particular antibody for its particular antigen. Variable domains comprise hypervariable regions. As used herein, the term “hypervariable region” refers to a region within a variable domain comprising amino acid residues responsible for antigen binding. The amino acids present within the hypervariable regions determine the structure of the complementarity determining regions (CDRs) that become part of the antigen-binding site of the antibody. As used herein, the term “CDR” refers to a region of an antibody comprising a structure that is complimentary to its target antigen or epitope. Other portions of the variable domain, not interacting with the antigen, are referred to as framework (FW) regions. The antigen-binding site (also known as the antigen combining site or paratope) comprises the amino acid residues necessary to interact with a particular antigen. The exact residues making up the antigen-binding site are typically elucidated by co-crystallography with bound antigen, however computational assessments based on comparisons with other antibodies can also be used (Strohl, W. R. Therapeutic Antibody Engineering. Woodhead Publishing, Philadelphia Pa. 2012. Ch. 3, p 47-54, the contents of which are herein incorporated by reference in their entirety). Determining residues that make up CDRs may include the use of numbering schemes including, but not limited to, those taught by Kabat (Wu et al., JFM, 1970, 132(2):211-250 and Johnson et al., Nucleic Acids Res. 2000, 28(1): 214-218, the contents of each of which are herein incorporated by reference in their entirety), Chothia (Chothia and Lesk, J. Mol. Biol. 1987, 196, 901, Chothia et al., Nature, 1989, 342, 877, and Al-Lazikani et al., J. Mol. Biol. 1997, 273(4): 927-948, the contents of each of which are herein incorporated by reference in their entirety), Lefranc (Lefranc et al., Immunome Res. 2005, 1:3) and Honegger (Honegger and Pluckthun, J. Mol. Biol. 2001, 309(3): 657-70, the contents of which are herein incorporated by reference in their entirety).

VH and VL domains have three CDRs each. VL CDRs are referred to herein as CDR-L1, CDR-L2 and CDR-L3, in order of occurrence when moving from N to C terminus along the variable domain polypeptide. VH CDRs are referred to herein as CDR-H1, CDR-H2 and CDR-H3, in order of occurrence when moving from N to C terminus along the variable domain polypeptide. Each of CDRs has favored canonical structures with the exception of the CDR-H3, which comprises amino acid sequences that may be highly variable in sequence and length between antibodies resulting in a variety of three-dimensional structures in antigen-binding domains (Nikoloudis, et al., PeerJ. 2014, 2: e456). In some cases, CDR-H3s may be analyzed among a panel of related antibodies to assess antibody diversity. Various methods of determining CDR sequences are known in the art and may be applied to known antibody sequences (Strohl, W. R. Therapeutic Antibody Engineering. Woodhead Publishing, Philadelphia Pa. 2012. Ch. 3, p 47-54, the contents of which are herein incorporated by reference in their entirety).

As used herein, the term “Fv” refers to an antibody fragment comprising the minimum fragment on an antibody needed to form a complete antigen-binding site. These regions consist of a dimer of one heavy chain and one light chain variable domain in tight, non-covalent association. Fv fragments can be generated by proteolytic cleavage but are largely unstable. Recombinant methods are known in the art for generating stable Fv fragments, typically through insertion of a flexible linker between the light chain variable domain and the heavy chain variable domain (to form a single chain Fv (scFv)) or through the introduction of a disulfide bridge between heavy and light chain variable domains (Strohl, W. R. Therapeutic Antibody Engineering. Woodhead Publishing, Philadelphia Pa. 2012. Ch. 3, p 46-4′7, the contents of which are herein incorporated by reference in their entirety).

As used herein, the term “light chain” refers to a component of an antibody from any vertebrate species assigned to one of two clearly distinct types, called kappa and lambda based on amino acid sequences of constant domains. Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2.

As used herein, the term “single chain Fv” or “scFv” refers to a fusion protein of VH and VL antibody domains, wherein these domains are linked together into a single polypeptide chain by a flexible peptide linker. In some embodiments, the Fv polypeptide linker enables the scFv to form the desired structure for antigen binding. In some embodiments, scFvs are utilized in conjunction with phage display, yeast display or other display methods where they may be expressed in association with a surface member (e.g. phage coat protein) and used in the identification of high affinity peptides for a given antigen.

Using molecular genetics, two scFvs can be engineered in tandem into a single polypeptide, separated by a linker domain, called a “tandem scFv” (tascFv). Construction of a tascFv with genes for two different scFvs yields a “bispecific single-chain variable fragments” (bis-scFvs). Only two tascFvs have been developed clinically by commercial firms; both are bispecific agents in active early phase development by Micromet for oncologic indications and are described as “Bispecific T-cell Engagers (BiTE).” Blinatumomab is an anti-CD19/anti-CD3 bispecific tascFv that potentiates T-cell responses to B-cell non-Hodgkin lymphoma in Phase 2. MT110 is an anti-EP-CAM/anti-CD3 bispecific tascFv that potentiates T-cell responses to solid tumors in Phase 1. Bispecific, tetravalent “TandAbs” are also being researched by Affimed (Nelson, A. L., MAbs., 2010, January-February; 2(1):77-83). maxibodies (bivalent scFv fused to the amino terminus of the Fc (CH2-CH3 domains) of IgG may also be included.

The term “intrabody” refers to a form of antibody that is not secreted from a cell in which it is produced, but instead targets one or more intracellular proteins. Intrabodies may be used to affect a multitude of cellular processes including, but not limited to intracellular trafficking, transcription, translation, metabolic processes, proliferative signaling and cell division. In some embodiments, methods of the present invention may include intrabody-based therapies. In some such embodiments, variable domain sequences and/or CDR sequences disclosed herein may be incorporated into one or more constructs for intrabody-based therapy.

As used herein, the term “monoclonal antibody” refers to an antibody obtained from a population of substantially homogeneous cells (or clones), i.e., the individual antibodies comprising the population are identical and/or bind the same epitope, except for possible variants that may arise during production of the monoclonal antibodies, such variants generally being present in minor amounts. In contrast to polyclonal antibody preparations that typically include different antibodies directed against different determinants (epitopes), each monoclonal antibody is directed against a single determinant on the antigen.

The modifier “monoclonal” indicates the character of the antibody as being obtained from a substantially homogeneous population of antibodies and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

As used herein, the term “humanized antibody” refers to a chimeric antibody comprising a minimal portion from one or more non-human (e.g., murine) antibody source(s) with the remainder derived from one or more human immunoglobulin sources. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and/or capacity. In one embodiment, the antibody may be a humanized full-length antibody.

As used herein, the term “antibody variant” refers to a modified antibody (in relation to a native or starting antibody) or a biomolecule resembling a native or starting antibody in structure and/or function (e.g., an antibody mimetic). Antibody variants may be altered in their amino acid sequence, composition or structure as compared to a native antibody. Antibody variants may include, but are not limited to, antibodies with altered isotypes (e.g., IgA, IgD, IgE, IgG1, IgG2, IgG3, IgG4, or IgM), humanized variants, optimized variants, multispecific antibody variants (e.g., bispecific variants), and antibody fragments.

In one embodiment, the antibody may comprise a modified Fc region. As a non-limiting example, the modified Fc region may be made by the methods or may be any of the regions described in US Patent Publication NO. US20150065690, the contents of which are herein incorporated by reference in its entirety.

In some embodiments, payloads may encode antibodies comprising a single antigen-binding domain. These molecules are extremely small, with molecular weights approximately one-tenth of those observed for full-sized mAbs.

In some embodiments, payloads of the invention may encode intrabodies. Intrabodies are a form of antibody that is not secreted from a cell in which it is produced, but instead targets one or more intracellular proteins. Intrabodies are expressed and function intracellularly and may be used to affect a multitude of cellular processes including, but not limited to intracellular trafficking, transcription, translation, metabolic processes, proliferative signaling and cell division. In some embodiments, methods described herein include intrabody-based therapies. In some such embodiments, variable domain sequences and/or CDR sequences disclosed herein are incorporated into one or more constructs for intrabody-based therapy. For example, intrabodies may target one or more glycated intracellular proteins or may modulate the interaction between one or more glycated intracellular proteins and an alternative protein.

The intracellular expression of intrabodies in different compartments of mammalian cells allows blocking or modulation of the function of endogenous molecules (Biocca, et al., EMBO J. 1990, 9: 101-108; Colby et al., Proc. Natl. Acad. Sci. U.S.A. 2004, 101: 17616-17621). Intrabodies can alter protein folding, protein-protein, protein-DNA, protein-RNA interactions and protein modification. They can induce a phenotypic knockout and work as neutralizing agents by direct binding to the target antigen, by diverting its intracellular trafficking or by inhibiting its association with binding partners. With high specificity and affinity to target antigens, intrabodies have advantages to block certain binding interactions of a particular target molecule, while sparing others.

In one embodiment, the antibody may be a conditionally active biologic protein. An antibody may be used to generate a conditionally active biologic protein which are reversibly or irreversibly inactivated at the wild type normal physiological conditions as well as to such conditionally active biologic proteins and uses of such conditional active biologic proteins are provided. In some embodiments, SREs and/or payloads of the invention may include a cell-penetrating antibody. Cell-penetrating antibodies are antibodies, or antibody fragments or variants, that are capable of passing through cellular membrane and entering into cells.

### Antibody Preparations

The preparation of antibodies, whether monoclonal or polyclonal, is known in the art. Techniques for the production of antibodies are well known in the art and described, e.g. in Harlow and Lane “Antibodies, A Laboratory Manual”, Cold Spring Harbor Laboratory Press, 1988; Harlow and Lane “Using Antibodies: A Laboratory Manual” Cold Spring Harbor Laboratory Press, 1999 and “Therapeutic Antibody Engineering: Current and Future Advances Driving the Strongest Growth Area in the Pharmaceutical Industry” Woodhead Publishing, 2012.

The antibodies and fragments and variants thereof as described herein can be produced using recombinant polynucleotides. In one embodiment, the polynucleotides have a modular design to encode at least one of the antibodies, fragments or variants thereof. As a non-limiting example, the polynucleotide construct may encode any of the following designs: (1) the heavy chain of an antibody, (2) the light chain of an antibody, (3) the heavy and light chain of the antibody, (4) the heavy chain and light chain separated by a linker, (5) the VH1, CH1, CH2, CH3 domains, a linker and the light chain or (6) the VH1, CH1, CH2, CH3 domains, VL region, and the light chain. Any of these designs may also comprise optional linkers between any domain and/or region. The polynucleotides of the present invention may be engineered to produce any standard class of immunoglobulins using an antibody described herein or any of its component parts as a starting molecule.

Recombinant antibody fragments may also be isolated from phage antibody libraries using techniques well known in the art and described in e.g. Clackson et al., 1991, Nature 352: 624-628; Marks et al., 1991, J. Mol. Biol. 222: 581-597. Recombinant antibody fragments may be derived from large phage antibody libraries generated by recombination in bacteria (Sblattero and Bradbury, 2000, Nature Biotechnology 18:75-80; the contents of which are incorporated herein by reference in its entirety).

### Antibodies Used for Immunotherapy

In some embodiments, payloads of the present invention may be antibodies, fragments and variants thereof which are specific to tumor specific antigens (TSAs) and tumor associated antigens (TAAs). Antibodies circulate throughout the body until they find and attach to the TSA/TAA. Once attached, they recruit other parts of the immune system, increasing ADCC (antibody dependent cell-mediated cytotoxicity) and ADCP (antibody dependent cell-mediated phagocytosis) to destroy tumor cells. As used herein, the term “tumor specific antigen (TSA)” means an antigenic substance produced in tumor cells, which can trigger an anti-tumor immune response in a host organism. In one embodiment, a TSA may be a tumor neoantigen. The tumor antigen specific antibody mediates complement-dependent cytotoxic response against tumor cells expressing the same antigen.

In some embodiments, the tumor specific antigens (TSAs), tumor associated antigens (TAAs), pathogen associated antigens, or fragments thereof can be expressed as a peptide or as an intact protein or portion thereof. The intact protein or a portion thereof can be native or mutagenized. Antigens associated with cancers or virus-induced cancers as described herein are well-known in the art. Such a TSA or TAA may be previously associated with a cancer or may be identified by any method known in the art.

In one embodiment, the antigen is CD19, a B-cell surface protein expressed throughout B-cell development. CD19 is a well-known B cell surface molecule, which upon B cell receptor activation enhances B-cell antigen receptor induced signaling and expansion of B cell populations. CD19 is broadly expressed in both normal and neoplastic B cells. Malignancies derived from B cells such as chronic lymphocytic leukemia, acute lymphocytic leukemia and many non-Hodgkin lymphomas frequently retain CD19 expression. This near universal expression and specificity for a single cell lineage has made CD19 an attractive target for immunotherapies. Human CD19 has 14 exons wherein exon 1-4 encode the extracellular portion of the CD19, exon 5 encodes the transmembrane portion of CD19 and exons 6-14 encode the cytoplasmic tail.

In one embodiment, payloads of the present invention may be antibodies, fragments and variants thereof which are specific to CD19 antigen.

In some embodiments, the immunotherapeutic agent may be an antibody that is specifically immunoreactive to an antigen selected from a tumor specific antigen (TSA), a tumor associated antigen (TAA), or an antigenic epitope.

In one aspect, the antigen may be an antigenic epitope. In some embodiments, the antigenic epitope may be CD19.

In some embodiments, the antibody may comprise a heavy chain variable region having an amino acid sequence independently selected from the group consisting of any of SEQ ID NOs. 9-40 and a light chain variable region having an amino acid sequence independently selected from the group consisting of any of SEQ ID NOs. 41-82. In one aspect, the antibody may comprise an amino acid sequence selected from the group consisting of any of SEQ ID NOs. 83-227 and 465.

In one aspect, the first effector module may comprise the amino acid sequence of any of SEQ ID NO. 475-489, 804-810, 813-817 and 948-1029.

A tumor specific antigen (TSA) may be a tumor neoantigen. A neoantigen is a mutated antigen that is only expressed by tumor cells because of genetic mutations or alterations in transcription which alter protein coding sequences, therefore creating novel, foreign antigens. The genetic changes result from genetic substitution, insertion, deletion or any other genetic changes of a native cognate protein (i.e. a molecule that is expressed in normal cells). In the context of CD19, neoantigens such as a transcript variant of CD19 lacking exon 2 or lacking exon 5-6 or both have been described (see International patent publication No. WO2016061368; the contents of which are incorporated herein by reference in their entirety). Since FMC63 binding epitope is in exon 2, CD19 neoantigen lacking exon 2 is not recognized by FMC63 antibody. Thus, in some embodiments, payloads of the invention may include FMC63-distinct antibodies, or fragments thereof. As used herein “FMC63-distinct” refers, to an antibody or fragment thereof that is immunologically specific and binds to an epitope of the CD19 antigen that is different or unlike the epitope of CD19 antigen that is bound by FMC63. In some instances, antibodies of the invention may include CD19 antibodies, antibody fragments or variants that recognize CD19 neoantigens including the CD19 neoantigen lacking exon2. In one embodiment, the antibody or fragment thereof is immunologically specific to the CD19 encoded by exon 1, 3 and/or 4. In one example, the antibody or fragment thereof is specific to the epitope that bridges the portion of CD19 encoded by exon 1 and the portion of CD19 encoded by exon 3.

### Chimeric Antigen Receptors (CARs)

In some embodiments, payloads of the present invention may be a chimeric antigen receptors (CARs) which when transduced into immune cells (e.g., T cells and NK cells), can re-direct the immune cells against the target (e.g., a tumor cell) which expresses a molecule recognized by the extracellular target moiety of the CAR.

As used herein, the term “chimeric antigen receptor (CAR)” refers to a synthetic receptor that mimics TCR on the surface of T cells. In general, a CAR is composed of an extracellular targeting domain, a transmembrane domain/region and an intracellular signaling/activation domain. In a standard CAR receptor, the components: the extracellular targeting domain, transmembrane domain and intracellular signaling/activation domain, are linearly constructed as a single fusion protein. The extracellular region comprises a targeting domain/moiety (e.g., a scFv) that recognizes a specific tumor antigen or other tumor cell-surface molecules. The intracellular region may contain a signaling domain of TCR complex (e.g., the signal region of CD3), and/or one or more costimulatory signaling domains, such as those from CD28, 4-1BB (CD137) and OX-40 (CD134). For example, a “first-generation CAR” only has the CD3 signaling domain. In an effort to augment T-cell persistence and proliferation, costimulatory intracellular domains are added, giving rise to second generation CARs having a CD3ξ signal domain plus one costimulatory signaling domain, and third generation CARs having CD3ξ signal domain plus two or more costimulatory signaling domains. A CAR, when expressed by a T cell, endows the T cell with antigen specificity determined by the extracellular targeting moiety of the CAR. Recently, it is also desirable to add one or more elements such as homing and suicide genes to develop a more competent and safer architecture of CAR, so called the fourth-generation CAR.

In some embodiments, the immunotherapeutic agent of the effector module is a chimeric antigen receptor (CAR). The chimeric antigen receptor may comprise an extracellular target moiety; a transmembrane domain; an intracellular signaling domain; and optionally, one or more co-stimulatory domains.

In some embodiments, the extracellular targeting domain is joined through the hinge (also called space domain or spacer) and transmembrane regions to an intracellular signaling domain. The hinge connects the extracellular targeting domain to the transmembrane domain which transverses the cell membrane and connects to the intracellular signaling domain. The hinge may need to be varied to optimize the potency of CAR transformed cells toward cancer cells due to the size of the target protein where the targeting moiety binds, and the size and affinity of the targeting domain itself. Upon recognition and binding of the targeting moiety to the target cell, the intracellular signaling domain leads to an activation signal to the CAR T cell, which is further amplified by the “second signal” from one or more intracellular costimulatory domains. The CAR T cell, once activated, can destroy the target cell.

In some embodiments, the CAR of the present invention may be split into two parts, each part is linked a dimerizing domain, such that an input that triggers the dimerization promotes assembly of the intact functional receptor.

In some embodiments, the CAR of the present invention may be designed as an inducible CAR.

According to the present invention, the payload of the present invention may be a first-generation CAR, or a second-generation CAR, or a third-generation CAR, or a fourth-generation CAR. In some embodiments, the payload of the present invention may be a full CAR construct composed of the extracellular domain, the hinge and transmembrane domain and the intracellular signaling region. In other embodiments, the payload of the present invention may be a component of the full CAR construct including an extracellular targeting moiety, a hinge region, a transmembrane domain, an intracellular signaling domain, one or more co-stimulatory domain, and other additional elements that improve CAR architecture and functionality including but not limited to a leader sequence, a homing element and a safety switch, or the combination of such components.

CARs regulated by biocircuits and compositions of the present invention are tunable and thereby offer several advantages. The reversible on-off switch mechanism allows management of acute toxicity caused by excessive CAR-T cell expansion. Pulsatile CAR expression using SREs of the present invention may be achieved by cycling ligand level. The ligand conferred regulation of the CAR may be effective in offsetting tumor escape induced by antigen loss, avoiding functional exhaustion caused by tonic signaling due to chronic antigen exposure and improving the persistence of CAR expressing cells in vivo.

In some embodiments, biocircuits and compositions of the invention may be utilized to down regulate CAR expression to limit on target on tissue toxicity caused by tumor lysis syndrome. Down regulating the expression of the CARs of the present invention following anti-tumor efficacy may prevent (1) On target off tumor toxicity caused by antigen expression in normal tissue, (2) antigen independent activation in vivo.

In one embodiment, selection of a CAR with a lower affinity may provide more T cell signaling and less toxicity.

### Extracellular Targeting Domain/Moiety

In accordance with the invention, the extracellular target moiety of a CAR may be any agent that recognizes and binds to a given target molecule, for example, a neoantigen on tumor cells, with high specificity and affinity. The target moiety may be an antibody and variants thereof that specifically binds to a target molecule on tumor cells, or a peptide aptamer selected from a random sequence pool based on its ability to bind to the target molecule on tumor cells, or a variant or fragment thereof that can bind to the target molecule on tumor cells, or an antigen recognition domain from native T-cell receptor (TCR) (e.g. CD4 extracellular domain to recognize HIV infected cells), or exotic recognition components such as a linked cytokine that leads to recognition of target cells bearing the cytokine receptor, or a natural ligand of a receptor.

In some embodiments, the targeting domain of a CAR may be a Ig NAR, a Fab fragment, a Fab′ fragment, a F(ab)′2 fragment, a F(ab)′3 fragment, Fv, a single chain variable fragment (scFv), a bis-scFv, a (scFv)2, a minibody, a diabody, a triabody, a tetrabody, a disulfide stabilized Fv protein (dsFv), a unitbody, a nanobody, or an antigen binding region derived from an antibody that specifically recognizes a target molecule, for example a tumor specific antigen (TSA). In one embodiment, the targeting moiety is a scFv. The scFv domain, when it is expressed on the surface of a CAR T cell and subsequently binds to a target protein on a cancer cell, is able to maintain the CAR T cell in proximity to the cancer cell and to trigger the activation of the T cell. A scFv can be generated using routine recombinant DNA technology techniques and is discussed in the present invention.

In some embodiments, natural ligands may be used as the targeting moieties of the CARs of the present invention. Such natural ligands may be capable of binding to the antigens with affinity in the range of the scFvs and can redirect T cells specificity and effector functions to target cells expressing the complementary receptor.

In one embodiment, the targeting moiety of the CAR may recognize CD19. CD19 is a well-known B cell surface molecule, which upon B cell receptor activation enhances B-cell antigen receptor induced signaling and expansion of B cell populations. CD19 is broadly expressed in both normal and neoplastic B cells. Malignancies derived from B cells such as chronic lymphocytic leukemia, acute lymphocytic leukemia and many non-Hodgkin lymphomas frequently retain CD19 expression. This near universal expression and specificity for a single cell lineage has made CD19 an attractive target for immunotherapies. Human CD19 has 14 exons wherein exon 1-4 encode the extracellular portion of the CD19, exon 5 encodes the transmembrane portion of CD19 and exons 6-14 encode the cytoplasmic tail. In one embodiment, the targeting moiety may comprise scFvs derived from the variable regions of the FMC63 antibody. FMC63 is an IgG2a mouse monoclonal antibody clone specific to the CD19 antigen that reacts with CD19 antigen on cells of the B lineage. The epitope of CD19 recognized by the FMC63 antibody is in exon 2 (Sotillo et al (2015) Cancer Discov; 5(12):1282-95; the contents of which are incorporated by reference in their entirety). In some embodiments, the targeting moiety of the CAR may be derived from the variable regions of other CD19 monoclonal antibody clones including but not limited to 4G7, SJ25C1, CVID3/429, CVID3/155, HIB19, and J3-119.

In some embodiments, the targeting moiety of a CAR may recognize a tumor specific antigen (TSA), for example a cancer neoantigen that is only expressed by tumor cells because of genetic mutations or alterations in transcription which alter protein coding sequences, therefore creating novel, foreign antigens. The genetic changes result from genetic substitution, insertion, deletion or any other genetic changes of a native cognate protein (i.e. a molecule that is expressed in normal cells). In the context of CD19, TSAs may include a transcript variant of human CD19 lacking exon 2 or lacking exon 5-6 or both (see International patent publication No. WO2016061368; the contents of which are incorporated herein by reference in their entirety). Since FMC63 binding epitope is in exon 2, CD19 lacking exon 2 is not recognized by FMC63 antibody. Thus, in some embodiments, the targeting moiety of the CAR may be an FMC63-distinct scFv. As used herein “FMC63-distinct” refers, to an antibody, scFv or a fragment thereof that is immunologically specific and binds to an epitope of the CD19 antigen that is different or unlike the epitope of CD19 antigen that is bound by FMC63. In some instances, targeting moiety may recognize a CD19 antigen lacking exon2. In one embodiment, the targeting moiety recognizes a fragment of CD19 encoded by exon 1, 3 and/or 4. In one example, the targeting moiety recognizes the epitope that bridges the portion of CD19 encoded by exon 1 and the portion of CD19 encoded by exon 3.

In one aspect, the extracellular target moiety may be an scFv derived from an antibody. In one aspect, the scFv may specifically bind to a CD19 antigen. In one aspect, the scFv of the CAR may be a CD19 scFv. In some embodiments, the CD19 scFv may comprise a heavy chain variable region having an amino acid sequence independently selected from the group consisting of SEQ ID NO. 49-80, and a light chain variable region having an amino acid sequence independently selected from the group consisting of any of SEQ ID NOs. 81-122. In some embodiments, the CD19 scFv may comprise an amino acid sequence selected from the group consisting of any of SEQ ID NOs. 123-267 and 624.

In some embodiments, the CD19 scFvs may be selected from any of the sequences described in Table 2. In Table 2, * at the end of the amino acid sequence indicates the translation of the stop codon, and X denotes any amino acid.

TABLE 2
CD19 scFv sequences

| scFv | | | Amino | Nucleic |
|---|---|---|---|---|
| clone | Target | Amino Acid | Acid | Acid |
| name | | Sequence | SEQ ID | SEQ ID |
| T1_G4 | CD19 | GAHAQPVLTQ | 1034 | 1037 |
| | | PPSVSVAPGQ | | |
| | | TAKITCGGNN | | |
| | | IGSKSVHWYQ | | |
| | | QKPGQAPVLV | | |
| | | VYDDSDRPSG | | |
| | | IPERFSGSNS | | |
| | | GNAATLTISR | | |
| | | VEAGDEADYY | | |
| | | CQVWDSSSGL | | |
| | | VFGTGTKVTV | | |
| | | LSGGSTITSY | | |
| | | NVYDTKLSSS | | |
| | | GTEVQLLESG | | |
| | | AEVKKPGESL | | |
| | | KISCKGSGYS | | |
| | | FTSYWIGWVR | | |
| | | QMPGKGLEWM | | |
| | | GIIYPGDSDT | | |
| | | RYSPSSQGQV | | |
| | | TISADKSIST | | |
| | | AYLRWSGLKA | | |
| | | SDTAMYYCAR | | |
| | | VSSDSGAFDI | | |
| | | WGQGTMVTVS | | |
| | | SASGKPIPNP | | |
| | | LLGLDSTHHH | | |
| | | HHH* | | |
| A8_F1 | CD19 | MKYLLPTAAA | 1035 | 1038 |
| | | GLLLLAASGA | | |
| | | HASYELTQPP | | |
| | | SVSVAPGKTA | | |
| | | TIPCGGNNIE | | |
| | | SKSVHWYQQR | | |
| | | PGQAPVLVIY | | |
| | | DDTDRPSGIP | | |
| | | ERFSGSNSGN | | |
| | | TATLTISGVE | | |
| | | AGDEADYFCQ | | |
| | | VWDSHSDHEV | | |
| | | FGGGTKLTVL | | |
| | | SGGSTITSYN | | |
| | | VYYTKLSSSG | | |
| | | SEVQLVETGG | | |
| | | GLVQPGGSLR | | |
| | | LSCAASGFTF | | |
| | | SSYEMNWVRQ | | |
| | | APGKGLEWVS | | |
| | | YISSSGSTIY | | |
| | | YADSVKGRFT | | |
| | | ISRDNAKNSL | | |
| | | YLQMNSLRAE | | |
| | | DTAVYYCARE | | |
| | | HEWEAGAFDI | | |
| | | WGQGTMVTVS | | |
| | | SASGKPIPNP | | |
| | | LLGLDSTHHH | | |
| | | HHH* | | |
| A8_F2 | CD19 | GAHASYELTQ | 1036 | |
| | | PPSVSVAPGK | | |
| | | TATIPCGGNN | | |
| | | IESKSVHWYQ | | |
| | | QRPGQAPVLV | | |
| | | IYDDTDRPSG | | |
| | | IPERFSGSNS | | |
| | | GNTATLTISG | | |
| | | VEAGDEADYF | | |
| | | CQVWDSHSDH | | |
| | | EVFGGGTKLT | | |
| | | VLSGGSTITS | | |
| | | YNVYYTKLSS | | |
| | | SGSEVQLVET | | |
| | | GGGLVQPGGS | | |
| | | LRLSCAASGF | | |
| | | TFSSYEMNWV | | |
| | | RQAPGKGLEW | | |
| | | VSYISSSGST | | |
| | | IYYADSVKGR | | |
| | | FTISRDNAKN | | |
| | | SLYLQMNSLR | | |
| | | AEDTAVYYCA | | |
| | | REHEWXAGAF | | |
| | | DIWGXGTMVT | | |
| | | VSSASGKPIP | | |
| | | NPLLGLDSTH | | |
| | | HHHHH* | | |

In some embodiments, scFvs with at least 70% identity to the amino acid sequence of the scFvs disclosed in Table 2, may be used in the present invention. In some embodiments, scFvs with at least 75%, at least 80%, at least 85%, at least 90%, and at least 95% identity to the amino acid sequence of the scFvs described in Table 2 may be useful in the present invention.

The antigen-binding site (also known as the antigen combining site or paratope) of the scFv described in Table 2 may comprise the amino acid residues necessary to interact with a CD19 antigen. The exact residues making up the antigen-binding site may be elucidated by co-crystallography with bound antigen, however computational assessments based on comparisons with other antibodies may also be used (Strohl, W. R. Therapeutic Antibody Engineering. Woodhead Publishing, Philadelphia Pa. 2012. Ch. 3, p 47-54, the contents of which are herein incorporated by reference in their entirety). Determining residues that make up CDRs may include the use of numbering schemes including, but not limited to, those taught by Kabat (Wu et al., JEM, 1970, 132(2):211-250 and Johnson et al., Nucleic Acids Res. 2000, 28(1): 214-218, the contents of each of which are herein incorporated by reference in their entirety), Chothia (Chothia and Lesk, J. Mol. Biol. 1987, 196, 901, Chothia et al., Nature, 1989, 342, 877, and A1-Lazikani et al., J. Mol. Biol. 1997, 273(4): 927-948, the contents of each of which are herein incorporated by reference in their entirety), Lefranc (Lefranc et al., Immunome Res. 2005, 1:3) and Honegger (Honegger and Pluckthun, J. Mol. Biol. 2001, 309(3): 657-70, the contents of which are herein incorporated by reference in their entirety).

VH and VL domains have three CDRs each. In some cases, CDR-H3s may be analyzed among a panel of related antibodies to assess antibody diversity. Various methods of determining CDR sequences are known in the art and may be applied to known antibody sequences (Strohl, W. R. Therapeutic Antibody Engineering. Woodhead Publishing, Philadelphia Pa. 2012. Ch. 3, p 47-54, the contents of which are herein incorporated by reference in their entirety). One or more nucleotide of the scFv clones described in Table 2 may be mutated to generate additional scFvs with enhanced affinity for CD19. In some embodiments, mutations may be engineered in one or more of the scFvs described in Table 2, to decrease the affinity of the scFv.

### Intracellular Signaling Domains

The intracellular domain of a CAR fusion polypeptide, after binding to its target molecule, transmits a signal to the immune effector cell, activating at least one of the normal effector functions of immune effector cells, including cytolytic activity (e.g., cytokine secretion) or helper activity. Therefore, the intracellular domain comprises an “intracellular signaling domain” of a T cell receptor (TCR).

In some aspects, the entire intracellular signaling domain can be employed. In other aspects, a truncated portion of the intracellular signaling domain may be used in place of the intact chain as long as it transduces the effector function signal.

In some embodiments, the intracellular signaling domain of the present invention may contain signaling motifs which are known as immunoreceptor tyrosine-based activation motifs (ITAMs). Examples of ITAM containing cytoplasmic signaling sequences include those derived from TCR CD3zeta, FcR gamma, FcR beta, CD3 gamma, CD3 delta, CD3 epsilon, CD5, CD22, CD79a, CD79b, and CD66d. In one example, the intracellular signaling domain is a CD3 zeta (CD3) signaling domain.

In some embodiments, the intracellular region of the present invention further comprises one or more costimulatory signaling domains which provide additional signals to the immune effector cells. These costimulatory signaling domains, in combination with the signaling domain can further improve expansion, activation, memory, persistence, and tumor-eradicating efficiency of CAR engineered immune cells (e.g., CAR T cells). In some cases, the costimulatory signaling region contains 1, 2, 3, or 4 cytoplasmic domains of one or more intracellular signaling and/or costimulatory molecules. The costimulatory signaling domain may be the intracellular/cytoplasmic domain of a costimulatory molecule, including but not limited to CD2, CD7, CD27, CD28, 4-1BB (CD137), OX40 (CD134), CD30, CD40, ICOS (CD278), GITR (glucocorticoid-induced tumor necrosis factor receptor), LFA-1 (lymphocyte function-associated antigen-1), LIGHT, NKG2C, B7-H3. In one example, the costimulatory signaling domain is derived from the cytoplasmic domain of CD28. In another example, the costimulatory signaling domain is derived from the cytoplasmic domain of 4-1BB (CD137).

In some embodiments, the intracellular region of the present invention may comprise a functional signaling domain from a protein selected from the group consisting of an MHC class I molecule, a TNF receptor protein, an immunoglobulin-like protein, a cytokine receptor, an integrin, a signaling lymphocytic activation protein (SLAM) such as CD48, CD229, 2B4, CD84, NTB-A, CRACC, BLAME, CD2F-10, SLAMF6, SLAMF7, an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), SLAMF7, NKp80 (KLRF1), NKp44, NKp30, NKp46, CD19, CD4, CD8alpha, CD8beta, IL2R beta, IL2R gamma, IL7R alpha, IL15Ra, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, NKD2C SLP76, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, CD270 (HVEM), GADS, SLP-76, PAG/Cbp, CD19a, a ligand that specifically binds with CD83, DAP 10, TRIM, ZAP70, Killer immunoglobulin receptors (KIRs) such as KIR2DL1, KIR2DL2/L3, KIR2DL4, KIR2DL5A, KIR2DL5B, KIR2DS1, KIR2DS2, KIR2DS3, KIR2DS4, KIR2DS5, KIR3DL1/S1, KIR3DL2, KIR3DL3, and KIR2DP1; lectin related NK cell receptors such as Ly49, Ly49A, and Ly49C.

In some embodiments, the intracellular signaling domain of the present invention may contain signaling domains derived from JAK-STAT. In other embodiments, the intracellular signaling domain of the present invention may contain signaling domains derived from DAP-12 (Death associated protein 12) (Topfer et al., Immunol., 2015, 194: 3201-3212; and Wang et al., Cancer Immunol., 2015, 3: 815-826). DAP-12 is a key signal transduction receptor in NK cells. The activating signals mediated by DAP-12 play important roles in triggering NK cell cytotoxicity responses toward certain tumor cells and virally infected cells. The cytoplasmic domain of DAP12 contains an Immunoreceptor Tyrosine-based Activation Motif (ITAM). Accordingly, a CAR containing a DAP12-derived signaling domain may be used for adoptive transfer of NK cells.

In some embodiments, T cells engineered with two or more CARs incorporating distinct co-stimulatory domains and regulated by distinct DD may be used to provide kinetic control of downstream signaling.

In some embodiments, intracellular signaling domain of the CAR may be a CD3 zeta signaling domain (SEQ ID NO. 299), encoded by any of the nucleotide sequence of SEQ ID NO. 501-505, and 786. In some embodiments, the intracellular signaling domain of the CAR may be a 4-1BB intracellular signaling domain (SEQ ID NO. 233), encoded by any of the nucleotide sequence of SEQ ID NO. 506-510, and 785.

In some embodiments, the GITR co-stimulatory domains may be useful in the CAR described herein. In some embodiments, the GITR domains may be capable of inducing T cell effector function and activating T cells. In some aspects, GITR domains described herein may be able to suppress inhibitory T regulatory cells that block immune response. In some embodiments, GITR intracellular domain containing CAR T cells can decrease the production of cytokines, which may reduce the cytokine release syndrome.

In some embodiments, the intracellular signaling domain of the present invention may be an intracellular domain comprising the amino acid and nucleotide sequences provided in Table 3.

TABLE 3
Additional intracellular signaling
and co-stimulatory domains

| | | Amino | Nucleic |
|---|---|---|---|
| | | acid | acid |
| | | SEQ | SEQ |
| | | ID | ID |
| Domain | Sequence | NO | NO. |
| 4-1 BB | KRGRKKLLYIFKQPFMRPV | 1171 | 1177; |
| intracellular | QTTQEEDGCSCRFPEEEE | | 1178 |
| domain | GGCEL | | |
| CD27 | QRRKYRSNKGESPVEPAEP | 1172 | 1179 |
| | CRYSCPREEEGSTIPIQED | | |
| | YRKPEPACSP | | |
| CD3 Zeta | RVKFSRSADAPAYKQGQNQ | 1173 | 1180- |
| | LYNELNLGRREEYDVLDKR | | 1181 |
| | RGRDPEMGGKPRRKNPQEG | | |
| | LYNELQKDKMAEAYSEIGM | | |
| | KGERRRGKGHDGLYQGLST | | |
| | ATKDTYDALHMQALPPR | | |
| CD3 Zeta | RVKFSRSADAPAYQQGQNQ | 1174 | 1182 |
| | LYNELNLGRREEYDVLDKR | | |
| | RGRDPEMGGKPRRKNPQEG | | |
| | LYNELQKDKMAE | | |
| | AYSEIGMKGERRRGKGHDG | | |
| | LYQGLSTATKDTYDALHMQ | | |
| | ALPPR | | |
| ICOS | TKKKYSSSVHDPNGEYMFM | 1175 | 1183 |
| intracellular | RAVNTAKKSRLTDVTL | | |
| domain (AA) | | | |
| CD28 | RSKRSRLLHSDYMNMTPRR | 1176 | 1184 |
| intracellular | PGPTRKHYQPYAPPRDF | | |
| domain (AA) | AAYRS | | |

### Transmembrane Domains

In some embodiments, the CAR of the present invention may comprise a transmembrane domain. As used herein, the term “Transmembrane domain (TM)” refers broadly to an amino acid sequence of about 15 residues in length which spans the plasma membrane. More preferably, a transmembrane domain includes at least 20, 21, 22, 23, 24, 25, 26, 27, 28, 29, 30, 31, 32, 33, 34, 35, 36, 37, 38, 39, 40, 41, 42, 43, 44, or 45 amino acid residues and spans the plasma membrane. In some embodiments, the transmembrane domain of the present invention may be derived either from a natural or from a synthetic source. The transmembrane domain of a CAR may be derived from any naturally membrane-bound or transmembrane protein. For example, the transmembrane region may be derived from (i.e. comprise at least the transmembrane region(s) of) the alpha, beta or zeta chain of the T-cell receptor, CD3 epsilon, CD4, CD5, CD8, CD8α, CD9, CD16, CD22, CD33, CD28, CD37, CD45, CD64, CD80, CD86, CD134, CD137, CD152, or CD154.

Alternatively, the transmembrane domain of the present invention may be synthetic. In some aspects, the synthetic sequence may comprise predominantly hydrophobic residues such as leucine and valine.

In some embodiments, the transmembrane domain of the present invention may be selected from the group consisting of a CD8α transmembrane domain, a CD4 transmembrane domain, a CD 28 transmembrane domain, a CTLA-4 transmembrane domain, a PD-1 transmembrane domain, and a human Ig_{G4} Fc region. As non-limiting examples, the transmembrane domain may be a CTLA-4 transmembrane domain comprising the amino acid sequences of SEQ ID NOs. 1-5 of International Patent Publication NO. WO2014/100385; and a PD-1 transmembrane domain comprising the amino acid sequences of SEQ ID NOs. 6-8 of International Patent Publication NO. WO2014100385; the contents of each of which are incorporated herein by reference in their entirety.

In some embodiments, the CAR of the present invention may comprise an optional hinge region (also called spacer). A hinge sequence is a short sequence of amino acids that facilitates flexibility of the extracellular targeting domain that moves the target binding domain away from the effector cell surface to enable proper cell/cell contact, target binding and effector cell activation (Patel et al., Gene Therapy, 1999; 6: 412-419). The hinge sequence may be positioned between the targeting moiety and the transmembrane domain. The hinge sequence can be any suitable sequence derived or obtained from any suitable molecule. The hinge sequence may be derived from all or part of an immunoglobulin (e.g., IgG1, IgG2, IgG3, IgG4) hinge region, i.e., the sequence that falls between the CHI and CH2 domains of an immunoglobulin, e.g., an IgG4 Fc hinge, the extracellular regions of type 1 membrane proteins such as CD8αCD4, CD28 and CD7, which may be a wild type sequence or a derivative. Some hinge regions include an immunoglobulin CH3 domain or both a CH3 domain and a CH2 domain. In certain embodiments, the hinge region may be modified from an IgG1, IgG2, IgG3, or IgG4 that includes one or more amino acid residues, for example, 1, 2, 3, 4 or 5 residues, substituted with an amino acid residue different from that present in an unmodified hinge.

In some embodiments, the transmembrane domain may be a CD8 transmembrane domain, comprising the amino acid sequence of SEQ ID NO. 1185, encoded by the nucleotide sequence of SEQ ID NO. 1186-1187. In one embodiment, the transmembrane domain may be a ICOS transmembrane domain, comprising the amino acid sequence of SEQ ID NO. 1188, encoded by the nucleotide sequence of SEQ ID NO. 1189.

In some embodiments, the hinge region may be a CD8 hinge region, comprising the amino acid sequence of SEQ ID NO. 1190, encoded by the nucleotide sequence of SEQ ID NO. 1191. In some embodiments, the hinge region may be an Ig4 hinge region, comprising the amino acid sequence of SEQ ID NO. 1192, encoded by the nucleotide sequence of SEQ ID NO. 1193 or an IgD hinge, comprising the amino acid sequence of SEQ ID NO. 1194, encoded by the nucleotide sequence of SEQ ID NO. 1195.

In some embodiments, the CAR of the present invention may comprise one or more linkers between any of the domains of the CAR. The linker may be between 1-30 amino acids long. In this regard, the linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length. In other embodiments, the linker may be flexible.

In some embodiments, the components including the targeting moiety, transmembrane domain and intracellular signaling domains of the present invention may be constructed in a single fusion polypeptide. The fusion polypeptide may be the payload of an effector module of the invention. In some embodiments, more than one CAR fusion polypeptides may be included in an effector module, for example, two, three or more CARs may be included in the effector module under the control of a single SRE (e.g., a DD).

In one embodiment of the present invention, the payload of the invention is a CD19 specific CAR targeting different B cell. In the context of the invention, an effector module may comprise a hDHFR DD, ecDHFR DD, or FKBP DD operably linked to a CD19 CAR fusion construct. In some instances, the promoter utilized to drive the expression of the effector module in the vector may be a CMV promoter or an EF1a. The efficiency of the promoter in driving the expression of the same construct may be compared. For example, two constructs that differ only by their promoter, CMV (in OT-001010 OT-CD19-001 or OT-CD19N-001) or EF1a promoter (in OT-001399 (OT-CD19-055)) may be compared. The amino acid sequences of CD19 CAR constructs and its components are presented in Table 4, and Table 5. In some embodiments, the constructs described herein may comprise two or more payloads and are herein referred to as “tandem constructs”. For example, the CD19 CAR IL12 tandem construct may comprise the both the CD19 CAR and the IL12 payloads operably linked to each. One or more payloads in a tandem construct may further be appended to an SRE to generate the biocircuits of the invention. The amino acid sequences of CD19 and IL15 tandem and CD19 and IL12 tandem expression constructs are also presented in Table 5.

In some embodiments, the CD19 constructs of the invention may be placed under the transcriptional control of the CMV promoter (SEQ ID NO. 556, 1100), an EF1a promoter (SEQ ID NO. 557, 708, 1099, 1103) or a PGK promoter (SEQ ID NO. 558, 1101, 1104).

The amino acid sequences in Table 4 and/or Table 5 may comprise a stop codon which is denoted in the table with a “*” at the end of the amino acid sequence. In some embodiments, the leader sequence derived from human CD8a may comprise amino acids 2-21 of the wild type human CD8a sequence. This may be referred to as an M1del mutation. In the tables below, “WT” means wild-type.

TABLE 4
Sequences of components of CD19 CARs

| | | Amino | |
|---|---|---|---|
| | | Acid | Nucleic |
| | | SEQ | Acid SEQ |
| Description | Amino Acid Sequence | ID NO | ID NO |
| CD19 scFv | DIQMTQTTSSLSASLGDRVTISCRASQDISKYLNWY | 465 | 467, 490- |
| | QQKPDGTVKLLIYHTSRLHSGVPSRFSGSGSGTDYS | | 494 |
| | LTISNLEQEDIATYFCQQGNTLPYTFGGGTKLEITG | | |
| | GGGSGGGGSGGGGSEVKLQESGPGLVAPSQSLSVT | | |
| | CTVSGVSLPDYGVSWIRQPPRKGLEWLGVIWGSET | | |
| | TYYNSALKSRLTIIKDNSKSQVFLKMNSLQTDDTAI | | |
| | YYCAKHYYYGGSYAMDYWGQGTSVTVSS | | |
| CD8α hinge TM | TTTPAPRPPTPAPTIASQPLSLRPEACRPAAGGAVH | 466 | 468, 495, |
| | TRGLDFACDIYIWAPLAGTCGVLLLSLVITLYC | | 782-784 |
| CD8α hinge | TTTPAPRPPTPAPTIASQPLSLRPEACRPAAGGAVH | 400 | 496-500 |
| | TRGLDFACD | | |
| CD3 zeta signaling | RVKFSRSADAPAYKQGQNQLYNELNLGRREEYDV | 299 | 501-505, |
| domain | LDKRRGRDPEMGGKPRRKNPQEGLYNELQKDKM | | 786 |
| | AEAYSEIGMKGERRRGKGHDGLYQGLSTATKDTY | | |
| | DALHMQALPPR | | |
| 4-1BB (41BB) | KRGRKKLLYIFKQPFMRPVQTTQEEDGCSCRFPEE | 233 | 506-510, |
| intracellular signaling | EEGGCEL | | 785 |
| domain | | | |
| CD8a Transmembrane | IYIWAPLAGTCGVLLLSLVITLYC | 369 | 790, 792 |
| domain | | | |
| CD8α leader | MALPVTALLLPLALLLHAARP | 469 | 511-515 |
| p40 signal sequence | MCHQQLVISWFSLVFLASPLVA | 559 | 567-575 |
| IgE leader | MDWTWILFLVAAATRVHS | 630 | 730 |
| p40 (WT) | MCHQQLVISWFSLVFLASPLVAIWELKKDVYVVEL | 1091 | 11094 |
| | DWYPDAPGEMVVLTCDTPEEDGITWTLDQSSEVL | | |
| | GSGKTLTIQVKEFGDAGQYTCHKGGEVLSHSLLLL | | |
| | HKKEDGIWSTDILKDQKEPKNKTFLRCEAKNYSGR | | |
| | FTCWWLTTISTDLTFSVKSSRGSSDPQGVTCGAAT | | |
| | LSAERVRGDNKEYEYSVECQEDSACPAAEESLPIE | | |
| | VMVDAVHKLKYENYTSSFFIRDIIKPDPPKNLQLKP | | |
| | LKNSRQVEVSWEYPDTWSTPHSYFSLTFCVQVQG | | |
| | KSKREKKDRVFTDKTSATVICRKNASISVRAQDRY | | |
| | YSSSWSEWASVPCS | | |
| p40 (23-328 of WT) | IWELKKDVYVVELDWYPDAPGEMVVLTCDTPEED | 563 | 472-474, |
| | GITWTLDQSSEVLGSGKTLTIQVKEFGDAGQYTCH | | 583-592 |
| | KGGEVLSHSLLLLHKKEDGIWSTDILKDQKEPKNK | | |
| | TFLRCEAKNYSGRFTCWWLTTISTDLTFSVKSSRGS | | |
| | SDPQGVTCGAATLSAERVRGDNKEYEYSVECQED | | |
| | SACPAAEESLPIEVMVDAVHKLKYENYTSSFFIRDII | | |
| | KPDPPKNLQLKPLKNSRQVEVSWEYPDTWSTPHS | | |
| | YFSLTFCVQVQGKSKREKKDRVFTDKTSATVICRK | | |
| | NASISVRAQDRYYSSSWSEWASVPCS | | |
| p35 (WT) | MWPPGSASQPPPSPAAATGLHPAARPVSLQCRLSM | 1093 | 1094 |
| | CPARSLLLVATLVLLDHLSLARNLPVATPDPGMFP | | |
| | CLHHSQNLLRAVSNMLQKARQTLEFYPCTSEEIDH | | |
| | EDITKDKTSTVEACLPLELTKNESCLNSRETSFITNG | | |
| | SCLASRKTSFMMALCLSSIYEDLKMYQVEFKTMN | | |
| | AKLLMDPKRQIFLDQNMLAVIDELMQALNFNSET | | |
| | VPQKSSLEEPDFYKTKIKLCILLHAFRIRAVTIDRV | | |
| | MSYLNAS | | |
| p35 (57-253 of WT) | RNLPVATPDPGMFPCLHHSQNLLRAVSNMLQKAR | 564 | 593-602, |
| | QTLEFYPCTSEEIDHEDITKDKTSTVEACLPLELTK | | 811 |
| | NESCLNSRETSFITNGSCLASRKTSFMMALCLSSIY | | |
| | EDLKMYQVEFKTMNAKLLMDPKRQIFLDQNMLA | | |
| | VIDELMQALNFNSETVPQKSSLEEPDFYKTKIKLCI | | |
| | LLHAFRIRAVTIDRVMSYLNAS | | |
| IL 15 (WT) | MRISKPHLRSISIQCYLCLLLNSHFLTEAGIHVFILG | 1095 | 1096 |
| | CFSAGLPKTEANWVNVISDLKKIEDLIQSMHIDATL | | |
| | YTESDVHPSCKVTAMKCFLLELQVISLESGDASIHD | | |
| | TVENLIILANNSLSSNGNVTESGCKECEELEEKNIKE | | |
| | FLQSFVHIVQMFINTS | | |
| IL15 (49-162 of WT) | NWVNVISDLKKIEDLIQSMHIDATLYTESDVHPSCK | 616 | 623-626, |
| | VTAMKCFLLELQVISLESGDASIHDTVENLIILANN | | 801 |
| | SLSSNGNVTESGCKECEELEEKNIKEFLQSFVHIVQ | | |
| | MFINTS | | |
| IL15Ra (WT; Uniprot | MAPRRARGCRTLGLPALLLLLLLRPPATRGITCPPP | 1097 | 1098 |
| ID: Q13261.1) | MSVEHADIWVKSYSLYSRERYICNSGFKRKAGTSS | | |
| | LTECVLNKATNVAHWTTPSLKCIRDPALVHQRPAP | | |
| | PSTVTTAGVTPQPESLSPSGKEPAASSPSSNNTAAT | | |
| | TAAIVPGSQLMPSKSPSTGTTEISSHESSHGTPSQTT | | |
| | AKNWELTASASHQPPGVYPQGHSDTTVAISTSTVL | | |
| | LCGLSAVSLLACYLKSRQTPPLASVEMEAMEALPV | | |
| | TWGTSSRDEDLENCSHHL | | |
| IL 15Ra (31-267 of | ITCPPPMSVEHADIWVKSYSLYSRERYICNSGFKRK | 632 | 639-640, |
| WT) | AGTSSLTECVLNKATNVAHWTTPSLKCIRDPALVH | | 803 |
| | QRPAPPSTVTTAGVTPQPESLSPSGKEPAASSPSSN | | |
| | NTAATTAAIVPGSQLMPSKSPSTGTTEISSHESSHGT | | |
| | PSQTTAKNWELTASASHQPPGVYPQGHSDTTVAIS | | |
| | TSTVLLCGLSAVSLLACYLKSRQTPPLASVEMEAM | | |
| | EALPVTWGTSSRDEDLENCSHHL | | |
| mCherry | MSKGEEDNMAIIKEFMRFKVHMEGSVNGHEFEIEG | 857 | 858 |
| | EGEGRPYEGTQTAKLKVTKGGPLPFAWDILSPQFM | | |
| | YGSKAYVKHPADIPDYLKLSFPEGFKWERVMNFE | | |
| | DGGVVTVTQDSSLQDGEFIYKVKLRGTNFPSDGPV | | |
| | MQKKTMGWEASSERMYPEDGALKGEIKQRLKLK | | |
| | DGGHYDAEVKTTYKAKKPVQLPGAYNVNIKLDIT | | |
| | SHNEDYTIVEQYERAEGRHSTGGMDELYK | | |
| mCherry (MIL) | LSKGEEDNMAIIKEFMRFKVHMEGSVNGHEFEIEG | 828 | 829 |
| | EGEGRPYEGTQTAKLKVTKGGPLPFAWDILSPQFM | | |
| | YGSKAYVKHPADIPDYLKLSFPEGFKWERVMNFE | | |
| | DGGVVTVTQDSSLQDGEFIYKVKLRGTNFPSDGPV | | |
| | MQKKTMGWEASSERMYPEDGALKGEIKQRLKLK | | |
| | DGGHYDAEVKTTYKAKKPVQLPGAYNVNIKLDIT | | |
| | SHNEDYTIVEQYERAEGRHSTGGMDELYK | | |
| IRES | — | — | 799 |
| Linker (GGSGG) | GGSGG | 470 | 516-520 |
| Linker (SG) | SG | — | AGTGGA |
| Linker ((G4S)3) | GGGGSGGGGSGGGGS | 560 | 710-715 |
| Linker (GGSG) | GGSG | 649 | 650; 1041; |
| | | | 1108 |
| CSF2R Signal | MLLLVTSLLLCELPHPAFLLIP | 830 | 831 |
| Sequence (leader) | | | |
| Linker | SGGGSGGGGSGGGGSGGGGSGGGSLQ | 631 | 638,716- |
| (SG3(SG4)3SG3SLQ) | | | 720, 802 |
| BamHI (GS) | GS | — | GGATCA, |
| | | | GGATCC, |
| | | | GGATCA |
| Linker (GSG) | GSG | — | GGATCT |
| (BamHl-Gly) | | | GGA |
| Linker (GSSG) | GSSG | 1109 | 1110 |
| Modified Furin | ESRRVRRNKRSK | 471 | 521-253 |
| Spacer | — | — | 800 |
| Spacer | — | — | 1042 |
| HA Tag | YPYDVPDYA | 823 | 824-826 |
| P2A cleavage site | ATNFSLLKQAGDVEENPGP | 576 | 1043 |
| P2A cleavage site | GATNFSLLKQAGDVEENPGP | 725 | 726 |
| FKBP (M1del, F37V, | GVQVETISPGDGRTFPKRGQTCVVHYTGMLEDGK | 6 | 524-526, |
| L107P) | KVDSSRDRNKPFKFMLGKQEVIRGWEEGVAQMSV | | 787, 789 |
| | GQRAKLTISPDYAYGATGHPGIIPPHATLVFDVELL | | |
| | KPE | | |
| FKBP (M1del, E32G, | GVQVETISPGDGRTFPKRGQTCVVHYTGMLGDGK | 7 | 528-531, |
| F37V, R72G, K106E) | KVDSSRDRNKPFKFMLGKQEVIRGWEEGVAQMSV | | 794, 812, |
| | GQGAKLTISPDYAYGATGHPGIIPPHATLVFDVELL | | 827 |
| | ELE | | |
| ecDHFR (M1del, | ISLIAALAVDYVIGMENAMPWNLPADLAWFKRNT | 4 | 532, 603, |
| R12Y, Y100I) | LNKPVIMGRHTWESIGRPLPGRKNIILSSQPGTODR | | 641, 527, |
| | VTWVKSVDEAIAACGDVPEIMVIGGGRVIEQFLPK | | 788, 791, |
| | AQKLYLTHIDAEVEGDTHFPDYEPDDWESVFSEFH | | 1111 |
| | DADAQNSHSYCFEILERR | | |
| ecDHFR (M1del, | ISLIAALAVDHVIGMENAMPWNLPADLAWFKRNT | 5 | 627, 642, |
| R12H, E129K) | LNKPVIMGRHTWESIGRPLPGRKNIILSSQPGTODR | | 793 |
| | VTWVKSVDEAIAACGDVPEIMVIGGGRVYEQFLPK | | |
| | AQKLYLTHIDAEVEGDTHFPDYKPDDWESVFSEFH | | |
| | DADAQNSHSYCFEILERR | | |
| hDHFR (M1del, | VGSLNCIVAVSQNMGIGKNGDLPWPPLRNEFRYFQ | 696 | 534, 795, |
| Y122I) | RMTTTSSVEGKQNLVIMGKKTWFSIPEKNRPLKGR | | 1112 |
| | INLVLSRELKEPPQGAHFLSRSLDDALKLTEQPELA | | |
| | NKVDMVWIVGGSSVIKEAMNHPGHLKLFVTRIMQ | | |
| | DFESDTFFPEIDLEKYKLLPEYPGVLSDVQEEKGIK | | |
| | YKFEVYEKND | | |
| hDHFR (M1del, | VGSLNCIVAVSQNMGIGKNGDLPWPPLRNEFRYFQ | 691 | 536, 773, |
| Y122I, A125F) | RMTTTSSVEGKQNLVIMGKKTWFSIPEKNRPLKGR | | 774, 796 |
| | INLVLSRELKEPPQGAHFLSRSLDDALKLTEQPELA | | |
| | NKVDMVWIVGGSSVIKEFMNHPGHLKLFVTRIMQ | | |
| | DFESDTFFPEIDLEKYKLLPEYPGVLSDVQEEKGIK | | |
| | YKFEVYEKND | | |
| hDHFR (M1del, | VGSLNCIVAVSQNMGIGKNGDLPWPPLRNEFRYFK | 781 | 538, 797 |
| Q36K, Y122I) | RMTTTSSVEGKQNLVIMGKKTWFSIPEKNRPLKGR | | |
| | INLVLSRELKEPPQGAHFLSRSLDDALKLTEQPELA | | |
| | NKVDMVWIVGGSSVIKEAMNHPGHLKLFVTRIMQ | | |
| | DFESDTFFPEIDLEKYKLLPEYPGVLSDVQEEKGIK | | |
| | YKFEVYEKND | | |
| hDHFR (M1del, | VGSLNCIVAVSQNMGIGKNGDLPWPPLRNEFRYFF | 692 | 540, 775, |
| Q36F, N65F, Y122I) | RMTTTSSVEGKQNLVIMGKKTWFSIPEKFRPLKGRI | | 776, 798 |
| | NLVLSRELKEPPQGAHFLSRSLDDALKLTEQPELA | | |
| | NKVDMVWIVGGSSVIKEAMNHPGHLKLFVTRIMQ | | |
| | DFESDTFFPEIDLEKYKLLPEYPGVLSDVQEEKGIK | | |
| | YKFEVYEKND | | |
| hDHFR (M1del, I17V, | VGSLNCIVAVSQNMGVGKNGDLPWPPLRNEFRYF | 688 | 1113 |
| Y122I) | QRMTTTSSVEGKQNLVIMGKKTWFSIPEKNRPLKG | | |
| | RINLVLSRELKEPPQGAHFLSRSLDDALKLTEQPEL | | |
| | ANKVDMVWIVGGSSVIKEAMNHPGHLKLFVTRIM | | |
| | QDFESDTFFPEIDLEKYKLLPEYPGVLSDVQEEKGI | | |
| | KYKFEVYEKND | | |
| hDHFR (M1del, | VGSLNCIVAVSQNMGAGKNGDLPWPPLRNEFRYF | 1114 | 1115 |
| I17A) | QRMTTTSSVEGKQNLVIMGKKTWFSIPEKNRPLKG | | |
| | RINLVLSRELKEPPQGAHFLSRSLDDALKLTEQPEL | | |
| | ANKVDMVWIVGGSSVYKEAMNHPGHLKLFVTRI | | |
| | MQDFESDTFFPEIDLEKYKLLPEYPGVLSDVQEEK | | |
| | GIKYKFEVYEKND | | |
| hDHFR (M1del, I17A, | VGSLNCIVAVSQNMGAGKNGDLPWPPLRNEFRYF | 1116 | 1117 |
| Y122I) | QRMTTTSSVEGKQNLVIMGKKTWFSIPEKNRPLKG | | |
| | RINLVLSRELKEPPQGAHFLSRSLDDALKLTEQPEL | | |
| | ANKVDMVWIVGGSSVIKEAMNHPGHLKLFVTRIM | | |
| | QDFESDTFFPEIDLEKYKLLPEYPGVLSDVQEEKGI | | |
| | KYKFEVYEKND | | |

TABLE 5
Sequences of CD19 CARs

| | | | Amino | Nucleic |
|---|---|---|---|---|
| | Sequence | | Acid | Acid |
| Construct | Descrip- | | SEQ | SEQ ID |
| Description | tion | Amino Acid Sequence | ID NO | NO |
| OT-001405 | Full | — | — | 818 |
| (Full | Construct | | | |
| Construct; | Encoded | MALPVTALLLPLALLLHAARPDIQMTQTTS | 1104 | 1106 |
| CD8a leader; | Protein 1 | SLSASLGDRVTISCRASQDISKYLNWYQQKP | | |
| CD19 scFv; | | DGTVKLLIYHTSRLHSGVPSRFSGSGSGTDY | | |
| CD8a Hinge | | SLTISNLEQEDIATYFCQQGNTLPYTFGGGT | | |
| and | | KLEITGGGGSGGGGSGGGGSEVKLQESGPG | | |
| Transmembrane | | LVAPSQSLSVTCTVSGVSLPDYGVSWIRQPP | | |
| Domain; 4- | | RKGLEWLGVIWGSETTYYNSALKSRLTIIKD | | |
| 1BB | | NSKSQVFLKMNSLQTDDTAIYYCAKHYYY | | |
| intracellular | | GGSYAMDYWGQGTSVTVSSTTTPAPRPPTP | | |
| domain; CD3 | | APTIASQPLSLRPEACRPAAGGAVHTRGLDF | | |
| zeta signaling | | ACDIYIWAPLAGTCGVLLLSLVITLYCKRGR | | |
| domain; stop; | | KKLLYIFKQPFMRPVQTTQEEDGCSCRFPEE | | |
| BamHI; IRES; | | EEGGCELRVKFSRSADAPAYKQGQNQLYN | | |
| spacer; p40 | | ELNLGRREEYDVLDKRRGRDPEMGGKPRR | | |
| signal | | KNPQEGLYNELQKDKMAEAYSEIGMKGER | | |
| sequence; p40 | | RRGKGHDGLYQGLSTATKDTYDALHMQAL | | |
| (23-328 of | | PPR* | | |
| WT); Linker | Encoded | MCHQQLVISWFSLVFLASPLVAIWELKKDV | 1105 | 1107 |
| ((G4S)3); p35 | Protein 2 | YVVELDWYPDAPGEMVVLTCDTPEEDGIT | | |
| (57-253 of | | WTLDQSSEVLGSGKTLTIQVKEFGDAGQYT | | |
| WT); Linker | | CHKGGEVLSHSLLLLHKKEDGIWSTDILKD | | |
| (GGSG); | | QKEPKNKTFLRCEAKNYSGRFTCWWLTTIS | | |
| FKBP (M1del, | | TDLTFSVKSSRGSSDPQGVTCGAATLSAER | | |
| E32G, F37V, | | VRGDNKEYEYSVECQEDSACPAAEESLPIE | | |
| R72G, | | VMVDAVHKLKYENYTSSFFIRDIIKPDPPKN | | |
| K106E); stop) | | LQLKPLKNSRQVEVSWEYPDTWSTPHSYFS | | |
| (Encoded | | LTFCVQVQGKSKREKKDRVFTDKTSATVIC | | |
| Protein 1: | | RKNASISVRAQDRYYSSSWSEWASVPCSGG | | |
| CD8a leader; | | GGSGGGGSGGGGSRNLPVATPDPGMFPCLH | | |
| CD19 scFv; | | HSQNLLRAVSNMLQKARQTLEFYPCTSEEI | | |
| CD8a Hinge | | DHEDITKDKTSTVEACLPLELTKNESCLNSR | | |
| and | | ETSFITNGSCLASRKTSFMMALCLSSIYEDL | | |
| Transmembrane | | KMYQVEFKTMNAKLLMDPKRQIFLDQNML | | |
| Domain; 4- | | AVTDELMQALNFNSETVPQKSSLEEPDFYKT | | |
| 1BB | | KIKLCILLHAFRIRAVTIDRVMSYLNASGGS | | |
| intracellular | | GGVQVETISPGDGRTFPKRGQTCVVHYTGM | | |
| domain; CD3 | | LGDGKKVDSSRDRNKPFKFMLGKQEVIRG | | |
| zeta signaling | | WEEGVAQMSVGQGAKLTISPDYAYGATGH | | |
| domain; stop) | | PGIIPPHATLVFDVELLELE* | | |
| (Encoded | | | | |
| Protein 2: p40 | | | | |
| signal | | | | |
| sequence; p40 | | | | |
| (23-328 of | | | | |
| WT); Linker | | | | |
| ((G4S)3); p35 | | | | |
| (57-253 of | | | | |
| WT); Linker | | | | |
| (GGSG); | | | | |
| FKBP (M1del, | | | | |
| E32G, F37V, | | | | |
| R72G, | | | | |
| K106E); stop) | | | | |
| (OT-CD19- | | | | |
| 039) | | | | |
| OT-001406 | Full | — | — | 819 |
| (Full | Construct | | | |
| Construct: | Encoded | MALPVTALLLPLALLLHAARPDIQMTQTTS | 1104 | 541 |
| CD8a leader; | Protein 1 | SLSASLGDRVTISCRASQDISKYLNWYQQKP | | |
| CD19 scFv; | | DGTVKLLIYHTSRLHSGVPSRFSGSGSGTDY | | |
| CD8a Hinge | | SLTISNLEQEDIATYFCQQGNTLPYTFGGGT | | |
| and | | KLEITGGGGSGGGGSGGGGSEVKLQESGPG | | |
| Transmembrane | | LVAPSQSLSVTCTVSGVSLPDYGVSWIRQPP | | |
| Domain; 4- | | RKGLEWLGVIWGSETTYYNSALKSRLTIIKD | | |
| 1BB | | NSKSQVFLKMNSLQTDDTAIYYCAKHYYY | | |
| intracellular | | GGSYAMDYWGQGTSVTVSSTTTPAPRPPTP | | |
| domain; CD3 | | APTIASQPLSLRPEACRPAAGGAVHTRGLDF | | |
| zeta signaling | | ACDIYIWAPLAGTCGVLLLSLVITLYCKRGR | | |
| domain; stop; | | KKLLYIFKQPFMRPVQTTQEEDGCSCRFPEE | | |
| BamH1 | | EEGGCELRVKFSRSADAPAYKQGQNQLYN | | |
| (GGATCC); | | ELNLGRREEYDVLDKRRGRDPEMGGKPRR | | |
| IRES; spacer; | | KNPQEGLYNELQKDKMAEAYSEIGMKGER | | |
| p40 signal | | RRGKGHDGLYQGLSTATKDTYDALHMQAL | | |
| sequence; p40 | | PPR* | | |
| (23-328 of | Encoded | MCHQQLVISWFSLVFLASPLVAIWELKKDV | 566 | 610 |
| WT); Linker | Protein 2 | YVVELDWYPDAPGEMVVLTCDTPEEDGIT | | |
| ((G4S)3); p35 | | WTLDQSSEVLGSGKTLTIQVKEFGDAGQYT | | |
| (57-253 of | | CHKGGEVLSHSLLLLHKKEDGIWSTDILKD | | |
| WT); stop) | | QKEPKNKTFLRCEAKNYSGRFTCWWLTTIS | | |
| (Encoded | | TDLTFSVKSSRGSSDPQGVTCGAATLSAER | | |
| Protein 1: | | VRGDNKEYEYSVECQEDSACPAAEESLPIE | | |
| CD8a leader; | | VMVDAVHKLKYENYTSSFFIRDIIKPDPPKN | | |
| CD19 scFv: | | LQLKPLKNSRQVEVSWEYPDTWSTPHSYFS | | |
| CD 8a Hinge | | LTFCVQVQGKSKREKKDRVFTDKTSATVIC | | |
| and | | RKNASISVRAQDRYYSSSWSEWASVPCSGG | | |
| Transmembrane | | GGSGGGGSGGGGSRNLPVATPDPGMFPCLH | | |
| Domain; 4- | | HSQNLLRAVSNMLQKARQTLEFYPCTSEEI | | |
| 1BB | | DHEDITKDKTSTVEACLPLELTKNESCLNSR | | |
| intracellular | | ETSFITNGSCLASRKTSFMMALCLSSIYEDL | | |
| domain; CD3 | | KMYQVEFKTMNAKLLMDPKRQIFLDQNML | | |
| zeta signaling | | AVTDELMQALNFNSETVPQKSSLEEPDFYKT | | |
| domain; stop) | | KIKLCILLHAFRIRAVTIDRVMSYLNAS* | | |
| (Encoded | | | | |
| Protein 2: p40 | | | | |
| signal | | | | |
| sequence; p40 | | | | |
| (23-328 of | | | | |
| WT); Linker | | | | |
| ((G4S)3); p35 | | | | |
| (57-253 of | | | | |
| WT); stop) | | | | |
| (OT-CD19- | | | | |
| 040) | | | | |
| OT-001407 | Full | MALPVTALLLPLALLLHAARPDIQMTQTTS | 810 | 835 |
| (CD8a leader- | Construct | SLSASLGDRVTISCRASQDISKYLNWYQQKP | | |
| CD19 scFV- | | DGTVKLLIYHTSRLHSGVPSRFSGSGSGTDY | | |
| CD8a-Tm- | | SLTISNLEQEDIATYFCQQGNTLPYTFGGGT | | |
| 41BB- | | KLEITGGGGSGGGGSGGGGSEVKLQESGPG | | |
| CD3zeta- | | LVAPSQSLSVTCTVSGVSLPDYGVSWIRQPP | | |
| stop) (OT- | | RKGLEWLGVIWGSETTYYNSALKSRLTIIKD | | |
| CD19-063) | | NSKSQVFLKMNSLQTDDTAIYYCAKHYYY | | |
| | | GGSYAMDYWGQGTSVTVSSTTTPAPRPPTP | | |
| | | APTIASQPLSLRPEACRPAAGGAVHTRGLDF | | |
| | | ACDIYIWAPLAGTCGVLLLSLVITLYCKRGR | | |
| | | KKLLYIFKQPFMRPVQTTQEEDGCSCRFPEE | | |
| | | EEGGCELRVKFSRSADAPAYKQGQNQLYN | | |
| | | ELNLGRREEYDVLDKRRGRDPEMGGKPRR | | |
| | | KNPQEGLYNELQKDKMAEAYSEIGMKGER | | |
| | | RRGKGHDGLYQGLSTATKDTYDALHMQAL | | |
| | | PPR* | | |
| OT-001356 | Full | — | — | 1070 |
| (Full | Construct | | | |
| Construct: | Encoded | MALPVTALLLPLALLLHAARPDIQMTQTTS | 1044 | 1071 |
| CD8a leader; | Protein 1 | SLSASLGDRVTISCRASQDISKYLNWYQQKP | | |
| CD 19 scFv; | | DGTVKLLIYHTSRLHSGVPSRFSGSGSGTDY | | |
| CD8a Hinge | | SLTISNLEQEDIATYFCQQGNTLPYTFGGGT | | |
| and | | KLEITGGGGSGGGGSGGGGSEVKLQESGPG | | |
| Transmembrane | | LVAPSQSLSVTCTVSGVSLPDYGVSWIRQPP | | |
| Domain; 4- | | RKGLEWLGVIWGSETTYYNSALKSRLTIIKD | | |
| 1BB | | NSKSQVFLKMNSLQTDDTAIYYCAKHYYY | | |
| intracellular | | GGSYAMDYWGQGTSVTVSSTTTPAPRPPTP | | |
| domain; CD3 | | APTIASQPLSLRPEACRPAAGGAVHTRGLDF | | |
| zeta signaling | | ACDIYIWAPLAGTCGVLLLSLVITLYCKRGR | | |
| domain; stop; | | KKLLYIFKQPFMRPVQTTQEEDGCSCRFPEE | | |
| spacer; IRES; | | EEGGCELRVKFSRSADAPAYKQGQNQLYN | | |
| spacer; p40 | | ELNLGRREEYDVLDKRRGRDPEMGGKPRR | | |
| signal | | KNPQEGLYNELQKDKMAEAYSEIGMKGER | | |
| sequence; p40 | | RRGKGHDGLYQGLSTATKDTYDALHMQAL | | |
| (23-328 of | | PPR* | | |
| WT); Linker | Encoded | MCHQQLVISWFSLVFLASPLVAIWELKKDV | 1072 | 1073 |
| ((G4S)3); p35 | Protein 2 | YVVELDWYPDAPGEMVVLTCDTPEEDGIT | | |
| (57-253 of | | WTLDQSSEVLGSGKTLTIQVKEFGDAGQYT | | |
| WT); BamHI | | CHKGGEVLSHSLLLLHKKEDGIWSTDILKD | | |
| (GS); stop) | | QKEPKNKTFLRCEAKNYSGRFTCWWLTTIS | | |
| (Encoded | | TDLTFSVKSSRGSSDPQGVTCGAATLSAER | | |
| Protein 1: | | VRGDNKEYEYSVECQEDSACPAAEESLPIE | | |
| CD8a leader; | | VMVDAVHKLKYENYTSSFFIRDIIKPDPPKN | | |
| CD 19 scFv; | | LQLKPLKNSRQVEVSWEYPDTWSTPHSYFS | | |
| CD8a Hinge | | LTFCVQVQGKSKREKKDRVFTDKTSATVIC | | |
| and | | RKNASISVRAQDRYYSSSWSEWASVPCSGG | | |
| Transmembrane | | GGSGGGGSGGGGSRNLPVATPDPGMFPCLH | | |
| Domain; 4- | | HSQNLLRAVSNMLQKARQTLEFYPCTSEEI | | |
| 1BB | | DHEDITKDKTSTVEACLPLELTKNESCLNSR | | |
| intracellular | | ETSFITNGSCLASRKTSFMMALCLSSIYEDL | | |
| domain; CD3 | | KMYQVEFKTMNAKLLMDPKRQIFLDQNML | | |
| zeta signaling | | AVIDELMQALNFNSETVPQKSSLEEPDFYKT | | |
| domain; stop) | | KIKLCILLHAFRIRAVTIDRVMSYLNASGS* | | |
| (Encoded | | | | |
| Protein 2: p40 | | | | |
| signal | | | | |
| sequence; p40 | | | | |
| (23-328 of | | | | |
| WT); Linker | | | | |
| ((G4S)3); p35 | | | | |
| (57-253 of | | | | |
| WT); BamHI | | | | |
| (GS); stop) | | | | |
| (OT-CD19- | | | | |
| IL12-009) | | | | |
| OT-001357 | Full | MALPVTALLLPLALLLHAARPDIQMTQTTS | 1074 | 1075 |
| (CD8a leader; | Construct | SLSASLGDRVTISCRASQDISKYLNWYQQKP | | |
| CD19 scFv; | | DGTVKLLIYHTSRLHSGVPSRFSGSGSGTDY | | |
| CD8a Hinge | | SLTISNLEQEDIATYFCQQGNTLPYTFGGGT | | |
| and | | KLEITGGGGSGGGGSGGGGSEVKLQESGPG | | |
| Transmembrane | | LVAPSQSLSVTCTVSGVSLPDYGVSWIRQPP | | |
| Domain; 4- | | RKGLEWLGVIWGSETTYYNSALKSRLTIIKD | | |
| 1BB | | NSKSQVFLKMNSLQTDDTAIYYCAKHYYY | | |
| intracellular | | GGSYAMDYWGQGTSVTVSSTTTPAPRPPTP | | |
| domain; CD3 | | APTIASQPLSLRPEACRPAAGGAVHTRGLDF | | |
| zeta signaling | | ACDIYIWAPLAGTCGVLLLSLVITLYCKRGR | | |
| domain; | | KKLLYIFKQPFMRPVQTTQEEDGCSCRFPEE | | |
| BamHI (GS); | | EEGGCELRVKFSRSADAPAYKQGQNQLYN | | |
| P2A Cleavage | | ELNLGRREEYDVLDKRRGRDPEMGGKPRR | | |
| Site; p40 | | KNPQEGLYNELQKDKMAEAYSEIGMKGER | | |
| signal | | RRGKGHDGLYQGLSTATKDTYDALHMQAL | | |
| sequence; p40 | | PPRGSATNFSLLKQAGDVEENPGPMCHQQL | | |
| (23-328 of | | VISWFSLVFLASPLVAIWELKKDVYVVELD | | |
| WT); Linker | | WYPDAPGEMVVLTCDTPEEDGITWTLDQSS | | |
| ((G4S)3); p35 | | EVLGSGKTLTIQVKEFGDAGQYTCHKGGEV | | |
| (57-253 of | | LSHSLLLLHKKEDGIWSTDILKDQKEPKNKT | | |
| WT); BamHI | | FLRCEAKNYSGRFTCWWLTTISTDLTFSVKS | | |
| (GS); stop) | | SRGSSDPQGVTCGAATLSAERVRGDNKEYE | | |
| (OT-CD19- | | YSVECQEDSACPAAEESLPIEVMVDAVHKL | | |
| IL12-010) | | KYENYTSSFFIRDIIKPDPPKNLQLKPLKNSR | | |
| | | QVEVSWEYPDTWSTPHSYFSLTFCVQVQGK | | |
| | | SKREKKDRVFTDKTSATVICRKNASISVRAQ | | |
| | | DRYYSSSWSEWASVPCSGGGGSGGGGSGG | | |
| | | GGSRNLPVATPDPGMFPCLHHSQNLLRAVS | | |
| | | NMLQKARQTLEFYPCTSEEIDHEDITKDKTS | | |
| | | TVEACLPLELTKNESCLNSRETSFITNGSCLA | | |
| | | SRKTSFMMALCLSSIYEDLKMYQVEFKTMN | | |
| | | AKLLMDPKRQIFLDQNMLAVIDELMQALNF | | |
| | | NSETVPQKSSLEEPDFYKTKIKLCILLHAFRI | | |
| | | RAVTIDRVMSYLNASGS* | | |
| OT-001386 | Full | — | — | 1079 |
| (Full | Construct | | | |
| Constract: | Encoded | MALPVTALLLPLALLLHAARPDIQMTQTTS | 1044 | 1071 |
| CD8a leader; | Protein 1 | SLSASLGDRVTISCRASQDISKYLNWYQQKP | | |
| CD19 scFv; | | DGTVKLLIYHTSRLHSGVPSRFSGSGSGTDY | | |
| CD8a Hinge | | SLTISNLEQEDIATYFCQQGNTLPYTFGGGT | | |
| and | | KLEITGGGGSGGGGSGGGGSEVKLQESGPG | | |
| Transmembrane | | LVAPSQSLSVTCTVSGVSLPDYGVSWIRQPP | | |
| Domain; 4- | | RKGLEWLGVIWGSETTYYNSALKSRLTIIKD | | |
| 1BB | | NSKSQVFLKMNSLQTDDTAIYYCAKHYYY | | |
| intracellular | | GGSYAMDYWGQGTSVTVSSTTTPAPRPPTP | | |
| domain; CD3 | | APTIASQPLSLRPEACRPAAGGAVHTRGLDF | | |
| zeta signaling | | ACDIYIWAPLAGTCGVLLLSLVITLYCKRGR | | |
| domain; stop; | | KKLLYIFKQPFMRPVQTTQEEDGCSCRFPEE | | |
| spacer; IRES; | | EEGGCELRVKFSRSADAPAYKQGQNQLYN | | |
| spacer; p40 | | ELNLGRREEYDVLDKRRGRDPEMGGKPRR | | |
| signal | | KNPQEGLYNELQKDKMAEAYSEIGMKGER | | |
| sequence; p40 | | RRGKGHDGLYQGLSTATKDTYDALHMQAL | | |
| (23-328 of | | PPR* | | |
| WT); Linker | Encoded | MCHQQLVISWFSLVFLASPLVAIWELKKDV | 1080 | 1081 |
| ((G4S)3); p35 | Protein 2 | YVVELDWYPDAPGEMVVLTCDTPEEDGIT | | |
| (57-253 of | | WTLDQSSEVLGSGKTLTIQVKEFGDAGQYT | | |
| WT); Linker | | CHKGGEVLSHSLLLLHKKEDGIWSTDILKD | | |
| (GGSG); | | QKEPKNKTFLRCEAKNYSGRFTCWWLTTIS | | |
| FKBP (M1del, | | TDLTFSVKSSRGSSDPQGVTCGAATLSAER | | |
| E32G, F37V, | | VRGDNKEYEYSVECQEDSACPAAEESLPIE | | |
| R72G, | | VMVDAVHKLKYENYTSSFFIRDIIKPDPPKN | | |
| K106E); stop) | | LQLKPLKNSRQVEVSWEYPDTWSTPHSYFS | | |
| (Encoded | | LTFCVQVQGKSKREKKDRVFTDKTSATVIC | | |
| Protein 1: | | RKNASISVRAQDRYYSSSWSEWASVPCSGG | | |
| CD8a leader; | | GGSGGGGSGGGGSRNLPVATPDPGMFPCLH | | |
| CD 19 scFv; | | HSQNLLRAVSNMLQKARQTLEFYPCTSEEI | | |
| CD8a Hinge | | DHEDITKDKTSTVEACLPLELTKNESCLNSR | | |
| and | | ETSFITNGSCLASRKTSFMMALCLSSIYEDL | | |
| Transmembrane | | KMYQVEFKTMNAKLLMDPKRQIFLDQNML | | |
| Domain; 4- | | AVTDELMQALNFNSETVPQKSSLEEPDFYKT | | |
| 1BB | | KIKLCILLHAFRIRAVTIDRVMSYLNASGGS | | |
| intracellular | | GGVQVETISPGDGRTFPKRGQTCVVHYTGM | | |
| domain; CD3 | | LGDGKKVDSSRDRNKPFKFMLGKQEVIRG | | |
| zeta signaling | | WEEGVAQMSVGQGAKLTISPDYAYGATGH | | |
| domain; stop) | | PGIIPPHATLVFDVELLELE* | | |
| (Encoded | | | | |
| Protein 2: p40 | | | | |
| signal | | | | |
| sequence; p40 | | | | |
| (23-328 of | | | | |
| WT); Linker | | | | |
| ((G4S)3); p35 | | | | |
| (57-253 of | | | | |
| WT); Linker | | | | |
| (GGSG); | | | | |
| FKBP (M1del, | | | | |
| E32G, F37V. | | | | |
| R72G, | | | | |
| K106E); stop) | | | | |
| (OT-CD19- | | | | |
| 1L12-011) | | | | |
| OT-001387 | Full | MALPVTALLLPLALLLHAARPDIQMTQTTS | 1084 | 1196 |
| (CD8a leader; | Construct | SLSASLGDRVTISCRASQDISKYLNWYQQKP | | |
| CD19 scFv; | | DGTVKLLIYHTSRLHSGVPSRFSGSGSGTDY | | |
| CD8a Hinge | | SLTISNLEQEDIATYFCQQGNTLPYTFGGGT | | |
| and | | KLEITGGGGSGGGGSGGGGSEVKLQESGPG | | |
| Transmembrane | | LVAPSQSLSVTCTVSGVSLPDYGVSWIRQPP | | |
| Domain; 4- | | RKGLEWLGVIWGSETTYYNSALKSRLTIIKD | | |
| 1BB | | NSKSQVFLKMNSLQTDDTAIYYCAKHYYY | | |
| intracellular | | GGSYAMDYWGQGTSVTVSSTTTPAPRPPTP | | |
| domain; CD3 | | APTIASQPLSLRPEACRPAAGGAVHTRGLDF | | |
| zeta signaling | | ACDIYIWAPLAGTCGVLLLSLVITLYCKRGR | | |
| domain; | | KKLLYIFKQPFMRPVQTTQEEDGCSCRFPEE | | |
| BamHI (GS); | | EEGGCELRVKFSRSADAPAYKQGQNQLYN | | |
| P2A Cleavage | | ELNLGRREEYDVLDKRRGRDPEMGGKPRR | | |
| Site; p40 | | KNPQEGLYNELQKDKMAEAYSEIGMKGER | | |
| signal | | RRGKGHDGLYQGLSTATKDTYDALHMQAL | | |
| sequence; p40 | | PPRGSATNFSLLKQAGDVEENPGPMCHQQL | | |
| (23-328 of | | VISWFSLVFLASPLVAIWELKKDVYVVELD | | |
| WT); Linker | | WYPDAPGEMVVLTCDTPEEDGITWTLDQSS | | |
| ((G4S)3); p35 | | EVLGSGKTLTIQVKEFGDAGQYTCHKGGEV | | |
| (57-253 of | | LSHSLLLLHKKEDGIWSTDILKDQKEPKNKT | | |
| WT); Linker | | FLRCEAKNYSGRFTCWWLTTISTDLTFSVKS | | |
| (GGSG) | | SRGSSDPQGVTCGAATLSAERVRGDNKEYE | | |
| FKBP (M1del, | | YSVECQEDSACPAAEESLPIEVMVDAVHKL | | |
| E32G, F37V, | | KYENYTSSFFIRDIIKPDPPKNLQLKPLKNSR | | |
| R72G, | | QVEVSWEYPDTWSTPHSYFSLTFCVQVQGK | | |
| K106E); stop) | | SKREKKDRVFTDKTSATVICRKNASISVRAQ | | |
| (OT-CD19- | | DRYYSSSWSEWASVPCSGGGGSGGGGSGG | | |
| IL12-013) | | GGSRNLPVATPDPGMFPCLHHSQNLLRAVS | | |
| | | NMLQKARQTLEFYPCTSEEIDHEDITKDKTS | | |
| | | TVEACLPLELTKNESCLNSRETSFITNGSCLA | | |
| | | SRKTSFMMALCLSSIYEDLKMYQVEFKTMN | | |
| | | AKLLMDPKRQIFLDQNMLAVIDELMQALNF | | |
| | | NSETVPQKSSLEEPDFYKTKIKLCILLHAFRI | | |
| | | RAVTIDRVMSYLNASGGSGGVQVETISPGD | | |
| | | GRTFPKRGQTCVVHYTGMLGDGKKVDSSR | | |
| | | DRNKPFKFMLGKQEVIRGWEEGVAQMSVG | | |
| | | QGAKLTISPDYAYGATGHPGIIPPHATLVFD | | |
| | | VELLELE* | | |
| OT-001618 | Full | — | — | 1143 |
| CD8 hinge | Construct | | | |
| and | Encoded | MALPVTALLLPLALLLHAARPDIQMTQTTS | 1120 | 1144 |
| transmembrane | Protein 1 | SLSASLGDRVTISCRASQDISKYLNWYQQKP | | |
| domain; 4- | | DGTVKLLIYHTSRLHSGVPSRFSGSGSGTDY | | |
| 1BB | | SLTISNLEQEDIATYFCQQGNTLPYTFGGGT | | |
| intracellular | | KLEITGGGGSGGGGSGGGGSEVKLQESGPG | | |
| signaling | | LVAPSQSLSVTCTVSGVSLPDYGVSWIRQPP | | |
| domain; CD3 | | RKGLEWLGVIWGSETTYYNSALKSRLTIIKD | | |
| zeta signaling | | NSKSQVFLKMNSLQTDDTAIYYCAKHYYY | | |
| domain; stop; | | GGSYAMDYWGQGTSVTVSSTTTPAPRPPTP | | |
| spacer | | APTIASQPLSLRPEACRPAAGGAVHTRGLDF | | |
| (ATCGGGCT | | ACDIYIWAPLAGTCGVLLLSLVITLYCKRGR | | |
| AGC); IRES; | | KKLLYIFKQPFMRPVQTTQEEDGCSCRFPEE | | |
| spacer | | EEGGCELRVKFSRSADAPAYKQGQNQLYN | | |
| (GCTTGCCA | | ELNLGRREEYDVLDKRRGRDPEMGGKPRR | | |
| CAACCCAC | | KNPQEGLYNELQKDKMAEAYSEIGMKGER | | |
| AAGGAGAC | | RRGKGHDGLYQGLSTATKDTYDALHMQAL | | |
| GACCTTCC); | | PPR* | | |
| p40 signal | Encoded | MCHQQLVISWFSLVFLASPLVAIWELKKDV | 1121 | 1143 |
| sequence; | Protein 2 | YVVELDWYPDAPGEMVVLTCDTPEEDGIT | | |
| 1L12B (p40) | | WTLDQSSEVLGSGKTLTIQVKEFGDAGQYT | | |
| (23-328 of | | CHKGGEVLSHSLLLLHKKEDGIWSTDILKD | | |
| WT); Linker | | QKEPKNKTFLRCEAKNYSGRFTCWWLTTIS | | |
| ((G4S)3); | | TDLTFSVKSSRGSSDPQGVTCGAATLSAER | | |
| Flexible G/S | | VRGDNKEYEYSVECQEDSACPAAEESLPIE | | |
| rich linker, | | VMVDAVHKLKYENYTSSFFIRDIIKPDPPKN | | |
| IL12A (p35) | | LQLKPLKNSRQVEVSWEYPDTWSTPHSYFS | | |
| (57-253 of | | LTFCVQVQGKSKREKKDRVFTDKTSATVIC | | |
| WT); | | RKNASISVRAQDRYYSSSWSEWASVPCSGG | | |
| Artificial | | GGSGGGGSGGGGSRNLPVATPDPGMFPCLH | | |
| hnker | | HSQNLLRAVSNMLQKARQTLEFYPCTSEEI | | |
| (GGSGG); | | DHEDITKDKTSTVEACLPLELTKNESCLNSR | | |
| hDHFR | | ETSFITNGSCLASRKTSFMMALCLSSIYEDL | | |
| (M1del, | | KMYQVEFKTMNAKLLMDPKRQIFLDQNML | | |
| Y122I, | | AVIDELMQALNFNSETVPQKSSLEEPDFYKT | | |
| N127Y); stop | | KIKLCILLHAFRIRAVTIDRVMSYLNASGGS | | |
| (OT-CD19- | | GGVGSLNCIVAVSQNMGIGKNGDLPWPPLR | | |
| IL12-019) | | NEFRYFQRMTTTSSVEGKQNLVIMGKKTW | | |
| | | FSIPEKNRPLKGRINLVLSRELKEPPQGAHFL | | |
| | | SRSLDDALKLTEQPELANKVDMVWIVGGSS | | |
| | | VIKEAMYHPGHLKLFVTRIMQDFESDTFFPE | | |
| | | IDLEKYKLLPEYPGVLSDVQEEKGIKYKFEV | | |
| | | YEKND* | | |
| OT-001617 | Full | — | — | 1146 |
| CD8 hinge | Construct | | | |
| and | Encoded | MALPVTALLLPLALLLHAARPDIQMTQTTS | 1122 | 1147 |
| transmembrane | Protein 1 | SLSASLGDRVTISCRASQDISKYLNWYQQKP | | |
| domain; 4- | | DGTVKLLIYHTSRLHSGVPSRFSGSGSGTDY | | |
| 1BB | | SLTISNLEQEDIATYFCQQGNTLPYTFGGGT | | |
| intracellular | | KLEITGGGGSGGGGSGGGGSEVKLQESGPG | | |
| signaling | | LVAPSQSLSVTCTVSGVSLPDYGVSWIRQPP | | |
| domain; CD3 | | RKGLEWLGVIWGSETTYYNSALKSRLTIIKD | | |
| zeta signaling | | NSKSQVFLKMNSLQTDDTAIYYCAKHYYY | | |
| domain; stop; | | GGSYAMDYWGQGTSVTVSSTTTPAPRPPTP | | |
| spacer | | APTIASQPLSLRPEACRPAAGGAVHTRGLDF | | |
| (ATCGGGCT | | ACDIYIWAPLAGTCGVLLLSLVITLYCKRGR | | |
| AGC); IRES; | | KKLLYIFKQPFMRPVQTTQEEDGCSCRFPEE | | |
| Spacer | | EEGGCELRVKFSRSADAPAYKQGQNQLYN | | |
| (gcttgccacaacc | | ELNLGRREEYDVLDKRRGRDPEMGGKPRR | | |
| cacaaggagacga | | KNPQEGLYNELQKDKMAEAYSEIGMKGER | | |
| ccttcc); p40 | | RRGKGHDGLYQGLSTATKDTYDALHMQAL | | |
| signal | | PPR* | | |
| sequence; | Encoded | MCHQQLVISWFSLVFLASPLVAIWELKKDV | 1123 | 1148 |
| IL12B (p40) | Protein 2 | YVVELDWYPDAPGEMVVLTCDTPEEDGIT | | |
| (23-328 of | | WTLDQSSEVLGSGKTLTIQVKEFGDAGQYT | | |
| WT); Linker | | CHKGGEVLSHSLLLLHKKEDGIWSTDILKD | | |
| ((G4S)3); | | QKEPKNKTFLRCEAKNYSGRFTCWWLTTIS | | |
| Flexible G/S | | TDLTFSVKSSRGSSDPQGVTCGAATLSAER | | |
| rich linker; | | VRGDNKEYEYSVECQEDSACPAAEESLPIE | | |
| IL12A (p35) | | VMVDAVHKLKYENYTSSFFIRDIIKPDPPKN | | |
| (57-253 of | | LQLKPLKNSRQVEVSWEYPDTWSTPHSYFS | | |
| WT); | | LTFCVQVQGKSKREKKDRVFTDKTSATVIC | | |
| Artificial | | RKNASISVRAQDRYYSSSWSEWASVPCSGG | | |
| linker | | GGSGGGGSGGGGSRNLPVATPDPGMFPCLH | | |
| (GSSG); | | HSQNLLRAVSNMLQKARQTLEFYPCTSEEI | | |
| ecDHFR | | DHEDITKDKTSTVEACLPLELTKNESCLNSR | | |
| (M1del, | | ETSFITNGSCLASRKTSFMMALCLSSIYEDL | | |
| RUY, | | KMYQVEFKTMNAKLLMDPKRQIFLDQNML | | |
| Y100I); stop) | | AVIDELMQALNFNSETVPQKSSLEEPDFYKT | | |
| (OT-CD19- | | KIKLCILLHAFRIRAVTIDRVMSYLNASGSS | | |
| IL12-026) | | GISLIAALAVDYVIGMENAMPWNLPADLA | | |
| (CD8a leader; | | WFKRNTLNKPVIMGRHTWESIGRPLPGRKN | | |
| CD19 scFv; | | IILSSQPGTDDRVTWVKSVDEAIAACGDVPE | | |
| | | IMVIGGGRVIEQFLPKAQKLYLTHIDAEVEG | | |
| | | DTHFPDYEPDDWESVFSEFHDADAQNSHSY | | |
| | | CFEILERR* | | |
| OT-001622 | Full | MALPVTALLLPLALLLHAARPDIQMTQTTS | 1124 | 1149 |
| (CD8a leader; | Construct | SLSASLGDRVTISCRASQDISKYLNWYQQKP | | |
| CD19 scFv; | | DGTVKLLIYHTSRLHSGVPSRFSGSGSGTDY | | |
| CD8 hinge | | SLTISNLEQEDIATYFCQQGNTLPYTFGGGT | | |
| and | | KLEITGGGGSGGGGSGGGGSEVKLQESGPG | | |
| transmembrane | | LVAPSQSLSVTCTVSGVSLPDYGVSWIRQPP | | |
| Domain; 4- | | RKGLEWLGVIWGSETTYYNSALKSRLTIIKD | | |
| 1BB | | NSKSQVFLKMNSLQTDDTAIYYCAKHYYY | | |
| intracellular | | GGSYAMDYWGQGTSVTVSSTTTPAPRPPTP | | |
| signaling | | APTIASQPLSLRPEACRPAAGGAVHTRGLDF | | |
| domain; CD3 | | ACDIYIWAPLAGTCGVLLLSLVITLYCKRGR | | |
| zeta signaling | | KKLLYIFKQPFMRPVQTTQEEDGCSCRFPEE | | |
| domain; | | EEGGCELRVKFSRSADAPAYKQGQNQLYN | | |
| Flexible G/S | | ELNLGRREEYDVLDKRRGRDPEMGGKPRR | | |
| rich linker; | | KNPQEGLYNELQKDKMAEAYSEIGMKGER | | |
| BamHI Site; | | RRGKGHDGLYQGLSTATKDTYDALHMQAL | | |
| P2A cleavage | | PPRGSATNFSLLKQAGDVEENPGPMCHQQL | | |
| site; p40 | | VISWFSLVFLASPLVAIWELKKDVYVVELD | | |
| signal | | WYPDAPGEMVVLTCDTPEEDGITWTLDQSS | | |
| sequence; | | EVLGSGKTLTIQVKEFGDAGQYTCHKGGEV | | |
| IL12B (p40) | | LSHSLLLLHKKEDGIWSTDILKDQKEPKNKT | | |
| (23-328 of | | FLRCEAKNYSGRFTCWWLTTISTDLTFSVKS | | |
| WT); Linker | | SRGSSDPQGVTCGAATLSAERVRGDNKEYE | | |
| ((G4S)3); | | YSVECQEDSACPAAEESLPIEVMVDAVHKL | | |
| Flexible G/S | | KYENYTSSFFIRDIIKPDPPKNLQLKPLKNSR | | |
| rich linker; | | QVEVSWEYPDTWSTPHSYFSLTFCVQVQGK | | |
| IL12A (p35) | | SKREKKDRVFTDKTSATVICRKNASISVRAQ | | |
| (57-253 of | | DRYYSSSWSEWASVPCSGGGGSGGGGSGG | | |
| WT); | | GGSRNLPVATPDPGMFPCLHHSQNLLRAVS | | |
| Artificial | | NMLQKARQTLEFYPCTSEEIDHEDITKDKTS | | |
| linker | | TVEACLPLELTKNESCLNSRETSFITNGSCLA | | |
| (GSSG); | | SRKTSFMMALCLSSIYEDLKMYQVEFKTMN | | |
| ecDHFR | | AKLLMDPKRQIFLDQNMLAVIDELMQALNF | | |
| (M1del, | | NSETVPQKSSLEEPDFYKTKIKLCILLHAFRI | | |
| RI2Y. | | RAVTIDRVMSYLNASGSSGISLIAALAVDYV | | |
| Y100I); stop) | | IGMENAMPWNLPADLAWFKRNTLNKPVIM | | |
| (OT-CD19- | | GRHTWESIGRPLPGRKNIILSSQPGTDDRVT | | |
| IL12-040) | | WVKSVDEAIAACGDVPEIMVIGGGRVIEQF | | |
| | | LPKAQKLYLTHIDAEVEGDTHFPDYEPDDW | | |
| | | ESVFSEFHDADAQNSHSYCFEILERR* | | |
| OT-001619 | Full | MALPVTALLLPLALLLHAARPDIQMTQTTS | 1125 | 1150 |
| (CD8a leader; | Construct | SLSASLGDRVTISCRASQDISKYLNWYQQKP | | |
| CD19 scFv; | | DGTVKLLIYHTSRLHSGVPSRFSGSGSGTDY | | |
| CD8 hinge | | SLTISNLEQEDIATYFCQQGNTLPYTFGGGT | | |
| and | | KLEITGGGGSGGGGSGGGGSEVKLQESGPG | | |
| transmembrane | | LVAPSQSLSVTCTVSGVSLPDYGVSWIRQPP | | |
| Domain; 4- | | RKGLEWLGVIWGSETTYYNSALKSRLTIIKD | | |
| 1BB | | NSKSQVFLKMNSLQTDDTAIYYCAKHYYY | | |
| intracellular | | GGSYAMDYWGQGTSVTVSSTTTPAPRPPTP | | |
| signaling | | APTIASQPLSLRPEACRPAAGGAVHTRGLDF | | |
| domain; CD3 | | ACDIYIWAPLAGTCGVLLLSLVITLYCKRGR | | |
| zeta signaling | | KKLLYIFKQPFMRPVQTTQEEDGCSCRFPEE | | |
| domain; | | EEGGCELRVKFSRSADAPAYKQGQNQLYN | | |
| Linker (GS); | | ELNLGRREEYDVLDKRRGRDPEMGGKPRR | | |
| BamHI Site; | | KNPQEGLYNELQKDKMAEAYSEIGMKGER | | |
| P2A cleavage | | RRGKGHDGLYQGLSTATKDTYDALHMQAL | | |
| site; p40 | | PPRGSATNFSLLKQAGDVEENPGPMCHQQL | | |
| signal | | VISWFSLVFLASPLVAIWELKKDVYVVELD | | |
| sequence; | | WYPDAPGEMVVLTCDTPEEDGITWTLDQSS | | |
| IL12B (p40) | | EVLGSGKTLTIQVKEFGDAGQYTCHKGGEV | | |
| (23-328 of | | LSHSLLLLHKKEDGIWSTDILKDQKEPKNKT | | |
| WT); Linker | | FLRCEAKNYSGRFTCWWLTTISTDLTFSVKS | | |
| ((G4S)3); | | SRGSSDPQGVTCGAATLSAERVRGDNKEYE | | |
| Flexible G/S | | YSVECQEDSACPAAEESLPIEVMVDAVHKL | | |
| rich linker; | | KYENYTSSFFIRDIIKPDPPKNLQLKPLKNSR | | |
| IL12A (p35) | | QVEVSWEYPDTWSTPHSYFSLTFCVQVQGK | | |
| (57-253 of | | SKREKKDRVFTDKTSATVICRKNASISVRAQ | | |
| WT); Linker | | DRYYSSSWSEWASVPCSGGGGSGGGGSGG | | |
| (GS); BamHI | | GGSRNLPVATPDPGMFPCLHHSQNLLRAVS | | |
| Site; hDHFR | | NMLQKARQTLEFYPCTSEEIDHEDITKDKTS | | |
| (M1del, | | TVEACLPLELTKNESCLNSRETSFITNGSCLA | | |
| I17V); stop) | | SRKTSFMMALCLSSIYEDLKMYQVEFKTMN | | |
| (OT-CD19- | | AKLLMDPKRQIFLDQNMLAVIDELMQALNF | | |
| IL12-036) | | NSETVPQKSSLEEPDFYKTKIKLCILLHAFRI | | |
| | | RAVTIDRVMSYLNASGSVGSLNCIVAVSQN | | |
| | | MGVGKNGDLPWPPLRNEFRYFQRMTTTSS | | |
| | | VEGKQNLVIMGKKTWFSIPEKNRPLKGRIN | | |
| | | LVLSRELKEPPQGAHFLSRSLDDALKLTEQP | | |
| | | ELANKVDMVWIVGGSSVYKEAMNHPGHL | | |
| | | KLFVTRIMQDFESDTFFPEIDLEKYKLLPEYP | | |
| | | GVLSDVQEEKGIKYKFEVYEKND* | | |
| OT-001620 | Full | MALPVTALLLPLALLLHAARPDIQMTQTTS | 1126 | 1151 |
| (CD8a leader; | Construct | SLSASLGDRVTISCRASQDISKYLNWYQQKP | | |
| CD19 scFv; | | DGTVKLLIYHTSRLHSGVPSRFSGSGSGTDY | | |
| CD8 hinge | | SLTISNLEQEDIATYFCQQGNTLPYTFGGGT | | |
| and | | KLEITGGGGSGGGGSGGGGSEVKLQESGPG | | |
| transmembrane | | LVAPSQSLSVTCTVSGVSLPDYGVSWIRQPP | | |
| Domain; 4- | | RKGLEWLGVIWGSETTYYNSALKSRLTIIKD | | |
| 1BB | | NSKSQVFLKMNSLQTDDTAIYYCAKHYYY | | |
| intracellular | | GGSYAMDYWGQGTSVTVSSTTTPAPRPPTP | | |
| signaling | | APTIASQPLSLRPEACRPAAGGAVHTRGLDF | | |
| domain; CD3 | | ACDIYIWAPLAGTCGVLLLSLVITLYCKRGR | | |
| zeta signaling | | KKLLYIFKQPFMRPVQTTQEEDGCSCRFPEE | | |
| domain; | | EEGGCELRVKFSRSADAPAYKQGQNQLYN | | |
| Linker (GS); | | ELNLGRREEYDVLDKRRGRDPEMGGKPRR | | |
| BamHI Site; | | KNPQEGLYNELQKDKMAEAYSEIGMKGER | | |
| P2A cleavage | | RRGKGHDGLYQGLSTATKDTYDALHMQAL | | |
| site; p40 | | PPRGSATNFSLLKQAGDVEENPGPMCHQQL | | |
| signal | | VISWFSLVFLASPLVAIWELKKDVYVVELD | | |
| sequence; | | WYPDAPGEMVVLTCDTPEEDGITWTLDQSS | | |
| IL12B (p40) | | EVLGSGKTLTIQVKEFGDAGQYTCHKGGEV | | |
| (23-328 of | | LSHSLLLLHKKEDGIWSTDILKDQKEPKNKT | | |
| WT); Linker | | FLRCEAKNYSGRFTCWWLTTISTDLTFSVKS | | |
| ((G4S)3); | | SRGSSDPQGVTCGAATLSAERVRGDNKEYE | | |
| Flexible G/S | | YSVECQEDSACPAAEESLPIEVMVDAVHKL | | |
| rich linker; | | KYENYTSSFFIRDIIKPDPPKNLQLKPLKNSR | | |
| IL12A (p35) | | QVEVSWEYPDTWSTPHSYFSLTFCVQVQGK | | |
| (57-253 of | | SKREKKDRVFTDKTSATVICRKNASISVRAQ | | |
| WT); Linker | | DRYYSSSWSEWASVPCSGGGGSGGGGSGG | | |
| (GS); BamHI | | GGSRNLPVATPDPGMFPCLHHSQNLLRAVS | | |
| Site; hDHFR | | NMLQKARQTLEFYPCTSEEIDHEDITKDKTS | | |
| (M1del, I17V, | | TVEACLPLELTKNESCLNSRETSFITNGSCLA | | |
| Y122I); stop) | | SRKTSFMMALCLSSIYEDLKMYQVEFKTMN | | |
| (OT-CD19- | | AKLLMDPKRQIFLDQNMLAVIDELMQALNF | | |
| IL12-037) | | NSETVPQKSSLEEPDFYKTKIKLCILLHAFRI | | |
| | | RAVTIDRVMSYLNASGSVGSLNCIVAVSQN | | |
| | | MGVGKNGDLPWPPLRNEFRYFQRMTTTSS | | |
| | | VEGKQNLVIMGKKTWFSIPEKNRPLKGRIN | | |
| | | LVLSRELKEPPQGAHFLSRSLDDALKLTEQP | | |
| | | ELANKVDMVWIVGGSSVIKEAMNHPGHLK | | |
| | | LFVTRIMQDFESDTFFPEIDLEKYKLLPEYPG | | |
| | | VLSDVQEEKGIKYKFEVYEKND* | | |
| OT-001621 | Full | MALPVTALLLPLALLLHAARPDIQMTQTTS | 1127 | 1152 |
| (CD8a leader; | Construct | SLSASLGDRVTISCRASQDISKYLNWYQQKP | | |
| CD19 scFv; | | DGTVKLLIYHTSRLHSGVPSRFSGSGSGTDY | | |
| CD8 hinge | | SLTISNLEQEDIATYFCQQGNTLPYTFGGGT | | |
| and | | KLEITGGGGSGGGGSGGGGSEVKLQESGPG | | |
| transmembrane | | LVAPSQSLSVTCTVSGVSLPDYGVSWIRQPP | | |
| Domain; 4- | | RKGLEWLGVIWGSETTYYNSALKSRLTIIKD | | |
| 1BB | | NSKSQVFLKMNSLQTDDTAIYYCAKHYYY | | |
| intracellular | | GGSYAMDYWGQGTSVTVSSTTTPAPRPPTP | | |
| signaling | | APTIASQPLSLRPEACRPAAGGAVHTRGLDF | | |
| domain; CD3 | | ACDIYIWAPLAGTCGVLLLSLVITLYCKRGR | | |
| zeta signaling | | KKLLYIFKQPFMRPVQTTQEEDGCSCRFPEE | | |
| domain; | | EEGGCELRVKFSRSADAPAYKQGQNQLYN | | |
| Linker (GS); | | ELNLGRREEYDVLDKRRGRDPEMGGKPRR | | |
| BamHI Site; | | KNPQEGLYNELQKDKMAEAYSEIGMKGER | | |
| P2A cleavage | | RRGKGHDGLYQGLSTATKDTYDALHMQAL | | |
| site; p40 | | PPRGSATNFSLLKQAGDVEENPGPMCHQQL | | |
| signal | | VISWFSLVFLASPLVAIWELKKDVYVVELD | | |
| sequence; | | WYPDAPGEMVVLTCDTPEEDGITWTLDQSS | | |
| IL12B (p40) | | EVLGSGKTLTIQVKEFGDAGQYTCHKGGEV | | |
| (23-328 of | | LSHSLLLLHKKEDGIWSTDILKDQKEPKNKT | | |
| WT); Linker | | FLRCEAKNYSGRFTCWWLTTISTDLTFSVKS | | |
| ((G4S)3); | | SRGSSDPQGVTCGAATLSAERVRGDNKEYE | | |
| Flexible G/S | | YSVECQEDSACPAAEESLPIEVMVDAVHKL | | |
| rich linker; | | KYENYTSSFFIRDIIKPDPPKNLQLKPLKNSR | | |
| IL12A (p35) | | QVEVSWEYPDTWSTPHSYFSLTFCVQVQGK | | |
| (57-253 of | | SKREKKDRVFTDKTSATVICRKNASISVRAQ | | |
| WT); Linker | | DRYYSSSWSEWASVPCSGGGGSGGGGSGG | | |
| (GS); BamHI | | GGSRNLPVATPDPGMFPCLHHSQNLLRAVS | | |
| Site; hDHFR | | NMLQKARQTLEFYPCTSEEIDHEDITKDKTS | | |
| (M1del, | | TVEACLPLELTKNESCLNSRETSFITNGSCLA | | |
| Y122I, | | SRKTSFMMALCLSSIYEDLKMYQVEFKTMN | | |
| N127Y); stop) | | AKLLMDPKRQIFLDQNMLAVIDELMQALNF | | |
| (OT-CD19- | | NSETVPQKSSLEEPDFYKTKIKLCILLHAFRI | | |
| IL12-038) | | RAVTIDRVMSYLNASGSVGSLNCIVAVSQN | | |
| | | MGIGKNGDLPWPPLRNEFRYFQRMTTTSSV | | |
| | | EGKQNLVIMGKKTWFSIPEKNRPLKGRINL | | |
| | | VLSRELKEPPQGAHFLSRSLDDALKLTEQPE | | |
| | | LANKVDMVWIVGGSSVIKEAMYHPGHLKL | | |
| | | FVTRIMQDFESDTFFPEIDLEKYKLLPEYPG | | |
| | | VLSDVQEEKGIKYKFEVYEKND* | | |
| OT-001612 | Full | — | — | 1153 |
| (CD8a leader; | Construct | | | |
| CD19 scFv: | Encoded | MALPVTALLLPLALLLHAARPDIQMTQTTS | 1132 | 1154 |
| CD8 hinge | Protein 1 | SLSASLGDRVTISCRASQDISKYLNWYQQKP | | |
| and | | DGTVKLLIYHTSRLHSGVPSRFSGSGSGTDY | | |
| transmembrane | | SLTISNLEQEDIATYFCQQGNTLPYTFGGGT | | |
| Domain; 4- | | KLEITGGGGSGGGGSGGGGSEVKLQESGPG | | |
| 1BB | | LVAPSQSLSVTCTVSGVSLPDYGVSWIRQPP | | |
| intracellular | | RKGLEWLGVIWGSETTYYNSALKSRLTIIKD | | |
| signaling | | NSKSQVFLKMNSLQTDDTAIYYCAKHYYY | | |
| domain; CD3 | | GGSYAMDYWGQGTSVTVSSTTTPAPRPPTP | | |
| zeta signaling | | APTIASQPLSLRPEACRPAAGGAVHTRGLDF | | |
| domain; stop; | | ACDIYIWAPLAGTCGVLLLSLVITLYCKRGR | | |
| spacer | | KKLLYIFKQPFMRPVQTTQEEDGCSCRFPEE | | |
| (ATCGGGCT | | EEGGCELRVKFSRSADAPAYKQGQNQLYN | | |
| AGC); IRES; | | ELNLGRREEYDVLDKRRGRDPEMGGKPRR | | |
| Spacer | | KNPQEGLYNELQKDKMAEAYSEIGMKGER | | |
| (gcttgccacaacc | | RRGKGHDGLYQGLSTATKDTYDALHMQAL | | |
| cacaaggagacga | | PPR* | | |
| ccttcc); p40 | Encoded | MCHQQLVISWFSLVFLASPLVAIWELKKDV | 1133 | 1155 |
| signal | Protein 2 | YVVELDWYPDAPGEMVVLTCDTPEEDGIT | | |
| sequence; | | WTLDQSSEVLGSGKTLTIQVKEFGDAGQYT | | |
| IL12B (p40) | | CHKGGEVLSHSLLLLHKKEDGIWSTDILKD | | |
| (23-328 of | | QKEPKNKTFLRCEAKNYSGRFTCWWLTTIS | | |
| WT); Linker | | TDLTFSVKSSRGSSDPQGVTCGAATLSAER | | |
| ((G4S)3); | | VRGDNKEYEYSVECQEDSACPAAEESLPIE | | |
| Flexible G/S | | VMVDAVHKLKYENYTSSFFIRDIIKPDPPKN | | |
| rich linker, | | LQLKPLKNSRQVEVSWEYPDTWSTPHSYFS | | |
| IL12A (p35) | | LTFCVQVQGKSKREKKDRVFTDKTSATVIC | | |
| (57-253 of | | RKNASISVRAQDRYYSSSWSEWASVPCSGG | | |
| WT); | | GGSGGGGSGGGGSRNLPVATPDPGMFPCLH | | |
| Artificial | | HSQNLLRAVSNMLQKARQTLEFYPCTSEEI | | |
| linker | | DHEDITKDKTSTVEACLPLELTKNESCLNSR | | |
| (GGSGG); | | ETSFITNGSCLASRKTSFMMALCLSSIYEDL | | |
| hDHFR | | KMYQVEFKTMNAKLLMDPKRQIFLDQNML | | |
| (M1del, | | AVTDELMQALNFNSETVPQKSSLEEPDFYKT | | |
| I17V); | | KIKLCILLHAFRIRAVTIDRVMSYLNASGGS | | |
| stop)(OT- | | GGVGSLNCIVAVSQNMGVGKNGDLPWPPL | | |
| CD19-IL12- | | RNEFRYFQRMTTTSSVEGKQNLVIMGKKT | | |
| 014) | | WFSIPEKNRPLKGRINLVLSRELKEPPQGAH | | |
| | | FLSRSLDDALKLTEQPELANKVDMVWIVGG | | |
| | | SSVYKEAMNHPGHLKLFVTRIMQDFESDTF | | |
| | | FPEIDLEKYKLLPEYPGVLSDVQEEKGIKYK | | |
| | | FEVYEKND* | | |
| OT-001613 | Full | — | — | 1156 |
| (CD8a leader; | Construct | | | |
| CD 19 scFv; | Encoded | MALPVTALLLPLALLLHAARPDIQMTQTTS | 1134 | 1157 |
| CDS hinge | Protein 1 | SLSASLGDRVTISCRASQDISKYLNWYQQKP | | |
| and | | DGTVKLLIYHTSRLHSGVPSRFSGSGSGTDY | | |
| transmembrane | | SLTISNLEQEDIATYFCQQGNTLPYTFGGGT | | |
| domain; 4- | | KLEITGGGGSGGGGSGGGGSEVKLQESGPG | | |
| 1BB | | LVAPSQSLSVTCTVSGVSLPDYGVSWIRQPP | | |
| intracellular | | RKGLEWLGVIWGSETTYYNSALKSRLTIIKD | | |
| signaling | | NSKSQVFLKMNSLQTDDTAIYYCAKHYYY | | |
| domain; CD3 | | GGSYAMDYWGQGTSVTVSSTTTPAPRPPTP | | |
| zeta signaling | | APTIASQPLSLRPEACRPAAGGAVHTRGLDF | | |
| domain; stop; | | ACDIYIWAPLAGTCGVLLLSLVITLYCKRGR | | |
| spacer | | KKLLYIFKQPFMRPVQTTQEEDGCSCRFPEE | | |
| (ATCGGGCT | | EEGGCELRVKFSRSADAPAYKQGQNQLYN | | |
| AGC); IRES; | | ELNLGRREEYDVLDKRRGRDPEMGGKPRR | | |
| Spacer | | KNPQEGLYNELQKDKMAEAYSEIGMKGER | | |
| (gcttgccacaacc | | RRGKGHDGLYQGLSTATKDTYDALHMQAL | | |
| cacaaggagacga | | PPR* | | |
| ccttcc); p40 | Encoded | MCHQQLVISWFSLVFLASPLVAIWELKKDV | 1135 | 1158 |
| signal | Protein 2 | YVVELDWYPDAPGEMVVLTCDTPEEDGIT | | |
| sequence; | | WTLDQSSEVLGSGKTLTIQVKEFGDAGQYT | | |
| IL12B (p40) | | CHKGGEVLSHSLLLLHKKEDGIWSTDILKD | | |
| (23-328 of | | QKEPKNKTFLRCEAKNYSGRFTCWWLTTIS | | |
| WT); Linker | | TDLTFSVKSSRGSSDPQGVTCGAATLSAER | | |
| Artificial | | VRGDNKEYEYSVECQEDSACPAAEESLPIE | | |
| linker | | VMVDAVHKLKYENYTSSFFIRDIIKPDPPKN | | |
| (GGSGG); | | LQLKPLKNSRQVEVSWEYPDTWSTPHSYFS | | |
| hDHFR | | LTFCVQVQGKSKREKKDRVFTDKTSATVIC | | |
| (M1del, | | RKNASISVRAQDRYYSSSWSEWASVPCSGG | | |
| Y122I); | | GGSGGGGSGGGGSRNLPVATPDPGMFPCLH | | |
| stop) (OT- | | HSQNLLRAVSNMLQKARQTLEFYPCTSEEI | | |
| CD19-IL12- | | DHEDITKDKTSTVEACLPLELTKNESCLNSR | | |
| 015) | | ETSFITNGSCLASRKTSFMMALCLSSIYEDL | | |
| | | KMYQVEFKTMNAKLLMDPKRQIFLDQNML | | |
| | | AVIDELMQALNFNSETVPQKSSLEEPDFYKT | | |
| | | KKLCILLHAFRIRAVTIDRVMSYLNASGGS | | |
| | | GGVGSLNCIVAVSQNMGIGKNGDLPWPPLR | | |
| | | NEFRYFQRMTTTSSVEGKQNLVIMGKKTW | | |
| | | FSIPEKNRPLKGRINLVLSRELKEPPQGAHFL | | |
| | | SRSLDDALKLTEQPELANKVDMVWIVGGSS | | |
| | | VKEAMNHPGHLKLFVTRIMQDFESDTFFPE | | |
| | | IDLEKYKLLPEYPGVLSDVQEEKGIKYKFEV | | |
| | | YEKND* | | |
| OT-001614 | Full | — | — | 1159 |
| (CD8a leader; | Construct | | | |
| CD 19 scFv; | Encoded | MALPVTALLLPLALLLHAARPDIQMTQTTS | 1136 | 1160 |
| CD8 liinge | Protein 1 | SLSASLGDRVTISCRASQDISKYLNWYQQKP | | |
| and | | DGTVKLLIYHTSRLHSGVPSRFSGSGSGTDY | | |
| transmembrane | | SLTISNLEQEDIATYFCQQGNTLPYTFGGGT | | |
| domain; 4- | | KLEITGGGGSGGGGSGGGGSEVKLQESGPG | | |
| 1BB | | LVAPSQSLSVTCTVSGVSLPDYGVSWIRQPP | | |
| intracellular | | RKGLEWLGVIWGSETTYYNSALKSRLTIIKD | | |
| signaling | | NSKSQVFLKMNSLQTDDTAIYYCAKHYYY | | |
| domain; CD3 | | GGSYAMDYWGQGTSVTVSSTTTPAPRPPTP | | |
| zeta signaling | | APTIASQPLSLRPEACRPAAGGAVHTRGLDF | | |
| domain; stop; | | ACDIYIWAPLAGTCGVLLLSLVITLYCKRGR | | |
| spacer | | KKLLYIFKQPFMRPVQTTQEEDGCSCRFPEE | | |
| (ATCGGGCT | | EEGGCELRVKFSRSADAPAYKQGQNQLYN | | |
| AGC); IRES; | | ELNLGRREEYDVLDKRRGRDPEMGGKPRR | | |
| Spacer | | KNPQEGLYNELQKDKMAEAYSEIGMKGER | | |
| (gcttgccacaacc | | RRGKGHDGLYQGLSTATKDTYDALHMQAL | | |
| cacaaggagacga | | PPR* | | |
| ccttcc); p40 | Encoded | MCHQQLVISWFSLVFLASPLVAIWELKKDV | 1137 | 1161 |
| signal | Protein 2 | YVVELDWYPDAPGEMVVLTCDTPEEDGIT | | |
| sequence; | | WTLDQSSEVLGSGKTLTIQVKEFGDAGQYT | | |
| IL12B (p40) | | CHKGGEVLSHSLLLLHKKEDGIWSTDILKD | | |
| (23-328 of | | QKEPKNKTFLRCEAKNYSGRFTCWWLTTIS | | |
| WT); Linker | | TDLTFSVKSSRGSSDPQGVTCGAATLSAER | | |
| ((G4S)3); | | VRGDNKEYEYSVECQEDSACPAAEESLPIE | | |
| Flexible G/S | | VMVDAVHKLKYENYTSSFFIRDIIKPDPPKN | | |
| rich linker, | | LQLKPLKNSRQVEVSWEYPDTWSTPHSYFS | | |
| 1L12A (p35) | | LTFCVQVQGKSKREKKDRVFTDKTSATVIC | | |
| (57-253 of | | RKNASISVRAQDRYYSSSWSEWASVPCSGG | | |
| WT); | | GGSGGGGSGGGGSRNLPVATPDPGMFPCLH | | |
| Artificial | | HSQNLLRAVSNMLQKARQTLEFYPCTSEEI | | |
| linker | | DHEDITKDKTSTVEACLPLELTKNESCLNSR | | |
| (GGSGG); | | ETSFITNGSCLASRKTSFMMALCLSSIYEDL | | |
| hDHFR | | KMYQVEFKTMNAKLLMDPKRQIFLDQNML | | |
| (M1del, | | AVIDELMQALNFNSETVPQKSSLEEPDFYKT | | |
| I17A); stop) | | KKLCILLHAFRIRAVTIDRVMSYLNASGGS | | |
| (OT-CD19- | | GGVGSLNCIVAVSQNMGAGKNGDLPWPPL | | |
| 1L12-016) | | RNEFRYFQRMTTTSSVEGKQNLVIMGKKT | | |
| | | WFSIPEKNRPLKGRINLVLSRELKEPPQGAH | | |
| | | FLSRSLDDALKLTEQPELANKVDMVWIVGG | | |
| | | SSVYKEAMNHPGHLKLFVTRIMQDFESDTF | | |
| | | FPEIDLEKYKLLPEYPGVLSDVQEEKGIKYK | | |
| | | FEVYEKND* | | |
| OT-001615 | Full | — | — | 1162 |
| (CD8a leader; | Construct | | | |
| CD 19 scFv; | | | | |
| CD8 liinge | Encoded | MALPVTALLLPLALLLHAARPDIQMTQTTS | 1138 | 1163 |
| and | Protein 1 | SLSASLGDRVTISCRASQDISKYLNWYQQKP | | |
| Transmembrane | | DGTVKLLIYHTSRLHSGVPSRFSGSGSGTDY | | |
| Domain; 4- | | SLTISNLEQEDIATYFCQQGNTLPYTFGGGT | | |
| 1BB | | KLEITGGGGSGGGGSGGGGSEVKLQESGPG | | |
| intracellular | | LVAPSQSLSVTCTVSGVSLPDYGVSWIRQPP | | |
| signaling | | RKGLEWLGVIWGSETTYYNSALKSRLTIIKD | | |
| domain; CD3 | | NSKSQVFLKMNSLQTDDTAIYYCAKHYYY | | |
| zeta signaling | | GGSYAMDYWGQGTSVTVSSTTTPAPRPPTP | | |
| domain; stop; | | APTIASQPLSLRPEACRPAAGGAVHTRGLDF | | |
| spacer | | ACDIYIWAPLAGTCGVLLLSLVITLYCKRGR | | |
| (ATCGGGCT | | KKLLYIFKQPFMRPVQTTQEEDGCSCRFPEE | | |
| AGC); IRES; | | EEGGCELRVKFSRSADAPAYKQGQNQLYN | | |
| Spacer | | ELNLGRREEYDVLDKRRGRDPEMGGKPRR | | |
| (gcttgccacaacc | | KNPQEGLYNELQKDKMAEAYSEIGMKGER | | |
| cacaaggagacga | | RRGKGHDGLYQGLSTATKDTYDALHMQAL | | |
| ccttcc); p40 | | PPR* | | |
| signal | Encoded | MCHQQLVISWFSLVFLASPLVAIWELKKDV | 1139 | 1164 |
| sequence; | Protein 2 | YVVELDWYPDAPGEMVVLTCDTPEEDGIT | | |
| IL12B (p40) | | WTLDQSSEVLGSGKTLTIQVKEFGDAGQYT | | |
| (23-328 of | | CHKGGEVLSHSLLLLHKKEDGIWSTDILKD | | |
| WT); Linker | | QKEPKNKTFLRCEAKNYSGRFTCWWLTTIS | | |
| ((G4S)3); | | TDLTFSVKSSRGSSDPQGVTCGAATLSAER | | |
| Flexible G/S | | VRGDNKEYEYSVECQEDSACPAAEESLPIE | | |
| rich linker, | | VMVDAVHKLKYENYTSSFFIRDIIKPDPPKN | | |
| IL12A (p35) | | LQLKPLKNSRQVEVSWEYPDTWSTPHSYFS | | |
| (57-253 of | | LTFCVQVQGKSKREKKDRVFTDKTSATVIC | | |
| WT); | | RKNASISVRAQDRYYSSSWSEWASVPCSGG | | |
| Artificial | | GGSGGGGSGGGGSRNLPVATPDPGMFPCLH | | |
| linker | | HSQNLLRAVSNMLQKARQTLEFYPCTSEEI | | |
| (GGSGG); | | DHEDITKDKTSTVEACLPLELTKNESCLNSR | | |
| hDHFR | | ETSFITNGSCLASRKTSFMMALCLSSIYEDL | | |
| (M1del, 117V, | | KMYQVEFKTMNAKLLMDPKRQIFLDQNML | | |
| Y1221); stop) | | AVIDELMQALNFNSETVPQKSSLEEPDFYKT | | |
| | | KIKLCILLHAFRIRAVTIDRVMSYLNASGGS | | |
| | | GGVGSLNCIVAVSQNMGVGKNGDLPWPPL | | |
| | | RNEFRYFQRMTTTSSVEGKQNLVIMGKKT | | |
| | | WFSIPEKNRPLKGRINLVLSRELKEPPQGAH | | |
| | | FLSRSLDDALKLTEQPELANKVDMVWIVGG | | |
| | | SSVIKEAMNHPGHLKLFVTRIMQDFESDTFF | | |
| | | PEIDLEKYKLLPEYPGVLSDVQEEKGIKYKF | | |
| | | EVYEKND* | | |
| OT-001616 | Full | — | — | 1165 |
| (CD8a leader; | Construct | | | |
| CD19 scFv; | Encoded | MALPVTALLLPLALLLHAARPDIQMTQTTS | 1140 | 1166 |
| CD8 lunge | Protein 1 | SLSASLGDRVTISCRASQDISKYLNWYQQKP | | |
| and | | DGTVKLLIYHTSRLHSGVPSRFSGSGSGTDY | | |
| transmembrane | | SLTISNLEQEDIATYFCQQGNTLPYTFGGGT | | |
| Domain; 4- | | KLEITGGGGSGGGGSGGGGSEVKLQESGPG | | |
| 1BB | | LVAPSQSLSVTCTVSGVSLPDYGVSWIRQPP | | |
| intracellular | | RKGLEWLGVIWGSETTYYNSALKSRLTIIKD | | |
| signaling | | NSKSQVFLKMNSLQTDDTAIYYCAKHYYY | | |
| domain; CD3 | | GGSYAMDYWGQGTSVTVSSTTTPAPRPPTP | | |
| zeta signaling | | APTIASQPLSLRPEACRPAAGGAVHTRGLDF | | |
| domain; stop; | | ACDIYIWAPLAGTCGVLLLSLVITLYCKRGR | | |
| spacer | | KKLLYIFKQPFMRPVQTTQEEDGCSCRFPEE | | |
| (ATCGGGCT | | EEGGCELRVKFSRSADAPAYKQGQNQLYN | | |
| AGC); IRES; | | ELNLGRREEYDVLDKRRGRDPEMGGKPRR | | |
| Spacer | | KNPQEGLYNELQKDKMAEAYSEIGMKGER | | |
| (gcttgccacaacc | | RRGKGHDGLYQGLSTATKDTYDALHMQAL | | |
| cacaaggagacga | | PPR* | | |
| ccttcc); p40 | Encoded | MCHQQLVISWFSLVFLASPLVAIWELKKDV | 1141 | 1167 |
| signal | Protein 2 | YVVELDWYPDAPGEMVVLTCDTPEEDGIT | | |
| sequence; | | WTLDQSSEVLGSGKTLTIQVKEFGDAGQYT | | |
| 1LI2B (p40) | | CHKGGEVLSHSLLLLHKKEDGIWSTDILKD | | |
| (23-328 of | | QKEPKNKTFLRCEAKNYSGRFTCWWLTTIS | | |
| WT); Linker | | TDLTFSVKSSRGSSDPQGVTCGAATLSAER | | |
| ((G4S)3); | | VRGDNKEYEYSVECQEDSACPAAEESLPIE | | |
| Flexible G/S | | VMVDAVHKLKYENYTSSFFIRDIIKPDPPKN | | |
| rich linker. | | LQLKPLKNSRQVEVSWEYPDTWSTPHSYFS | | |
| IL12A (p35) | | LTFCVQVQGKSKREKKDRVFTDKTSATVIC | | |
| (57-253 of | | RKNASISVRAQDRYYSSSWSEWASVPCSGG | | |
| WT); | | GGSGGGGSGGGGSRNLPVATPDPGMFPCLH | | |
| Artificial | | HSQNLLRAVSNMLQKARQTLEFYPCTSEEI | | |
| linker | | DHEDITKDKTSTVEACLPLELTKNESCLNSR | | |
| (GGSGG); | | ETSFITNGSCLASRKTSFMMALCLSSIYEDL | | |
| hDHFR | | KMYQVEFKTMNAKLLMDPKRQIFLDQNML | | |
| (M1del, I17A, | | AVIDELMQALNFNSETVPQKSSLEEPDFYKT | | |
| YI22I); stop) | | KIKLCILLHAFRIRAVTIDRVMSYLNASGGS | | |
| (OT-CD19- | | GGVGSLNCIVAVSQNMGAGKNGDLPWPPL | | |
| IL12-018) | | RNEFRYFQRMTTTSSVEGKQNLVIMGKKT | | |
| | | WFSIPEKNRPLKGRINLVLSRELKEPPQGAH | | |
| | | FLSRSLDDALKLTEQPELANKVDMVWIVGG | | |
| | | SSVIKEAMNHPGHLKLFVTRIMQDFESDTFF | | |
| | | PEIDLEKYKLLPEYPGVLSDVQEEKGIKYKF | | |
| | | EVYEKND* | | |
| OT-001458 | Full | MALPVTALLLPLALLLHAARPDIQMTQTTS | 1142 | 1168 |
| (CD8a leader; | Construct | SLSASLGDRVTISCRASQDISKYLNWYQQKP | | |
| CD19 scFv; | | DGTVKLLIYHTSRLHSGVPSRFSGSGSGTDY | | |
| CD8 hinge | | SLTISNLEQEDIATYFCQQGNTLPYTFGGGT | | |
| and | | KLEITGGGGSGGGGSGGGGSEVKLQESGPG | | |
| transmembrane | | LVAPSQSLSVTCTVSGVSLPDYGVSWIRQPP | | |
| domain; 4- | | RKGLEWLGVIWGSETTYYNSALKSRLTIIKD | | |
| 1BB | | NSKSQVFLKMNSLQTDDTAIYYCAKHYYY | | |
| intracellular | | GGSYAMDYWGQGTSVTVSSTTTPAPRPPTP | | |
| signaling | | APTIASQPLSLRPEACRPAAGGAVHTRGLDF | | |
| domain; CD3 | | ACDIYIWAPLAGTCGVLLLSLVITLYCKRGR | | |
| zeta signaling | | KKLLYIFKQPFMRPVQTTQEEDGCSCRFPEE | | |
| domain; | | EEGGCELRVKFSRSADAPAYKQGQNQLYN | | |
| Linker (GSG); | | ELNLGRREEYDVLDKRRGRDPEMGGKPRR | | |
| BamHl-Gly | | KNPQEGLYNELQKDKMAEAYSEIGMKGER | | |
| Site; P2A | | RRGKGHDGLYQGLSTATKDTYDALHMQAL | | |
| cleavage site; | | PPRGSGATNFSLLKQAGDVEENPGPMDWT | | |
| IgE Leader; | | WILFLVAAATRVHSNWVNVISDLKKIEDLIQ | | |
| IL15 (49-162 | | SMHIDATLYTESDVHPSCKVTAMKCFLLEL | | |
| ofWT); | | QVISLESGDASIHDTVENLIILANNSLSSNGN | | |
| Linker (SG3- | | VTESGCKECEELEEKNIKEFLQSFVHIVQMFI | | |
| (SG4)3-SG3- | | NTSSGGGSGGGGSGGGGSGGGGSGGGSLQI | | |
| SLQ)); | | TCPPPMSVEHADIWVKSYSLYSRERYICNSG | | |
| IL15Ra (31- | | FKRKAGTSSLTECVLNKATNVAHWTTPSLK | | |
| 267 of WT); | | CIRDPALVHQRPAPPSTVTTAGVTPQPESLS | | |
| stop) (OT- | | PSGKEPAASSPSSNNTAATTAAIVPGSQLMP | | |
| CD19-IL15- | | SKSPSTGTTEISSHESSHGTPSQTTAKNWELT | | |
| 007) | | ASASHQPPGVYPQGHSDTTVAISTSTVLLCG | | |
| | | LSAVSLLACYLKSRQTPPLASVEMEAMEAL | | |
| | | PVTWGTSSRDEDLENCSHHL* | | |

Constructs disclosed in Table 4, and Table 5, which are transcriptionally controlled by a CMV promoter, in some instances may be placed under the transcriptional control of a different promoter to test the role of promoters in CD19 CAR expression. In one embodiment, the CMV promoter may be replaced by an EF1a promoter. In one embodiment, the CMV promoter of the, OT-001010 OT-CD19-001) construct, may be replaced to generate OT-001399 (OT-CD19-055) construct, with a EF1a promoter.

In one embodiment, the construct is OT-001992 (OT-CD19-IL12-012) comprising the nucleotide sequence of SEQ ID NO. 1197. This construct's nucleotide sequence comprises a CAR component (SEQ ID NO. 1198 (which encodes SEQ ID NO. 1200)) and a FKBP DD regulated IL12 component (SEQ ID NO. 1199 (which encodes SEQ ID NO. 1201)) with an intervening IRES sequence (SEQ ID NO. 1213). The CAR component of OT-001992 comprises a CD8a leader (SEQ ID NO. 469, encoded by SEQ ID NO. 511), CD19 scFv (SEQ ID NO. 465, encoded by SEQ ID NO. 467), CD8a hinge-TM domain (SEQ ID NO.466, encoded by SEQ ID NO. 784), 4-1BB intracellular domain (SEQ ID NO. 233, encoded by SEQ ID NO. 785), and CD3 zeta signaling domain (SEQ ID NO. 299, encoded by SEQ ID NO. 786), stop region.

The FKBP DD regulated IL12 component comprises an Interleukin-12 subunit beta (p40) leader (SEQ ID NO. 559, encoded by SEQ ID NO. 572), Interleukin-12 subunit beta (p40) (23-328 of WT) (SEQ ID NO. 563, encoded by SEQ ID NO. 473), Linker ((G4S)3) (SEQ ID NO. 560, encoded by SEQ ID NO. 713), Interleukin-12 subunit alpha (p35) (57-253 of WT) (SEQ ID NO. 564 encoded by SEQ ID NO. 1214), Linker (GGS) (GGS, encoded by GGTGGATCC), Modified Furin Cleavage site (SEQ ID NO. 471, encoded by SEQ ID NO. 523), Linker (GSW) (GSW, encoded by GGATCCTGG), FKBP (M1del, E32G, F37V, R72G, K106E) (SEQ ID NO. 7, encoded by SEQ ID NO. 812).

In one embodiment, the CAR construct comprises a CD19 scFV (e.g., CAT13.1E10 or FMC63), a CD8a spacer or transmembrane domain, and a 4-1BB and CD3 endodomain. These constructs with CAT13.1E10 may have increased proliferation after stimulation in vitro, increased cytotoxicity against the CD19+ targets, and increased effector and target interactions as compared to constructs with FMC63.

In some embodiments, the payloads of the present invention may be tuned using the catalytic domains of the E3 ubiquitin ligases. The catalytic domains of E3 ligases may be fused to an antibody or a fragment of the antibody. The payload is fused to the antigen recognized by the antibody or a fragment of the antibody that is fused to the E3 ligases catalytic domain. The E3 ligases useful in the present invention include, but are not limited to Ring E3 ligase, HECT E3 ligases and RBR E3 ligases.

In some embodiments, the payload of the invention may be any of the co-stimulatory molecules and/or intracellular domains described herein. In some embodiments, one or more co-stimulatory molecules, each under the control of different SRE may be used in the present invention. SRE regulated co-stimulatory molecules may also be expressed in conjunction with a first-generation CAR, a second-generation CAR, a third generation CAR, a fourth generation, or any other CAR design described herein.

In one embodiment of the present invention, the payload of the invention is a CD33 specific CAR. The CD33 heavy and light chain may be combined with any of the signal peptides, transmembrane domains, costimulatory domains, intracellular domains and destabilizing domains described herein.

In some embodiments, the CAR of the present invention may be a tandem chimeric antigen receptor (TanCAR) which is able to target two, three, four, or more tumor specific antigens. In some aspects, The CAR is a bispecific TanCAR including two targeting domains which recognize two different TSAs on tumor cells. The bispecific CAR may be further defined as comprising an extracellular region comprising a targeting domain (e.g., an antigen recognition domain) specific for a first tumor antigen and a targeting domain (e.g., an antigen recognition domain) specific for a second tumor antigen. In other aspects, the CAR is a multispecific TanCAR that includes three or more targeting domains configured in a tandem arrangement. The space between the targeting domains in the TanCAR may be between about 5 and about 30 amino acids in length, for example, 6, 7, 8, 9, 10, 11, 12, 13, 14, 15, 16, 17, 18, 19, 20, 21, 22, 23, 24, 25, 26, 27, 28, 29 and 30 amino acids.

### Split CAR

In some embodiments, the components including the targeting moiety, transmembrane domain and intracellular signaling domains of the present invention may be split into two or more parts such that it is dependent on multiple inputs that promote assembly of the intact functional receptor. In one embodiment, the split synthetic CAR system can be constructed in which the assembly of an activated CAR receptor is dependent on the binding of a ligand to the SRE (e.g. a small molecule) and a specific antigen to the targeting moiety. As a non-limiting example, the split CAR consists of two parts that assemble in a small molecule-dependent manner; one part of the receptor features an extracellular antigen binding domain (e.g. scFv) and the other part has the intracellular signaling domains, such as the CD3ξ intracellular domain.

In other aspects, the split parts of the CAR system can be further modified to increase signal. In one example, the second part of cytoplasmic fragment may be anchored to the plasma membrane by incorporating a transmembrane domain (e.g., CD8αtransmembrane domain) to the construct. An additional extracellular domain may also be added to the second part of the CAR system, for instance an extracellular domain that mediates homo-dimerization. These modifications may increase receptor output activity, i.e., T cell activation.

In some aspects, the two parts of the split CAR system contain heterodimerization domains that conditionally interact upon binding of a heterodimerizing small molecule. As such, the receptor components are assembled in the presence of the small molecule, to form an intact system which can then be activated by antigen engagement. Any known heterodimerizing components can be incorporated into a split CAR system. Other small molecule dependent heterodimerization domains may also be used, including, but not limited to, gibberellin-induced dimerization system (GID1-GAI), trimethoprim-SLF induced ecDHFR and FKBP dimerization (Czlapinski et al., J Am Chem Soc., 2008, 130(40): 13186-13187) and ABA (abscisic acid) induced dimerization of PP2C and PYL domains (Cutler et al., Annu Rev Plant Biol. 2010, 61: 651-679). The dual regulation using inducible assembly (e.g., ligand dependent dimerization) and degradation (e.g., destabilizing domain induced CAR degradation) of the split CAR system may provide more flexibility to control the activity of the CAR modified T cells.

### Switchable CAR

In some embodiments, the CAR of the invention may be a switchable CAR. In this CAR design, a system is directly integrated in the hinge domain that separate the scFv domain from the cell membrane domain in the CAR. Such system is possible to split or combine different key functions of a CAR such as activation and costimulation within different chains of a receptor complex, mimicking the complexity of the TCR native architecture. This integrated system can switch the scFv and antigen interaction between on/off states controlled by the absence/presence of the stimulus.

### Reversible CAR

In other embodiments, the CAR of the invention may be a reversible CAR system. In this CAR architecture, a LID domain (ligand-induced degradation) is incorporated into the CAR system. The CAR can be temporarily down-regulated by adding a ligand of the LID domain. The combination of LID and DD mediated regulation provides tunable control of continuingly activated CAR T cells, thereby reducing CAR mediated tissue toxicity.

### Activation-Conditional CAR

In some embodiments, payloads of the invention may be an activation-conditional chimeric antigen receptor, which is only expressed in an activated immune cell. The expression of the CAR may be coupled to activation conditional control region which refers to one or more nucleic acid sequences that induce the transcription and/or expression of a sequence e.g. a CAR under its control. Such activation conditional control regions may be promoters of genes that are unregulated during the activation of the immune effector cell e.g. IL2 promoter or NFAT binding sites. In some embodiments, activation of the immune cell may be achieved by a constitutively expressed CAR.

### Universal CAR

In some embodiments, the payload of the present invention may be a Split Universal Programmable (SUPRA) CAR. A SUPRA CAR may be a two-component receptor system comprising of a universal receptor (zip CAR) expressed on T cells and a tumor-targeting scFv adaptor. The zip CAR universal receptor may be generated by the fusion of intracellular signaling domains and a leucine zipper as the extracellular domain. The tumor-targeting scFv adaptor molecule or zipFv, may be generated by the fusion of a cognate leucine zipper and an scFv. The scFv of the zipFv may bind to a tumor antigen, and the leucine zipper may bind and activate the zip CAR on the T cells. Unlike the conventional fixed CAR design, the SUPRA CAR modular design allows targeting of multiple antigens without further genetic manipulations of the immune cells.

### Cytokines, Chemokines and Other Soluble Factors

In accordance with the present invention, CARs of the present invention may be utilized along with other payloads of the present invention may be cytokines, chemokines, growth factors, and soluble proteins produced by immune cells, cancer cells and other cell types, which act as chemical communicators between cells and tissues within the body. These proteins mediate a wide range of physiological functions, from effects on cell growth, differentiation, migration and survival, to a number of effector activities. For example, activated T cells produce a variety of cytokines for cytotoxic function to eliminate tumor cells.

In some embodiments, payloads of the present invention may be cytokines, and fragments, variants, analogs and derivatives thereof, including but not limited to interleukins, tumor necrosis factors (TNFs), interferons (IFNs), TGF beta and chemokines. It is understood in the art that certain gene and/or protein nomenclature for the same gene or protein may be inclusive or exclusive of punctuation such as a dash “—” or symbolic such as Greek letters. Whether these are included or excluded herein, the meaning is not meant to be changed as would be understood by one of skill in the art. For example, IL2, IL2 and IL2 refer to the same interleukin. Likewise, TNF alpha, TNFα, TNF-alpha, TNF-α, TNF alpha and TNF a all refer to the same protein. In some embodiments, payloads of the present invention may be cytokines that stimulate immune responses. In other embodiments, payloads of the invention may be antagonists of cytokines that negatively impact anti-cancer immune responses.

In some embodiments, payloads of the present invention may be cytokine receptors, recombinant receptors, variants, analogs and derivatives thereof; or signal components of cytokines.

In some embodiments, cytokines of the present invention may be utilized to improve expansion, survival, persistence, and potency of immune cells such as CD8+T_{EM}, natural killer cells and tumor infiltrating lymphocytes (TIL) cells used for immunotherapy. In other embodiments, T cells engineered with two or more DD regulated cytokines are utilized to provide kinetic control of T cell activation and tumor microenvironment remodeling. In one aspect, the present invention provides biocircuits and compositions to minimize toxicity related to cytokine therapy. Despite its success in mitigating tumor burden, systemic cytokine therapy often results in the development of severe dose limiting side effects. Two factors contribute to the observed toxicity (a) Pleiotropism, wherein cytokines affect different cells types and sometimes produce opposing effects on the same cells depending on the context (b) Cytokines have short serum half-life and thus need to be administered at high doses to achieve therapeutic effects, which exacerbates the pleiotropic effects. In one aspect, cytokines of the present invention may be utilized to modulate cytokine expression in the event of adverse effects. In some embodiments, cytokines of the present invention may be designed to have prolonged life span or enhanced specificity to minimize toxicity.

In some embodiments, the payload of the present invention may be an interleukin (IL) cytokine. Interleukins (ILs) are a class of glycoproteins produced by leukocytes for regulating immune responses. As used herein, the term “interleukin (IL)” refers to an interleukin polypeptide from any species or source and includes the full-length protein as well as fragments or portions of the protein. In some aspects, the interleukin payload is selected from IL′, IL1alpha (also called hematopoietin-1), IL1beta (catabolin), IL1 delta, IL1epsilon, IL1eta, IL1 zeta, interleukin-1 family member 1 to 11 (IL1F1 to IL1F11), interleukin-1 homolog 1 to 4 (IL1H1 to IL1H4), IL1 related protein 1 to 3 (IL1RP1 to IL1RP3), IL2, IL3, IL4, IL5, IL6, IL7, IL8, IL9, IL10, IL10C, IL10D, IL11, IL11a, IL11b, IL12, IL13, IL14, IL15, IL16, IL17, IL17A, 1117B, IL17C, IL17E, IL17F, IL18, IL19, IL20, IL20 like (IL20L), 1121, IL22, IL23, IL23A, IL23-p19, IL23-p40, IL24, 1125, IL26, IL27, IL28A, IL28B, IL29, IL30, IL31, IL32, IL33, IL34, IL35, IL36 alpha, IL36 beta, IL36 gamma, IL36RN, IL37, IL37a, IL37b, IL37c, IL37d, IL37e and IL38. In other aspects, the payload of the present invention may be an interleukin receptor selected from CD121a, CDw121b, IL2Ra/CD25, IL2R13/CD122, IL2Ry/CD132, CDw131, CD124, CD131, CDw125, CD126, CD130, CD127, CDw210, IL8RA, IL11Ra, CD212, CD213α1, CD213α2, IL14R, IL15Ra, CDw217, IL18Rα, IL18Rβ, IL20Rα, and IL20Rβ.

In one embodiment, the payload of the invention may comprise IL12. IL12 is a heterodimeric protein of two subunits (p35, p40) that is secreted by antigen presenting cells, such as macrophages and dendritic cells. IL12 is type 1 cytokine that acts on natural killer (NK) cells, macrophages, CD8⁺ Cytotoxic T cells, and CD4⁺ T helper cells through STAT4 pathway to induce IFN-γ production in these effector immune cells (reviewed by Trinchieri G, Nat Rev Immunol. 2003; 3(2): 133-146). IL12 can promote the cytotoxic activity of NK cells and CD8⁺ T cells, therefore has anti-tumor function. Intravenous injection of recombinant IL12 exhibited modest clinical efficacy in a handful of patients with advanced melanoma and renal cell carcinoma (Gollob et al., Clin. Cancer Res. 2000; 6(5):1678-1692). IL12 has been used as an adjuvant to enhance cytotoxic immunity using a melanoma antigen vaccine, or using peptide pulsed peripheral blood mononuclear cells; and to promote NK cell activity in breast cancer with trastuzumab treatment. Local delivery of IL12 to the tumor microenvironment promotes tumor regression in several tumor models. These studies all indicate that locally increased IL12 level can promote anti-tumor immunity. One major obstacle of systemic or local administration of recombinant IL12 protein, or through oncolytic viral vectors is the severe side effects when IL12 is presented at high level. Developing a system that tightly controls IL12 level may provide a safe use of IL12 in cancer treatment.

In one aspect, the effector module of the invention may be a DD-IL12 fusion polypeptide. This regulatable DD-IL12 fusion polypeptide may be directly used as an immunotherapeutic agent or be transduced into an immune effector cell (T cells and TIL cells) to generate modified T cells with greater in vivo expansion and survival capabilities for adoptive cell transfer. The need for harsh preconditioning regimens in current adoptive cell therapies may be minimized using regulated IL12 DD-IL12 may be utilized to modify tumor microenvironment and increase persistence in solid tumors that are currently refractory to tumor antigen targeted therapy. In some embodiments, CAR expressing T cells may be armored with DD regulated IL12 to relieve immunosuppression without systemic toxicity.

In some embodiments, the IL12 may be a Flexi IL12, wherein both p35 and p40 subunits, are encoded by a single cDNA that produces a single chain polypeptide. The single chain polypeptide may be generated by placing p35 subunit at the N terminus or the C terminus of the single chain polypeptide. Similarly, the p40 subunit may be at the N terminus or C terminus of the single chain polypeptide. In some embodiments, the IL12 constructs of the invention may be placed under the transcriptional control of the CMV promoter (SEQ ID NO. 556, 1100), an EF1a promoter (SEQ ID NO. 557, 708, 1099, 1103) or a PGK promoter (SEQ ID NO. 558, 1101, 1102). Any portion of IL12 that retains one or more functions of full length or mature IL12 may be useful in the present invention. In some embodiments, IL12 constructs may be generated by different permutations and combinations of any of the linkers, promoters, cleavage sites and DDs described herein. In some embodiments, the DD may be placed at the N terminus of the construct. In some aspects, the DD may be placed at the C terminus of the construct.

In some aspects, the DD-IL12 comprises the amino acid sequences listed in Table 6. The amino acid sequences in Table 6 may comprise a stop codon which is denoted in the table with a “*” at the end of the amino acid sequence. In Table 6, “del” means deletion and “WT” means wild-type.

TABLE 6
DD-IL12 constructs

| | | | Amino | Nucleic |
|---|---|---|---|---|
| | | | acid | Acid |
| | | | SEQ ID | SEQ ID |
| Description | Promoter | Amino acid Sequence | NO | NO |
| p40 signal | — | MCHQQLVISWFSLVFLASPLVA | 559 | 567-575; |
| sequence | | | | 1215 |
| Linker | — | GGSGG | 470 | 519-520 |
| Linker | — | GGGGSGGGGSGGGGS | 560 | 710-715 |
| Linker | | GS | — | GGATCC |
| P2A | — | ATNFSLLKQAGDVEENPGP | 576 | 577 |
| Cleavable | | | | |
| Peptide | | | | |
| Furin | — | SARNRQKRS | 561 | 581 |
| cleavage site | | | | |
| Furin | — | ARNRQKRS | 562 | 582 |
| cleavage site | | | | |
| Modified | — | ESRRVRRNKRSK | 471 | 521-522 |
| Furin | | | | |
| P2A | — | GATNFSLLKQAGDVEENPGP | 725 | 726 |
| Cleavable | | | | |
| Peptide | | | | |
| p40 (WT) | | MCHQQLVISWFSLVFLASPLVAIWELKKDV | 1091 | 1092 |
| | | YVVELDWYPDAPGEMVVLTCDTPEEDGITW | | |
| | | TLDQSSEVLGSGKTLTIQVKEFGDAGQYTCH | | |
| | | KGGEVLSHSLLLLHKKEDGIWSTDILKDQKE | | |
| | | PKNKTFLRCEAKNYSGRFTCWWLTTISTDLT | | |
| | | FSVKSSRGSSDPQGVTCGAATLSAERVRGDN | | |
| | | KEYEYSVECQEDSACPAAEESLPIEVMVDAV | | |
| | | HKLKYENYTSSFFIRDIIKPDPPKNLQLKPL | | |
| | | NSRQVEVSWEYPDTWSTPHSYFSLTFCVQV | | |
| | | QKGKSKREKKDRVFTDKTSATVICRKNASIS | | |
| | | VRAQDRYYSSSWSEWASVPCS | | |
| p40 (23-328 | — | IWELKKDVYVVELDWYPDAPGEMVVLTCDT | 563 | 583-791, |
| of WT) | | PEEDGITWTLDQSSEVLGSGKTLTIQVKEFG | | 472-474 |
| | | DAGQYTCHKGGEVLSHSLLLLHKKEDGIWS | | |
| | | TDILKDQKEPKNKTFLRCEAKNYSGRFTCW | | |
| | | WLTTISTDLTFSVKSSRGSSDPQGVTCGAAT | | |
| | | LSAERVRGDNKEYEYSVECQEDSACPAAEES | | |
| | | LPIEVMVDAVHKLKYENYTSSFFIRDIIKPDP | | |
| | | PKNLQLKPLKNSRQVEVSWEYPDTWSTPHS | | |
| | | YFSLTFCVQVQGKSKREKKDRVFTDKTSAT | | |
| | | VICRKNASISVRAQDRYYSSSWSEWASVPCS | | |
| p40 (23-328 | — | IWELKKDVYVVELDWYPDAPGEMVVLTCD | 578 | 579 |
| of WT) | | TPEEDGITWTLDQSSEVLGSGKTLTIQVKEFG | | |
| (K217N) | | DAGQYTCHKGGEVLSHSLLLLHKKEDGIWS | | |
| | | TDILKDQKEPKNKTFLRCEAKNYSGRFTCW | | |
| | | WLTTISTDLTFSVKSSRGSSDPQGVTCGAAT | | |
| | | LSAERVRGDNKEYEYSVECQEDSACPAAEES | | |
| | | LPIEVMVDAVHKLKYENYTSSFFIRDIIKPDP | | |
| | | PNNLQLKPLKNSRQVEVSWEYPDTWSTPHS | | |
| | | YFSLTFCVQVQGKSKREKKDRVFTDKTSAT | | |
| | | VICRKNASISVRAQDRYYSSSWSEWASVPCS | | |
| p35 (WT) | — | MWPPGSASQPPPSPAAATGLHPAARPVSLQC | 1093 | 1094 |
| | | RLSMCPARSLLLVATLVLLDHLSLARNLPVA | | |
| | | TPDPGMFPCLHHSQNLLRAVSNMLQKARQT | | |
| | | LEFYPCTSEEIDHEDITKDKTSTVEACLPLELT | | |
| | | KNESCLNSRETSFITNGSCLASRKTSFMMAL | | |
| | | CLSSIYEDLKMYQVEFKTMNAKLLMDPKRQ | | |
| | | IFLDQNMLAVIDELMQALNFNSETVPQKSSL | | |
| | | EEPDFYKTKIKLCILLHAFRIRAVTIDRVMSY | | |
| | | LNAS | | |
| p35 (57-253 | — | RNLPVATPDPGMFPCLHHSQNLLRAVSNML | 564 | 593-602, |
| of WT) | | QKARQTLEFYPCTSEEIDHEDITKDKTSTVEA | | 811 |
| | | CLPLELTKNESCLNSRETSFITNGSCLASRKT | | |
| | | SFMMALCLSSIYEDLKMYQVEFKTMNAKLL | | |
| | | MDPKRQIFLDQNMLAVIDELMQALNFNSET | | |
| | | VPQKSSLEEPDFYKTKIKLCILLHAFRIRAVTI | | |
| | | DRVMSYLNAS | | |
| ecDHFR | — | ISLIAALAVDYVIGMENAMPWNLPADLAWF | 4 | 532, 603, |
| (M1del, | | KRNTLNKPVIMGRHTWESIGRPLPGRKNIILS | | 641, 527, |
| R12Y, | | SQPGTDDRVTWVKSVDEAIAACGDVPEIMVI | | 788, 791 |
| Y100I) | | GGGRVIEQFLPKAQKLYLTHIDAEVEGDTHF | | |
| | | PDYEPDDWESVFSEFHDADAQNSHSYCFEIL | | |
| | | ERR | | |
| FKBP | — | GVQVETISPGDGRTFPKRGQTCVVHYTGML | 6 | 524-526, |
| (M1del, | | EDGKKVDSSRDRNKPFKFMLGKQEVIRGWE | | 787, 789 |
| F37V, | | EGVAQMSVGQRAKLTISPDYAYGATGHPGII | | |
| L107P) | | PPHATLVFDVELLKPE | | |
| FKBP | — | GVQVETISPGDGRTFPKRGQTCVVHYTGML | 7 | 528-531, |
| (M1del, | | GDGKKVDSSRDRNKPFKFMLGKQEVIRGWE | | 794, 812, |
| E32G, | | EGVAQMSVGQGAKLTISPDYAYGATGHPGII | | 827; 1236 |
| F37V, | | PPHATLVFDVELLELE | | |
| R72G, | | | | |
| K106E) | | | | |
| hDHFR | — | VGSLNCIVAVSQNMGIGKNGDLPWPPLRNEF | 8 | 604 |
| (M1del, | | RYFFRMTTTSSVEGKQNLVIMGKKTWFSIPE | | |
| Q36F, | | KNRPLKGRINLVLSRELKEPPQGAHFLSRSLD | | |
| Y122I, | | DALKLTEQPELANKVDMVWIVGGSSVIKEF | | |
| A125F) | | MNHPGHLKLFVTRIMQDFESDTFFPEIDLEK | | |
| | | YKLLPEYPGVLSDVQEEKGIKYKFEVYEKND | | |
| hDHFR | — | VGSLNCIVAVSQNMGVGKNGDLPWPPLRNE | 695 | 779 |
| (M1del, | | FRYFQRMTTTSSVEGKQNLVIMGKKTWFSIP | | |
| I17V) | | EKNRPLKGRINLVLSRELKEPPQGAHFLSRSL | | |
| | | DDALKLTEQPELANKVDMVWIVGGSSVYKE | | |
| | | AMNHPGHLKLFVTRIMQDFESDTFFPEIDLE | | |
| | | KYKLLPEYPGVLSDVQEEKGIKYKFEVYEKN | | |
| | | D | | |
| hDHFR | — | VGSLNCIVAVSQNMGIGKNGDLPWPPLRNEF | 696 | 534, 795 |
| (M1del, | | RYFQRMTTTSSVEGKQNLVIMGKKTWFSIPE | | |
| Y122I) | | KNRPLKGRINLVLSRELKEPPQGAHFLSRSLD | | |
| | | DALKLTEQPELANKVDMVWIVGGSSVIKEA | | |
| | | MNHPGHLKLFVTRIMQDFESDTFFPEIDLEK | | |
| | | YKLLPEYPGVLSDVQEEKGIKYKFEVYEKND | | |
| OT-001098 | EF1a | MCHQQLVISWFSLVFLASPLVAIWELKKDV | 566 | 610 |
| (p40 signal | | YVVELDWYPDAPGEMVVLTCDTPEEDGITW | | |
| sequence; | | TLDQSSEVLGSGKTLTIQVKEFGDAGQYTCH | | |
| p40 (23-328 | | KGGEVLSHSLLLLHKKEDGIWSTDILKDQKE | | |
| of WT); | | PKNKTFLRCEAKNYSGRFTCWWLTTISTDLT | | |
| linker | | FSVKSSRGSSDPQGVTCGAATLSAERVRGDN | | |
| (G4S)3; p35 | | KEYEYSVECQEDSACPAAEESLPIEVMVDAV | | |
| (57-253 of | | HKLKYENYTSSFFIRDIIKPDPPKNLQLKPLK | | |
| WT); stop) | | NSRQVEVSWEYPDTWSTPHSYFSLTFCVQV | | |
| (OT-IL12— | | QGKSKREKKDRVFTDKTSATVICRKNASISV | | |
| 020) | | RAQDRYYSSSWSEWASVPCSGGGGSGGGGS | | |
| | | GGGGSRNLPVATPDPGMFPCLHHSQNLLRA | | |
| | | VSNMLQKARQTLEFYPCTSEEIDHEDITKDK | | |
| | | TSTVEACLPLELTKNESCLNSRETSFITNGSC | | |
| | | LASRKTSFMMALCLSSIYEDLKMYQVEFKT | | |
| | | MNAKLLMDPKRQIFLDQNMLAVIDELMQAL | | |
| | | NFNSETVPQKSSLEEPDFYKTKIKLCILLHAF | | |
| | | RIRAVTIDRVMSYLNAS* | | |
| OT-001104 | PGK | MCHQQLVISWFSLVFLASPLVAIWELKKDV | 565 | 609 |
| (p40 signal | | YVVELDWYPDAPGEMVVLTCDTPEEDGITW | | |
| sequence; | | TLDQSSEVLGSGKTLTIQVKEFGDAGQYTCH | | |
| p40 (23-328 | | KGGEVLSHSLLLLHKKEDGIWSTDILKDQKE | | |
| of WT); | | PKNKTFLRCEAKNYSGRFTCWWLTTISTDLT | | |
| linker | | FSVKSSRGSSDPQGVTCGAATLSAERVRGDN | | |
| (G4S)3; p35 | | KEYEYSVECQEDSACPAAEESLPIEVMVDAV | | |
| (57-253 of | | HKLKYENYTSSFFIRDIIKPDPPKNLQLKPLK | | |
| WT); linker | | NSRQVEVSWEYPDTWSTPHSYFSLTFCVQV | | |
| (GGSG); | | QGKSKREKKDRVFTDKTSATVICRKNASISV | | |
| FKBP | | RAQDRYYSSSWSEWASVPCSGGGGSGGGGS | | |
| (M1del, | | GGGGSRNLPVATPDPGMFPCLHHSQNLLRA | | |
| E32G, | | VSNMLQKARQTLEFYPCTSEEIDHEDITKDK | | |
| F37V, | | TSTVEACLPLELTKNESCLNSRETSFITNGSC | | |
| R72G, | | LASRKTSFMMALCLSSIYEDLKMYQVEFKT | | |
| K106E); | | MNAKLLMDPKRQIFLDQNMLAVIDELMQAL | | |
| stop) (OT— | | NFNSETVPQKSSLEEPDFYKTKIKLCILLHAF | | |
| IL12-025) | | RIRAVTIDRVMSYLNASGGSGGVQVETISPG | | |
| | | DGRTFPKRGQTCVVHYTGMLGDGKKVDSS | | |
| | | RDRNKPFKFMLGKQEVIRGWEEGVAQMSV | | |
| | | GQGAKLTISPDYAYGATGHPGIIPPHATLVFD | | |
| | | VELLELE* | | |
| OT-001105 | EF1a | MCHQQLVISWFSLVFLASPLVAIWELKKDV | 565 | 609 |
| (p40 signal | | YVVELDWYPDAPGEMVVLTCDTPEEDGITW | | |
| sequence; | | TLDQSSEVLGSGKTLTIQVKEFGDAGQYTCH | | |
| p40 (23-328 | | KGGEVLSHSLLLLHKKEDGIWSTDILKDQKE | | |
| of WT); | | PKNKTFLRCEAKNYSGRFTCWWLTTISTDLT | | |
| linker | | FSVKSSRGSSDPQGVTCGAATLSAERVRGDN | | |
| (G4S)3; p35 | | KEYEYSVECQEDSACPAAEESLPIEVMVDAV | | |
| (57-253 of | | HKLKYENYTSSFFIRDIIKPDPPKNLQLKPLK | | |
| WT); linker | | NSRQVEVSWEYPDTWSTPHSYFSLTFCVQV | | |
| (GGSG); | | QGKSKREKKDRVFTDKTSATVICRKNASISV | | |
| FKBP | | RAQDRYYSSSWSEWASVPCSGGGGSGGGGS | | |
| (M1del, | | GGGGSRNLPVATPDPGMFPCLHHSQNLLRA | | |
| E32G, | | VSNMLQKARQTLEFYPCTSEEIDHEDITKDK | | |
| F37V, | | TSTVEACLPLELTKNESCLNSRETSFITNGSC | | |
| R72G, | | LASRKTSFMMALCLSSIYEDLKMYQVEFKT | | |
| K106E); | | MNAKLLMDPKRQIFLDQNMLAVIDELMQAL | | |
| stop) (OT— | | NFNSETVPQKSSLEEPDFYKTKIKLCILLHAF | | |
| IL12-026) | | RIRAVTIDRVMSYLNASGGSGGVQVETISPG | | |
| | | DGRTFPKRGQTCVVHYTGMLGDGKKVDSS | | |
| | | RDRNKPFKFMLGKQEVIRGWEEGVAQMSV | | |
| | | GQGAKLTISPDYAYGATGHPGIIPPHATLVFD | | |
| | | VELLELE* | | |
| OT-001444 | EF1a | MCHQQLVISWFSLVFLASPLVAIWELKKDVY | 842 | 847 |
| (p40 signal | | VVELDWYPDAPGEMVVLTCDTPEEDGITWT | | |
| sequence; | | LDQSSEVLGSGKTLTIQVKEFGDAGQYTCHK | | |
| p40 (23-328 | | GGEVLSHSLLLLHKKEDGIWSTDILKDQKEP | | |
| of WT); | | KNKTFLRCEAKNYSGRFTCWWLTTISTDLTF | | |
| linker | | SVKSSRGSSDPQGVTCGAATLSAERVRGDN | | |
| (G4S)3; p35 | | KEYEYSVECQEDSACPAAEESLPIEVMVDAV | | |
| (57-253 of | | HKLKYENYTSSFFIRDIIKPDPPKNLQLKPLK | | |
| WT); | | NSRQVEVSWEYPDTWSTPHSYFSLTFCVQV | | |
| BamH1 Site | | QGKSKREKKDRVFTDKTSATVICRKNASISV | | |
| (GS); | | RAQDRYYSSSWSEWASVPCSGGGGSGGGGS | | |
| hDHFR | | GGGGSRNLPVATPDPGMFPCLHHSQNLLRA | | |
| (M1del, | | VSNMLQKARQTLEFYPCTSEEIDHEDITKDK | | |
| I17V); stop) | | TSTVEACLPLELTKNESCLNSRETSFITNGSCL | | |
| (OT-IL12— | | ASRKTSFMMALCLSSIYEDLKMYQVEFKTM | | |
| 078) | | NAKLLMDPKRQIFLDQNMLAVIDELMQALN | | |
| | | FNSETVPQKSSLEEPDFYKTKIKLCILLHAFRI | | |
| | | RAVTIDRVMSYLNASGSVGSLNCIVAVSQN | | |
| | | MGVGKNGDLPWPPLRNEFRYFQRMTTTSSV | | |
| | | EGKQNLVIMGKKTWFSIPEKNRPLKGRINLV | | |
| | | LSRELKEPPQGAHFLSRSLDDALKLTEQPELA | | |
| | | NKVDMVWIVGGSSVYKEAMNHPGHLKLFV | | |
| | | TRIMQDFESDTFFPEIDLEKYKLLPEYPGVLS | | |
| | | DVQEEKGIKYKFEVYEKND* | | |
| OT-001445 | EF1a | MCHQQLVISWFSLVFLASPLVAIWELKKDVY | 844 | 848 |
| (p40 signal | | VVELDWYPDAPGEMVVLTCDTPEEDGITWT | | |
| sequence; | | LDQSSEVLGSGKTLTIQVKEFGDAGQYTCHK | | |
| p40 (23-328 | | GGEVLSHSLLLLHKKEDGIWSTDILKDQKEP | | |
| of WT); | | KNKTFLRCEAKNYSGRFTCWWLTTISTDLTF | | |
| linker | | SVKSSRGSSDPQGVTCGAATLSAERVRGDN | | |
| (G4S)3; p35 | | KEYEYSVECQEDSACPAAEESLPIEVMVDAV | | |
| (57-253 of | | HKLKYENYTSSFFIRDIIKPDPPKNLQLKPLK | | |
| WT); Gly; | | NSRQVEVSWEYPDTWSTPHSYFSLTFCVQV | | |
| Modified | | QGKSKREKKDRVFTDKTSATVICRKNASISV | | |
| furin | | RAQDRYYSSSWSEWASVPCSGGGGSGGGGS | | |
| (ESRRVRR | | GGGGSRNLPVATPDPGMFPCLHHSQNLLRA | | |
| NKRSK); | | VSNMLQKARQTLEFYPCTSEEIDHEDITKDK | | |
| BamH1 Site | | TSTVEACLPLELTKNESCLNSRETSFITNGSCL | | |
| (GS); | | ASRKTSFMMALCLSSIYEDLKMYQVEFKTM | | |
| hDHFR | | NAKLLMDPKRQIFLDQNMLAVIDELMQALN | | |
| (M1del, | | FNSETVPQKSSLEEPDFYKTKIKLCILLHAF | | |
| I17V); stop) | | RIRAVTIDRVMSYLNASGESRRVRRNKRSKG | | |
| (OT-IL12— | | SVGSLNCIVAVSQNMGVGKNGDLPWPPLRNE | | |
| 079) | | FRYFQRMTTTSSVEGKQNLVIMGKKTWFSIP | | |
| | | EKNRPLKGRINLVLSRELKEPPQGAHFLSRSL | | |
| | | DDALKLTEQPELANKVDMVWIVGGSSVYKE | | |
| | | AMNHPGHLKLFVTRIMQDFESDTFFPEIDLE | | |
| | | KYKLLPEYPGVLSDVQEEKGIKYKFEVYEKN | | |
| | | D* | | |
| OT-001446 | EF1a | MCHQQLVISWFSLVFLASPLVAIWELKKDVY | 844 | 849 |
| (p40 signal | | VVELDWYPDAPGEMVVLTCDTPEEDGITWT | | |
| sequence; | | LDQSSEVLGSGKTLTIQVKEFGDAGQYTCHK | | |
| p40 (23-328 | | GGEVLSHSLLLLHKKEDGIWSTDILKDQKEP | | |
| of WT); | | KNKTFLRCEAKNYSGRFTCWWLTTISTDLTF | | |
| linker | | SVKSSRGSSDPQGVTCGAATLSAERVRGDN | | |
| (G4S)3; p35 | | KEYEYSVECQEDSACPAAEESLPIEVMVDAV | | |
| (57-253 of | | HKLKYENYTSSFFIRDIIKPDPPKNLQLKPLK | | |
| WT); | | NSRQVEVSWEYPDTWSTPHSYFSLTFCVQV | | |
| BamH1 Site | | QGKSKREKKDRVFTDKTSATVICRKNASISV | | |
| (GS); | | RAQDRYYSSSWSEWASVPCSGGGGSGGGGS | | |
| hDHFR | | GGGGSRNLPVATPDPGMFPCLHHSQNLLRA | | |
| (M1del, | | VSNMLQKARQTLEFYPCTSEEIDHEDITKDK | | |
| Y122I); | | TSTVEACLPLELTKNESCLNSRETSFITNGSC | | |
| stop) (OT— | | LASRKTSFMMALCLSSIYEDLKMYQVEFKTM | | |
| IL12-082— | | NAKLLMDPKRQIFLDQNMLAVIDELMQALNF | | |
| 002 or OT— | | NSETVPQKSSLEEPDFYKTKIKLCILLHAFR | | |
| IL12-082) | | IRAVTIDRVMSYLNASGSVGSLNCIVAVSQN | | |
| | | MGIGKNGDLPWPPLRNEFRYFQRMTTTSSVE | | |
| | | GKQNLVIMGKKTWFSIPEKNRPLKGRINLVL | | |
| | | SRELKEPPQGAHFLSRSLDDALKLTEQPELA | | |
| | | NKVDMVWIVGGSSVIKEAMNHPGHLKLFVT | | |
| | | RIMQDFESDTFFPEIDLEKYKLLPEYPGVLSD | | |
| | | VQEEKGIKYKFEVYEKND* | | |
| OT-001447 | EF1a | MCHQQLVISWFSLVFLASPLVAIWELKKDVY | 845 | 848 |
| (p40 signal | | VVELDWYPDAPGEMVVLTCDTPEEDGITWT | | |
| sequence; | | LDQSSEVLGSGKTLTIQVKEFGDAGQYTCHK | | |
| p40 (23-328 | | GGEVLSHSLLLLHKKEDGIWSTDILKDQKEP | | |
| of WT); | | KNKTFLRCEAKNYSGRFTCWWLTTISTDLTF | | |
| linker | | SVKSSRGSSDPQGVTCGAATLSAERVRGDN | | |
| (G4S)3; p35 | | KEYEYSVECQEDSACPAAEESLPIEVMVDAV | | |
| (57-253 of | | HKLKYENYTSSFFIRDIIKPDPPKNLQLKPLK | | |
| WT); Gly; | | NSRQVEVSWEYPDTWSTPHSYFSLTFCVQV | | |
| Modified | | QGKSKREKKDRVFTDKTSATVICRKNASISV | | |
| furin | | RAQDRYYSSSWSEWASVPCSGGGGSGGGGS | | |
| (ESRRVRR | | GGGGSRNLPVATPDPGMFPCLHHSQNLLRA | | |
| NKRSK); | | VSNMLQKARQTLEFYPCTSEEIDHEDITKDK | | |
| BamH1 Site | | TSTVEACLPLELTKNESCLNSRETSFITNGSCL | | |
| (GS); | | ASRKTSFMMALCLSSIYEDLKMYQVEFKTM | | |
| hDHFR | | NAKLLMDPKRQIFLDQNMLAVIDELMQALN | | |
| (M1del, | | FNSETVPQKSSLEEPDFYKTKIKLCILLHAFRI | | |
| Y122I); | | RAVTIDRVMSYLNASGESRRVRRNKRSKGS | | |
| stop) (OT— | | VGSLNCIVAVSQNMGIGKNGDLPWPPLRNEF | | |
| IL12-083— | | RYFQRMTTTSSVEGKQNLVIMGKKTWFSIPE | | |
| 002 or OT— | | KNRPLKGRINLVLSRELKEPPQGAHFLSRSLD | | |
| IL12-083) | | DALKLTEQPELANKVDMVWIVGGSSVIKEA | | |
| | | MNHPGHLKLFVTRIMQDFESDTFFPEIDLEK | | |
| | | YKLLPEYPGVLSDVQEEKGIKYKFEVYEKND | | |
| | | * | | |
| OT-001442 | EF1a | MCHQQLVISWFSLVFLASPLVAIWELKKDVY | 846 | 851 |
| (p40 signal | | VVELDWYPDAPGEMVVLTCDTPEEDGITWT | | |
| sequence; | | LDQSSEVLGSGKTLTIQVKEFGDAGQYTCHK | | |
| p40 (23-328 | | GGEVLSHSLLLLHKKEDGIWSTDILKDQKEP | | |
| of WT); | | KNKTFLRCEAKNYSGRFTCWWLTTISTDLTF | | |
| linker | | SVKSSRGSSDPQGVTCGAATLSAERVRGDN | | |
| (G4S)3; p35 | | KEYEYSVECQEDSACPAAEESLPIEVMVDAV | | |
| (57-253 of | | HKLKYENYTSSFFIRDIIKPDPPKNLQLKPLK | | |
| WT); | | NSRQVEVSWEYPDTWSTPHSYFSLTFCVQV | | |
| BamH1 Site | | QGKSKREKKDRVFTDKTSATVICRKNASISV | | |
| (GS); stop) | | RAQDRYYSSSWSEWASVPCSGGGGSGGGGS | | |
| (OT-IL12— | | GGGGSRNLPVATPDPGMFPCLHHSQNLLRA | | |
| 096-002 or | | VSNMLQKARQTLEFYPCTSEEIDHEDITKDK | | |
| OT-IL12— | | TSTVEACLPLELTKNESCLNSRETSFITNGSCL | | |
| 096) | | ASRKTSFMMALCLSSIYEDLKMYQVEFKTM | | |
| | | NAKLLMDPKRQIFLDQNMLAVIDELMQALN | | |
| | | FNSETVPQKSSLEEPDFYKTKIKLCILLHAFRI | | |
| | | RAVTIDRVMSYLNASGS* | | |

In some embodiments, DD regulated IL12 compositions of the invention may be utilized to minimize the cytotoxicities associated with systemic IL12 administration. Treatment with IL12 has been associated with systemic flu-like symptoms (fever, chills, fatigue, arthromyalgia, headache), toxic effects on the bone marrow, and liver. Hematologic toxicity observed most commonly included neutropenia and thrombocytopenia; hepatic dysfunction manifested in transient (dose dependent) increase in transaminases, hyperbilirubinemia and hypoalbuminemia. In some instances, toxicity is also associated with inflammation of the mucus membranes (oral mucositis, stomatitis or colitis). These toxic effects of IL12 were related to the secondary production of IFNgamma, TNFalpha, and chemokines such as IP10, and MIG. In certain aspects of the invention, DD regulated IL12 may be utilized to prevent the toxic effects associated with elevated production of secondary messengers. In some embodiments, DD regulated Flexi IL12 constructs may be used to improve the efficacy of the CARs, especially in solid tumor settings, by providing a controlled local signal for tumor microenvironment remodeling and epitope spreading. DD regulation also provides rapid, dose dependent, and local production of Flexi IL12.

In some embodiments, the IL12 expression may be tuned to generate a Th1 response in vivo. CD4+T cells differentiate into effector Th1 cells that are involved in Th1 response. Th1 cells produce IL2 and interferon gamma, which are involved in cell mediated responses. In some embodiments, compositions of the invention may be tuned to achieve low basal expression in the absence of the stimulus and IL12 levels sufficient to generate Th1 response. In some embodiments, compositions of the invention may be tuned to achieve low basal expression in the absence of stimulus and then expression is induced at least 1×, 2×, 3×, 4×, 5×, 6×, 7×, 8×, 9×, 10×, or more than 10× upon the addition of the drug.

The format of the IL12 constructs utilized as payload of the present invention may be optimized. In one embodiment, the payload of the invention may be a bicistronic IL12 containing p40 and p35 subunits separated by an internal ribosome entry site or a cleavage site such as P2A or Furin to allow independent expression of both subunits from a single vector. This results in a configuration of secreted IL12 that is more akin to the naturally occurring IL12 than the flexi IL12 construct, the payload of the invention may be the p40 subunit of the IL12. DD regulated p40 may be co-expressed with constitutive p35 construct to generate “regulatable IL12” expression. Alternatively, the DD regulated p40 may heterodimerize with the endogenous p35. p40 has been shown to stabilize p35 expression and stimulate the export of p35 (Jalah R, et al. (2013). Journal of Biol. Chem. 288, 6763-6776 (the contents of which are incorporated by reference in its entirety).

In some embodiments, modified forms of IL12 may be utilized as the payload. These modified forms of IL12 may be engineered to have shortened half-life in vivo compared to the non-modified form of especially when used in combination with tunable systems described herein.

Human flexi IL12 has a reported half-life of 5-19 hours which, when administered as a therapeutic compound, can result in systemic cytotoxicity (Car et al. (1999) The Toxicology of Interleukin-12: A Review” Toxicologic Path. 27 A, 58-63; Robertson et al. (1999) “Immunological Effects of Interleukin 12 Administered by Bolus Intravenous Injection to Patients with Cancer” Clin. Cancer Res. 5:9-16; Atkins et al. (1997)“Phase I Evaluation of Intravenous Recombinant Human Interleukin 12 in Patients with Advance Malignancies” Clin. Cancer Res. 3:409-417). The ligand inducible control of IL12 can regulate production in a dose dependent fashion, the time from cessation of ligand dosing to cessation of protein synthesis and IL12 clearance may be insufficient to prevent toxic accumulation of IL12 in plasma.

In one embodiment, the modified form of IL12 utilized as the payload may be a Topo-scIL12 which have the configuration as follows from N to C terminus (i) a first IL12 p40 domain (p40N), (ii) an optional first peptide linker, (iii) an IL12 p35 domain, (iv) an optional second peptide linker, and (v) a second IL12 p40 domain (p40C). In one embodiment, modified topo-sc-IL12 polypeptides exhibit increased susceptibility to proteolysis. Topo-sc IL12 is described in International Patent Publication No. WO2016048903; the contents of which are incorporated herein by reference in its entirety.

IL12 polypeptide may also be modified (e.g. genetically, synthetically, or recombinantly engineered) to increase susceptibility to proteinases to reduce the biologically active half-life of the IL12 complex, compared to a corresponding IL12 lacking proteinases susceptibility. Proteinase susceptible forms of IL12 are described in International Patent Publication No. WO2017062953; the contents of which are incorporated by reference in its entirety.

In some embodiments, the pharmacokinetic/pharmacodynamic measurements of IL12 in vivo may be assessed by measuring serum IL12 levels and/or downstream mediators of IL12 such as IL16, IL6 and IL10.

IL12 systemic toxicity may also be limited or tightly controlled via mechanisms involving tethering IL12 to the cell surface to limit its therapeutic efficacy to the tumor site. Membrane tethered IL12 forms have been described previously using Glycosyl phosphatidylinositol (GPI) signal peptide or using CD80 transmembrane domain (Nagarajan S, et al. (2011) J Biomed Mater Res A. 99(3):410-7; Bozeman E N, et al. (2013) Vaccine. 7; 31(20):2449-56; Wen-Yu Pan et al. (2012), Mol. Ther. 20:5, 927-937; the contents of each of which are incorporated by reference in their entirety).

In one embodiment, the payload of the invention may comprise IL15. Interleukin 15 is a potent immune stimulatory cytokine and an essential survival factor for T cells, and Natural Killer cells. Preclinical studies comparing IL2 and IL15, have shown than IL15 is associated with less toxicity than IL2. In some embodiments, the effector module of the invention may be a DD-IL15 fusion polypeptide. IL15 polypeptide may also be modified to increase its binding affinity for the IL15 receptor. For example, the asparagine may be replaced by aspartic acid at position 72 of IL15 (SEQ ID NO. 2 of US patent publication US20140134128A1; the contents of which are incorporated by reference in their entirety). In some embodiments, the IL15 constructs of the invention may be placed under the transcriptional control of the CMV promoter (SEQ ID NO. 556, 1100), an EF1a promoter (SEQ ID NO. 557, 708, 1099, 1103) or a PGK promoter (SEQ ID NO. 558, 1101, 1102). In some aspects, the DD-IL15 comprises the amino acid sequences listed in Table 7. The amino acid sequences in Table 7 may comprise a stop codon which is denoted in the table with a “*” at the end of the amino acid sequence. In Table 7, “del” means deletion and “WT” means wild-type.

TABLE 7
DD-IL15 constructs

| | | | Amino | Nucleic |
|---|---|---|---|---|
| | | | Acid | Acid SEQ |
| Description/ | | | SEQ ID | ID |
| Construct ID | Promoter | Amino Acid Sequence | NO | NO |
| IL2 signal | — | MYRMQLLSCIALSLALVTNS | 614 | 617-620 |
| sequence | | | | |
| IgE signal | — | MDWTWILFLVAAATRVHS | 630 | 637, 730, |
| sequence | | | | 731 |
| (Leader) | | | | |
| Linker | — | EFSTEF | 615 | 621-622 |
| Linker | — | GGSGG | 470 | 516-520 |
| HA Tag | — | YPYDVPDYA | 823 | 824-826 |
| BamH1 | — | GS | — | GGATCC |
| P2A Cleavable | — | GATNFSLLKQAGDVEENPGP | 725 | 726 |
| Peptide | | | | |
| mCherry (MIL) | — | LSKGEEDNMAIIKEFMRFKVHMEGSVNG | 828 | 829 |
| | | HEFEIEGEGEGRPYEGTQTAKLKVTKGG | | |
| | | PLPFAWDILSPQFMYGSKAYVKHPADIP | | |
| | | DYLKLSFPEGFKWERVMNFEDGGVVTV | | |
| | | TQDSSLQDGEFIYKVKLRGTNFPSDGPV | | |
| | | MQKKTMGWEASSERMYPEDGALKGEIK | | |
| | | QRLKLKDGGHYDAEVKTTYKAKKPVQL | | |
| | | PGAYNVNIKLDITSHNEDYTIVEQYERAE | | |
| | | GRHSTGGMDELYK | | |
| IL15 (WT) | — | MRISKPHLRSISIQCYLCLLLNSHFLTEAG | 1095 | 1096 |
| | | IHVFILGCFSAGLPKTEANWVNVISDLKK | | |
| | | IEDLIQSMHIDATLYTESDVHPSCKVTAM | | |
| | | KCFLLELQVISLESGDASIHDTVENLIILA | | |
| | | NNSLSSNGNVTESGCKECEELEEKNIKEF | | |
| | | LQSFVHIVQMFINTS | | |
| IL15 (Amino | — | NWVNVISDLKKIEDLIQSMHIDATLYTES | 616 | 624-626, |
| acid 49-162 of | | DVHPSCKVTAMKCFLLELQVISLESGDA | | 801 |
| WT) | | SIHDTVENLIILANNSLSSNGNVTESGCKE | | |
| | | CEELEEKNIKEFLQSFVHIVQMFINTS* | | |
| ecDHFR | — | ISLIAALAVDYVIGMENAMPWNLPADLA | 4 | 532, 603, |
| (M1del, R12Y, | | WFKRNTLNKPVIMGRHTWESIGRPLPGR | | 641, 527, |
| Y100I) | | KNIILSSQPGTDDRVTWVKSVDEAIAAC | | 788, 791 |
| | | GDVPEIMVIGGGRVIEQFLPKAQKLYLTH | | |
| | | IDAEVEGDTHFPDYEPDDWESVFSEFHD | | |
| | | ADAQNSHSYCFEILERR* | | |
| hDHFR | — | VGSLNCIVAVSQNMGIGKNGDLPWPPLR | 696 | 534, 535 |
| (M1del, Y122I) | | NEFRYFQRMTTTSSVEGKQNLVIMGKKT | | |
| | | WFSIPEKNRPLKGRINLVLSRELKEPPQG | | |
| | | AHFLSRSLDDALKLTEQPELANKVDMV | | |
| | | WIVGGSSVIKEAMNHPGHLKLFVTRIMQ | | |
| | | DFESDTFFPEIDLEKYKLLPEYPGVLSDV | | |
| | | QEEKGIKYKFEVYEKND | | |

A unique feature of IL15 mediated activation is the mechanism of trans-presentation in which IL15 is presented as a complex with the alpha subunit of IL15 receptor (IL15Ra) that binds to and activates membrane bound IL15 beta/gamma receptor, either on the same cell or a different cell. The IL15/IL15Ra complex is more effective in activating IL15 signaling, than IL15 by itself. Thus, in some embodiments, the effector module of the invention may include a DD-IL15/IL15Ra fusion polypeptide. In one embodiment, the payload may be IL15/IL15Ra fusion polypeptide described in US Patent Publication NO. US20160158285A1 (the contents of which are incorporated herein by reference in their entirety). The IL15 receptor alpha comprises an extracellular domain called the sushi domain which contains most of the structural elements necessary for binding to IL15. Thus, in some embodiments, payload may be the IL15/IL15Ra sushi domain fusion polypeptide described in US Patent Publication NO. US20090238791A1 (the contents of which are incorporated herein by reference in their entirety).

Regulated IL15/IL15Ra may be used to promote expansion, survival and potency of CD8T_{EM} cell populations without impacting regulatory T cells, NK cells and TIL cells.

In some embodiments, compositions of the invention may be used to tune the persistence of the immune cells. In the context of immune cells, the term persistence refers to continued or prolonged existence of the immune cells in vitro or in vivo. In some embodiments, persistence of the immune cell may be linked to the increased longevity of the immune cells. In some embodiments, immune cell persistence may be associated with increased proliferation of the immune cells. In some aspects, persistence may be associated with a change in the differentiation status of the cell. In one embodiments, the persistence of the immune cell may be achieved by using IL15-IL15Ra as the payload of the invention. Persistence of the least differentiated memory T cell, the T-memory stem cell, has been shown to be promoted by signaling induced by a membrane-bound IL-15-IL15Ra cytokine-fusion molecule. This may in turn, promote the therapeutic efficacy of CAR-based immunotherapies of patients with advanced cancer.

In some embodiments, the immunotherapeutic agent of the composition may be a cytokine. The cytokine may be an interleukin, an interferon, a tumor necrosis factor, a transforming growth factor B, a CC chemokine, a CXC chemokine, a CX3C chemokine or a growth factor.

In one aspect, the interleukin may be a whole or a portion of a IL15 and may comprise the amino acid sequence of SEQ ID NO. 1095. In one aspect, the IL15 may be modified. In some embodiments, the modifications may comprise fusing SEQ ID NO. 1095 to the whole or a portion of, a transmembrane domain. The IL15 may optionally be modified by incorporating a hinge domain.

In some embodiment, the immunotherapeutic agent of the composition may be a cytokine receptor. In one aspect, the cytokine receptor may be IL15Ra and may comprise the amino acid sequence of SEQ ID NO. 1097. In one aspect, the IL15Ra may be modified. In some embodiments, the modifications may comprise fusing SEQ ID NO. 1097 to the whole or a portion of, a transmembrane domain. The IL15Ra may optionally be modified by incorporating a hinge domain.

The present invention also provides methods for enhancing the expansion and/or survival of immune cells, comprising contacting the immune cells with the compositions of the invention, the polynucleotides of the invention, and/or the vectors of the invention.

Also provided herein, is a method for inducing an immune response in a subject, administering the compositions of the invention, the polynucleotides of the invention, and/or the immune cells of the invention to the subject.

In one embodiment, DD-IL15/IL15Ra may be utilized to enhance CD19 directed T cell therapies in B cell leukemia and lymphomas. In one aspect, IL15/IL15Ra may be used as payload of the invention to reduce the need for pre-conditioning regimens in current CAR-T treatment paradigms.

The effector modules containing DD-IL15, DD-IL15/IL15Ra and/or DD-IL15/IL15Ra sushi domain may be designed to be secreted (using e.g. IL2 signal sequence) or membrane bound (using e.g. IgE or CD8a signal sequence).

In some aspects, the DD-IL115/IL15Ra comprises the amino acid sequences provided in Table 8 and Table 9. In Table 8 and Table 9, “del” means deletion and “WT” means wild-type.

TABLE 8
DD-IL15/IL15Ra construct sequences

| | | Amino Acid | Nucleic |
|---|---|---|---|
| Description/ | | SEQ ID | Acid SEQ |
| Construct ID | Amino Acid Sequence | NO | ID NO |
| IgE leader | MDWTWILFLVAAATRVHS | 630 | 637, 730, |
| | | | 731 |
| IL15RA Leader | MAPRRARGCRTLGLPALLLLLLLRPPATRG | 732 | 733 |
| Linker (SG3- | SGGGSGGGGSGGGGSGGGGSGGGSLQ | 631 | 638,716- |
| (SG4)3SG3SLQ) | | | 720, 802 |
| Linker (SG3S) | SGGGS | 654 | 655, 670, |
| | | | 709 |
| Linker | SGGGSGGGGSGGGGSGGGGSGGGGSGGGGSGG | 721 | 723 |
| (SG3(SG4)5SG3S) | GS | | |
| Linker (SG3- | SGGGSGGGGSGGGGSGGGGS | 722 | 724 |
| (SG4)3)S | | | |
| BamH1 (Linker) | GS | - | GGTTCC, |
| | | | GGATCC |
| Linker | SG | - | AGCGGC |
| Linker | GSG | - | GGATCC |
| | | | GGA or |
| | | | GGATCC |
| | | | GGT |
| Spacer | | | 727-729, |
| | | | 800, |
| | | | TCGCGA |
| | | | ATG, |
| | | | TCGCA |
| CD8a Hinge- | TTTPAPRPPTPAPTIASQPLSLRPEACRPAAGGAV | 403 | 468 |
| Transmembrane | HTRGLDFACDIYIWAPLAGTCGVLLLSLVITLYC | | |
| Domain (TM) | | | |
| IL15 (WT) | MRISKPHLRSISIQCYLCLLLNSHFLTEAGIHVFIL | 1095 | 1096 |
| | GCFSAGLPKTEANWVNVISDLKKIEDLIQSMHID | | |
| | ATLYTESDVHPSCKVTAMKCFLLELQVISLESGD | | |
| | ASIHDTVENLIILANNSLSSNGNVTESGCKECEEL | | |
| | EEKNIKEFLQSFVHIVQMFINTS | | |
| IL15 (49-162 of | NWVNVISDLKKIEDLIQSMHIDATLYTESDVHPS | 616 | 623-626, |
| WT) | CKVTAMKCFLLELQVISLESGDASIHDTVENLIIL | | 801 |
| | ANNSLSSNGNVTESGCKECEELEEKNIKEFLQSF | | |
| | VHIVQMFINTS* | | |
| IL15Ra(WT; | MAPRRARGCRTLGLPALLLLLLLRPPATRGITCP | 1097 | 1098 |
| Uniprot ID: | PPMSVEHADIWVKSYSLYSRERYICNSGFKRKA | | |
| Q13261.1) | GTSSLTECVLNKATNVAHWTTPSLKCIRDPALV | | |
| | HQRPAPPSTVTTAGVTPQPESLSPSGKEPAASSPS | | |
| | SNNTAATTAAIVPGSQLMPSKSPSTGTTEISSHES | | |
| | SHGTPSQTTAKNWELTASASHQPPGVYPQGHSD | | |
| | TTVAISTSTVLLCGLSAVSLLACYLKSRQTPPLAS | | |
| | VEMEAMEALPVTWGTSSRDEDLENCSHHL | | |
| IL15Ra (31-267 of | ITCPPPMSVEHADIWVKSYSLYSRERYICNSGFK | 632 | 639-640, |
| WT) | RKAGTSSLTECVLNKATNVAHWTTPSLKCIRDP | | 803 |
| | ALVHQRPAPPSTVTTAGVTPQPESLSPSGKEPAA | | |
| | SSPSSNNTAATTAAIVPGSQLMPSKSPSTGTTEIS | | |
| | SHESSHGTPSQTTAKNWELTASASHQPPGVYPQG | | |
| | HSDTTVAISTSTVLLCGLSAVSLLACYLKSRQTPP | | |
| | LASVEMEAMEALPVTWGTSSRDEDLENCSHHL* | | |
| IL15Ra (31-205 of | ITCPPPMSVEHADIWVKSYSLYSRERYICNSGFK | 855 | 856 |
| WT) | RKAGTSSLTECVLNKATNVAHWTTPSLKCIRDP | | |
| | ALVHQRPAPPSTVTTAGVTPQPESLSPSGKEPAA | | |
| | SSPSSNNTAATTAAIVPGSQLMPSKSPSTGTTEISS | | |
| | HESSHGTPSQTTAKNWELTASASHQPPGVYPQG | | |
| | HSDTT | | |
| mCherry | MSKGEEDNMAIIKEFMRFKVHMEGSVNGHEFEI | 857 | 858 |
| | EGEGEGRPYEGTQTAKLKVTKGGPLPFAWDILSP | | |
| | QFMYGSKAYVKHPADIPDYLKLSFPEGFKWERV | | |
| | MNFEDGGVVTVTQDSSLQDGEFIYKVKLRGTNF | | |
| | PSDGPVMQKKTMGWEASSERMYPEDGALKGEI | | |
| | KQRLKLKDGGHYDAEVKTTYKAKKPVQLPGAY | | |
| | NVNIKLDITSHNEDYTIVEQYERAEGRHSTGGM | | |
| | DELYK* | | |
| mCherry (MIL) | LSKGEEDNMAIIKEFMRFKVHMEGSVNGHEFEIE | 828 | 829 |
| | GEGEGRPYEGTQTAKLKVTKGGPLPFAWDILSP | | |
| | QFMYGSKAYVKHPADIPDYLKLSFPEGFKWERV | | |
| | MNFEDGGVVTVTQDSSLQDGEFIYKVKLRGTNF | | |
| | PSDGPVMQKKTMGWEASSERMYPEDGALKGEI | | |
| | KQRLKLKDGGHYDAEVKTTYKAKKPVQLPGAY | | |
| | NVNIKLDITSHNEDYTIVEQYERAEGRHSTGGMD | | |
| | ELYK | | |
| HA Tag | YPYDVPDYA | 823 | 824-826 |
| Flag | DYKDDDDK | 1030 | - |
| P2A Cleavable | GATNFSLLKQAGDVEENPGP | 725 | 726 |
| Peptide | | | |
| ecDHFR (M1del, | ISLIAALAVDYVIGMENAMPWNLPADLAWFKRN | 4 | 532, 603, |
| RI2Y, Y100I) | TLNKPVIMGRHTWESIGRPLPGRKNIILSSQPGTD | | 641, 527, |
| | DRVTWVKSVDEAIAACGDVPEIMVIGGGRVIEQ | | 788, 791 |
| | FLPKAQKLYLTHIDAEVEGDTHFPDYEPDDWES | | |
| | VFSEFHDADAQNSHSYCFEILERR* | | |
| ecDHFR (M1del, | ISLIAALAVDHVIGMENAMPWNLPADLAWFKRN | 5 | 627, 842, |
| RI2H, E129K) | TLNKPVIMGRHTWESIGRPLPGRKNIILSSQPGT | | 793 |
| | DDRVTWVKSVDEAIAACGDVPEIMVIGGGRVYEQ | | |
| | FLPKAQKLYLTHIDAEVEGDTHFPDYKPDDWES | | |
| | VFSEFHDADAQNSHSYCFEILERR* | | |
| FKBP (E32G, | GVQVETISPGDGRTFPKRGQTCVVHYTGMLGDG | 7 | 528-531, |
| F37V, R72G, | KKVDSSRDRNKPFKFMLGKQEVIRGWEEGVAQ | | 794, 812, |
| K106E) | MSVGQGAKLTISPDYAYGATGHPGIIPPHATLVF | | 827 |
| | DVELLELE* | | |
| hDHFR (M1del, | VGSLNCIVAVSQNMGIGKNGDLPWPPLRNEFRY | 691 | 536, 773, |
| Y122I, A125F) | FQRMTTTSSVEGKQNLVIMGKKTWFSIPEKNRPL | | 774, 796 |
| | KGRINLVLSRELKEPPQGAHFLSRSLDDALKLTE | | |
| | QPELANKVDMVWIVGGSSVIKEFMNHPGHLKLF | | |
| | VTRIMQDFESDTFFPEIDLEKYKLLPEYPGVLS | | |
| | DVQEEKGIKYKFEVYEKND* | | |
| hDHFR (M1del, | VGSLNCIVAVSQNMGIGKNGDLPWPPLRNEFRY | 692 | 540, 775, |
| Q36F, N65F, | FFRMTTTSSVEGKQNLVIMGKKTWFSIPEKFRPL | | 776, 798 |
| Y122I) | KGRINLVLSRELKEPPQGAHFLSRSLDDALKLTE | | |
| | QPELANKVDMVWIVGGSSVIKEAMNHPGHLKL | | |
| | FVTRIMQDFESDTFFPEIDLEKYKLLPEYPGVLSD | | |
| | VQEEKGIKYKFEVYEKND | | |
| hDHFR (M1del, | VGSLNCIVAVSQNMGIGKNGDLPWPPLRNEFRY | 690 | 772 |
| K185E) | FQRMTTTSSVEGKQNLVIMGKKTWFSIPEKNRPL | | |
| | KGRINLVLSRELKEPPQGAHFLSRSLDDALKLTE | | |
| | QPELANKVDMVWIVGGSSVYKEAMNHPGHLKLF | | |
| | VTRIMQDFESDTFFPEIDLEKYKLLPEYPGVLSD | | |
| | VQEEKGIKYKFEVYEEND* | | |
| hDHFR (M1del, | VGSLNCIVAVSQNMGIGKNGDLPWPPLRNEFRY | 689 | 770-772 |
| E162G, I176F) | FQRMTTTSSVEGKQNLVIMGKKTWFSIPEKNRPL | | |
| | KGRINLVLSRELKEPPQGAHFLSRSLDDALKLTE | | |
| | QPELANKVDMVWIVGGSSVYKEAMNHPGHLKLF | | |
| | VTRIMQDFESDTFFPEIDLEKYKLLPGYPGVLSD | | |
| | VQEEKGFKYKFEVYEKND* | | |
| hDHFR (M1del, | VGSLNCIVAVSQNMGIGKNGDLPWPPLRNEFRY | 693 | 777 |
| N127Y) | FQRMTTTSSVEGKQNLVIMGKKTWFSIPEKNRPL | | |
| | KGRINLVLSRELKEPPQGAHFLSRSLDDALKLTE | | |
| | QPELANKVDMVWIVGGSSVYKEAMYHPGHLKLF | | |
| | VTRIMQDFESDTFFPEIDLEKYKLLPEYPGVLSD | | |
| | VQEEKGIKYKFEVYEKND | | |
| hDHFR (M1del, | VGSLNCIVAVSQNMGVGKNGDLPWPPLRNEFR | 695 | 779 |
| I17V) | YFQRMTTTSSVEGKQNLVIMGKKTWFSIPEKNR | | |
| | PLKGRINLVLSRELKEPPQGAHFLSRSLDDALKL | | |
| | TEQPELANKVDMVWIVGGSSVYKEAMNHPGHLK | | |
| | LFVTRIMQDFESDTFFPEIDLEKYKLLPEYPGVL | | |
| | SDVQEEKGIKYKFEVYEKND | | |
| hDHFR (M1del, | VGSLNCIVAVSQNMGVGKNGDLPWPPLRNEFR | 688 | 769 |
| I17V, Y122I) | YFQRMTTTSSVEGKQNLVIMGKKTWFSIPEKNR | | |
| | PLKGRINLVLSRELKEPPQGAHFLSRSLDDALKL | | |
| | TEQPELANKVDMVWIVGGSSVIKEAMNHPGHLK | | |
| | LFVTRIMQDFESDTFFPEIDLEKYKLLPEYPGVL | | |
| | SDVQEEKGIKYKFEVYEKND | | |
| hDHFR (M1del, | VGSLNCIVAVSQNMGIGKNGDLPWPPLRNEFRY | 694 | 778 |
| H131R, E144G) | FQRMTTTSSVEGKQNLVIMGKKTWFSIPEKNRPL | | |
| | KGRINLVLSRELKEPPQGAHFLSRSLDDALKLTE | | |
| | QPELANKVDMVWIVGGSSVYKEAMNHPGRLKLF | | |
| | VTRIMQDFGSDTFFPEIDLEKYKLLPEYPGVLSD | | |
| | VQEEKGIKYKFEVYEKND | | |

TABLE 9
DD-IL15/IL15Ra constructs

| | | | Amino | Nucleic |
|---|---|---|---|---|
| | | | Acid | Acid |
| | | | SEQ ID | SEQ ID |
| Description | Promoter | Amino acid sequences | NO | NO |
| OT-001111 and OT- | EF1a | MDWTWILFLVAAATRVHSNWVNVISD | 636 | 646 |
| 001471 (IgE signal | | LKKIEDLIQSMHIDATLYTESDVHPSCK | | |
| sequence; IL15 (49- | | VTAMKCFLLELQVISLESGDASIHDTVE | | |
| 162 of WT); linker | | NLIILANNSLSSNGNVTESGCKECEELE | | |
| (SG3-(SG4)3-SG3- | | EKNIKEFLQSFVHIVQMFINTSSGGGSG | | |
| SLQ); IL15Ra(31- | | GGGSGGGGSGGGGSGGGSLQITCPPPMS | | |
| 267 of WT); Linker | | VEHADIWVKSYSLYSRERYICNSGFKR | | |
| (SG); ecDHFR | | KAGTSSLTECVLNKATNVAHWTTPSLK | | |
| (M1del, R12Y, | | CIRDPALVHQRPAPPSTVTTAGVTPQPE | | |
| 1001); stop) (OT- | | SLSPSGKEPAASSPSSNNTAATTAAIVPG | | |
| IL15-009 and OT- | | SQLMPSKSPSTGTTEISSHESSHGTPSQT | | |
| IL15-073) | | TAKNWELTASASHQPPGVYPQGHSDTT | | |
| | | VAISTSTVLLCGLSAVSLLACYLKSRQT | | |
| | | PPLASVEMEAMEALPVTWGTSSRDEDL | | |
| | | ENCSHHLSGISLIAALAVDYVIGMENA | | |
| | | MPWNLPADLAWFKRNTLNKPVIMGRH | | |
| | | TWESIGRPLPGRKNIILSSQPGTDDRVT | | |
| | | WVKSVDEAIAACGDVPEIMVIGGGRVI | | |
| | | EQFLPKAQKLYLTHIDAEVEGDTHFPD | | |
| | | YEPDDWESVFSEFHDADAQNSHSYCFEI | | |
| | | LERR* | | |
| OT-001418 and OT- | EF1a | MDWTWILFLVAAATRVHSNWVNVISD | 635 | 645 |
| 001422 (IgE signal | | LKKIEDLIQSMHIDATLYTESDVHPSCK | | |
| sequence; IL15 (49- | | VTAMKCFLLELQVISLESGDASIHDTVE | | |
| 162 of WT); Linker | | NLIILANNSLSSNGNVTESGCKECEELEE | | |
| (SG3-(SG4)3-SG3- | | KNIKEFLQSFVHIVQMFINTSSGGGSGG | | |
| SLQ); IL15Ra(31- | | GGSGGGGSGGGGSGGGSLQITCPPPMS | | |
| 267 of WT); stop) | | VEHADIWVKSYSLYSRERYICNSGFKR | | |
| (OT-IL15-064 and | | KAGTSSLTECVLNKATNVAHWTTPSLK | | |
| OT-IL15-071) | | CIRDPALVHQRPAPPSTVTTAGVTPQPE | | |
| | | SLSPSGKEPAASSPSSNNTAATTAAIVPG | | |
| | | SQLMPSKSPSTGTTEISSHESSHGTPSQT | | |
| | | TAKNWELTASASHQPPGVYPQGHSDTT | | |
| | | VAISTSTVLLCGLSAVSLLACYLKSRQT | | |
| | | PPLASVEMEAMEALPVTWGTSSRDEDL | | |
| | | ENCSHHL* | | |

In some embodiments, payloads of the present invention may comprise inhibitory molecules that block inhibitory cytokines. The inhibitors may be blocking antibodies specific to an inhibitory cytokine, and antagonists against an inhibitory cytokine, or the like.

In some aspects, payloads of the present invention may comprise an inhibitor of a secondary cytokine IL35. IL35 belongs to the interleukin-12 (IL12) cytokine family, and is a heterodimer composed of the IL27 β chain Ebi3 and the IL12 achain p35. Secretion of bioactive IL35 has been described only in forkhead box protein 3 (Foxp3)⁺ regulatory T cells (Tregs) (resting and activated Tregs). Unlike other membranes in the family, IL35 appears to function solely in an anti-inflammatory fashion by inhibiting effector T cell proliferation and perhaps other parameters (Collison et al., Nature, 2007, 450(7169): 566-569).

In some embodiments, payloads of the present invention may comprise fusion proteins wherein a cytokine, chemokine and/or other soluble factor may be fused to other biological molecules such as antibodies and or ligands for a receptor. Such fusion molecules may increase the half-life of the cytokines, reduce systemic toxicity, and increase local concentration of the cytokines at the tumor site. Fusion proteins containing two or more cytokines, chemokines and or other soluble factors may be utilized to obtain synergistic therapeutic benefits.

### Safety Switch

In some embodiments, payloads of the present invention may comprise SRE regulated safety switches that can eliminate adoptively transferred cells in the case of severe toxicity, thereby mitigating the adverse effects of T cell therapy. Adoptively transferred T cells in immunotherapy may attack normal cells in response to normal tissue expression of TAA. Even on-tumor target activity of adoptively transferred T cells can result in toxicities such as tumor lysis syndrome, cytokine release syndrome and the related macrophage activation syndrome. Safety switches may be utilized to eliminate inappropriately activated adoptively transferred cells by induction of apoptosis or by immunosurveillance.

### Regulatory Switch

The utility of adoptive cell therapy (ACT) has been limited by the high incidence of graft versus host disease (GVHD). GVHD occurs when adoptively transferred T cells elicit an immune response resulting in host tissue damage. Recognition of host antigens by the graft cells triggers a proinflammatory cytokine storm cascade that signifies acute GVHD. GVHD is characterized as an imbalance between the effector and the regulatory arms of the immune system. In some embodiments, the payloads of the present invention may be used as regulatory switches. As used herein “regulatory switch” refers proteins, which when expressed in target cells increase tolerance to the graft by enhancing the regulatory arm of the immune system.

### Antigen

As used herein, any molecule capable of being recognized by one or more constituents of the immune system is called an “antigen.” In some embodiments, the effector modules described herein may comprise antigen. Antigens, as described herein, may be used as the SRE and/or as the payloads of the invention. Antigens of the invention may be whole protein, a truncated protein, a fragment of a protein or a peptide. Antigens may be naturally occurring, genetically engineered variants of the protein, or may be codon optimized for expression in a particular mammalian subject or host. Further, the antigens of the present invention, may include modifications, such as deletions, additions and substitutions, generally conservative in nature, to the naturally occurring sequence, so long as the protein maintains its ability to elicit an immunological response. The modifications may be intentional, as through site directed mutagenesis, or may be accidental such as through mutations in the hosts which produce the antigens. Antigens of the present invention may also be codon optimized to improve their expression or immunogenicity in the host.

Antigens may comprise one or more epitopes, which refers to the portion of the antigen that is recognized by the immune systems, specifically, the antibodies, B cells or T cells. Normally, an epitope will include between about 7 and 15 amino acids, such as, 9, 10, 12 or 15 amino acids. The term “antigen” denotes both subunit antigens, (i.e., antigens which are separate and discrete from a whole organism with which the antigen is associated in nature). Antibodies such as anti-idiotype antibodies, or fragments thereof, and synthetic peptide mimotopes, that is synthetic peptides which can mimic an antigen or antigenic determinant, are also captured under the definition of antigen as used herein.

In some embodiments, the antigens are recognized by the innate immune system. In such instances, the antigens are the pathogen associated molecular patterns recognized by the pattern recognition receptors expressed on macrophages, dendritic cells and NK cells. NK cells express an array of additional sets of receptors that recognize unconventional antigens. In some embodiments, the antigens are recognized by the adaptive immune system, the B lymphocyte-expressed immunoglobulin and T lymphocyte expressed T cell receptor recognize either specific conformation on the antigen or the amino acid sequence in the peptide respectively.

In one embodiment, the antigen may be a tolerogen. Antigens, which induce tolerogenic or allergic responses are called tolerogens. In some embodiments, antigens that do not elicit immune responses may be useful in the present invention. In other embodiments, antigens that induce or elicit an immune response may be preferred, and such antigens are referred to as immunogens.

In some embodiments, the antigens of the present invention may be incomplete antigens, which are also referred to as hapten. Haptens referred to molecules that can interact with components of the immune system, but do not elicit an immune response. The haptens may be combined with a carrier to prepare a complete antigen.

Antigens useful in the present invention may be either exogenous antigens or endogenous antigens. Exogenous antigens refer to the antigens that enter the body by inhalation, ingestion or injection. Such antigens are taken up by the antigen presenting cells (APCs) and degraded into peptides. APCs then present such antigens to CD4+ helper T cells using Class II MHC molecules. Endogenous antigens as used herein refers to antigens that are produced within the cell as a result of normal cellular metabolism, or an infection. Such antigens are presented on the cell surface using MHC type I molecules to CD8+ Cytotoxic T cells.

Antigens of the present invention may also be classified based on the source of the antigen. In some embodiments, the antigen of the present invention may be a xenoantigen, wherein the antigen is derived from a different species e.g. bacteria or viruses; an alloantigen, wherein the antigen is derived from a different individual of the same species e.g. a blood group antigen; an autoantigen, wherein the antigen is derived from the same individual e.g. tumor antigen; or a heterophile antigen, wherein the antigen is common and shared by different species.

In some embodiments, antigens of the present invention may also be selected based on their ability to induce T cell responses. T cell dependent (TD) antigen or a T cell independent (TI) antigen. TD antigens are structurally complex antigens that require T cells to generate an immune response. They are immunogenic over a wide dose range and do not cause tolerance.

Such antigens require processing by the APCs and are capable of producing immunologic memory. In contrast, TI antigens of the present invention are able to directly stimulate the B cells to produce antibodies without the participation of T cells. These antigens are structurally simple, and are composed of a limiting number of repeating epitopes. TI antigens do not induce tolerance, are less immunogenic and do not produce immunological memory.

In some embodiments, the antigens of the present invention may be derived from a specific subcellular location. In some embodiments, sub cellular location from which the antigen is derived may be the plasma membrane, the cell surface, the nucleus, the cytosol, the lysosome, the endosome, the mitochondria, the peroxisome, the Golgi body, the endoplasmic reticulum or any other organelle within the cell. In some embodiments, the cell may be a eukaryotic or a prokaryotic cell.

Antigens of the present invention may be infectious disease antigens, which herein refers to antigens associated with infectious diseases causing agents or microorganisms. In some embodiments, the antigen is an infectious disease antigen associated with an infectious disease. In some embodiments, the antigen is a viral antigen derived from a virus (e.g., and thereby expressing one or more viral antigens) and/or virus-like particle; or a bacterial antigen derived from a bacterium; a protozoan antigen derived from a protozoan; a prion antigen derived from a prion particle; or a fungal antigen derived from a fungus.

In some embodiments, antigen size may be used to identify antigens useful in the present invention. In general, antigens larger than 5000 Da are considered to be more immunogenic than antigens that are smaller than 5000 Da. In some embodiments, antigens, that are larger than 5000 Da may be preferred, such as 6000 Da, 7000 Da, 8000 Da, 9000 Da, 10,000 Da and larger. In some embodiments, antigens that are smaller than 5000 Da may be preferred to induce a weak immune response or no immune response.

In some embodiments, antigens of the present invention are preferably macromolecules. Such molecules have been shown to be more immunogenic than micromolecules. In some embodiments, the SREs and/or payloads of the invention may be less immunogenic antigens such polypeptides, glycoproteins, nucleic acids, and lipids.

In some embodiments, antigens of the present invention, may be classified based on the tissues from which the antigen originates. Such antigens may be a nervous tissue antigen, wherein the antigen is specific to the neurological tissue such as the brain, spinal cord, the central nervous system, the peripheral nervous system, including the sympathetic and parasympathetic nervous system. Such antigens may also originate from neuronal cell types such as Schwann cells, the axon, the motor or the ganglioside neuron, the glial cells, the astrocytes, progenitor cells, oligodendrocytes.

In some embodiments, the antigen is a connective tissue antigen, implying that the antigen is expressed by cells that bind other cells, and organs of the body together. Connective tissue antigens may be derived from loose connective tissues such as areolar connective tissue, adipose tissue, reticular tissue; dense connective tissue such as dense regular connective tissue or dense irregular connective tissue; or special connective tissue such as cartilage, bone and blood.

In other embodiments, the antigen is a muscle antigen. Such muscle antigens may be derived from skeletal (voluntary) muscle tissue, smooth muscle tissue and/or cardiac muscle tissue.

In one embodiment, the tissue antigen of the present invention may be an epithelium antigen derived from the epithelium that covers the exterior surface of the body and lines the internal cavities and passageways as well as forms certain glands. Antigens of the present invention may be derived from cuboidal epithelium, squamous epithelium and/or columnar epithelium.

In some embodiments, SREs and/or payloads of the present invention may be tumor specific antigens (TSAs), tumor associated antigens (TAAs). The antigen can be expressed as a peptide or as an intact protein or portion thereof. The intact protein or a portion thereof can be native or mutagenized. Antigens associated with cancers or virus-induced cancers as described herein are well-known in the art. Such a TSA or TAA may be previously associated with a cancer or may be identified by any method known in the art.

A tumor associated antigen (TAA) may be an overexpressed or accumulated antigen that is expressed by both normal and neoplastic tissue, with the level of expression highly elevated in cancer tissues. Numerous proteins (e.g. oncogenes) are up-regulated in tumor tissues, including but not limited to adipophilin, AIM-2, ALDH1A1, BCLX(L), BING-4, CALCA, CD45, CD274, CPSF, cyclin D1, DKK1, ENAH, epCAM, ephA3, EZH2, FGFS, G250, HER-2/neu, HLA-DOB, Hepsin, IDO1, IGFB3, IL13Ralpha2, Intestinal carboxyl esterase, kallikrein 4, KIF20A, lengsin, M-CSF, MCSP, mdm-2, Meloe, Midkine, MMP-2, MMP-7, MUC-1, MUCSAC, p53, Pax5, PBF, PRAME, PSMA, RAGE-1, RGSS, RhoC, RNF43, RU2A5, SECERNIN 1, SOX10, STEAP1, survivin, Telomerase, TPBG, VEGF, and WT1.

A TAA may be a cancer-testis antigen that is expressed only by cancer cells and adult reproductive tissues such as testis and placenta, including, but limited to antigens from BAGE family, CAGE family, HAGE family, GAGE family, MAGE family (e.g., MAGE-A1, MAGE-A2, MAGE-A3, MAGE-A6 and MAGE-A13), SAGE family, XAGE family, MCAK, NA88-A (cancer/testis antigen 88), PSAD1, SSX-2, and SLLP-1.

A TAA may be a lineage restricted antigen that is expressed largely by a single cancer histotype, such as Melan-A/MART-1, Gp100/pme117, Tyrosinase, TRP-1/-2, P. polypeptide, MC1R in melanoma; and prostate specific antigen (PSA) in prostate cancer.

A TAA may be an oncoviral antigen that is encoded by tumorigenic transforming viruses (also called oncogenic viruses). Oncogenic viruses, when they infect host cells, can insert their own DNA (or RNA) into that of the host cells. When the viral DNA or RNA affects the host cell's genes, it can push the cell toward becoming cancer. Oncogenic viruses include, but are not limited to, RNA viruses, and DNA viruses. Some examples of commonly known oncoviruses include human papilloma viruses (HPVs) which are main causes of cervical cancer, Epstein-Barr virus (EBV) which may cause nasopharyngeal cancer, certain types of fast-growing lymphomas (e.g., Burkitt lymphoma) and stomach cancer, hepatitis B, C and D viruses (HBV, HCV and HDV) in hepatocellular carcinoma (HCC), human immunodeficiency virus (HIV) which increases the risk of getting many types of cancer (e.g., liver cancer, anal cancer and Hodgkin cancer), Kaposi sarcoma herpes virus (KSHV; also known as human herpes virus 8 (HHV8)) which is linked to lymphoma, human T-lymphotrophic virus (HTLV-1) and merkel cell polymavirus (MCV). A viral antigen can be any defined antigen of a virus that is associated with a cancer in a human. For example, antigens from EBV may include but are not limited to, Epstein-Barr nuclear antigen-1 (EBNA1), latent membrane protein 1 (LMP1), or latent membrane protein 2 (LMP2).

A TAA may be an idiotypic antigen that is generated from highly polymorphic genes where a tumor cell expresses a specific “clonotype”, i.e., as in B cell, T cell lymphoma/leukemia resulting from clonal aberrancies, such as Immunoglobulin and T cell receptors (TCRs). Idiotypic antigens are a class of non-pathogen-associated neoantigens. For example, the malignant B cells express rearranged and multiply mutated surface immunoglobulins (Ig). Tumor specific idiotypes (e.g., immunoglobulin idiotypes) are regarded as particularly attractive tumor-specific antigens that can be successfully targeted by immunotherapy (e.g., Alejandro et al., Front Oncol., 2012, 2: 159).

### HLA Antigens

Human leukocyte antigens (HLA) are antigens expressed on all cell types of a subject and are of particular significance in the context of transplantation, wherein the transplantation recipient's immune system recognizes the HLA antigens of the donor and attacks the donor tissue causing transplant rejection. HLA antigens may either be class I or class II.

In some embodiments, antigens of the present invention may be HLA class I molecule consists of a 45-kDa glycoprotein (heavy chain) non-covalently associated with a 12-kDa polypeptide, β2-microglobulin β2m). Association of β2m with newly synthesized class I heavy chains is required in order for the HLA molecule to transport and present the peptide (Krangel et al., Cell 18: 979, 1979). However, β2m free class I heavy chains were identified on activated T lymphocytes (Schnabl et al., J. Exp. Med. 171:1431, 1990) and other cell surfaces (Bix & Raulet, J. Exp. Med. 176(3) 829-34, 1992). Properly conformed β2m free class I heavy chains were identified on the cells and were believed to have functional importance. β2m can be dissociated from a HLA class I complex on a cell surface by acid treatment (Sugawara et al., J. Immunol. Methods, 100(1-2):83-90, 1987). β2m can also be dissociated from HLA Class I complex coated on microbeads using the similar method of low pH treatment. (Pei et al. Visuals Clinical Histocompatability Workshop 2000, 9-10). Those β2m-free HLA heavy chains are referred to as “denatured antigens.”

### Immune Signaling

Treatment with immunotherapeutic agents may induce immune cell signaling, leading to the activation of cell-type specific immune activities, ultimately resulting in an immune response. In some embodiments, payloads of the present invention may be immune signaling biomolecules used to achieve exogenous control of signaling pathways.

In some embodiments, payloads of the present invention may be one or more coat proteins of the viruses, inserted transgenes, other factors that can increase intratumoral virus replication and the combinations.

In some instance, two or more oncolytic viruses may also be used as payload within the same SRE or in two or more SREs to achieve synergistic killing of target cancer cells.

### Additional Effector Module Features

The effector module of the present invention may further comprise a signal sequence which regulates the distribution of the payload of interest, a cleavage and/or processing feature which facilitate cleavage of the payload from the effector module construct, a targeting and/or penetrating signal which can regulate the cellular localization of the effector module, a tag, and/or one or more linker sequences which link different components of the effector module.

### Signal Sequences

In addition to the SRE (e.g., DD) and payload region, effector modules of the invention may further comprise one or more signal sequences. Signal sequences (sometimes referred to as signal peptides, targeting signals, target peptides, localization sequences, transit peptides, leader sequences or leader peptides) direct proteins (e.g., the effector module of the present invention) to their designated cellular and/or extracellular locations. Protein signal sequences play a central role in the targeting and translocation of nearly all secreted proteins and many integral membrane proteins.

A signal sequence is a short (5-30 amino acids long) peptide present at the N terminus of the majority of newly synthesized proteins that are destined towards a particular location. Signal sequences can be recognized by signal recognition particles (SRPs) and cleaved using type I and type II signal peptide peptidases. Signal sequences derived from human proteins can be incorporated as a regulatory module of the effector module to direct the effector module to a particular cellular and/or extracellular location. These signal sequences are experimentally verified and can be cleaved (Zhang et al., Protein Sci. 2004, 13:2819-2824).

In some embodiments, a signal sequence may be, although not necessarily, located at the N terminus or C terminus of the effector module, and may be, although not necessarily, cleaved off the desired effector module to yield a “mature” payload, i.e., an immunotherapeutic agent as discussed herein.

In some embodiments, the signal sequence used herein may exclude the methionine at the position 1 of amino acid sequence of the signal sequence. This may be referred to as an M1del mutation.

In some examples, a signal sequence may be a secreted signal sequence derived from a naturally secreted protein, and its variant thereof. In some instances, the secreted signal sequences may be cytokine signal sequences such as, but not limited to, IL2 signal sequence comprising amino acid of SEQ ID NO. 614, encoded by the nucleotide of SEQ ID NO. 617-620 and/or p40 signal sequence comprising the amino acid sequence of SEQ ID NO. 559, encoded by the nucleotide of SEQ ID NO. 567-575.

In some instances, signal sequences directing the payload of interest to the surface membrane of the target cell may be used. Expression of the payload on the surface of the target cell may be useful to limit the diffusion of the payload to non-target in vivo environments, thereby potentially improving the safety profile of the payloads. Additionally, the membrane presentation of the payload may allow for physiologically and qualitative signaling as well as stabilization and recycling of the payload for a longer half-life. Membrane sequences may be the endogenous signal sequence of the N terminal component of the payload of interest. Optionally, it may be desirable to exchange this sequence for a different signal sequence. Signal sequences may be selected based on their compatibility with the secretory pathway of the cell type of interest so that the payload is presented on the surface of the T cell. In some embodiments, the signal sequence may be IgE signal sequence comprising amino acid SEQ ID NO. 630 and nucleotide sequence of SEQ ID NO. 637, 730, or 731, CD8αsignal sequence (also referred to as CD8αleader) comprising amino acid SEQ ID NO. 469 and nucleotide sequence of SEQ ID NO. 511-515, or IL15Ra signal sequence (also referred to as IL15Ra leader) comprising amino acid SEQ ID NO. 732 and nucleotide sequence of SEQ ID NO. 733 or M1del CD8αsignal sequence (also referred to as M1del CD8 leader sequence) comprising amino acid sequence of SEQ ID NO. 1039 and nucleotide sequence of SEQ ID NO. 1040.

Signal sequences may also include nuclear localization signals (NLSs), nuclear export signals (NESs), polarized cell tubulo-vesicular structure localization signals (See, e.g., U.S. Pat. No. 8,993,742; Cour et al., Nucleic Acids Res. 2003, 31(1): 393-396; the contents of each of which are incorporated herein by reference in their entirety), extracellular localization signals, signals to subcellular locations (e.g. lysosome, endoplasmic reticulum, golgi, mitochondria, plasma membrane and peroxisomes, etc.) (See, e.g., U.S. Pat. No. 7,396,811; and Negi et al., Database, 2015, 1-7; the contents of each of which are incorporated herein by reference in their entirety).

In some embodiments, signal sequence may be a CD8 Leader sequence, comprising an amino acid sequence of SEQ ID NO. 469, encoded by the nucleic acid sequence of SEQ ID NO. 1202-1203, a GMCSFR Signal Peptide, comprising an amino acid sequence of SEQ ID NO. 1204, an IL2 Signal Peptide, comprising an amino acid sequence of SEQ ID NO. 1205, an 1 gK chain Signal Peptide, comprising an amino acid sequence of SEQ ID NO. 1206, an NPC2 Signal Peptide, comprising an amino acid sequence of SEQ ID NO. 1207, LAMB1 Signal Peptide of, comprising an amino acid sequence of SEQ ID NO. 1208, P31P1 Signal Peptide, comprising an amino acid sequence of SEQ ID NO. 1209, DMKN Signal Peptide, comprising an amino acid sequence of SEQ ID NO. 1210, TPA Signal Peptide, comprising an amino acid sequence of SEQ ID NO. 1211, PCSK9 Signal peptide, comprising an amino acid sequence of SEQ ID NO. 1212.

### Cleavage Sites

In some embodiments, the effector module comprises a cleavage and/or processing feature. The effector module of the present invention may include at least one protein cleavage signal/site. The protein cleavage signal/site may be located at the N terminus, the C terminus, at any space between the N and the C termini such as, but not limited to, half-way between the N and C termini, between the N terminus and the half-way point, between the half-way point and the C terminus, and combinations thereof.

The effector module may include one or more cleavage signal(s)/site(s) of any proteinases. The proteinases may be a serine proteinase, a cysteine proteinase, an endopeptidase, a dipeptidase, a metalloproteinase, a glutamic proteinase, a threonine proteinase and an aspartic proteinase. In some aspects, the cleavage site may be a signal sequence of furin, actinidain, calpain-1, carboxypeptidase A, carboxypeptidase P, carboxypeptidase Y, caspase-1, caspase-2, caspase-3, caspase-4, caspase-5, caspase-6, caspase-7, caspase-8, caspase-9, caspase-10, cathepsin B, cathepsin C, cathepsin G, cathepsin H, cathepsin K, cathepsin L, cathepsin S, cathepsin V, clostripain, chymase, chymotrypsin, elastase, endoproteinase, enterokinase, factor Xa, formic acid, granzyme B, Matrix metallopeptidase-2, Matrix metallopeptidase-3, pepsin, proteinase K, SUMO protease, subtilisin, TEV protease, thermolysin, thrombin, trypsin and TAGZyme.

In one embodiment, the cleavage site is a furin cleavage site comprising the amino acid sequence SARNRQKRS (SEQ ID NO. 561), encoded by nucleotide sequence of SEQ ID NO. 581; or a revised furin cleavage site comprising the amino acid sequence ARNRQKRS (SEQ ID NO. 562), encoded by nucleotide sequence of SEQ ID NO. 582; or a modified furin site comprising the amino acid sequence ESRRVRRNKRSK (SEQ ID NO. 471), encoded by nucleotide sequence of SEQ ID NO. 521-523.

In some embodiments, the cleavage site may be selected from, but is not limited to, a Granzyme B Cleavage site (I-E-P-D-X consensus motif) comprising the amino acid sequence of SEQ ID NO. 1216, an Enterokinase Cleavage site (Asp-Asp-Asp-Asp-Lys) comprising the amino acid sequence of SEQ ID NO. 1217, a Genenase Cleavage site (Pro-Gly-Ala-Ala-His-Tyr) comprising the amino acid sequence of SEQ ID NO. 1218, a PreScission Cleavage site (with a consensus motif) comprising the amino acid sequence of SEQ ID NO. 1219, a Thrombin Cleavage site comprising the amino acid sequence of SEQ ID NO. 1220, a TEV protease cleavage site (E-N-L-Y-F-Q-G motif) comprising the amino acid sequence of SEQ ID NO. 1221, and an Elastase cleavage site ([AGSV]-x motif), comprising the amino acid sequence of AX, GX, SX, or VX, wherein X is any amino acid.

In some embodiments, the cleavage site may be a furin cleavage site, comprising an amino acid sequence of SEQ ID NO. 1222-1231.

In some embodiments, the cleavage site may be a T2A cleavage site (SEQ ID NO. 1232), a P2A cleavage site (SEQ ID NO. 1233), E2A cleavage site (SEQ ID NO. 1234), or an F2A cleavage site (SEQ ID NO. 1235).

### Protein Tags

In some embodiments, the effector module of the invention may comprise a protein tag. The protein tag may be used for detecting and monitoring the process of the effector module. The effector module may include one or more tags such as an epitope tag (e.g., a FLAG or hemagglutinin (HA) tag). A large number of protein tags may be used for the present effector modules. They include, but are not limited to, self-labeling polypeptide tags (e.g., haloalkane dehalogenase (halotag2 or halotag7), ACP tag, clip tag, MCP tag, snap tag), epitope tags (e.g., FLAG, HA, His, and Myc), fluorescent tags (e.g., green fluorescent protein (GFP), red fluorescent protein (RFP), yellow fluorescent protein (YFP), and its variants), bioluminescent tags (e.g. luciferase and its variants), affinity tags (e.g., maltose-binding protein (MBP) tag, glutathione-S-transferase (GST) tag), immunogenic affinity tags (e.g., protein A/G, IRS, AU1, AUS, glu-glu, KT3, S-tag, HSV, VSV-G, Xpress and V5), and other tags (e.g., biotin (small molecule), StrepTag (StrepII), SBP, biotin carboxyl carrier protein (BCCP), eXact, CBP, CYD, HPC, CBD intein-chitin binding domain, Trx, NorpA, and NusA.

In some aspects, a multiplicity of protein tags, either the same or different tags, may be used; each of the tags may be located at the same N or C terminus, whereas in other cases these tags may be located at each terminus.

### Targeting Peptides

In some embodiments, the effector module of the invention may further comprise a targeting and/or penetrating peptide. Small targeting and/or penetrating peptides that selectively recognize cell surface markers (e.g. receptors, trans-membrane proteins, and extra-cellular matrix molecules) can be employed to target the effector module to the desired organs, tissues or cells. Short peptides (5-50 amino acid residues) synthesized in vitro and naturally occurring peptides, or analogs, variants, derivatives thereof, may be incorporated into the effector module for homing the effector module to the desired organs, tissues and cells, and/or subcellular locations inside the cells.

In some embodiments, a targeting sequence and/or penetrating peptide may be included in the effector module to drive the effector module to a target organ, or a tissue, or a cell (e.g., a cancer cell). In other embodiments, a targeting and/or penetrating peptide may direct the effector module to a specific subcellular location inside a cell.

A targeting peptide has any number of amino acids from about 6 to about 30 inclusive. The peptide may have 6, 7, 8, 9, 10, 11, 12, 13, 14, 15, 16, 17, 18, 19, 20, 21, 22, 23, 24, 25, 26, 27, 28, 29 or 30 amino acids. Generally, a targeting peptide may have 25 or fewer amino acids, for example, 20 or fewer, for example 15 or fewer.

### Linkers

In some embodiments, the effector module of the invention may further comprise a linker sequence. The linker region serves primarily as a spacer between two or more polypeptides within the effector module. The “linker” or “spacer”, as used herein, refers to a molecule or group of molecules that connects two molecules, or two parts of a molecule such as two domains of a recombinant protein.

In some embodiments, “Linker” (L) or “linker domain” or “linker region” or “linker module” or “peptide linker” as used herein refers to an oligo- or polypeptide region of from about 1 to 100 amino acids in length, which links together any of the domains/regions of the effector module (also called peptide linker). The peptide linker may be 1-40 amino acids in length, or 2-30 amino acids in length, or 20-80 amino acids in length, or 50-100 amino acids in length. Linker length may also be optimized depending on the type of payload utilized and based on the crystal structure of the payload. In some instances, a shorter linker length may be preferably selected. In some aspects, the peptide linker is made up of amino acids linked together by peptide bonds, preferably from 1 to 20 amino acids linked by peptide bonds, wherein the amino acids are selected from the 20 naturally occurring amino acids: Glycine (G), Alanine (A), Valine (V), Leucine (L), Isoleucine (I), Serine (S), Cysteine (C), Threonine (T), Methionine (M), Proline (P), Phenylalanine (F), Tyrosine (Y), Tryptophan (W), Histidine (H), Lysine (K), Arginine (R), Aspartate (D), Glutamic acid (E), Asparagine (N), and Glutamine (Q). One or more of these amino acids may be glycosylated, as is understood by those in the art. In some aspects, amino acids of a peptide linker may be selected from Alanine (A), Glycine (G), Proline (P), Asparagine (R), Serine (S), Glutamine (Q) and Lysine (K).

In one example, an artificially designed peptide linker may preferably be composed of a polymer of flexible residues like Glycine (G) and Serine (S) so that the adjacent protein domains are free to move relative to one another. Longer linkers may be used when it is desirable to ensure that two adjacent domains do not interfere with one another. The choice of a particular linker sequence may concern if it affects biological activity, stability, folding, targeting and/or pharmacokinetic features of the fusion construct. Examples of peptide linkers include, but are not limited to: MH, SG, GGSG (SEQ ID NO. 649; encoded by the nucleotide sequence SEQ ID NO. 650; 1041), GGSGG (SEQ ID NO. 470; encoded by any of the nucleotide sequences SEQ ID NO. 516-520), GGSGGG (SEQ ID NO. 651; encoded by any of the nucleotide sequences SEQ ID NO. 652-653), SGGGS (SEQ ID NO. 654; encoded by the nucleotide sequence SEQ ID NO. 655, 670, 709), GGSGGGSGG (SEQ ID NO. 656; encoded by the nucleotide sequence SEQ ID NO. 657), GGGGG (SEQ ID NO. 658), GGGGS (SEQ ID NO. 659) or (GGGGS)n (n=1 (SEQ ID NO. 659), 2 (SEQ ID NO. 660), 3 (SEQ ID NO. 560, encoded by the nucleotide sequence SEQ ID NO. 710-715), 4 (SEQ ID NO. 661), 5 (SEQ ID NO. 662), or 6 (SEQ ID NO. 663)), SSSSG (SEQ ID NO. 664) or (SSSSG)n (n=1 (SEQ ID NO. 664), 2 (SEQ ID NO. 665), 3 (SEQ ID NO. 666), 4 (SEQ ID NO. 667), 5 (SEQ ID NO. 668), or 6 (SEQ ID NO. 669)), SGGGSGGGGSGGGGSGGGGSGGGSLQ (SEQ ID NO. 631; encoded by the nucleotide sequence SEQ ID NO. 638, 716-720, 802), EFSTEF (SEQ ID NO. 615; encoded by any of the nucleotide sequences SEQ ID NO. 621-622), GKSSGSGSESKS (SEQ ID NO. 671), GGSTSGSGKSSEGKG (SEQ ID NO. 672), GSTSGSGKSSSEGSGSTKG (SEQ ID NO. 673), GSTSGSGKPGSGEGSTKG (SEQ ID NO. 674), VDYPYDVPDYALD (SEQ ID NO. 675; encoded by nucleotide sequence SEQ ID NO. 676), EGKSSGSGSESKEF (SEQ ID NO. 677), SGGGSGGGGSGGGGSGGGGSGGGGSGGGGSGGGS (SEQ ID NO. 721; encoded by SEQ ID NO. 723 SGGGSGGGGSGGGGSGGGGS (SEQ ID NO. 722; encoded by SEQ ID NO. 724), GS (encoded by GGTTCC), SG (encoded by AGCGGC), GSG (encoded by GGATCCGGA or GGATCCGGT), or MLLLVTSLLLCELPHPAFLLIP (SEQ ID NO. 830; encoded by SEQ ID NO. 831).

In other examples, a peptide linker may be made up of a majority of amino acids that are sterically unhindered, such as Glycine (G) and Alanine (A). Exemplary linkers are polyglycines (such as (G)4 (SEQ ID NO. 1031), (G)5 (SEQ ID NO. 658), (G)8) (SEQ ID NO. 1032), poly(GA), and polyalanines. The linkers described herein are exemplary, and linkers that are much longer and which include other residues are contemplated by the present invention.

A linker sequence may be a natural linker derived from a multi-domain protein. A natural linker is a short peptide sequence that separates two different domains or motifs within a protein.

In some aspects, linkers may be flexible or rigid. In other aspects, linkers may be cleavable or non-cleavable. As used herein, the terms “cleavable linker domain or region” or “cleavable peptide linker” are used interchangeably. In some embodiments, the linker sequence may be cleaved enzymatically and/or chemically. Examples of enzymes (e.g., proteinase/peptidase) useful for cleaving the peptide linker include, but are not limited, to Arg-C proteinase, Asp-N endopeptidase, chymotrypsin, clostripain, enterokinase, Factor Xa, glutamyl endopeptidase, Granzyme B, Achromobacter proteinase I, pepsin, proline endopeptidase, proteinase K, Staphylococcal peptidase I, thermolysin, thrombin, trypsin, and members of the Caspase family of proteolytic enzymes (e.g. Caspases 1-10). Chemical sensitive cleavage sites may also be included in a linker sequence. Examples of chemical cleavage reagents include, but are not limited to, cyanogen bromide, which cleaves methionine residues; N-chloro succinimide, iodobenzoic acid or BNPS-skatole (2-(2-nitrophenylsulfenyl)-3-methylindole), which cleaves tryptophan residues; dilute acids, which cleave at aspartyl-prolyl bonds; and e aspartic acid-proline acid cleavable recognition sites (i.e., a cleavable peptide linker comprising one or more D-P dipeptide moieties). The fusion module may include multiple regions encoding peptides of interest separated by one or more cleavable peptide linkers.

In other embodiments, a cleavable linker may be a “self-cleaving” linker peptide, such as 2A linkers (for example T2A), 2A-like linkers or functional equivalents thereof and combinations thereof. In some embodiments, the linkers include the picornaviral 2A-like linker, CHYSEL sequences of porcine teschovirus (P2A), Thosea asigna virus (T2A) or combinations, variants and functional equivalents thereof. Other linkers will be apparent to those skilled in the art and may be used in connection with alternate embodiments of the invention. In some embodiments, the biocircuits of the present invention may include 2A peptides. The 2A peptide is a sequence of about 20 amino acid residues from a virus that is recognized by a protease (2A peptidases) endogenous to the cell. The 2A peptide was identified among picornaviruses, a typical example of which is the Foot-and Mouth disease virus (Robertson B H, et. al., J Virol 1985, 54:651-660). 2A-like sequences have also been found in Picornaviridae like equine rhinitis A virus, as well as unrelated viruses such as porcine teschovirus-1 and the insect Thosea asigna virus (TaV). In such viruses, multiple proteins are derived from a large polyprotein encoded by an open reading frame. The 2A peptide mediates the co-translational cleavage of this polyprotein at a single site that forms the junction between the virus capsid and replication polyprotein domains. The 2A sequences contain the consensus motif D-V/I-E-X—N-P-G-P (SEQ ID NO. 1033). These sequences are thought to act co-translationally, preventing the formation of a normal peptide bond between the glycine and last proline, resulting in the ribosome skipping of the next codon (Donnelly M L et al. (2001). J Gen Virol, 82:1013-1025). After cleavage, the short peptide remains fused to the C terminus of the protein upstream of the cleavage site, while the proline is added to the N terminus of the protein downstream of the cleavage site. Of the 2A peptides identified to date, four have been widely used namely FMDV 2A (abbreviated herein as F2A); equine rhinitis A virus (ERAV) 2A (E2A); porcine teschovirus-1 2A (P2A) and Thoseaasigna virus 2A (T2A). In some embodiments, the 2A peptide sequences useful in the present invention are selected from SEQ ID NO.8-11 of International Patent Publication WO2010042490, the contents of which are incorporated by reference in its entirety.

As a non-limiting example, the P2A cleavable peptide may be GATNFSLLKQAGDVEENPGP (SEQ ID NO. 725; encoded by SEQ ID NO. 726).

The linkers of the present invention may also be non-peptide linkers. For example, alkyl linkers such as —NH—(CH₂) a-C(O)—, wherein a=2-20 can be used. These alkyl linkers may further be substituted by any non-sterically hindering group such as lower alkyl (e.g., C₁-C₆) lower acyl, halogen (e.g., Cl, Br), CN, NH₂, phenyl, etc.

In one embodiment, the linker may be a spacer region of one or more nucleotides. Non-limiting examples of spacers are TCTAGATAATACGACTCACTAGAGATCC (SEQ ID NO. 727), TATGGCCACAACCATG (SEQ ID NO. 728), AATCTAGATAATACGACTCACTAGAGATCC (SEQ ID NO. 729), GCTTGCCACAACCCACAAGGAGACGACCTTCC (SEQ ID NO. 800), TCGCGAATG, TCGCGA, or ATCGGGCTAGC (SEQ ID NO. 1042).

In one embodiment, the linker may be a BamHI site. As a non-limiting example, the BamHI site has the amino acid sequence GS and/or the DNA sequence GGATCC.

### Embedded Stimulus, Signals and Other Regulatory Features

In some embodiments, the effector module of the present invention may further comprise one or more microRNAs, microRNA binding sites, promotors and tunable elements. In one embodiment, microRNA may be used in support of the creation of tunable biocircuits. Each aspect or tuned modality may bring to the effector module or biocircuit a differentially tuned feature. For example, a destabilizing domain may alter cleavage sites or dimerization properties or half-life of the payload, and the inclusion of one or more microRNA or microRNA binding site may impart cellular detargeting or trafficking features. Consequently, the present invention embraces biocircuits which are multifactorial in their tenability. Such biocircuits and effector modules may be engineered to contain one, two, three, four or more tuned features.

In some embodiments, compositions of the invention may include optional proteasome adaptors. As used herein, the term “proteasome adaptor” refers to any nucleotide/amino acid sequence that targets the appended payload for degradation. In some aspects, the adaptors target the payload for degradation directly thereby circumventing the need for ubiquitination reactions. Proteasome adaptors may be used in conjunction with destabilizing domains to reduce the basal expression of the payload. Exemplary proteasome adaptors include the UbL domain of Rad23 or hHR23b, HPV E7 which binds to both the target protein Rb and the S4 subunit of the proteasome with high affinity, which allows direct proteasome targeting, bypassing the ubiquitination machinery; the protein gankyrin which binds to Rb and the proteasome subunit S6.

### Degrons

In some embodiments, the effector modules of the present invention may include degrons at their C termini. The degrons may comprise -GG, -RG, -KG, -QG, -WG, -PG, and -AG as the penultimate and the ultimate amino acids of the SREs. Furthermore, certain -2 amino acids (D, E, V, I and L) may be more enriched in the C terminus of the of the effector modules. Other degrons include, but are not limited, to RxxG motif, wherein x is any amino acid, C-terminal twin glutamic acid (EE) motif, and motifs that comprise an arginine at the -3 positions. Degrons may also be selected from the R-3 motif, G-end, R at -3, A-end, A at −2, V at −2 positions. Any of the degrons described in Koren et al., 2018, Cell 173, 1-14, may be useful in the present invention (the contents of which are incorporated by reference in their entirety). In some aspects, the expression of the effector module may be tuned by altering its overall amino acid composition. In some aspects, the amino acid composition of the effector module may be tuned to reduce basal expression. In some embodiments, basal expression may be tuned by increasing the number of bulky aromatic residues such as tryptophan (W), phenylalanine (F), and tyrosine (Y) in the effector module. Such bulky amino acids are known to reduce protein stability. In some embodiments, the amino acid composition of the SREs may be enriched with acidic residues such as, but not limited to, aspartic acid (D) and glutamic acid (E), and positively charged lysine (K), if an increase in the basal expression of the SRE is desired.

### Polynucleotides

The term “polynucleotide” or “nucleic acid molecule” in its broadest sense, includes any compound and/or substance that comprise a polymer of nucleotides, e.g., linked nucleosides. These polymers are often referred to as polynucleotides. Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

In one aspect, the polynucleotides may be a DNA or RNA molecule. In one aspect, the polynucleotides may comprise spatiotemporally selected codons. In one aspect, the polynucleotides of the invention may be a DNA molecule. In some embodiments, the polynucleotides may be an RNA molecule. In one aspect, the RNA molecule may be a messenger molecule. In some embodiments, the RNA molecule may be chemically modified.

In some embodiments, polynucleotides of the invention may be a messenger RNA (mRNA) or any nucleic acid molecule and may or may not be chemically modified. In one aspect, the nucleic acid molecule is an mRNA. As used herein, the term “messenger RNA (mRNA)” refers to any polynucleotide which encodes a polypeptide of interest and which is capable of being translated to produce the encoded polypeptide of interest in vitro, in vivo, in situ or ex vivo.

Traditionally, the basic components of an mRNA molecule include at least a coding region, a 5′UTR, a 3′UTR, a 5′ cap and a poly-A tail. Building on this wild type modular structure, the present invention expands the scope of functionality of traditional mRNA molecules by providing payload constructs which maintain a modular organization, but which comprise one or more structural and/or chemical modifications or alterations which impart useful properties to the polynucleotide, for example tenability of function. As used herein, a “structural” feature or modification is one in which two or more linked nucleosides are inserted, deleted, duplicated, inverted or randomized in a polynucleotide without significant chemical modification to the nucleosides themselves. Because chemical bonds will necessarily be broken and reformed to affect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In some embodiments, polynucleotides of the present invention may harbor 5′UTR sequences which play a role in translation initiation. 5′UTR sequences may include features such as Kozak sequences which are commonly known to be involved in the process by which the ribosome initiates translation of genes, Kozak sequences have the consensus XCCR(A/G) CCAUG, where R is a purine (adenine or guanine) three bases upstream of the start codon (AUG) and X is any nucleotide. In one embodiment, the Kozak sequence is ACCGCC. By engineering the features that are typically found in abundantly expressed genes of target cells or tissues, the stability and protein production of the polynucleotides of the invention can be enhanced.

Further provided are polynucleotides, which may contain an internal ribosome entry site (IRES) which play an important role in initiating protein synthesis in the absence of 5′ cap structure in the polynucleotide. An IRES may act as the sole ribosome binding site or may serve as one of the multiple binding sites. Polynucleotides of the invention containing more than one functional ribosome binding site may encode several peptides or polypeptides that are translated independently by the ribosomes giving rise to bicistronic and/or multicistronic nucleic acid molecules.

In some embodiments, polynucleotides encoding biocircuits, effector modules, SREs and payloads of interest such as immunotherapeutic agents may include from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000 nucleotides). In some aspects, polynucleotides of the invention may include more than 10,000 nucleotides.

Regions of the polynucleotides which encode certain features such as cleavage sites, linkers, trafficking signals, tags or other features may range independently from 10-1,000 nucleotides in length (e.g., greater than 20, 30, 40, 45, 50, 55, 60, 70, 80, 90, 100, 120, 140, 160, 180, 200, 250, 300, 350, 400, 450, 500, 600, 700, 800, and 900 nucleotides or at least 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, and 1,000 nucleotides).

In some embodiments, polynucleotides of the present invention may further comprise embedded regulatory moieties such as microRNA binding sites within the 3′UTR of nucleic acid molecules which when bind to microRNA molecules, down-regulate gene expression either by reducing nucleic acid molecule stability or by inhibiting translation. Conversely, for the purposes of the polynucleotides of the present invention, microRNA binding sites can be engineered out of (i.e. removed from) sequences in which they naturally occur in order to increase protein expression in specific tissues. For example, miR-142 and miR-146 binding sites may be removed to improve protein expression in the immune cells. In some embodiments, any of the encoded payloads may be regulated by an SRE and then combined with one or more regulatory sequences to generate a dual or multi-tuned effector module or biocircuit system.

In some embodiments, polynucleotides of the present invention may encode fragments, variants, derivatives of polypeptides of the inventions. In some aspects, the variant sequence may keep the same or a similar activity. Alternatively, the variant may have an altered activity (e.g., increased or decreased) relative to the start sequence. Generally, variants of a particular polynucleotide or polypeptide of the invention will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen et al., Gapped BLAST and PSI-BLAST: a new generation of protein database search programs, Nucleic Acids Res., 1997, 25:3389-3402.)

In some embodiments, polynucleotides of the present invention may be modified. As used herein, the terms “modified”, or as appropriate, “modification” refers to chemical modification with respect to A, G, U (T in DNA) or C nucleotides. Modifications may be on the nucleoside base and/or sugar portion of the nucleosides which comprise the polynucleotide. In some embodiments, multiple modifications are included in the modified nucleic acid or in one or more individual nucleoside or nucleotide. For example, modifications to a nucleoside may include one or more modifications to the nucleobase and the sugar. Modifications to the polynucleotides of the present invention may include any of those taught in, for example, International Publication NO. WO2013052523, the contents of which are incorporated herein by reference in its entirety.

As described herein “nucleoside” is defined as a compound containing a sugar molecule (e.g., a pentose or ribose) or a derivative thereof in combination with an organic base (e.g., a purine or pyrimidine) or a derivative thereof (also referred to herein as “nucleobase”). As described herein, “nucleotide” is defined as a nucleoside including a phosphate group.

In some embodiments, the modification may be on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates). Other modifications which may be used are taught in, for example, International Application NO. WO2013052523, the contents of which are incorporated herein by reference in their entirety.

Chemical modifications and/or substitution of the nucleotides or nucleobases of the polynucleotides of the invention which are useful in the present invention include any modified substitutes known in the art, for example, (±)1-(2-Hydroxypropyl)pseudouridine TP, (2R)-1-(2-Hydroxypropyl)pseudouridine TP, 1-(4-Methoxy-phenyl)pseudo-UTP, 2′-O-dimethyladenosine, 1,2′-O-dimethylguanosine, 1,2′-O-dimethylinosine, 1-Hexyl-pseudo-UTP, 1-Homoallylpseudouridine TP, 1-Hydroxymethylpseudouridine TP, 1-iso-propyl-pseudo-UTP, 1-Me-2-thio-pseudo-UTP, 1-Me-4-thio-pseudo-UTP, 1-Me-alphα-thio-pseudo-UTP, 1-Me-GTP, 2′-Amino-2′-deoxy-ATP, 2′-Amino-2′-deoxy-CTP, 2′-Amino-2′-deoxy-GTP, 2′-Amino-2′-deoxy-UTP, 2′-Azido-2′-deoxy-ATP, tubercidine, under modified hydroxywybutosine, uridine 5-oxyacetic acid, uridine 5-oxyacetic acid methyl ester, wybutosine, wyosine, xanthine, Xanthosine-5′-TP, xylo-adenosine, zebularine, α-thio-adenosine, α-thio-cytidine, α-thio-guanosine, and/or α-thio-uridine.

Polynucleotides of the present invention may comprise one or more of the modifications taught herein. Different sugar modifications, base modifications, nucleotide modifications, and/or internucleoside linkages (e.g., backbone structures) may exist at various positions in the polynucleotide of the invention. One of ordinary skill in the art will appreciate that the nucleotide analogs or other modification(s) may be located at any position(s) of a polynucleotide such that the function of the polynucleotide is not substantially decreased. A modification may also be a 5′ or 3′ terminal modification. The polynucleotide may contain from about 1% to about 100% modified nucleotides (either in relation to overall nucleotide content, or in relation to one or more types of nucleotide, i.e. any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, one or more codons of the polynucleotides of the present invention may be replaced with other codons encoding the native amino acid sequence to tune the expression of the SREs, through a process referred to as codon selection. Since mRNA codon, and tRNA anticodon pools tend to vary among organisms, cell types, sub cellular locations and over time, the codon selection described herein is a spatiotemporal (ST) codon selection.

In some embodiments of the invention, certain polynucleotide features may be codon optimized. For example, a preferred region for codon optimization may be upstream (5′) or downstream (3′) to a region which encodes a polypeptide. These regions may be incorporated into the polynucleotide before and/or after codon optimization of the payload encoding region or open reading frame (ORF).

Spatiotemporal codon selection may impact the expression of the polynucleotides of the invention, since codon composition determines the rate of translation of the mRNA species and its stability. For example, tRNA anticodons to optimized codons are abundant, and thus translation may be enhanced. In contrast, tRNA anticodons to less common codons are fewer and thus translation may proceed at a slower rate. Presnyak et al. have shown that the stability of an mRNA species is dependent on the codon content, and higher stability and thus higher protein expression may be achieved by utilizing optimized codons (Presnyak et al. (2015) Cell 160, 1111-1124; the contents of which are incorporated herein by reference in their entirety). Thus, in some embodiments, ST codon selection may include the selection of optimized codons to enhance the expression of the SRES, effector modules and biocircuits of the invention. In other embodiments, spatiotemporal codon selection may involve the selection of codons that are less commonly used in the genes of the host cell to decrease the expression of the compositions of the invention. The ratio of optimized codons to codons less commonly used in the genes of the host cell may also be varied to tune expression.

In some embodiments, certain regions of the polynucleotide may be preferred for codon selection. For example, a preferred region for codon selection may be upstream (5′) or downstream (3′) to a region which encodes a polypeptide. These regions may be incorporated into the polynucleotide before and/or after codon selection of the payload encoding region or open reading frame (ORF).

The stop codon of the polynucleotides of the present invention may be modified to include sequences and motifs to alter the expression levels of the SREs, payloads and effector modules of the present invention. Such sequences may be incorporated to induce stop codon readthrough, wherein the stop codon may specify amino acids e.g. selenocysteine or pyrrolysine. In other instances, stop codons may be skipped altogether to resume translation through an alternate open reading frame. Stop codon read through may be utilized to tune the expression of components of the effector modules at a specific ratio (e.g. as dictated by the stop codon context). Examples of preferred stop codon motifs include UGAN, UAAN, and UAGN, where N is either C or U. Polynucleotide modifications and manipulations can be accomplished by methods known in the art such as, but not limited to, site directed mutagenesis and recombinant technology. The resulting modified molecules may then be tested for activity using in vitro or in vivo assays such as those described herein, or any other suitable screening assay known in the art.

In some embodiments, polynucleotides of the invention may comprise two or more effector module sequences, or two or more payloads of interest sequences, which are in a pattern such as ABABAB or AABBAABBAABB or ABCABCABC or variants thereof repeated once, twice, or more than three times. In these patterns, each letter, A, B, or C represent a different effector module component.

In yet another embodiment, polynucleotides of the invention may comprise two or more effector module component sequences with each component having one or more SRE sequences (DD sequences), or two or more payload sequences. As a non-limiting example, the sequences may be in a pattern such as ABABAB or AABBAABBAABB or ABCABCABC or variants thereof repeated once, twice, or more than three times in each of the regions. As another non-limiting example, the sequences may be in a pattern such as ABABAB or AABBAABBAABB or ABCABCABC or variants thereof repeated once, twice, or more than three times across the entire polynucleotide. In these patterns, each letter, A, B, or C represent a different sequence or component.

According to the present invention, polynucleotides encoding distinct biocircuits, effector modules, SREs and payloads may be linked together through the 3′-end using nucleotides which are modified at the 3′ terminus. Chemical conjugation may be used to control the stoichiometry of delivery into cells. Polynucleotides can be designed to be conjugated to other polynucleotides, dyes, intercalating agents (e.g. acridines), cross-linkers (e.g. psoralene, mitomycin C), porphyrins (TPPC4, texaphyrin, sapphyrin), polycyclic aromatic hydrocarbons (e.g., phenazine, dihydrophenazine), artificial endonucleases (e.g. EDTA), alkylating agents, phosphate, amino, mercapto, PEG (e.g., PEG-40K), MPEG, (MPEG)₂, polyamino, alkyl, substituted alkyl, radiolabeled markers, enzymes, haptens (e.g. biotin), transport/absorption facilitators (e.g., aspirin, vitamin E, folic acid), synthetic ribonucleases, proteins, e.g., glycoproteins, or peptides, e.g., molecules having a specific affinity for a co-ligand, or antibodies e.g., an antibody, that binds to a specified cell type such as a cancer cell, endothelial cell, or bone cell, hormones and hormone receptors, non-peptidic species, such as lipids, lectins, carbohydrates, vitamins, cofactors, or a drug. As non-limiting examples, they may be conjugates with other immune conjugates.

In some embodiments, the compositions of the polynucleotides of the invention may generated by combining the various components of the effector modules using the Gibson assembly method. The Gibson assembly reaction consists of three isothermal reactions, each relying on a different enzymatic activity including a 5′ exonuclease which generates long overhangs, a polymerase which fills in the gaps of the annealed single strand regions and a DNA ligase which seals the nicks of the annealed and filled-in gaps. Polymerase chain reactions are performed prior to Gibson assembly which may be used to generate PCR products with overlapping sequence. These methods can be repeated sequentially, to assemble larger and larger molecules. For example, the method can comprise repeating a method as above to join a second set of two or more DNA molecules of interest to one another, and then repeating the method again to join the first and second set DNA molecules of interest, and so on. At any stage during these multiple rounds of assembly, the assembled DNA can be amplified by transforming it into a suitable microorganism, or it can be amplified in vitro (e.g., with PCR).

In some embodiments, polynucleotides of the present invention may encode a fusion polypeptide comprising a destabilizing domain (DD) and at least one immunotherapeutic agent taught herein. The DD domain may be a FKBP mutant encoded by nucleotide sequence of SEQ ID NO. 524-526, 528-531, 787-789, 794, 812, and/or 827, an ecDHFR mutant encoded by nucleotide sequence of SEQ ID NO. 527, 532, 603, 627, 641-642, 788, 791, and/or 793, hDHFR mutant encoded by nucleotide sequence of SEQ ID NO. 533-540, 604, 678-683 and/or 734-780, and/or 795-798.

In some embodiments, the polynucleotides of the invention may encode effector modules comprising the CD19 CAR as the payload comprising the nucleotide sequence of SEQ ID NO. 541-555 and/or 818-841, or IL12 as the payload comprising the nucleotide sequence of SEQ ID NO. 605-613 and/or 847-852, or IL15 as the payload comprising the nucleotide sequence of SEQ ID NO. 580, 628-629, and/or 853-854, or IL15/IL15Ra fusion polypeptide as the payload comprising the nucleotide sequence of SEQ ID NO. 643-648, 884-911, 918, and/or 921.

### Cells

In accordance with the present invention, cells genetically modified to express at least one biocircuit, SRE (e.g, DD), effector module and immunotherapeutic agent of the invention, are provided. Cells of the invention may include, without limitation, immune cells, stem cells and tumor cells. In some embodiments, immune cells are immune effector cells, including, but not limiting to, T cells such as CD8⁺ T cells and CD4⁺ T cells (e.g., Th1, Th2, Th17, Foxp3+ cells), memory T cells such as T memory stem cells, central T memory cells, and effector memory T cells, terminally differentiated effector T cells, natural killer (NK) cells, NK T cells, tumor infiltrating lymphocytes (TILs), cytotoxic T lymphocytes (CTLs), regulatory T cells (Tregs), and dendritic cells (DCs), other immune cells that can elicit an effector function, or the mixture thereof. T cells may be Tarβ cells and Tγδ cells. In some embodiments, stem cells may be from human embryonic stem cells, mesenchymal stem cells, and neural stem cells. In some embodiments, T cells may be depleted endogenous T cell receptors (See U.S. Pat. Nos. 9,273,283; 9,181,527; and 9,028,812; the contents of each of which are incorporated herein by reference in their entirety).

In some embodiments, cells of the invention may be autologous, allogeneic, syngeneic, or xenogeneic in relation to a particular individual subject.

In some embodiments, cells of the invention may be mammalian cells, particularly human cells. Cells of the invention may be primary cells or immortalized cell lines.

Engineered immune cells can be accomplished by transducing a cell, compositions with a polypeptide of a biocircuit, an effector module, a SRE and/or a payload of interest (i.e., immunotherapeutic agent), or a polynucleotide encoding said polypeptide, or a vector comprising said polynucleotide. The vector may be a viral vector such as a lentiviral vector, a gamma-retroviral vector, a recombinant AAV, an adenoviral vector and an oncolytic viral vector. In other aspects, non-viral vectors for example, nanoparticles and liposomes may also be used. In some embodiments, immune cells of the invention are genetically modified to express at least one immunotherapeutic agent of the invention which is tunable using a stimulus. In some examples, two, three or more immunotherapeutic agents constructed in the same biocircuit and effector module are introduced into a cell. In other examples, two, three, or more biocircuits, effector modules, each of which comprises an immunotherapeutic agent, may be introduced into a cell.

In some embodiments, immune cells of the invention may be T cells modified to express an antigen-specific T cell receptor (TCR), or an antigen specific chimeric antigen receptor (CAR) taught herein (known as CAR T cells). Accordingly, at least one polynucleotide encoding a CAR system (or a TCR) described herein, or a vector comprising the polynucleotide is introduced into a T cell. The T cell expressing the CAR or TCR binds to a specific antigen via the extracellular targeting moiety of the CAR or TCR, thereby a signal via the intracellular signaling domain (s) is transmitted into the T cell, and as a result, the T cell is activated. The activated CAR T cell changes its behavior including release of a cytotoxic cytokine (e.g., a tumor necrosis factor, and lymphotoxin, etc.), improvement of a cell proliferation rate, change in a cell surface molecule, or the like. Such changes cause destruction of a target cell expressing the antigen recognized by the CAR or TCR. In addition, release of a cytokine or change in a cell surface molecule stimulates other immune cells, for example, a B cell, a dendritic cell, a NK cell, and a macrophage.

The CAR introduced into a T cell may be a first-generation CAR including only the intracellular signaling domain from TCR CD3zeta, or a second-generation CAR including the intracellular signaling domain from TCR CD3zeta and a costimulatory signaling domain, or a third-generation CAR including the intracellular signaling domain from TCR CD3zeta and two or more costimulatory signaling domains, or a split CAR system, or an on/off switch CAR system. In one example, the expression of the CAR or TCR is controlled by a destabilizing domain (DD) such as a hDHFR mutant, in the effector module of the invention. The presence or absence of hDHFR binding ligand such as TMP is used to tune the CAR or TCR expression in transduced T cells or NK cells.

In some embodiments, CAR T cells of the invention may be further modified to express another one, two, three or more immunotherapeutic agents. The immunotherapeutic agents may be another CAR or TCR specific to a different target molecule; a cytokine such as IL2, IL12, IL15 and IL18, or a cytokine receptor such as IL15Ra; a chimeric switch receptor that converts an inhibitory signal to a stimulatory signal; a homing receptor that guides adoptively transferred cells to a target site such as the tumor tissue; an agent that optimizes the metabolism of the immune cell; or a safety switch gene (e.g., a suicide gene) that kills activated T cells when a severe event is observed after adoptive cell transfer or when the transferred immune cells are no-longer needed. These molecules may be included in the same effector module or in separate effector modules.

In one embodiment, the CAR T cell (including TCR T cell) of the invention may be an “armed” CAR T cell which is transformed with an effector module comprising a CAR and an effector module comprising a cytokine. The inducible or constitutively secrete active cytokines further armor CAR T cells to improve efficacy and persistence. In this context, such CAR T cell is also referred to as “armored CAR T cell”. The “armor” molecule may be selected based on the tumor microenvironment and other elements of the innate and adaptive immune systems. In some embodiments, the molecule may be a stimulatory factor such as IL2, IL12, IL15, IL18, type I IFN, CD40L and 4-1BBL which have been shown to further enhance CAR T cell efficacy and persistence in the face of a hostile tumor microenvironment via different mechanisms (Yeku et al., Biochem Soc Trans., 2016, 44(2): 412-418).

Chimeric Antigen Receptor engineered T cells (CAR-T) therapies have yet to be successfully applied to solid tumors. Enhancing CAR-T cell functionality and selectively delivering cargo to the site of solid tumors represent key tactics to achieve effective CAR-T therapy for solid tumors. In one embodiment, Interleukin 12 (IL12) may be utilized to enhance the effectiveness of CAR-T cells, especially since it has the potential to remodel the tumor microenvironment. IL12 has been previously shown to be effective in enhancing efficacy of CAR or TCR modified T-cells as well as tumor infiltrating lymphocytes (TILs) in preclinical and clinical models. However, constitutive production of IL12 can compromise safety and/or efficacy; therefore, on demand, local delivery of the cytokine may be a preferred approach. In some embodiments, biocircuits of the present invention may be utilized to exogenously control IL12 expression to enable the use of IL12 in adoptive cell therapy.

In some embodiments, DD regulated Flexi IL12 constructs may be used to improve the efficacy of the CARs, especially in solid tumor settings, by providing a controlled local signal for tumor microenvironment remodeling and epitope spreading. DD regulation also provides rapid, dose dependent, and local production of Flexi IL12.

In some aspects, the armed CAR T cell of the invention is modified to express a CD19 CAR and IL12. Such T cells, after CAR mediated activation in the tumor, release inducible IL12 which augments T-cell activation and attracts and activates innate immune cells to eliminate CD19-negative cancer cells.

In one embodiment, T cells of the invention may be modified to express an effector module comprising a CAR and an effector module comprising a suicide gene.

In one embodiment, the CAR T cell (including TCR T cell) of the invention may be transformed with effector modules comprising a cytokine and a safety switch gene (e.g., suicide gene). The suicide gene may be an inducible caspase such as caspase 9 which induces apoptosis, when activated by an extracellular stimulus of a biocircuit system. Such induced apoptosis eliminates transferred cell as required to decrease the risk of direct toxicity and uncontrolled cell proliferation.

In some embodiments, immune cells of the invention may be NK cells modified to express an antigen-specific T cell receptor (TCR), or an antigen specific chimeric antigen receptor (CAR) taught herein.

Natural killer (NK) cells are members of the innate lymphoid cell family and characterized in humans by expression of the phenotypic marker CD56 (neural cell adhesion molecule) in the absence of CD3 (T-cell co-receptor). NK cells are potent effector cells of the innate immune system which mediate cytotoxic attack without the requirement of prior antigen priming, forming the first line of defense against diseases including cancer malignancies and viral infection.

NK cell activation is characterized by an array of receptors with activating and inhibitory functions. The important activation receptors on NK cells include CD94/NKG2C and NKG2D (the C-type lectin-like receptors), and the natural cytotoxicity receptors (NCR) NKp30, NKp44 and NKp46, which recognize ligands on tumor cells or virally infected cells. NK cell inhibition is essentially mediated by interactions of the polymorphic inhibitory killer cell immunoglobulin-like receptors (KIRs) with their cognate human—leukocyte—antigen (HLA) ligands via the alpha-1 helix of the HLA molecule. The balance between signals that are generated from activating receptors and inhibitory receptors mainly determines the immediate cytotoxic activation.

NK cells may be isolated from peripheral blood mononuclear cells (PBMCs) or derived from human embryonic stem (ES) cells and induced pluripotent stem cells (iPSCs). The primary NK cells isolated from PBMCs may be further expanded for adoptive immunotherapy. Strategies and protocols useful for the expansion of NK cells may include interleukin 2 (IL2) stimulation and the use of autologous feeder cells, or the use of genetically modified allogeneic feeder cells. In some aspects, NK cells can be selectively expanded with a combination of stimulating ligands including IL15, IL21, IL2, 41BBL, IL12, IL18, MICA, 2B4, LFA-1, and BCM1/SLAMF2 (e.g., US patent publication NO. US20150190471).

Immune cells expressing effector modules comprising a CAR and/or other immunotherapeutic agents can be used as cancer immunotherapy. The immunotherapy comprises the cells expressing a CAR and/or other immunotherapeutic agents as an active ingredient and may further comprise a suitable excipient. Examples of the excipient may include the aforementioned pharmaceutically acceptable excipients, including various cell culture media, and isotonic sodium chloride.

In some embodiments, cells of the present invention may be dendritic cells that are genetically modified to express the compositions of the invention. Such cells may be used as cancer vaccines.

In some embodiments, the composition comprising

(a) an effector module, said effector module comprising a stimulus response element (SRE) operably linked to an immunotherapeutic agent, wherein
(i) the immunotherapeutic agent is a cytokine or a cytokine-cytokine receptor fusion protein; and
(ii) the SRE is a DD, said DD derived from a parent protein or a mutant protein having one, two, three or more amino acid mutations compared to said parent protein, wherein the parent protein is selected from
(i′) human DHFR (hDHFR) (SEQ ID NO. 1);
(ii′) E. coli DHFR (ecDHFR) (SEQ ID NO. 2); and
(iii′) human protein FKBP (SEQ ID NO. 3; 1087);

(b) a chimeric antigen receptor (CAR), wherein the chimeric antigen receptor is operably linked to said effector module.

In some embodiments, the immunotherapeutic agent is a cytokine and wherein said cytokine is IL12.

In some embodiments, the IL12 is a fusion protein comprising a p40 subunit, a linker, and a p35 subunit.

In some embodiments, the p40 subunit is a p40 (23-328 of WT) (SEQ ID NO. 563), a p40 (WT) (SEQ ID NO.1091) or a p40 (23-328 of WT) (K217N) (SEQ ID NO. 578).

In some embodiments, the p40 subunit is p40 (23-328 of WT) (SEQ ID NO. 563).

In some embodiments, the p35 subunit is a p35 (57-253 of WT) (SEQ ID NO. 564) or p35 (WT) (SEQ ID NO. 1093).

In some embodiments, the p35 subunit is a p35 (57-253 of WT) (SEQ ID NO. 564).

In some embodiments, the immunotherapeutic agent is a cytokine-cytokine receptor fusion protein.

In some embodiments, the cytokine-cytokine receptor fusion polypeptide comprises the whole or a portion of SEQ. ID NO. 616, 632 fused to the whole or a portion of any of SEQ. ID NOs. 632; 855, 1097 to produce a IL15-IL15 receptor fusion polypeptide.

In some embodiments, the parent protein is hDHFR and the DD comprises a mutant protein having:

(a) a single mutation selected from the group consisting of hDHFR (I17V), hDHFR (F59S), hDHFR (N65D), hDHFR (K81R), hDHFR (A107V), hDHFR (Y122I), hDHFR (N127Y), hDHFR (M1400, hDHFR (K185E), hDHFR (N186D), and hDHFR (M1400;

(b) a double mutation selected from the group consisting of hDHFR (M1del, I17A), hDHFR (M1del, N127Y), hDHFR (M1del, I17V), hDHFR (M1del, Y122I), hDHFR (M1del, K185E), hDHFR (C7R, Y163C), hDHFR (A10V, H88Y), hDHFR (Q36K, Y122I), hDHFR (M53T, R138I), hDHFR (T57A, I72A), hDHFR (E63G, I176F), hDHFR (G21T, Y122I), hDHFR (L74N, Y122I), hDHFR (V75F, Y122I), hDHFR (L94A, T147A), DHFR (V121A, Y22I), hDHFR (Y122I, A125F), hDHFR (H131R, E144G), hDHFR (T137R, F143L), hDHFR (Y178H, E18IG), hDHFR (Y183H, K185E), hDHFR (E162G, I176F), and hDHFR (M1del, M1400;

(c) a triple mutation selected from the group consisting of hDHFR (V9A, S93R, P150L), hDHFR (I8V, K133E, Y163C), hDHFR (L23S, V121A, Y157C), hDHFR (K19E, F89L, E181G), hDHFR (Q36F, N65F, Y122I), hDHFR (G54R, M140V, S168C), hDHFR (V110A, V136M, K177R), hDHFR (Q36F, Y122I, A125F), hDHFR (N49D, F59S, D153G), hDHFR (G21E, I72V, I176T), hDHFR (M1del, 117A, Y122I), hDHFR (M1del, I17V, Y122I), hDHFR (M1del, N127Y, Y122I), hDHFR (M1del, E162G, I176F), hDHFR (M1del, H131R, E144G), and hDHFR (M1del, Y122I, A125F); or

(d) a quadruple or higher mutation selected from the group consisting of hDHFR (M1del, Q36F, Y122I, A125F), hDHFR (M1del, Y122I, H131R, E144G), hDHFR (M1del, E31D, F32M, V116I), hDHFR (M1del, Q36F, N65F, Y122I), hDHFR (V2A, R33G, Q36R, L100P, K185R), hDHFR (M1del, D22S, F32M, R33S, Q36S, N65S), hDHFR (I17N, L98S, K99R, M112T, E151G, E162G, E172G), hDHFR (G16S, I17V, F89L, D96G, K123E, M140V, D146G, K156R), hDHFR (K81R, K99R, L100P, E102G, N108D, K123R, H128R, D142G, F180L, K185E), hDHFR (R138G, D142G, F143S, K156R, K158E, E162G, V166A, K177E, Y178C, K185E, N186S), hDHFR (N14S, P24S, F35L, M53T, K56E, R92G, S93G, N127S, H128Y, F135L, F143S, L159P, L160P, E173A, F180L), hDHFR (F35L, R37G, N65A, L68S, K69E, R71G, L80P, K99G, G117D, L132P, I139V, M140I, D142G, D146G, E173G, D187G), hDHFR (L28P, N30H, M38V, V44A, L68S, N73G, R78G, A97T, K99R, A107T, K109R, D111N, L134P, F135V, T147A, I152V, K158R, E172G, V182A, E184R), hDHFR (V2A, I17V, N30D, E31G, Q36R, F59S, K69E, I72T, H88Y, F89L, N108D, K109E, V110A, 1115V, Y122D, L132P, F135S, M140V, E144G, T147A, Y157C, V170A, K174R, N186S), hDHFR (L100P, E102G, Q103R, P104S, E105G, N108D, V113A, W114R, Y122C, M126I, N127R, H128Y, L132P, F135P, I139T, F148S, F149L, I152V, D153A, D169G, V170A, I176A, K177R, V182A, K185R, N186S), and hDHFR (A10T, Q13R, N14S, N20D, P24S, N30S, M38T, T40A, K47R, N49S, K56R, I61T, K64R, K69R, I72A, R78G, E82G, F89L, D96G, N108D, M112V, W114R, Y122D, K123E, I139V, Q141R, D142G, F148L, E151G, E155G, Y157R, Q171R, Y183C, E184G, K185del, D187N).

In some embodiments, the DD comprises the mutant protein having three mutations hDHFR (M1del, Y122I, N127Y).

In some embodiments, the DD comprises the mutant protein having three mutations hDHFR (M1del, I17V, Y122I).

In some embodiments, the DD comprises the mutant protein having two mutations hDHFR (M1del, I17V).

In some embodiments, the CAR comprises

(a) an extracellular target moiety;

(b) a transmembrane domain;

(c) an intracellular signaling domain; and

(d) optionally, one or more co-stimulatory domains.

In some embodiments, the extracellular target moiety is selected from any of:

a single chain variable fragment (scFv),

an Ig NAR,

a Fab fragment,

a Fab′ fragment,

a F(ab)′2 fragment,

a F(ab)′3 fragment,

an Fv,

a bis-scFv, a (scFv)2,

a minibody,

a diabody,

a triabody,

a tetrabody,

an intrabody,

a disulfide stabilized Fv protein (dsFv),

a unibody,

a nanobody, and

an antigen binding region derived from an antibody that specifically binds to any of a protein of interest, a ligand, a receptor, a receptor fragment or a peptide aptamer.

In some embodiments, the extracellular target moiety is a scFv derived from an antibody that specifically binds a CD19 antigen.

In some embodiments, the scFv is a CD19 scFv is selected from the group consisting of:

(a) an amino acid sequence selected from the group consisting of SEQ ID NOs: 465; 83-227; 1034-1036; or

(b) a heavy chain variable region having an amino acid sequence independently selected from the group consisting of SEQ ID NO: 9-40, 1169, and a light chain variable region having an amino acid sequence independently selected from the group consisting of SEQ ID NOs: 41-82, 1170.

In some embodiments, (a) the intracellular signaling domain of the CAR is the signaling domain derived from T cell receptor CD3zeta or a cell surface molecule selected from the group consisting of FcR gamma, FcR beta, CD3 gamma, CD3 delta, CD3 epsilon, CD5, CD22, CD79a, CD79b, and CD66d; and

(b) the co-stimulatory domain is present and is selected from the group consisting of 4-1BB (CD137), 2B4, HVEM, ICOS, LAG3, DAP10, DAP12, CD27, CD28, OX40 (CD134), CD30, CD40, ICOS (CD278), glucocorticoid-induced tumor necrosis factor receptor (GITR), lymphocyte function-associated antigen-1 (LFA-1), CD2, CD7, LIGHT, NKG2C, and B7-H3.

In some embodiments, the intracellular signaling domain of the CAR is a T cell receptor CD3zeta signaling domain comprising the amino acid sequence of SEQ ID NO: 229.

In some embodiments, the intracellular signaling domain of the CAR is a T cell receptor CD3zeta signaling domain comprising the amino acid sequence of SEQ ID NO: 467 and the co-stimulatory domain is present, said co-stimulatory domain being selected from amino acid sequence of SEQ ID NOs: 233, 228-232, and 234-334.

In some embodiments, the transmembrane domain is derived from any of the members of the group consisting of:

(a) a molecule selected from the group consisting of CD8α, CD4, CD5, CD8, CD8α, CD9, CD16, CD22, CD33, CD28, CD37, CD45, CD64, CD80, CD86, CD148, DAP 10, EpoRI, GITR, LAG3, ICOS, Her2, OX40 (CD134), 4-1BB (CD137), CD152, CD154, PD-1, or CTLA-4

(b) a transmembrane region of an alpha, beta or zeta chain of a T-cell receptor;

(c) the CD3 epsilon chain of a T-cell receptor; and

(d) an immunoglobulin selected from IgG1, IgD, IgG4, and an IgG4 Fc region.

In some embodiments, the transmembrane domain comprises an amino acid sequence selected from the group consisting of SEQ ID NOs. 369, 335-368, 370-385 and 697-707.

In some embodiments, the CAR further comprises a hinge region near the transmembrane domain, said hinge region comprising an amino acid sequence selected from the group consisting of SEQ ID NOs. 400, 386-399, and 401-464.

In some embodiments, the SRE is responsive to or interacts with at least one stimulus.

In some embodiments, the stimulus is Trimethoprim (TMP) or Methotrexate (MTX).

In some embodiments, the invention comprises (a) an effector module is selected from the group consisting of SEQ ID NO. 1121, 1123, 1129, 1131, 1133, 1135, 1137, 1139, and 1141; and

(b) the CAR is selected from the group consisting of SEQ ID NO. 1120, 1122, 1128, 1130, 1132, 1134, 1136, 1138, and 1140.

In some embodiments, the composition described herein comprises the amino acid sequence selected from the group consisting of SEQ ID NO. 1127, 1125, 1126, 1082, 1118, 1119, 1124, or 1127.

In some embodiments, the invention comprises a polynucleotide encoding any of the compositions described herein.

In some embodiments, the invention comprises a vector comprising a polynucleotide described herein.

In some embodiments, the inventive comprises an immune cell for adoptive cell transfer (ACT), which expresses the compositions described herein, the polynucleotides described herein, and/or is transduced or transfected with the vector described herein.

In some embodiments, the invention comprises a method of inducing an immune response in a subject comprising (a) preparing an immune cell comprising the compositions described herein; and (b) administering an effective amount of the immune cells to the subject thereby inducing an immune response.

In some embodiments, the invention comprises a method of inducing the expression of T cell activation markers comprising administering to a cell or a subject, an effective amount of any the compositions described herein.

In some embodiments, the invention or compositions comprises an engineered cell comprising:

(a) a first recombinant protein comprising an effector module, said effector module comprising:
(i) a stimulus response element (SRE) linked to at least one recombinant protein selected from: a cytokine, a cytokine-cytokine receptor fusion protein, and a CD19 chimeric antigen receptor (CD19 CAR); and
(ii) the SRE comprises a DD, wherein said DD is derived from a parent protein or a mutant protein having one or more amino acid mutations in the amino acid sequence of the DD compared to said parent protein, wherein the parent protein is selected from the group consisting of:
(i) human DHFR (hDHFR) (SEQ ID NO: 1);
(ii) E. coli DHFR (ecDHFR) (SEQ ID NO: 2); and
(iii) human protein FKBP (SEQ ID NOs: 3 or 1087); and

(b) optionally, a second recombinant protein comprising a CD19 chimeric antigen receptor (CAR).

In some embodiments, the cytokine comprises IL12, IL15, or combinations thereof.

In some embodiments, the IL12 is a fusion protein comprising a p40 subunit, a linker, and a p35 subunit.

In some embodiments, the p40 subunit is a p40 (23-328 of WT) (SEQ ID NO: 563), a p40 (WT) (SEQ ID NO:1091) or a p40 (23-328 of WT) (K217N) (SEQ ID NO: 578).

In further embodiments, the p40 subunit is p40 (23-328 of WT) (SEQ ID NO: 563).

In some embodiments, the p35 subunit is a p35 (57-253 of WT) (SEQ ID NO: 564) or p35 (WT) (SEQ ID NO: 1093).

In further embodiments, the p35 subunit is a p35 (57-253 of WT) (SEQ ID NO: 564).

In some embodiments, the cytokine-cytokine receptor fusion polypeptide comprises the whole or a portion of SEQ. ID NOs: 616, 632 fused to the whole or a portion of any of SEQ. ID NOs: 632; 855, or 1097 to produce a IL15-1L15 receptor fusion polypeptide.

In some embodiments, the parent protein is a human DHFR (hDHFR), and the DD comprises one or more mutations selected from the group consisting of: Mdel1, I17A, I17V, Q36F, Q36K, N65F, Y122I, N127Y, and A125F.

In some embodiments, the parent protein is a human DHFR (hDHFR), and the DD comprises one or more mutations selected from:

a single mutation selected from the group consisting of: Mdel1, I17A, I17V, Q36F, Q36K, N65F, Y122I, and A125F;

a double mutation selected from the group consisting of: (M1del, I17A), (M1del, I17V), and (M1del, Y122I);

a triple mutation selected from the group consisting of: (M1del, Y122I, A125F), (M1del, Q36K, Y122I), (M1del, I17V, Y122I), and (M1del, I17A, Y122I); and

a quadruple or higher mutation selected from the group consisting of: (M1del, Q36F, N65F, Y122I).

In some embodiments, the DD comprises an hDHFR mutant protein having three mutations (M1del, Y122I, N127Y).

In some embodiments, the DD comprises an hDHFR mutant protein having three mutations (M1del, I17V, Y122I).

In some embodiments, the DD comprises an hDHFR mutant protein having two mutations (M1 del, I17V).

In some embodiments, the CD19 CAR is linked to the effector module.

In other embodiments, the CD19 CAR is not linked to the effector module.

In some embodiments, the CD19 CAR comprises:

(a) a CD19 binding moiety;

(b) a transmembrane domain;

(c) an intracellular signaling domain; and

(d) optionally, one or more co-stimulatory domains.

In some embodiments, the CD19 binding moiety is selected from:

a single chain variable fragment (scFv),

an Ig NAR,

a Fab fragment,

a Fab′ fragment,

a F(ab)′2 fragment,

a F(ab)′3 fragment,

an Fv,

a bis-scFv, a (scFv)2,

a minibody,

a diabody,

a triabody,

a tetrabody,

an intrabody,

a disulfide stabilized Fv protein (dsFv),

a unibody,

a nanobody, and

an antigen binding region derived from any one of (a) to (p) that binds to CD19.

In some embodiments, the CD19 binding moiety is a scFv that specifically binds a CD19 antigen.

In some embodiments, the scFv is a CD19 scFv comprising an amino acid sequence of SEQ ID NO: 465.

In some embodiments, the cytokine, cytokine-cytokine receptor fusion protein or CAR component is further linked to at least one of:

(a) a leader sequence;

(b) a signal peptide:

(c) a linker;

(d) a spacer;

(e) a cleavage site;

(f) a tag;

(g) a co-stimulatory domain;

(h) a fluorescence protein; and

(i) a hinge.

In some embodiments, the intracellular signaling domain of the CD19 CAR is the signaling domain derived from T cell receptor CD3zeta or a cell surface molecule selected from the group consisting of FcR gamma, FcR beta, CD3 gamma, CD3 delta, CD3 epsilon, CD5, CD22, CD79a, CD79b, and CD66d; and the co-stimulatory domain is present and is selected from the group consisting of 4-1BB (CD137), 2B4, HVEM, ICOS, LAG3, DAP10, DAP12, CD27, CD28, OX40 (CD134), CD30, CD40, ICOS (CD278), glucocorticoid-induced tumor necrosis factor receptor (GITR), lymphocyte function-associated antigen-1 (LFA-1), CD2, CD7, LIGHT, NKG2C, and B7-H3.

In some embodiments, the intracellular signaling domain of the CD19 CAR comprises a T-cell receptor CD3zeta signaling domain comprising the amino acid sequence of SEQ ID NO: 299.

In some embodiments, the intracellular signaling domain of the CD19 CAR is a T-cell receptor CD3zeta signaling domain comprising the amino acid sequence of SEQ ID NO: 467 and when the co-stimulatory domain is present, the co-stimulatory domain has an amino acid sequence selected from SEQ ID NOs: 233, 228-232, and 234-334.

In some embodiments, the transmembrane domain is derived from any of the members of the group consisting of:

(a) a molecule selected from the group consisting of CD8α, CD4, CD5, CD8, CD8α, CD9, CD16, CD22, CD33, CD28, CD37, CD45, CD64, CD80, CD86, CD148, DAP 10, EpoRI, GITR, LAG3, ICOS, Her2, OX40 (CD134), 4-1BB (CD137), CD152, CD154, PD-1, or CTLA-4

(b) a transmembrane region of an alpha, beta or zeta chain of a T-cell receptor;

(c) the CD3 epsilon chain of a T-cell receptor; and

(d) an immunoglobulin selected from IgG1, IgD, IgG4, and an IgG4 Fc region.

In some embodiments, the transmembrane domain comprises an amino acid sequence selected from the group consisting of SEQ ID NOs: 369, 335-368, 370-385 and 697-707.

In some embodiments, the CAR further comprises a hinge region near the transmembrane domain, said hinge region comprising an amino acid sequence selected from the group consisting of SEQ ID NOs: 400, 386-399, and 401-464.

In some embodiments, the SRE is responsive to or interacts with at least one stimulus.

In some embodiments, the stimulus is Trimethoprim (TMP) or Methotrexate (MTX).

In some embodiments, (a) the effector module is selected from the group consisting of SEQ ID NOs: 1121, 1123, 1129, 1131, 1133, 1135, 1137, 1139, and 1141; and (b) the CD19 CAR is selected from the group consisting of SEQ ID NOs: 1120, 1122, 1128, 1130, 1132, 1134, 1136, 1138, and 1140.

In some embodiments, the cell comprises at least one recombinant protein comprising an amino acid sequence selected from the group consisting of SEQ ID NOs: 1127, 1125, 1126, 1082, 1118, 1119, 1124, and 1127.

In some embodiments, the cell is a T-cell.

In some embodiments, the invention comprises a nucleic acid molecule, comprising:

(1) a first polynucleotide, optionally a first expression cassette, encoding a first recombinant protein comprising a stimulus response element (SRE) linked to at least one of a cytokine, a cytokine-cytokine receptor fusion protein, and a CD19 CAR; wherein the SRE comprises a DD, wherein said DD is derived from a parent protein or a mutant protein having one or more amino acid mutations in the amino acid sequence of the DD compared to said parent protein, wherein the parent protein is selected from the group consisting of:
(i) human DHFR (hDHFR) (SEQ ID NO: 1);
(ii) E. coli DHFR (ecDHFR) (SEQ ID NO: 2); and
(iii) human protein FKBP (SEQ ID Nos: 3 or 1087); and

(2) optionally, a second polynucleotide, optionally a second expression cassette, encoding a second recombinant protein comprising a CD19 chimeric antigen receptor (CD19 CAR).

In some embodiments, the first and second polynucleotides, optionally the first and second expression cassettes, are operably linked to the same or different promoters.

In some embodiments, the CD19 CAR is under control of the SRE.

In some embodiments, the CD19 CAR is not under control of the SRE.

In some embodiments, the first polynucleotide and the optional second polynucleotide encode a recombinant protein as set forth in any one of claims 1-30.

In some embodiments, the nucleic acid molecule is isolated.

In some embodiments, the invention comprises a vector comprising a nucleic acid molecule described herein.

In some embodiments, the vector is a plasmid, lentiviral vector, retroviral vector, adenoviral vector, or adeno-associated viral vector.

In some embodiments, the vector is integrase defective.

In some embodiments, the invention comprises a T-cell, comprising the nucleic acid molecule or a vector described herein.

In some embodiments, the T-cell is a CD4+ or CD8+ T-cell.

In some embodiments, the T-cell is a human T-cell.

In some embodiments, the T-cell is isolated.

In some embodiments, the invention comprises a pharmaceutical composition, comprising the cell or T-cell described herein and a pharmaceutically acceptable carrier.

In some embodiments, the invention comprises a method of producing a genetically engineered T-cell, comprising:

introducing into a T-cell:

(i) a first polynucleotide encoding a stimulus response element (SRE) linked to at least one recombinant protein selected from: a cytokine, a cytokine-cytokine receptor fusion protein, and a CD19 CAR; wherein the SRE comprises a DD, wherein said DD is derived from a parent protein or a mutant protein having one or more amino acid mutations in the amino acid sequence of the DD compared to said parent protein, wherein the parent protein is selected from the group consisting of:
(i) human DHFR (hDHFR) (SEQ ID NO: 1);
(ii) E. coli DHFR (ecDHFR) (SEQ ID NO: 2); and
(iii) human protein FKBP (SEQ ID NOs: 3 or 1087); and

(ii) optionally a second polynucleotide encoding a CD19 chimeric antigen receptor (CAR);

wherein the first polynucleotide encodes the at least one cytokine, cytokine-cytokine receptor fusion protein, and CD19 CAR, and at least one of the at least one cytokine, cytokine-cytokine receptor fusion protein, and CD19 CAR encoded by the first polynucleotide is under control of the SRE.

In some embodiments, the invention comprises a method of regulating expression of an immunotherapeutic agent in a genetically engineered T-cell, comprising introducing into a T-cell, a first polynucleotide encoding a stimulus response element (SRE) linked to at least one recombinant protein selected from: a cytokine, a cytokine-cytokine receptor fusion protein, and a CD19 chimeric antigen receptor (CD19 CAR); wherein the SRE comprises a DD, wherein said DD is derived from a parent protein or a mutant protein having one or more amino acid mutations in the amino acid sequence of the DD compared to said parent protein, wherein the parent protein is selected from the group consisting of:

(i) human DHFR (hDHFR) (SEQ ID NO: 1);

(ii) E. coli DHFR (ecDHFR) (SEQ ID NO: 2); and

(iii) human protein FKBP (SEQ ID NOs: 3 or 1087); and optionally a second polynucleotide encoding a CD19 chimeric antigen receptor (CAR);

wherein the DD is stabilized in the presence of a stimulus and enables expression of the at least one cytokine, cytokine-cytokine receptor fusion protein, and a CD19 CAR, and wherein expression of the at least one cytokine, cytokine-cytokine receptor fusion protein, and a CD19 CAR in the T-cell is significantly increased in the presence of the stimulus as compared to expression of the at least one cytokine, cytokine-cytokine receptor fusion protein and a CD19 CAR in the absence of the stimulus.

### Methods of CD19 Antibody Development and Characterization

In some embodiments, the present invention provides methods of producing CD19 antibodies, antibody fragments or variants. Such methods may include the steps of: (1) preparing a composition with CD19, (2) contacting a library of antibodies or antibody fragments or variable with the composition, and (3) identifying one or more CD19 antibodies. Also, provided herein are methods for identifying FMC63-distinct CD19 antibodies, antibody fragments or variable.

The present invention also provides methods for identifying anti-CD19 scFvs. The identifying method may comprise (a) expressing CD19 in a cell line, said cell line having none or low levels of endogenously expressed CD19; (b) incubating the cells expression CD19 of (a) with phages of a phage library that has been pre-cleared of non-specific binding phages; (c) recovering from the incubated mixture of (b) phages bound to CD19 expressed from the cell line of (a) thereby identifying anti-CD19 scFv from the bound phages.

Also provided herein are methods for identifying FMC63-distinct binding domains and using CD19 antigens in which the FMC63 binding epitope is masked or absent. In some embodiments, the FMC63 binding domain may be included in the payloads and effector modules of the invention.

In some embodiments, the present invention provides methods of identifying CD19 scFvs. Such methods may involve screening phagemid libraries for CD19 scFvs. Phagemid libraries expressing recombinant scFvs associated with the surface of bacteria or bacteriophages are useful in the present inventions. Phagemid libraries may be generated by PCR implication of the polynucleotides encoding the heavy chain and the kappa light chain of the immunoglobulin IgM and infecting Cre recombinase positive bacteria with the vectors containing the PCR products at a high multiplicity of infection (MOI). The high MOI results in bacteria containing multiple phagemids, each of which encodes a different VH and VL genes, which can be recombined by the Cre recombinase. The resulting library that may be generated by recombination is approximately 10⁸ unique scFvs. In some instances, libraries of CD19 scFvs formatted into chimeric antigen receptor constructs may be screened to identify CD19scFvs useful in the present invention.

In some embodiments, scFvs immunologically specific to CD19 may be identified using cells that ectopically express full length, a fragment or a portion of CD19. Cell lines with low endogenous CD19 expression may be selected for ectopic expression. In some embodiments, the CD19 may be a naturally occurring isoform of human CD19.

In some embodiments, fusion proteins comprising the extracellular domains of CD19 (i.e. exon 1-exon 4) fused to the Fc region of human IgG1 (CD19sIg) are utilized to identify CD19 specific scFvs. Such fusion proteins have been described by Oliveira et al (2013) Journal of Translational Medicine 11:23; the contents of which are incorporated herein by reference in their entirety.

Also, provided herein are methods to identify FMC63-distinct scFvs, which include scFvs that are immunologically specific to and bind to an epitope of the CD19 antigen that is different or unlike the epitope of CD19 antigen that is bound by FMC63. In some embodiments, FMC63-distinct scFvs are identified by screening the scFv library with a complex consisting of human CD19 bound to FMC63. The CD19 of Rhesus macaque (Macaca mulatta) herein referred to as Rhesus CD19, bears 88% homology to the human CD19. Despite this high degree of homology, the Rhesus CD19 is not recognized by FMC63, indicating that the FMC63 epitope is in the region of human CD19 that is non-homologous to Rhesus CD19. Thus, in some embodiments, Rhesus CD19 may be used to screen scFv libraries for FMC63-distinct scFvs. Mutations in the region of Rhesus CD19 that is non-homologous to the human CD19 have been previously utilized to identify residues of human CD19 that confer binding to FMC63 (Sommermeyer et al. (2017) Leukemia February 16. doi: 10.1038/leu. 2017.57). In some embodiments, the mutational analysis described by Sommermeyer et al. may be utilized to design human CD19 mutants that are unable to bind to FMC63. Such mutants may include human CD19 (H218R, A237D, M243V, E244D, P250T) and human CD19 (H218R, A237D) and may be utilized to screen scFv libraries for FMC63-distinct scFvs. Sotillo et al have identified a splice variant of human CD19 lacking exon 2 in cancer patients (Sotillo et al. (2015) Cancer Discov. 2015 Dec. 5(12):1282-95). The splice variant lacking exon 2 is not recognized by FMC63 and may also be used to screen scFv libraries for FMC63-distinct scFvs.

CD19 IgG fusion molecules generated by fusing the Fc region of human IgG1 with the human CD19-complete extracellular domains, i.e., exons 1˜4 (CD19sIgG1-4) or extracellular domains lacking exon 2, i.e., exons 1, 3 and 4 (CD19sIgG1,3,4) may also be utilized to screen scFv libraries for FMC63-distinct scFvs.

### III. Pharmaceutical Compositions and Formulations

The present invention further provides pharmaceutical compositions comprising one or more biocircuits, effector modules, human administration, preparations should meet sterility, pyrogenicity, general safety and purity standards as required by FDA Office of Biological Standards.

A pharmaceutical composition and formulation in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

The compositions of the present invention may be formulated in any manner suitable for delivery. The formulation may be, but is not limited to, nanoparticles, poly (lactic-co-glycolic acid) (PLGA) microspheres, lipidoids, lipoplex, liposome, polymers, carbohydrates (including simple sugars), cationic lipids and combinations thereof.

In one embodiment, the formulation is a nanoparticle which may comprise at least one lipid. The lipid may be selected from, but is not limited to, DLin-DMA, DLin-K-DMA, 98N12-5, C₁₂-200, DLin-MC3-DMA, DLin-KC2-DMA, DODMA, PLGA, PEG, PEG-DMG and PEGylated lipids. In another aspect, the lipid may be a cationic lipid such as, but not limited to, DLin-DMA, DLin-D-DMA, DLin-MC3-DMA, DLin-KC2-DMA and DODMA.

For polynucleotides of the invention, the formulation may be selected from any of those taught, for example, in International Application PCT/US2012/069610, the contents of which are incorporated herein by reference in its entirety.

Relative amounts of the active ingredient, the pharmaceutically acceptable excipient or inert ingredient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1 and 100, e.g., between 0.5 and 50, between 1-30, between 5-80, at least 80 (w/w) active ingredient.

Efficacy of treatment or amelioration of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. In connection with the administration of compositions of the present invention, “effective against” for example a cancer, indicates that administration in a clinically appropriate manner results in a beneficial effect for at least a statistically significant fraction of patients, such as an improvement of symptoms, a cure, a reduction in disease load, reduction in tumor mass or cell numbers, extension of life, improvement in quality of life, or other effect generally recognized as positive by medical doctors familiar with treating the particular type of cancer.

A treatment or preventive effect is evident when there is a statistically significant improvement in one or more parameters of disease status, or by a failure to worsen or to develop symptoms where they would otherwise be anticipated. As an example, a favorable change of at least 10 in a measurable parameter of disease, and preferably at least 20, 30, 40, 50 or more can be indicative of effective treatment. Efficacy for a given composition or formulation of the present invention can also be judged using an experimental animal model for the given disease as known in the art. When using an experimental animal model, efficacy of treatment is evidenced when a statistically significant change is observed.

### IV. Applications

In one aspect of the present invention, methods for reducing a tumor volume or burden are provided. The methods comprise administering a pharmaceutically effective amount of a pharmaceutical composition comprising at least one biocircuit system, effector module, DD, and/or payload of interest (i.e., an immunotherapeutic agent), at least one vector, or cells to a subject having a tumor. The biocircuit system and effector module having any immunotherapeutic agent as described herein may be in forms of a polypeptide, or a polynucleotide such as mRNA, or a viral vector comprising the polynucleotide, or a cell modified to express the biocircuit, effector module, DD, and payload of interest (i.e., immunotherapeutic agent).

In another aspect of the present invention, methods for inducing an anti-tumor immune response in a subject are provided. The methods comprise administering a pharmaceutically effective amount of a pharmaceutical composition comprising at least one biocircuit system, effector module, DD, and/or payload of interest (i.e., an immunotherapeutic agent), at least one vector, or cells to a subject having a tumor. The biocircuit and effector module having any immunotherapeutic agent as described herein may be in forms of a polypeptide, or a polynucleotide such as mRNA, or a viral vector comprising the polynucleotide, or a cell modified to express the biocircuit, effector module, DD, and payload of interest (i.e., immunotherapeutic agent).

The methods, according to the present invention, may be adoptive cell transfer (ACT) using genetically engineered cells such as immune effector cells of the invention, cancer vaccines comprising biocircuit systems, effector modules, DDs, payloads of interest (i.e., immunotherapeutic agents) of the invention, or compositions that manipulate the tumor immunosuppressive microenvironment, or the combination thereof. These treatments may be further employed with other cancer treatment such as chemotherapy and radiotherapy.

### 1. Adoptive Cell Transfer (Adoptive Immunotherapy)

In some embodiments, cells which are genetically modified to express at least one biocircuit system, effector module, DD, and/or payload of interest (immunotherapeutic agent) may be used for adoptive cell therapy (ACT). As used herein, Adoptive cell transfer refers to the administration of immune cells (from autologous, allogenic or genetically modified hosts) with direct anticancer activity. ACT has shown promise in clinical application against malignant and infectious disease. For example, T cells genetically engineered to recognize CD19 have been used to treat follicular B cell lymphoma (Kochenderfer et al., Blood, 2010, 116:4099-4102; and Kochenderfer and Rosenberg, Nat Rev Clin Oncol., 2013, 10(5): 267-276) and ACT using autologous lymphocytes genetically-modified to express anti-tumor T cell receptors has been used to treat metastatic melanoma (Rosenberg and Dudley, Curr. Opin. Immunol. 2009, 21: 233-240).

According to the present invention, the biocircuits and systems may be used in the development and implementation of cell therapies such as adoptive cell therapy. Certain effector modules useful in cell therapy are given in FIGS. 7-12 in International Publication No. WO2017/180587, the contents of which are herein incorporated by reference in their entirety. The biocircuits, their components, effector modules and their SREs and payloads may be used in cell therapies to effect CAR therapies, in the manipulation or regulation of TILs, in allogeneic cell therapy, in combination T cell therapy with other treatment lines (e.g. radiation, cytokines), to encode engineered TCRs, or modified TCRs, or to enhance T cells other than TCRs (e.g. by introducing cytokine genes, genes for the checkpoint inhibitors PD1, CTLA4).

Provided herein are methods for use in adoptive cell therapy. The methods involve preconditioning a subject in need thereof, modulating immune cells with SRE, biocircuits and compositions of the present invention, administering to a subject, engineered immune cells expressing compositions of the invention and the successful engraftment of engineered cells within the subject.

In some embodiments, SREs, biocircuits and compositions of the present invention may be used to minimize preconditioning regimens associated with adoptive cell therapy. As used herein “preconditioning” refers to any therapeutic regimen administered to a subject to improve the outcome of adoptive cell therapy. Preconditioning strategies include but are not limited to total body irradiation and/or lymphodepleting chemotherapy. Adoptive therapy clinical trials without preconditioning have failed to demonstrate any clinical benefit, indicating its importance in ACT. Yet, preconditioning is associated with significant toxicity and limits the subject cohort that is suitable for ACT. In some instances, immune cells for ACT may be engineered to express cytokines such as IL12 and IL15 as payload using SREs of the present invention to reduce the need for preconditioning (Pengram et al. (2012) Blood 119 (18): 4133-41; the contents of which are incorporated by reference in their entirety).

In some embodiments, immune cells for ACT may be dendritic cells, T cells such as CD8+ T cells and CD4+ T cells, natural killer (NK) cells, NK T cells, Cytotoxic T lymphocytes (CTLs), tumor infiltrating lymphocytes (TILs), lymphokine activated killer (LAK) cells, memory T cells, regulatory T cells (Tregs), helper T cells, cytokine-induced killer (CIK) cells, and any combination thereof. In other embodiments, immune stimulatory cells for ACT may be generated from embryonic stem cell (ESC) and induced pluripotent stem cell (iPSC). In some embodiments, autologous or allogeneic immune cells are used for ACT.

In some embodiments, cells used for ACT may be T cells engineered to express CARs comprising an antigen-binding domain specific to an antigen on tumor cells of interest. In other embodiments, cells used for ACT may be NK cells engineered to express CARs comprising an antigen-binding domain specific to an antigen on tumor cells of interest. In addition to adoptive transfer of genetically modified T cells (e.g., CART cells) for immunotherapy, alternate types of CAR-expressing leukocytes, either alone, or in combination with CAR T cells may be used for adoptive immunotherapy. In one example, a mixture of T cells and NK cells may be used for ACT. The expression level of CARs in T cells and NK cells, according to the present invention, is tuned and controlled by a small molecule that binds to the DD(s) operably linked to the CAR in the effector module.

In some embodiments, the CARs of the present invention may be placed under the transcriptional control of the T cell receptor alpha constant (TRAC) locus in the T cells to achieve uniform CAR expression while enhancing T cell potency. The TRAC locus may be disrupted using the CRISPR/Cas 9, zinc finger nucleases (ZFNs), TALENs followed by the insertion of the CAR construct. Methods of engineering CAR constructs directed to the TRAC locus are described in Eyquem J. et al (2017) Nature. 543(7643):113-117 (the contents of which are incorporated herein by reference in their entirety).

In some embodiments, NK cells engineered to express the present compositions may be used for ACT. NK cell activation induces perforin/granzyme-dependent apoptosis in target cells. NK cell activation also induces cytokine secretion such as IFN γ, TNF-α and GM-CSF. These cytokines enhance the phagocytic function of macrophages and their antimicrobial activity and augment the adaptive immune response via up-regulation of antigen presentation by antigen presenting cells such as dendritic cells (DCs) (Reviewed by Vivier et al., Nat. Immunol., 2008, 9(5): 503-510).

Other examples of genetic modification may include the introduction of chimeric antigen receptors (CARs) and the down-regulation of inhibitory NK cell receptors such as NKG2A.

NK cells may also be genetically reprogrammed to circumvent NK cell inhibitory signals upon interaction with tumor cells. For example, using CRISPR, ZFN, or TALEN to genetically modify NK cells to silence their inhibitory receptors may enhance the anti-tumor capacity of NK cells.

Immune cells can be isolated and expanded ex vivo using a variety of methods known in the art. For example, methods of isolating and expanding cytotoxic T cells are described in U.S. Pat. Nos. 6,805,861 and 6,531,451; US Patent Publication NO. US20160348072A1 and International Patent Publication NO. WO2016168595A1; the contents of each of which are incorporated herein by reference in their entirety. Isolation and expansion of NK cells is described in US Patent Publication NO. US20150152387A1, U.S. Pat. No. 7,435,596; and Oyer, J. L. (2016). Cytotherapy. 18(5):653-63; the contents of each of which are incorporated by reference herein in its entirety. Specifically, human primary NK cells may be expanded in the presence of feeder cells e.g. a myeloid cell line that has been genetically modified to express membrane bound IL15, IL21, IL12 and 4-1BBL.

In some instances, sub populations of immune cells may be enriched for ACT. Methods for immune cell enrichment are taught in International Patent Publication NO. WO2015039100A1. In another example, T cells positive for B and T lymphocyte attenuator marker BTLA) may be used to enrich for T cells that are anti-cancer reactive as described in U.S. Pat. No. 9,512,401 (the content of each of which are incorporated herein by reference in their entirety).

In some embodiments, immune cells for ACT may be depleted of select sub populations to enhance T cell expansion. For example, immune cells may be depleted of Foxp3+T lymphocytes to minimize the ant-tumor immune response using methods taught in US Patent Publication NO. US 20160298081A1; the contents of which are incorporated by reference herein in their entirety.

In some embodiments, activation and expansion of T cells for ACT is achieved antigenic stimulation of a transiently expressed Chimeric Antigen Receptor (CAR) on the cell surface. Such activation methods are taught in International Patent NO. WO2017015427, the content of which are incorporated herein by reference in their entirety.

In some embodiments, immune cells may be activated by antigens associated with antigen presenting cells (APCs). In some embodiments, the APCs may be dendritic cells, macrophages or B cells that antigen specific or nonspecific. The APCs may autologous or homologous in their organ. In some embodiments, the APCs may be artificial antigen presenting cells (aAPCs) such as cell based aAPCs or acellular aAPCs. Cell based aAPCs are may be selected from either genetically modified allogeneic cells such as human erythroleukemia cells or xenogeneic cells such as murine fibroblasts and Drosophila cells. Alternatively, the APCs maybe be acellular wherein the antigens or costimulatory domains are presented on synthetic surfaces such as latex beads, polystyrene beads, lipid vesicles or exosomes.

In some embodiments, cells of the invention, specifically T cells may be expanded using artificial cell platforms. In one embodiment, the mature T cells may be generated using artificial thymic organoids (ATOS) described by Seet C S et al. 2017. Nat Methods. 14, 521-530 (the contents of which are incorporated herein by reference in their entirety). ATOs are based on a stromal cell line expressing delta like canonical notch ligand (DLL1). In this method, stromal cells are aggregated with hematopoietic stem and progenitor cells by centrifugation and deployed on a cell culture insert at the air—fluid interface to generate organoid cultures. ATO-derived T cells exhibit naive phenotypes, a diverse T cell receptor (TCR) repertoire and TCR-dependent function.

In some embodiments, adoptive cell therapy is carried out by autologous transfer, wherein the cells are derived from a subject in need of a treatment and the cells, following isolation and processing are administered to the same subject. In other instances, ACT may involve allogenic transfer wherein the cells are isolated and/or prepared from a donor subject other than the recipient subject who ultimately receives cell therapy. The donor and recipient subject may be genetically identical, or similar or may express the same HLA class or subtype.

In some embodiments, the multiple immunotherapeutic agents introduced into the immune cells for ACT (e.g., T cells and NK cells) may be controlled by the same biocircuit system. In one example, a cytokine such as IL12 and a CAR construct such as CD19 CAR are linked to the same hDHFR destabilizing domain. The expression of IL12 and CD19 CAR is tuned using TMP simultaneously. In other embodiments, the multiple immunotherapeutic agents introduced into the immune cells for ACT (e.g., T cells and NK cells) may be controlled by different biocircuit systems. In one example, a cytokine such as IL12 and a CAR construct such as CD19 CAR are linked to different DDs in two separate effector modules, thereby can be tuned separately using different stimuli. In another example, a suicide gene and a CAR construct may be linked to two separate effector modules.

Following genetic modulation using SREs, biocircuits and compositions of the invention, cells are administered to the subject in need thereof. Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338; the contents of each of which are incorporated herein by reference in their entirety.

In some embodiments, immune cells for ACT may be modified to express one or more immunotherapeutic agents which facilitate immune cells activation, infiltration, expansion, survival and anti-tumor functions. The immunotherapeutic agents may be a second CAR or TCR specific to a different target molecule; a cytokine or a cytokine receptor; a chimeric switch receptor that converts an inhibitory signal to a stimulatory signal; a homing receptor that guides adoptively transferred cells to a target site such as the tumor tissue; an agent that optimizes the metabolism of the immune cell; or a safety switch gene (e.g., a suicide gene) that kills activated T cells when a severe event is observed after adoptive cell transfer or when the transferred immune cells are no-longer needed.

In some embodiments, immune cells used for adoptive cell transfer can be genetically manipulated to improve their persistence, cytotoxicity, tumor targeting capacity, and ability to home to disease sites in vivo, with the overall aim of further improving upon their capacity to kill tumors in cancer patients. One example is to introduce effector modules of the invention comprising cytokines such as gamma-cytokines (IL2 and IL15) into immune cells to promote immune cell proliferation and survival. Transduction of cytokine genes (e.g., gamma-cytokines IL2 and IL15) into cells will be able to propagate immune cells without addition of exogenous cytokines and cytokine expressing NK cells have enhanced tumor cytotoxicity.

In some embodiments, biocircuits, their components, SREs or effector modules may be utilized to prevent T cell exhaustion. As used herein, “T cell exhaustion” refers to the stepwise and progressive loss of T cell function caused by chronic T cell activation. T cell exhaustion is a major factor limiting the efficacy of antiviral and antitumor immunotherapies. Exhausted T cells have low proliferative and cytokine producing capabilities concurrent with high rates of apoptosis and high surface expression of multiple inhibitory receptors. T cell activation leading to exhaustion may occur either in the presence or absence of the antigen.

In some embodiments, the biocircuits, and their components may be utilized to prevent T cell exhaustion in the context of Chimeric Antigen Receptor-T cell therapy (CAR-T). In this context, exhaustion in some instances, may be caused by the oligomerization of the scFvs of the CAR on the cell surface which leads to continuous activation of the intracellular domains of the CAR. As a non-limiting example, CARs of the present invention may include scFvs that are unable to oligomerize. As another non-limiting example, CARs that are rapidly internalized and re-expressed following antigen exposure may also be selected to prevent chronic scFv oligomerization on cell surface. In one embodiment, the framework region of the scFvs may be modified to prevent constitutive CAR signaling (Long et al. 2014. Cancer Research. 74(19) S1; the contents of which are incorporated by reference in their entirety). Tunable biocircuit systems of the present invention may also be used to regulate the surface expression of the CAR on the T cell surface to prevent chronic T cell activation. The CARs of the invention may also be engineered to minimize exhaustion. As a non-limiting example, the 41-BB signaling domain may be incorporated into CAR design to ameliorate T cell exhaustion. In some embodiments, any of the strategies disclosed by Long H A et al. may be utilized to prevent exhaustion (Long A H et al. (2015) Nature Medicine 21, 581-590; the contents of which are incorporated herein by reference in their entirety).

In some embodiments, the tunable nature of the biocircuits of the present invention may be utilized to reverse human T cell exhaustion observed with tonic CAR signaling. Reversibly silencing the biological activity of adoptively transferred cells using compositions of the present invention may be used to reverse tonic signaling which, in turn, may reinvigorate the T cells. Reversal of exhaustion may be measured by the downregulation of multiple inhibitory receptors associated with exhaustion.

In some embodiments, T cell metabolic pathways may be modified to diminish the susceptibility of T cells to exhaustion. Metabolic pathways may include, but are not limited to glycolysis, urea cycle, citric acid cycle, beta oxidation, fatty acid biosynthesis, pentose phosphate pathway, nucleotide biosynthesis, and glycogen metabolic pathways. As a non-limiting example, payloads that reduce the rate of glycolysis may be utilized to restrict or prevent T cell exhaustion (Long et al. Journal for Immunotherapy of Cancer 2013, 1 (Suppl 1): P21; the contents of which are incorporated by reference in their entirety). In one embodiment, T cells of the present invention may be used in combination with inhibitors of glycolysis such as 2-deoxyglucose, and rapamycin.

In some embodiments, effector modules of the present invention, useful for immunotherapy may be placed under the transcriptional control of the T cell receptor alpha locus constant (TRAC) locus in the T cells. Eyquem et al. have shown that expression of the CAR from the TRAC locus prevents T cell exhaustion and the accelerated differentiation of T cells caused by excessive T cell activation (Eyquem J. et al (2017) Nature. 543(7643):113-117; the contents of which are incorporated herein by reference in their entirety).

In some embodiments, payloads of the invention may be used in conjunction with antibodies or fragments that target T cell surface markers associated with T cell exhaustion. T-cell surface markers associated with T cell exhaustion that may be used include, but are not limited to, CTLA-1, PD-1, TGIT, LAG-3, 2B4, BTLA, TIM3, VISTA, and CD96.

In one embodiment, the payload of the invention may be a CD276 CAR (with CD28, 4-IBB, and CD3 zeta intracellular domains), that does not show an upregulation of the markers associated with early T cell exhaustion (see International patent publication No. WO2017044699; the contents of which are incorporated by reference in their entirety).

In some embodiments, the compositions of the present invention may be utilized to alter TIL (tumor infiltrating lymphocyte) populations in a subject. In one embodiment, any of the payloads described herein may be utilized to change the ratio of CD4 positive cells to CD8 positive populations. In some embodiments, TILs may be sorted ex vivo and engineered to express any of the cytokines described herein. Payloads of the invention may be used to expand CD4 and/or CD8 populations of TILs to enhance TIL mediated immune response.

The present invention provides method of inducing an immune response in a cell. As used herein the term “immune response” refers to the activity of the cells of the immune system in response to stimulus, or an antigen. In some embodiments, the antigen may be a cancer antigen. In some aspects, the methods of inducing an immune response may involve administering to the cell, a therapeutically effective amount of any of the compositions described herein. In one aspect, the method may involve administering the pharmaceutical compositions described herein. In one aspect, the method may involve administering the polynucleotides, vectors. In some embodiments, induction of the immune response occurs due to the expression and or function of the immunotherapeutic agents described herein. Methods of inducing immune response further may involve administering to the cell, an effective amount of the stimulus to tune the expression of the immunotherapeutic agent. In some embodiments, the immunotherapeutic agent is capable of inducing an immune response in response to the stimulus. The induction of the immune response may occur in a cell within a subject i.e. in vivo, ex vivo or in vitro. The induction of an immune response may be evaluated by measuring the release of cytokine such as IL2 and IFNγ from the cells. In some embodiments, the induction of an immune response may be measured by evaluating the cell markers such as but not limited to CD3, CD4, CD8, CD 14, CD20, CD11b, CD16, CD45 and HLA-DR, CD 69, CD28, CD44, IFN gamma, Granzyme, Tbet, pSTAT4, CD25, and ICOS using methods known in the art such as FACS. In some embodiments, Granzyme, Tbet, pSTAT4, CD25, and ICOS as described herein are also referred to as T cell activation markers. Examples of cell markers for antigen presenting cells include, but are not limited to, MHC class I, MHC Class II, CD40, CD45, B7-1, B7-2, IFN γ receptor and IL2 receptor, ICAM-1 and/or Fcγ receptor. Examples of cell surface markers for dendritic cells include, but are not limited to, MHC class I, MHC Class II, B7-2, CD18, CD29, CD31, CD43, CD44, CD45, CD54, CD58, CD83, CD86, CMRF-44, CMRF-56, DCIR and/or Dectin-1 and the like; while in some cases also having the absence of CD2, CD3, CD4, CD8, CD14, CD15, CD16, CD 19, CD20, CD56, and/or CD57. Examples of cell surface markers for NK cells include, but are not limited to, CCL3, CCL4, CCL5, Granulysin, Granzyme B, Granzyme K, IL10, IL22, IFNg, LAP, Perforin, and TNFa. In one some embodiments, the cell markers induces at least a 2-fold, 3-fold, 4-fold, 5-fold, 6-fold, 7-fold, 8-fold, 9-fold, 10-fold, 100-fold, 1000-fold.

In some embodiments, the polynucleotides may further comprise, at least one additional feature selected from, but not limited to, a promoter, a linker, a signal peptide, a tag, a cleavage site and a targeting peptide.

The present invention also provides vectors comprising polynucleotides described herein. In one aspect, the vector may be a viral vector. In some embodiments, the viral vector may be a retroviral vector, a lentiviral vector, a gamma retroviral vector, a recombinant AAV vector, an adeno viral vector, and an oncolytic viral vector.

The present invention also provides immune cells for adoptive cell transfer (ACT) which may express the compositions of the invention, the polynucleotides described herein. In one aspect, the immune cells may be infected or transfected with the vectors described herein. The immune cells for ACT may be selected from, but not limited to a CD8+ T cell, a CD4+ T cell, a helper T cell, a natural killer (NK) cell, a NKT cell, a cytotoxic T lymphocyte (CTL), a tumor infiltrating lymphocyte (TIL), a memory T cell, a regulatory T (Treg) cell, a cytokine-induced killer (CIK) cell, a dendritic cell, a human embryonic stem cell, a mesenchymal stem cell, a hematopoietic stem cell, or a mixture thereof.

In some embodiments, the immune cells may be autologous, allogeneic, syngeneic, or xenogeneic in relation to a particular individual subject.

The present invention provides methods for reducing a tumor volume or burden in a subject comprising contacting the subject with the immune cells of the invention. Also provided herein, is a method for inducing an anti-tumor immune response in a subject, comprising administering the immune cells of the system to the subject.

The tunable system and agent described herein may be a biocircuit system comprising at least one effector module that is responsive to at least one stimulus. The biocircuit system may be, but is not limited to, a destabilizing domain (DD) biocircuit system, a dimerization biocircuit system, a receptor biocircuit system, and a cell biocircuit system. These systems are further taught in co-owned U.S. Provisional Patent Application No. 62/320,864 filed Apr. 11, 2016, 62/466,596 filed Mar. 3, 2017 and the International Publication WO2017/180587 (the contents of each of which are herein incorporated by reference in their entirety).

The present invention provides compositions for inducing immune responses in a cell or a subject. In one embodiment, the compositions may include a first effector module. The effector module may comprise a first stimulus response element (SRE) operably linked to a first immunotherapeutic agent. In some aspects, the first effector module may be further operably linked to a second immunotherapeutic agent.

In some embodiments, the composition may comprise a first immunotherapeutic agent operably linked to a second immunotherapeutic agent.

In some embodiments, the first immunotherapeutic agent may be a cytokine or a cytokine receptor fusion protein.

In one embodiment, the first immunotherapeutic agent is a cytokine. In one aspect, the cytokine is IL12. The IL12 may be a fusion protein comprising a p40 subunit, a linker, and a p35 subunit.

In one aspect, the first immunotherapeutic agent may be a cytokine-cytokine receptor fusion polypeptide. The cytokine-cytokine receptor fusion polypeptide may include the whole or a portion of SEQ ID NO. 632; 855-1097 thereby generating an IL15-IL15 receptor fusion polypeptide.

In some embodiments, the second immunotherapeutic agent may be selected from but is not limited an scFv comprising at least 70% sequence identity to a sequence selected from the group consisting of SEQ ID NO. 1034-1036. In some embodiments, the first immunotherapeutic agent may be a chimeric antigen receptor comprising the scFv.

In some respects, the scFv may specifically bind a CD19 antigen. In one aspect, the SRE of the composition may be responsive to or interact with at least one stimulus.

In one aspect, the CAR may be selected from, but is not limited to a standard CAR, a split CAR, an off-switch CAR, an on-switch CAR, a first-generation CAR, a second-generation CAR, a third-generation CAR, or a fourth-generation CAR.

The chimeric antigen may further comprise a transmembrane domain; an intracellular signaling domain; and optionally, one or more co-stimulatory domains.

In some embodiments, the intracellular signaling domain of the CAR may be a signaling domain derived from T cell receptor CD3zeta. In some embodiments, the intracellular signaling domain may be selected from a cell surface molecule selected from the group consisting of FcR gamma, FcR beta, CD3 gamma, CD3 delta, CD3 epsilon, CD5, CD22, CD79a, CD79b, and CD66d. In one aspect, the CAR may include a co-stimulatory domain. In some embodiments, the co-stimulatory domain may be selected from the group consisting of 2B4, HVEM, ICOS, LAG3, DAP10, DAP12, CD27, CD28, 4-1BB (CD137), OX40 (CD134), CD30, CD40, ICOS (CD278), glucocorticoid-induced tumor necrosis factor receptor (GITR), lymphocyte function-associated antigen-1 (LFA-1), CD2, CD7, LIGHT, NKG2C, and B7-H3.

The co-stimulatory domain is present and is selected from the group consisting of 2B4, HVEM, ICOS, LAG3, DAP10, DAP12, CD27, CD28, 4-1BB (CD137), OX40 (CD134), CD30, CD40, ICOS (CD278), glucocorticoid-induced tumor necrosis factor receptor (GITR), lymphocyte function-associated antigen-1 (LFA-1), CD2, CD7, LIGHT, NKG2C, and B7-H3.

In some embodiments, the intracellular signaling domain of the CAR may be a T cell receptor CD3zeta signaling domain, which may comprise the amino acid sequence of SEQ ID NO. 299.

In some embodiments, T cell receptor CD3zeta signaling domain of the CAR, comprising the amino acid sequence of SEQ ID NO. 467 may further comprise at least one co-stimulatory domain. The co-stimulatory domain may comprise an amino acid sequence of SEQ ID NOs. 228-334.

In one embodiment, the transmembrane domain of the CAR may be derived from a transmembrane region of an alpha, beta or zeta chain of a T-cell receptor. In one aspect, the transmembrane domain may be derived from the CD3 epsilon chain of a T-cell receptor. In one embodiment, the transmembrane domain may be derived from a molecule selected from CD4, CD5, CD8, CD8α, CD9, CD16, CD22, CD33, CD28, CD37, CD45, CD64, CD80, CD86, CD148, DAP 10, EpoRI, GITR, LAG3, ICOS, Her2, OX40 (CD134), 4-1BB (CD137), CD152, CD154, PD-1, or CTLA-4. In another embodiment, the transmembrane domain may be derived from an immunoglobulin selected from IgG1, IgD, IgG4, and an IgG4 Fc region. In one aspect, the transmembrane domain may comprise an amino acid sequence selected from the group consisting of any of SEQ ID NOs. 335-385 and 697-707.

In some embodiments, the CAR of the effector module may further comprise a hinge region near the transmembrane domain. In one aspect, the hinge region may comprise an amino acid sequence selected from the group consisting of any of SEQ ID NOs. 386-464.

In some embodiments, the SRE may comprise a destabilizing domain (DD). The DD may be derived from a parent protein or from a mutant protein having one, two, there, or more amino acid mutations compared to the parent protein. In some embodiments, the parent protein may be selected from, but is not limited to, human protein FKBP, comprising the amino acid sequence of SEQ ID NO. 3 and 1087; human DHFR (hDHFR), comprising the amino acid sequence of SEQ ID NO. 2; or E. coli DHFR (ecDHFR), comprising the amino acid sequence of SEQ ID NO. 1.

In one aspect, the parent protein is hDHFR and the DD comprises a mutant protein. The mutant protein may comprise a single mutation and may be selected from, but not limited to hDHFR (I17V), hDHFR (M1del, I17A), hDHFR (F59S), hDHFR (N65D), hDHFR (K81R), hDHFR (A107V), hDHFR (Y1220, hDHFR (N127Y), hDHFR (M140I), hDHFR (K185E), hDHFR (N186D), and hDHFR (M140I). In some embodiments, the mutant protein may comprise two mutations and may be selected from, but not limited to, hDHFR (M1del, N127Y), hDHFR (M1del, I17V), hDHFR (M1del, Y122I), hDHFR (M1del, K185E), hDHFR (C7R, Y163C), hDHFR (A10V, H88Y), hDHFR (Q36K, Y122I), hDHFR (M53T, R138I), hDHFR (T57A, I72A), hDHFR (E63G, I176F), hDHFR (G21T, Y122I), hDHFR (L74N, Y122I), hDHFR (V75F, Y122I), hDHFR (L94A, T147A), DHFR (V121A, Y22I), hDHFR (Y122I, A125F), hDHFR (H131R, E144G), hDHFR (T137R, F143L), hDHFR (Y178H, E18IG), and hDHFR (Y183H, K185E), hDHFR (E162G, I176F)

In some embodiments, the mutant may comprise three mutations and the mutant may be selected from hDHFR (M1del, 117A, Y122I), hDHFR (M1del, I17V, Y122I), hDHFR (M1del, Y122I, M140I), hDHFR (M1del, N127Y, Y122I), hDHFR (M1del, E162G, I176F), and hDHFR (M1del, H131R, E144G), hDHFR (M1del, Y122I, A125F), hDHFR (V9A, S93R, P150L), hDHFR (I8V, K133E, Y163C), hDHFR (L23S, V121A, Y157C), hDHFR (K19E, F89L, E181G), hDHFR (Q36F, N65F, Y122I), hDHFR (G54R, M140V, S168C), hDHFR (V110A, V136M, K177R), hDHFR (Q36F, Y122I, A125F), hDHFR (N49D, F59S, D153G), and hDHFR (G21E, I72V, I176T). In some embodiments, the mutant may comprise four or more mutations and the mutant may be selected from hDHFR (M1del, Q36F, Y122I, A125F), hDHFR (M1del, Y122I, H131R, E144G), hDHFR (M1del, E31D, F32M, V116I), hDHFR (M1del, Q36F, N65F, Y122I), hDHFR (V2A, R33G, Q36R, L100P, K185R), hDHFR (M1del, D22S, F32M, R33S, Q36S, N65S), hDHFR (I17N, L98S, K99R, M112T, E151G, E162G, E172G), hDHFR (G16S, I17V, F89L, D96G, K123E, M140V, D146G, K156R), hDHFR (K81R, K99R, L100P, E102G, N108D, K123R, H128R, D142G, F180L, K185E), hDHFR (R138G, D142G, F143S, K156R, K158E, E162G, V166A, K177E, Y178C, K185E, N186S), hDHFR (N14S, P24S, F35L, M53T, K56E, R92G, S93G, N127S, H128Y, F135L, F143S, L159P, L160P, E173A, F180L), hDHFR (F35L, R37G, N65A, L68S, K69E, R71G, L80P, K99G, G117D, L132P, I139V, M140I, D142G, D146G, E173G, D187G), hDHFR (L28P, N30H, M38V, V44A, L68S, N73G, R78G, A97T, K99R, A107T, K109R, D111N, L134P, F135V, T147A, I152V, K158R, E172G, V182A, E184R), hDHFR (V2A, I17V, N30D, E31G, Q36R, F59S, K69E, I72T, H88Y, F89L, N108D, K109E, V110A, 1115V, Y122D, L132P, F135S, M140V, E144G, T147A, Y157C, V170A, K174R, N186S), hDHFR (L100P, E102G, Q103R, P104S, E105G, N108D, V113A, W114R, Y122C, M126I, N127R, H128Y, L132P, F135P, I139T, F148S, F149L, I152V, D153A, D169G, V170A, I176A, K177R, V182A, K185R, N186S), and hDHFR (A10T, Q13R, N14S, N20D, P24S, N30S, M38T, T40A, K47R, N49S, K56R, I61T, K64R, K69R, I72A, R78G, E82G, F89L, D96G, N108D, M112V, W114R, Y122D, K123E, I139V, Q141R, D142G, F148L, E151G, E155G, Y157R, Q171R, Y183C, E184G, K185del, D187N).

In one aspect, the stimulus of the SRE may be Trimethoprim or Methotrexate.

The present invention also provides polynucleotides comprising the compositions of the invention.

In one embodiment, the compositions of the invention may include SEQ ID NO. 1124-1127, and 1142.

In some aspects, the compositions of the invention may include a first effector module, which may be selected from the group consisting of SEQ ID NO. 1121, 1123, 1129, 1131, 1133, 1135, 1137, 1139, and 1141; and a second immunotherapeutic agent which may be selected from the group consisting of SEQ ID NO. 1120, 1122, 1128, 1130, 1132, 1134, 1136, 1138, and 1140.

The present invention also provides polynucleotides encoding the compositions described herein; and vectors comprising polynucleotides described herein. Also provided herein, are the methods of reducing tumor burden and methods of inducing an immune response.

### 2. Cancer Vaccines

In some embodiments, biocircuits, effector modules, payloads of interest (immunotherapeutic agents), vectors, cells and compositions of the present invention may be used in conjunction with cancer vaccines.

In some embodiments, cancer vaccine may comprise peptides and/or proteins derived from tumor associated antigen (TAA). Such strategies may be utilized to evoke an immune response in a subject, which in some instances may be a cytotoxic T lymphocyte (CTL) response. Peptides used for cancer vaccines may also modified to match the mutation profile of a subject. For example, EGFR derived peptides with mutations matched to the mutations found in the subject in need of therapy have been successfully used in patients with lung cancer (Li F et al. (2016) Oncoimmunology. October 7; 5(12): e1238539; the contents of which are incorporated herein by reference in their entirety).

In one embodiment, cancer vaccines of the present invention may superagonist altered peptide ligands (APL) derived from TAAs. These are mutant peptide ligands deviate from the native peptide sequence by one or more amino acids, which activate specific CTL clones more effectively than native epitopes. These alterations may allow the peptide to bind better to the restricting Class I MHC molecule or interact more favorably with the TCR of a given tumor-specific CTL subset. APLs may be selected using methods taught in US Patent Publication NO. US20160317633A1, the contents of which are incorporated herein by reference in their entirety.

### 3. Combination Treatments

In some embodiments, it is desirable to combine compositions, vectors and cells of the invention for administration to a subject. Compositions of the invention comprising different immunotherapeutic agents may be used in combination for enhancement of immunotherapy.

In some embodiments, it is desirable to combine compositions of the invention with adjuvants, that can enhance the potency and longevity of antigen-specific immune responses. Adjuvants used as immunostimulants in combination therapy include biological molecules or delivery carriers that deliver antigens. As non-limiting examples, the compositions of the invention may be combined with biological adjuvants such as cytokines, Toll Like Receptors, bacterial toxins, and/or saponins. In other embodiments, the compositions of the present invention may be combined with delivery carriers. Exemplary delivery carriers include, polymer microspheres, immune stimulating complexes, emulsions (oil-in-water or water-in-oil), aluminum salts, liposomes or virosomes.

In some embodiments, immune effector cells modified to express biocircuits, effector modules, DDs and payloads of the invention may be combined with the biological adjuvants described herein. Dual regulation of CAR and cytokines and ligands to segregate the kinetic control of target-mediated activation from intrinsic cell T cell expansion. Such dual regulation also minimizes the need for pre-conditioning regimens in patients. As a non-limiting example, DD regulated CAR e.g. CD19 CAR may be combined with cytokines e.g. IL12 to enhance the anti-tumor efficacy of the CAR (Pegram H. J., et al. Tumor-targeted T cells modified to secrete IL12 eradicate systemic tumors without need for prior conditioning. Blood. 2012; 119:4133-41; the contents of each of which are incorporated herein by reference in their entirety). As another non-limiting example, Merchant et al. combined dendritic cell-based vaccinations with recombinant human IL7 to improve outcome in high-risk pediatric sarcomas patients (Merchant, M. S. et. al. Adjuvant immunotherapy to Improve Outcome in High-Risk Pediatric Sarcomas. Clin Cancer Res. 2016. 22(13):3182-91; the contents of each of which are incorporated herein by reference in their entirety).

In some embodiments, immune effector cells modified to express one or more antigen-specific TCRs or CARs may be combined with compositions of the invention comprising immunotherapeutic agents that convert the immunosuppressive tumor microenvironment.

In one aspect, effector immune cells modified to express CARs specific to different target molecules on the same cell may be combined. In another aspect, different immune cells modified to express the same CAR construct such as NK cells and T cells may be used in combination for a tumor treatment, for instance, a T cell modified to express a CD19 CAR may be combined with a NK cell modified to express the same CD19 CAR to treat B cell malignancy.

In other embodiments, immune cells modified to express CARs may be combined with checkpoint blockade agents.

In some embodiments, immune effector cells modified to expressed biocircuits, effector modules, DDs and payloads of the invention may be combined with cancer vaccines of the invention.

In some embodiments, methods of the invention may include combination of the compositions of the invention with other agents effective in the treatment of cancers, infection diseases and other immunodeficient disorders, such as anti-cancer agents. As used herein, the term “anti-cancer agent” refers to any agent which is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

In some embodiments, anti-cancer agent or therapy may be a chemotherapeutic agent, or radiotherapy, immunotherapeutic agent, surgery, or any other therapeutic agent which, in combination with the present invention, improves the therapeutic efficacy of treatment.

In one embodiment, an effector module comprising a CD19 CAR may be used in combination with amino pyrimidine derivatives such as the Burkit's tyrosine receptor kinase (BTK) inhibitor using methods taught in International Patent Application NO. WO2016164580, the contents of which are incorporated herein by reference in their entirety.

In some embodiments, compositions of the present invention may be used in combination with immunotherapeutics other than the inventive therapy described herein, such as antibodies specific to some target molecules on the surface of a tumor cell.

Radiotherapeutic agents and factors include radiation and waves that induce DNA damage for example, γ-irradiation, X-rays, UV-irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

In some embodiments, the chemotherapeutic agent may be an immunomodulatory agent such as lenalidomide (LEN). Recent studies have demonstrated that lenalidomide can enhance antitumor functions of CAR modified T cells (Otahal et al., Oncoimmunology, 2015, 5(4): e1115940). Some examples of anti-tumor antibodies include tocilizumab, siltuximab.

Other agents may be used in combination with compositions of the invention may also include, but not limited to, agents that affect the upregulation of cell surface receptors and their ligands such as Fas/Fas ligand, DR4 or DR5/TRAIL and GAP junctions, cytostatic and differentiation agents, inhibitors of cell adhesion such as focal adhesion kinase (FAKs) inhibitors and Lovastatin, or agents that increase the sensitivity of the hyper proliferative cells to apoptotic inducers such as the antibody C225.

The combinations may include administering the compositions of the invention and other agents at the same time or separately. Alternatively, the present immunotherapy may precede or follow the other agent/therapy by intervals ranging from minutes, days, weeks to months.

### 4. Diseases

Provided in the present invention is a method of reducing a tumor volume or burden in a subject in need, the method comprising introducing into the subject a composition of the invention.

The present invention also provides methods for treating a cancer in a subject, comprising administering to the subject an effective amount of an immune effector cell genetically modified to express at least one effector module of the invention.

### Cancer

Various cancers may be treated with pharmaceutical compositions, biocircuits, biocircuit components, effector modules including their SREs or payloads of the present invention. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

Types of carcinomas which may be treated with the compositions of the present invention include, but are not limited to, papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma.

Types of carcinomas which may be treated with the compositions of the present invention include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

As a non-limiting example, the carcinoma which may be treated may be Acute granulocytic leukemia, Acute lymphocytic leukemia, Acute myelogenous leukemia, Adenocarcinoma, Adenosarcoma, Adrenal cancer, Adrenocortical carcinoma, Anal cancer, Anaplastic astrocytoma, Angiosarcoma, Appendix cancer, Astrocytoma, Basal cell carcinoma, B-Cell lymphoma), Bile duct cancer, Bladder cancer, Bone cancer, Bowel cancer, Brain cancer, Brain stem glioma, Brain tumor, Breast cancer, Carcinoid tumors, Cervical cancer, Cholangiocarcinoma, Chondrosarcoma, Chronic lymphocytic leukemia, Chronic myelogenous leukemia, Colon cancer, Colorectal cancer, Craniopharyngioma, Cutaneous lymphoma, Cutaneous melanoma, Diffuse astrocytoma, Ductal carcinoma in situ, Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors, General, Germ cell tumor, Glioblastoma multiforme, Glioma, Hairy cell leukemia, Head and neck cancer, Hemangioendothelioma, Hodgkin lymphoma, Hodgkin's disease, Hodgkin's lymphoma, Hypopharyngeal cancer, Infiltrating ductal carcinoma, Infiltrating lobular carcinoma, Inflammatory breast cancer, Intestinal Cancer, Intrahepatic bile duct cancer, Invasive/infiltrating breast cancer, Islet cell cancer, Jaw cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Nasal cavity cancer, Nasopharyngeal cancer, Neck cancer, Neuroblastoma, Neuroendocrine tumors, Non-Hodgkin lymphoma, Non-Hodgkin's lymphoma, Non-small cell lung cancer, Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Paget's disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pineoblastoma, Pituitary gland cancer, Primary central nervous system lymphoma, Prostate cancer, Rectal cancer, Renal cell cancer, Renal pelvis cancer, Rhabdomyosarcoma, Salivary gland cancer, Sarcoma, Sarcoma, bone, Sarcoma, soft tissue, Sarcoma, uterine, Sinus cancer, Skin cancer, Small cell lung cancer, Small intestine cancer, Soft tissue sarcoma, Spinal cancer, Spinal column cancer, Spinal cord cancer, Spinal tumor, Squamous cell carcinoma, Stomach cancer, Synovial sarcoma, T-cell lymphoma), Testicular cancer, Throat cancer, Thymoma/thymic carcinoma, Thyroid cancer, Tongue cancer, Tonsil cancer, Transitional cell cancer, Transitional cell cancer, Transitional cell cancer, Triple-negative breast cancer, Tubal cancer, Tubular carcinoma, Ureteral cancer, Ureteral cancer, Urethral cancer, Uterine adenocarcinoma, Uterine cancer, Uterine sarcoma, Vaginal cancer, and Vulvar cancer.

The present invention also provides methods of reducing tumor burden in a subject. In some embodiments. As used herein, “tumor burden” refers to the number of cancer cells, or the amount of cancer in a subject. In some aspects tumor burden also refers to tumor load. In some embodiments, the tumor may be disseminated throughout the body of the subject. In one aspect, the tumor may be a liquid tumor such as leukemia or a lymphoma. The methods of reducing tumor burden may involve administering to the subject, a therapeutically effective amount of the immune cells. Immune cells may be engineered to express the compositions described herein. In some embodiments, the immune cells expressing the compositions of the invention may be administered to the subject via any of the routes of delivery described herein. Also provided herein are dosing regimens for administering the immune cells. In some embodiments, the subject may also be administered a therapeutically effective amount of the stimulus to tune the expression of the immunotherapeutic agent. In some aspects, the immunotherapeutic agents may be capable of reducing the tumor burden. Regimens for ligand/stimulus dosing are also provided. Reduction in tumor burden may be measured by any of the methods known in the art including tumor imaging, and measurement of marker proteins. In some aspects, bioluminescent imaging may be used to measure tumor burden. Bioluminescence imaging utilizes native light emission from bioluminescent proteins such as luciferase. Such bioluminescent proteins can participate in chemical reactions that release photons by the addition of suitable substrates. The release of photons can be captured by sensitive detection methods and quantified. Tumor cells may be engineered to express luciferase and the efficacy of the compositions described herein to reduce tumor burden may quantified by imaging. In some aspects, the tumor burden may be measured by the flux of photons (photons per sec). In some embodiments, photon flux positively correlates with tumor burden.

### Infectious Diseases

In some embodiment, biocircuits of the invention may be used for the treatment of infectious diseases. Biocircuits of the invention may be introduced in cells suitable for adoptive cell transfer such as macrophages, dendritic cells, natural killer cells, and or T cells. Infectious diseases treated by the biocircuits of the invention may be diseases caused by viruses, bacteria, fungi, and/or parasites. IL15-IL15Ra payloads of the invention may be used to increase immune cell proliferation and/or persistence of the immune cells useful in treating infectious diseases.

“Infection diseases” herein refer to diseases caused by any pathogen or agent that infects mammalian cells, preferably human cells and causes a disease condition. Examples thereof include bacteria, yeast, fungi, protozoans, mycoplasma, viruses, prions, and parasites. Examples include those involved in (a) viral diseases such as, for example, diseases resulting from infection by an adenovirus, a herpesvirus (e.g., HSV-I, HSV-II, CMV, or VZV), a poxvirus (e-g-, an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenza virus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV); (b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella; (c) other infectious diseases, such chlamydia, fungal diseases including but not limited to candidiasis, aspergillosis, histoplasmosis, cryptococcal meningitis, parasitic diseases including but not limited to malaria, Pneumocystis carnii pneumonia, leishmaniasis, cryptosporidiosis, toxoplasmosis, and trypanosome infection and prions that cause human disease such as Creutzfeldt-Jakob Disease (CJD), variant Creutzfeldt-Jakob Disease (vCJD), Gerstmann-Straüssler-Scheinker syndrome, Fatal Familial Insomnia and kuru.

### 5. Microbiome

Alterations in the composition of the microbiome may impact the action of anti-cancer therapies. A diverse community of symbiotic, commensal and pathogenic microorganisms exist in all environmentally exposed sites in the body and is herein referred to as the “Microbiome.” Environmentally exposed sites of the body that may be inhabited by a microbiome include the skin, nasopharynx, the oral cavity, respiratory tract, gastrointestinal tract, and the reproductive tract.

In some embodiments, microbiome native or engineered with immunotherapeutic agents may be used to improve the efficacy of the anti-cancer immunotherapies. Methods of using microbiome to improve responsive to immunotherapeutic agents have been described by Sivan et al (Sivan A., et al. Commensal Bifidobacterium promotes antitumor immunity and facilitates anti-PD-L1 efficacy. Science 2015; 350:1084-9; the contents of which are incorporated herein by reference in their entirety). In one embodiment, protein, RNA and/or other biomolecules derived from the microbiome may be used as a payload to influence the efficacy of the anti-cancer immunotherapies.

### 6. Tools and Agents for Making Therapeutics

Provided in the present invention are tools and agents that may be used in generating immunotherapeutics for reducing a tumor volume or burden in a subject in need. A considerable number of variables are involved in producing a therapeutic agent, such as structure of the payload, type of cells, method of gene transfers, method and time of ex vivo expansion, pre-conditioning and the amount and type of tumor burden in the subject. Such parameters may be optimized using tools and agents described herein.

### Cell Lines

The present disclosure provides a mammalian cell that has been genetically modified with the compositions of the invention. Suitable mammalian cells include primary cells and immortalized cell lines. Suitable mammalian cell lines include, but are not limited to Human embryonic kidney cell line 293, fibroblast cell line NIH 3T3, human colorectal carcinoma cell line HCT116, ovarian carcinoma cell line SKOV-3, immortalized T cell lines (e.g. Jurkat cells and SupT1 cells), lymphoma cell line Raji cells, NALM-6 cells, K562 cells, HeLa cells, PC12 cells, HL-60 cells, NK cell lines (e.g. NKL, NK92, NK962, and YTS), and the like. In some instances, the cell is not an immortalized cell line, but instead a cell obtained from an individual and is herein referred to as a primary cell. For example, the cell is a T lymphocyte obtained from an individual. Other examples include, but are not limited to cytotoxic cells, stem cells, peripheral blood mononuclear cells or progenitor cells obtained from an individual.

### Tracking SREs, Biocircuits and Cell Lines

In some embodiments, it may be desirable to track the compositions of the invention or the cells modified by the compositions of the invention. Tracking may be achieved by using reporter moieties, which, as used herein, refers to any protein capable of creating a detectable signal, in response to an input. Examples include alkaline phosphatase, β-galactosidase, chloramphenicol acetyltransferase, β-glucuronidase, peroxidase, β-lactamase, catalytic antibodies, bioluminescent proteins e.g. luciferase, and fluorescent proteins such as Green fluorescent protein (GFP).

Reporter moieties may be used to monitor the response of the DD upon addition of the ligand corresponding to the DD. In other instances, reporter moieties may be used to track cell survival, persistence, cell growth, and/or localization in vitro, in vivo, or ex vivo.

In some embodiments, the preferred reporter moiety may be luciferase proteins. In one embodiment, the reporter moiety is the Renilla luciferase (SEQ ID NO. 684 encoded by nucleic acid sequence of SEQ ID NO. 685), or a firefly luciferase (SEQ ID NO. 686, encoded by nucleic acid sequence of SEQ ID NO. 687).

In some embodiments, the preferred reporter moiety may be luciferase proteins. In one embodiment, the reporter moiety is the Renilla luciferase, or a firefly luciferase.

### Animal Models

The utility and efficacy of the compositions of the present invention may be tested in vivo animal models, preferably mouse models. Mouse models used to may be syngeneic mouse models wherein mouse cells are modified with compositions of the invention and tested in mice of the same genetic background. Examples include pMEL-1 and 4T1 mouse models. Alternatively, xenograft models where human cells such as tumor cells and immune cells are introduced into immunodeficient mice may also be utilized in such studies. Immunodeficient mice used may be CByJ.Cg-Foxnlnu/J, B6; 12957-Rag1tm1Mom/J, B6.12957-Rag1tm1Mom/J, B6. CB17-Prkdcscid/SzJ, NOD.12957(B6)-Rag1tm1Mom/J, NOD.Cg-Rag1tm1MomPrf1tm1Sdz/Sz, NOD.CB17-Prkdcscid/SzJ, NOD.Cg-PrkdcscidB2mtmlUnc/J, NOD-scid IL2Rgnull, Nude (nu) mice, SCID mice, NOD mice, RAG1/RAG2 mice, NOD-Scid mice, IL2rgnull mice, b2mnull mice, NOD-scid IL2r□null mice, NOD-scid-B2mnull mice, beige mouse, and HLA transgenic mice.

### 7. Cellular Assays

In some embodiments, the effectiveness of the compositions of the inventions as immunotherapeutic agents may be evaluated using cellular assays. Levels of expression and/or identity of the compositions of the invention may be determined according to any methods known in the art for identifying proteins and/or quantitating proteins levels. In some embodiments, such methods may include Western Blotting, flow cytometry, and immunoassays.

Provided herein are methods for functionally characterizing cells expressing SRE, biocircuits and compositions of the invention. In some embodiments, functional characterization is carried out in primary immune cells or immortalized immune cell lines and may be determined by expression of cell surface markers. Examples of cell surface markers for T cells include, but are not limited to, CD3, CD4, CD8, CD 14, CD20, CD11b, CD16, CD45 and HLA-DR, CD 69, CD28, CD44, IFNgamma. Markers for T cell exhaustion include PD1, TIM3, BTLA, CD160, 2B4, CD39, and LAG3. Examples of cell surface markers for antigen presenting cells include, but are not limited to, MHC class I, MHC Class II, CD40, CD45, B7-1, B7-2, IFN γ receptor and IL2 receptor, ICAM-1 and/or Fcγ receptor. Examples of cell surface markers for dendritic cells include, but are not limited to, MHC class I, MHC Class II, B7-2, CD18, CD29, CD31, CD43, CD44, CD45, CD54, CD58, CD83, CD86, CMRF-44, CMRF-56, DCIR and/or Dectin-1 and the like; while in some cases also having the absence of CD2, CD3, CD4, CD8, CD14, CD15, CD16, CD 19, CD20, CD56, and/or CD57. Examples of cell surface markers for NK cells include, but are not limited to, CCL3, CCL4, CCL5, CCR4, CXCR4, CXCR3, NKG2D, CD71, CD69, CCR5, Phospho JAK/STAT, phospho ERK, phospho p38/MAPK, phospho AKT, phospho STAT3, Granulysin, Granzyme B, Granzyme K, IL10, IL22, IFNg, LAP, Perforin, and TNFa.

### 8. Diagnostics

In some embodiments, scFvs, CARs and compositions of the invention may be used as diagnostics. In some cases, scFvs, CARs and/compositions of the invention may be used to identify, label or stain cells, tissues, organs, etc. expressing target antigens. In further embodiments, scFvs, CARs and/compositions of the invention may be used to identify CD19 antigen present in tissue sections (i.e., histological tissue sections), including tissue known or suspected of having cancerous cells. Such methods of using scFvs of the invention may in some cases be used to identify cancerous cells or tumors in tissue sections. Tissue sections may be from any tissue or organ including, but not limited to breast, colon, pancreatic, ovarian, brain, liver, kidney, spleen, lung, skin, stomach, intestine, esophagus, and bone. scFvs, CARs and/compositions of the present invention may also be used to identify blood samples suspected to have or known to be cancerous blood sample and distinguish it from the normal tissue.

Diagnostics described herein can be used to determine whether a subject should be treated with a wild type CD19 CAR therapy or a CAR that recognizes mutant CD19. In a particular embodiment, the method comprises determining whether the cancer cell expresses a wild-type CD19 and/or a CD19 isoform, wherein the presence of a CD19 isoform and/or absence of wild type CD19 indicates that the cancer will be refractory to a wild type CD19 CAR therapy. Methods of determining whether a cancer cell expresses wild-type CD19 or a CD19 isoform or variant are described herein and include, without limitation, sequencing (e.g., all or part (e.g., ectodomain) of CD 19), isoform specific PCR, isoform-specific oligonucleotide or probe screening methods, recognition by isoform specific antibodies, etc.

### 9. Stem Cell Applications

The biocircuits of the present invention and/or any of their components may be utilized in the regulated reprogramming of cells, stem cell engraftment or other application where controlled or tunable expression of such reprogramming factors are useful.

The biocircuits of the present invention may be used in reprogramming cells including stem cells or induced stem cells. Induction of induced pluripotent stem cells (iPSC) was first achieved by Takahashi and Yamanaka (Cell, 2006. 126(4):663-76; herein incorporated by reference in its entirety) using viral vectors to express KLF4, c-MYC, OCT4 and SOX2 otherwise collectively known as KMOS.

Excisable lentiviral and transposon vectors, repeated application of transient plasmid, episomal and adenovirus vectors have also been used to try to derive iPSC (Chang, C. W., et al., Stem Cells, 2009. 27(5):1042-1049; Kaji, K., et al., Nature, 2009. 458(7239):771-5; Okita, K., et al., Science, 2008. 322(5903):949-53; Stadtfeld, M., et al., Science, 2008. 322(5903):945-9; Woltjen, K., et al., Nature, 2009; Yu, J., et al., Science, 2009:1172482; Fusaki, N., et al., Proc Jpn Acad Ser B Phys Biol Sci, 2009. 85(8):348-62; each of which is herein incorporated by reference in its entirety).

DNA-free methods to generate human iPSC has also been derived using serial protein transduction with recombinant proteins incorporating cell-penetrating peptide moieties (Kim, D., et al., Cell Stem Cell, 2009. 4(6): 472-476; Zhou, H., et al., Cell Stem Cell, 2009. 4(5):381-4; each of which is herein incorporated by reference in its entirety), and infectious transgene delivery using the Sendai virus (Fusaki, N., et al., Proc Jpn Acad Ser B Phys Biol Sci, 2009. 85(8): p. 348-62; herein incorporated by reference in its entirety).

The effector modules of the present invention may include a payload comprising any of the genes including, but not limited to, OCT such as OCT4, SOX such as SOX1, SOX2, SOX3, SOX15 and SOX18, NANOG, KLF such as KLF1, KLF2, KLF4 and KLF5, MYC such as c-MYC and n-MYC, REM2, TERT and LIN28 and variants thereof in support of reprogramming cells. Sequences of such reprogramming factors are taught in for example International Application PCT/US2013/074560, the contents of which are incorporated herein by reference in their entirety.

In some embodiments, the payload of the present invention may be cardiac lineage specification factors such as eomesodermin (EOMES), a T-box transcription factor; WNT signaling pathway components such as WNT3 and WNT 3A. EOMES is crucially required for the development of the heart. Cardiomyocyte programming by EOMES involves autocrine activation of the canonical WNT signaling pathway and vice versa. Under conditions that are conducive to promoting cardiac lineage, WNT signaling activates EOMES and EOMES in turn promotes WNT signaling creating a self-sustaining loop that promotes the cardiac lineage. An activation loop that is too weak or too strong promotes non-cardiac fates such as endodermal and other mesodermal fates respectively. The DDs of the present invention may be used to tune EOMES and WNT payload levels to generate an activation loop that initiate and/or sustain cardiac specification during gastrulation.

### V. Delivery Modalities and/or Vectors

### Vectors

The present invention also provides vectors that package polynucleotides of the invention encoding biocircuits, effector modules, SREs (DDs) and payloads, and combinations thereof. Vectors of the present invention may also be used to deliver the packaged polynucleotides to a cell, a local tissue site or a subject. These vectors may be of any kind, including DNA vectors, RNA vectors, plasmids, viral vectors and particles. Viral vector technology is well known and described in Sambrook et al. (2001, Molecular Cloning: A Laboratory Manual, Cold Spring Harbor Laboratory, New York). Viruses, which are useful as vectors include, but are not limited to lentiviral vectors, adenoviral vectors, adeno-associated viral (AAV) vectors, herpes simplex viral vectors, retroviral vectors, oncolytic viruses, and the like.

In general, vectors contain an origin of replication functional in at least one organism, a promoter sequence and convenient restriction endonuclease site, and one or more selectable markers e.g. a drug resistance gene.

As used herein a promoter is defined as a DNA sequence recognized by transcription machinery of the cell, required to initiate specific transcription of the polynucleotide sequence of the present invention. Vectors can comprise native or non-native promoters operably linked to the polynucleotides of the invention. The promoters selected may be strong, weak, constitutive, inducible, tissue specific, development stage-specific, and/or organism specific. One example of a suitable promoter is the immediate early cytomegalovirus (CMV) promoter sequence. This promoter sequence is a strong constitutive promoter sequence capable of driving high levels of expression of polynucleotide sequence that is operatively linked to it. Another example of a preferred promoter is Elongation Growth Factor-1. Alpha (EF-1. alpha). Other constitutive promoters may also be used, including, but not limited to simian virus 40 (SV40), mouse mammary tumor virus (MMTV), human immunodeficiency virus (HIV), long terminal repeat (LTR), promoter, an avian leukemia virus promoter, an Epstein-Barr virus immediate early promoter, a Rous sarcoma virus promoter as well as human gene promoters including, but not limited to the phosphoglycerate kinase (PGK) promoter, actin promoter, the myosin promoter, the hemoglobin promoter, the Ubiquitin C (Ubc) promoter, the human U6 small nuclear protein promoter and the creatine kinase promoter. In some instances, inducible promoters such as but not limited to metallothionine promoter, glucocorticoid promoter, a progesterone promoter, and a tetracycline promoter may be used. In some embodiments, the promoter may be selected from the SEQ ID NO. 556-558.

In some embodiments, the optimal promoter may be selected based on its ability to achieve minimal expression of the SREs and payloads of the invention in the absence of the ligand and detectable expression in the presence of the ligand.

Additional promoter elements e.g. enhancers may be used to regulate the frequency of transcriptional initiation. Such regions may be located 10-100 base pairs upstream or downstream of the start site. In some instances, two or more promoter elements may be used to cooperatively or independently activate transcription.

In some embodiments, the recombinant expression vector may comprise regulatory sequences, such as transcription and translation initiation and termination codons, which are specific to the type of host cell into which the vector is to be introduced.

Promoter selection for expression of SREs in T cells is described in Example 24 of International Patent Publication, WO2018/161017, Example 14 of International Patent Publication, WO2018/160993; and Example 16 of International Patent Publication, WO2018/161026; the contents of each of which are incorporated by reference in their entirety. The effect of PGK Promoter and N-terminal FKBP is described in Example 42 of International Patent Publication, WO2018/161017 and Example 36 of International Patent Publication, WO2018/161038; the contents of each of which are incorporated by reference in their entirety.

### 1. Lentiviral Vectors

In some embodiments, lentiviral vectors/particles may be used as vehicles and delivery modalities. Lentiviruses are subgroup of the Retroviridae family of viruses, named because reverse transcription of viral RNA genomes to DNA is required before integration into the host genome. As such, the most important features of lentiviral vehicles/particles are the integration of their genetic material into the genome of a target/host cell. Some examples of lentivirus include the Human Immunodeficiency Viruses: HIV-1 and HIV-2, the Simian Immunodeficiency Virus (SIV), feline immunodeficiency virus (FIV), bovine immunodeficiency virus (BIV), Jembrana Disease Virus (JDV), equine infectious anemia virus (EIAV), equine infectious anemia virus, visna-maedi and caprine arthritis encephalitis virus (CAEV).

Typically, lentiviral particles making up the gene delivery vehicle are replication defective on their own (also referred to as “self-inactivating”). Lentiviruses are able to infect both dividing and non-dividing cells by virtue of the entry mechanism through the intact host nuclear envelope (Naldini L et al., Curr. Opin. Biotechnol, 1998, 9: 457-463). Recombinant lentiviral vehicles/particles have been generated by multiply attenuating the HIV virulence genes, for example, the genes Env, Vif, Vpr, Vpu, Nef and Tat are deleted making the vector biologically safe. Correspondingly, lentiviral vehicles, for example, derived from HIV-1/HIV-2 can mediate the efficient delivery, integration and long-term expression of transgenes into non-dividing cells. As used herein, the term “recombinant” refers to a vector or other nucleic acid containing both lentiviral sequences and non-lentiviral retroviral sequences.

Lentiviral particles may be generated by co-expressing the virus packaging elements and the vector genome itself in a producer cell such as human HEK293T cells. These elements are usually provided in three (in second generation lentiviral systems) or four separate plasmids (in third generation lentiviral systems). The producer cells are co-transfected with plasmids that encode lentiviral components including the core (i.e. structural proteins) and enzymatic components of the virus, and the envelope protein(s) (referred to as the packaging systems), and a plasmid that encodes the genome including a foreign transgene, to be transferred to the target cell, the vehicle itself (also referred to as the transfer vector). In general, the plasmids or vectors are included in a producer cell line. The plasmids/vectors are introduced via transfection, transduction or infection into the producer cell line. Methods for transfection, transduction or infection are well known by those of skill in the art. As non-limiting example, the packaging and transfer constructs can be introduced into producer cell lines by calcium phosphate transfection, lipofection or electroporation, generally together with a dominant selectable marker, such as neo, DHFR, Gln synthetase or ADA, followed by selection in the presence of the appropriate drug and isolation of clones.

The producer cell produces recombinant viral particles that contain the foreign gene, for example, the effector module of the present invention. The recombinant viral particles are recovered from the culture media and titrated by standard methods used by those of skill in the art. The recombinant lentiviral vehicles can be used to infect target cells.

Cells that can be used to produce high-titer lentiviral particles may include, but are not limited to, HEK293T cells, 293G cells, STAR cells (Relander et al., Mol. Ther 2005, 11: 452-459), FreeStyle™ 293 Expression System (ThermoFisher, Waltham, Mass.), and other HEK293T-based producer cell lines (e.g., Stewart et al., Hum Gene Ther. 2011, 22 (3):357-369; Lee et al., Biotechnol Bioeng, 2012, 10996): 1551-1560; Throm et al., Blood. 2009, 113(21): 5104-5110; the contents of each of which are incorporated herein by reference in their entirety).

In some aspects, the envelope proteins may be heterologous envelop proteins from other viruses, such as the G protein of vesicular stomatitis virus (VSV G) or baculoviral gp64 envelop proteins. The VSV-G glycoprotein may especially be chosen among species classified in the vesiculovirus genus: Carajas virus (CJSV), Chandipura virus (CHPV), Cocal virus (COCV), Isfahan virus (ISFV), Maraba virus (MARAV), Piry virus (PIRYV), Vesicular stomatitis Alagoas virus (VSAV), Vesicular stomatitis Indiana virus (VSIV) and Vesicular stomatitis New Jersey virus (VSNJV) and/or stains provisionally classified in the vesiculovirus genus as Grass carp rhabdovirus, BeAn 157575 virus (BeAn 157575), Boteke virus (BTKV), Calchaqui virus (CQIV), Eel virus American (EVA), Gray Lodge virus (GLOV), Jurona virus (JURY), Klamath virus (KLAV), Kwatta virus (KWAV), La Joya virus (LJV), Malpais Spring virus (MSPV), Mount Elgon bat virus (MEBV), Perinet virus (PERV), Pike fry rhabdovirus (PFRV), Porton virus (PORV), Radi virus (RADIV), Spring viremia of carp virus (SVCV), Tupaia virus (TUPV), Ulcerative disease rhabdovirus (UDRV) and Yug Bogdanovac virus (YBV). The gp64 or other baculoviral env protein can be derived from Autographa californica nucleopolyhedrovirus (AcMNPV), Anagrapha falcifera nuclear polyhedrosis virus, Bombyx mori nuclear polyhedrosis virus, Choristoneura fumiferana nucleopolyhedrovirus, Orgyia pseudotsugata single capsid nuclear polyhedrosis virus, Epiphyas postvittana nucleopolyhedrovirus, Hyphantria cunea nucleopolyhedrovirus, Galleria mellonella nuclear polyhedrosis virus, Dhori virus, Thogoto virus, Antheraea pemyi nucleopolyhedrovirus or Batken virus.

Additional elements provided in lentiviral particles may comprise retroviral LTR (long-terminal repeat) at either 5′ or 3′ terminus, a retroviral export element, optionally a lentiviral reverse response element (RRE), a promoter or active portion thereof, and a locus control region (LCR) or active portion thereof. Other elements include central polypurine tract (cPPT) sequence to improve transduction efficiency in non-dividing cells, Woodchuck Hepatitis Virus (WHP) Posttranscriptional Regulatory Element (WPRE) which enhances the expression of the transgene and increases titer. The effector module is linked to the vector.

Methods for generating recombinant lentiviral particles are discussed in the art, for example, U.S. Pat. Nos. 8,846,385; 7,745,179; 7,629,153; 7,575,924; 7,179, 903; and 6,808,905; the contents of each of which are incorporated herein by reference in their entirety.

Lentivirus vectors used may be selected from, but are not limited to pLVX, pLenti, pLenti6, pLJM1, FUGW, pWPXL, pWPI, pLenti CMV puro DEST, pLJM1-EGFP, pULTRA, pInducer20, pHIV-EGFP, pCW57.1, pTRPE, pELPS, pRRL, and pLionll.

Lentiviral vehicles known in the art may also be used (See, U.S. Pat. Nos. 9,260,725; 9,068,199; 9,023,646; 8,900,858; 8,748,169; 8,709,799; 8,420,104; 8,329,462; 8,076,106; 6,013,516; and 5,994,136; International Patent Publication NO. WO2012079000; the contents of each of which are incorporated herein by reference in their entirety).

### 2. Retroviral Vectors (γ-Retroviral Vectors)

In some embodiments, retroviral vectors may be used to package and deliver the biocircuits, biocircuit components, effector modules, SREs or payloads of the present invention. Retroviral vectors (RVs) allow the permanent integration of a transgene in target cells. In addition to lentiviral vectors based on complex HIV-1/2, retroviral vectors based on simple gamma-retroviruses have been widely used to deliver therapeutic genes and demonstrated clinically as one of the most efficient and powerful gene delivery systems capable of transducing a broad range of cell types. Example species of Gamma retroviruses include the murine leukemia viruses (MLVs) and the feline leukemia viruses (FeLV).

In some embodiments, gamma-retroviral vectors derived from a mammalian gamma-retrovirus such as murine leukemia viruses (MLVs), are recombinant. The MLV families of gamma retroviruses include the ecotropic, amphotropic, xenotropic and polytropic subfamilies. Ecotropic viruses are able to infect only murine cells using mCAT-1 receptor. Examples of ecotropic viruses are Moloney MLV and AKV. Amphotropic viruses infect murine, human and other species through the Pit-2 receptor. One example of an amphotropic virus is the 4070A virus. Xenotropic and polytropic viruses utilize the same (Xpr1) receptor but differ in their species tropism. Xenotropic viruses such as NZB-9-1 infect human and other species but not murine species, whereas polytropic viruses such as focus-forming viruses (MCF) infect murine, human and other species.

Gamma-retroviral vectors may be produced in packaging cells by co-transfecting the cells with several plasmids including one encoding the retroviral structural and enzymatic (gag-pol) polyprotein, one encoding the envelope (env) protein, and one encoding the vector mRNA comprising polynucleotide encoding the compositions of the present invention that is to be packaged in newly formed viral particles.

In some aspects, the recombinant gamma-retroviral vectors are pseudotyped with envelope proteins from other viruses. Envelope glycoproteins are incorporated in the outer lipid layer of the viral particles which can increase/alter the cell tropism. Exemplary envelop proteins include the gibbon ape leukemia virus envelope protein (GALV) or vesicular stomatitis virus G protein (VSV-G), or Simian endogenous retrovirus envelop protein, or Measles Virus H and F proteins, or Human immunodeficiency virus gp120 envelope protein, or cocal vesiculovirus envelop protein (See, e.g., U.S. application publication NO. 2012/164118; the contents of which are incorporated herein by reference in its entirety). In other aspects, envelope glycoproteins may be genetically modified to incorporate targeting/binding ligands into gamma-retroviral vectors, binding ligands including, but not limited to, peptide ligands, single chain antibodies and growth factors (Waehler et al., Nat. Rev. Genet. 2007, 8(8):573-587; the contents of which are incorporated herein by reference in its entirety). These engineered glycoproteins can retarget vectors to cells expressing their corresponding target moieties. In other aspects, a “molecular bridge” may be introduced to direct vectors to specific cells. The molecular bridge has dual specificities: one end can recognize viral glycoproteins, and the other end can bind to the molecular determinant on the target cell. Such molecular bridges, for example ligand-receptor, avidin-biotin, and chemical conjugations, monoclonal antibodies and engineered fusogenic proteins, can direct the attachment of viral vectors to target cells for transduction (Yang et al., Biotechnol. Bioeng., 2008, 101(2): 357-368; and Maetzig et al., Viruses, 2011, 3, 677-713; the contents of each of which are incorporated herein by reference in their entirety).

In some embodiments, the recombinant gamma-retroviral vectors are self-inactivating (SIN) gammaretroviral vectors. The vectors are replication incompetent. SIN vectors may harbor a deletion within the 3′ U3 region initially comprising enhancer/promoter activity. Furthermore, the 5′ U3 region may be replaced with strong promoters (needed in the packaging cell line) derived from Cytomegalovirus or RSV, or an internal promoter of choice, and/or an enhancer element. The choice of the internal promoters may be made according to specific requirements of gene expression needed for a particular purpose of the invention.

In some embodiments, polynucleotides encoding the biocircuit, biocircuit components, effector module, SRE are inserted within the recombinant viral genome. The other components of the viral mRNA of a recombinant gamma-retroviral vector may be modified by insertion or removal of naturally occurring sequences (e.g., insertion of an IRES, insertion of a heterologous polynucleotide encoding a polypeptide or inhibitory nucleic acid of interest, shuffling of a more effective promoter from a different retrovirus or virus in place of the wild-type promoter and the like). In some examples, the recombinant gamma-retroviral vectors may comprise modified packaging signal, and/or primer binding site (PBS), and/or 5′-enhancer/promoter elements in the U3-region of the 5′-long terminal repeat (LTR), and/or 3′-SIN elements modified in the U3-region of the 3′-LTR. These modifications may increase the titers and the ability of infection.

Gamma retroviral vectors suitable for delivering biocircuit components, effector modules, SREs or payloads of the present invention may be selected from those disclosed in U.S. Pat. Nos. 8,828,718; 7,585,676; 7,351,585; U.S. application publication NO. 2007/048285; PCT application publication NOs. WO2010/113037; WO2014/121005; WO2015/056014; and EP Pat. NOs. EP1757702; EP1757703 (the contents of each of which are incorporated herein by reference in their entirety).

### 3. Adeno-Associated Viral Vectors (AAV)

In some embodiments, polynucleotides of present invention may be packaged into recombinant adeno-associated viral (rAAV) vectors. Such vectors or viral particles may be designed to utilize any of the known serotype capsids or combinations of serotype capsids. The serotype capsids may include capsids from any identified AAV serotypes and variants thereof, for example, AAV1, AAV2, AAV2G9, AAV3, AAV4, AAV4-4, AAV5, AAV6, AAV7, AAV8, AAV9, AAV10, AAV11, AAV12 and AAVrh10.

In one embodiment, the AAV serotype may have a sequence as described in Pulicherla et al. (Molecular Therapy, 2011, 19(6):1070-1078), U.S. Pat. Nos. 6,156,303; 7,198,951; U.S. Patent Publication NOs. US2015/0159173 and US2014/0359799; and International Patent Publication NOs. WO1998/011244, WO2005/033321 and WO2014/14422; the contents of each of which are incorporated herein by reference in their entirety.

AAV vectors include not only single stranded vectors but self-complementary AAV vectors (scAAVs). scAAV vectors contain DNA which anneals together to form double stranded vector genome. By skipping second strand synthesis, scAAVs allow for rapid expression in the cell.

The rAAV vectors may be manufactured by standard methods in the art such as by triple transfection, in sf9 insect cells or in suspension cell cultures of human cells such as HEK293 cells.

The biocircuits, biocircuit components, effector modules, SREs or payloads may be encoded in one or more viral genomes to be packaged in the AAV capsids taught herein.

Such vectors or viral genomes may also include, in addition to at least one or two ITRs (inverted terminal repeats), certain regulatory elements necessary for expression from the vector or viral genome. Such regulatory elements are well known in the art and include for example promoters, introns, spacers, stuffer sequences, and the like.

In some embodiments, more than one effector module or SRE (e.g. DD) may be encoded in a viral genome.

### 4. Oncolytic Viral Vector

In some embodiments, polynucleotides of present invention may be packaged into oncolytic viruses, such as vaccine viruses. Oncolytic vaccine viruses may include viral particles of a thymidine kinase (TK)-deficient, granulocyte macrophage (GM)-colony stimulating factor (CSF)-expressing, replication-competent vaccinia virus vector sufficient to induce oncolysis of cells in the tumor (e.g., U.S. Pat. No. 9,226,977).

5. Messenger RNA (mRNA)

In some embodiments, the effector modules of the invention may be designed as a messenger RNA (mRNA). As used herein, the term “messenger RNA” (mRNA) refers to any polynucleotide which encodes a polypeptide of interest and which is capable of being translated to produce the encoded polypeptide of interest in vitro, in vivo, in situ or ex vivo.

In some embodiments, the effector modules may be designed as self-amplifying RNA. “Self-amplifying RNA” as used herein refers to RNA molecules that can replicate in the host resulting in the increase in the amount of the RNA and the protein encoded by the RNA. Such self-amplifying RNA may have structural features or components of any of those taught in International Patent Application Publication No. WO2011005799 (the contents of which are incorporated herein by reference in their entirety).

### VI. Dosing, Delivery and Administrations

The compositions of the invention may be delivered to a cell or a subject through one or more routes and modalities. The viral vectors containing one or more effector modules, SREs, immunotherapeutic agents and other components described herein may be used to deliver them to a cell and/or a subject. Other modalities may also be used such as mRNAs, plasmids, and as recombinant proteins.

### 1. Delivery to Cells

In another aspect of the invention, polynucleotides encoding biocircuits, effector modules, SREs (e.g., DDs), payloads of interest (immunotherapeutic agents) and compositions of the invention and vectors comprising said polynucleotides may be introduced into cells such as immune effector cells.

In one aspect of the invention, polynucleotides encoding biocircuits, effector modules, SREs (e.g., DDs), payloads of interest (immunotherapeutic agents) and compositions of the invention, may be packaged into viral vectors or integrated into viral genomes allowing transient or stable expression of the polynucleotides. Preferable viral vectors are retroviral vectors including lentiviral vectors. In order to construct a retroviral vector, a polynucleotide molecule encoding a biocircuit, an effector module, a DD or a payload of interest (i.e. an immunotherapeutic agent) is inserted into the viral genome in the place of certain viral sequences to produce a virus that is replication-defective. The recombinant viral vector is then introduced into a packaging cell line containing the gag, pol, and env genes, but without the LTR and packaging components. The recombinant retroviral particles are secreted into the culture media, then collected, optionally concentrated, and used for gene transfer. Lentiviral vectors are especially preferred as they are capable of infecting both dividing and non-dividing cells.

Vectors may also be transferred to cells by non-viral methods by physical methods such as needles, electroporation, sonoporation, hyrdoporation; chemical carriers such as inorganic particles (e.g. calcium phosphate, silica, gold) and/or chemical methods. In some embodiments, synthetic or natural biodegradable agents may be used for delivery such as cationic lipids, lipid nano emulsions, nanoparticles, peptide-based vectors, or polymer-based vectors.

In some embodiments, the polypeptides of the invention may be delivered to the cell directly. In one embodiment, the polypeptides of the invention may be delivered using synthetic peptides comprising an endosomal leakage domain (ELD) fused to a cell penetration domain (CLD). The polypeptides of the invention are co introduced into the cell with the ELD-CLD-synthetic peptide. ELDs facilitate the escape of proteins that are trapped in the endosome, into the cytosol. Such domains are derived proteins of microbial and viral origin and have been described in the art. CPDs allow the transport of proteins across the plasma membrane and have also been described in the art. The ELD-CLD fusion proteins synergistically increase the transduction efficiency when compared to the co-transduction with either domain alone. In some embodiments, a histidine rich domain may optionally be added to the shuttle construct as an additional method of allowing the escape of the cargo from the endosome into the cytosol. The shuttle may also include a cysteine residue at the N or C terminus to generate multimers of the fusion peptide. Multimers of the ELD-CLD fusion peptides generated by the addition of cysteine residue to the terminus of the peptide show even greater transduction efficiency when compared to the single fusion peptide constructs. The polypeptides of the invention may also be appended to appropriate localization signals to direct the cargo to the appropriate sub-cellular location e.g. nucleus. In some embodiments any of the ELDs, CLDs or the fusion ELD-CLD synthetic peptides taught in the International Patent Publication, WO2016161516 and WO2017175072 may be useful in the present invention (the contents of each of which are herein incorporated by reference in their entirety).

### 2. Dosing

The present invention provides methods comprising administering any one or more compositions for immunotherapy to a subject in need thereof. These may be administered to a subject using any amount and any route of administration effective for preventing or treating a clinical condition such as cancer, infection diseases and other immunodeficient diseases.

Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically effective, or prophylactically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, previous or concurrent therapeutic interventions and rate of excretion of the specific compound employed; the duration of the treatment; drugs used in combination or coincidental with the specific compound employed; and like factors well known in the medical arts.

Compositions of the invention may be used in varying doses to avoid T cell energy, prevent cytokine release syndrome and minimize toxicity associated with immunotherapy. For example, low doses of the compositions of the present invention may be used to initially treat patients with high tumor burden, while patients with low tumor burden may be treated with high and repeated doses of the compositions of the invention to ensure recognition of a minimal tumor antigen load. In another instance, the compositions of the present invention may be delivered in a pulsatile fashion to reduce tonic T cell signaling and enhance persistence in vivo. In some aspects, toxicity may be minimized by initially using low doses of the compositions of the invention, prior to administering high doses. Dosing may be modified if serum markers such as ferritin, serum C-reactive protein, IL6, IFN-γ, and TNF-α are elevated.

In some embodiments, the neurotoxicity may be associated with CAR or TIL therapy. Such neurotoxicity may be associated CD19-CARs. Toxicity may be due to excessive T cell infiltration into the brain. In some embodiments, neurotoxicity may be alleviated by preventing the passage of T cells through the blood brain barrier. This can be achieved by the targeted gene deletion of the endogenous alpha-4 integrin inhibitors such as tysabri/natalizumab may also be useful in the present invention.

Also provided herein are methods of administering ligands in accordance with the invention to a subject in need thereof. The ligand may be administered to a subject or to cells, using any amount and any route of administration effective for tuning the biocircuits of the invention. The exact amount required will vary from subject to subject, depending on the species, age, and general condition of the subject, the severity of the disease, the particular composition, its mode of administration, its mode of activity, and the like. The subject may be a human, a mammal, or an animal. Compositions in accordance with the invention are typically formulated in unit dosage form for ease of administration and uniformity of dosage. It will be understood, however, that the total daily usage of the compositions of the present invention may be decided by the attending physician within the scope of sound medical judgment. In certain embodiments, the ligands in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, from about 10 mg/kg to about 100 mg/kg, from about 50 mg/kg to about 500 mg/kg, from about 100 mg/kg to about 1000 mg/kg, of subject body weight per day, one or more times a day, to obtain the desired effect. In some embodiments, the dosage levels may be 1 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg or mg/kg of subject body weight per day, or more times a day, to obtain the desired effect.

The present disclosure provides methods for delivering to a cell or tissue any of the ligands described herein, comprising contacting the cell or tissue with said ligand and can be accomplished in vitro, ex vivo, or in vivo. In certain embodiments, the ligands in accordance with the present invention may be administered to cells at dosage levels sufficient to deliver from about 1 nM to about 10 nM, from about 5 nM to about 50 nM, from about 10 nM to about 100 nM, from about 50 nM to about 500 nM, from about 100 nM to about 1000 nM, from about 1 μM to about 10 μM, from about 5 μM to about 50 μM from about 10 μM to about 100 μM from about 25 μM to about 250 μM from about 50 μM to about 500 μM. In some embodiments, the ligand may be administered to cells at doses selected from but not limited to 0.00064 μM, 0.0032 μM, 0.016 μM, 0.08 μM, 0.4 μM, 1 μM 2 μM, 10 μM, 50 μM, 75, μM, 100 μM, 150 μM, 175 μM, 200 μM, 250 μM.

The desired dosage of the ligands of the present invention may be delivered only once, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of “single unit dose” or total daily dose into two or more doses, e.g., two or more administrations of the “single unit dose”. As used herein, a “single unit dose” is a dose of any therapeutic administered in one dose/at one time/single route/single point of contact, i.e., single administration event. The desired dosage of the ligand of the present invention may be administered as a “pulse dose” or as a “continuous flow”. As used herein, a “pulse dose” is a series of single unit doses of any therapeutic administered with a set frequency over a period of time. As used herein, a “continuous flow” is a dose of therapeutic administered continuously for a period of time in a single route/single point of contact, i.e., continuous administration event. A total daily dose, an amount given or prescribed in 24-hour period, may be administered by any of these methods, or as a combination of these methods, or by any other methods suitable for a pharmaceutical administration.

### 3. Administration

In some embodiments, the compositions for immunotherapy may be administered to cells ex vivo and subsequently administered to the subject. Immune cells can be isolated and expanded ex vivo using a variety of methods known in the art. For example, methods of isolating cytotoxic T cells are described in U.S. Pat. Nos. 6,805,861 and 6,531,451; the contents of each of which are incorporated herein by reference in their entirety. Isolation of NK cells is described in U.S. Pat. No. 7,435,596; the contents of which are incorporated by reference herein in its entirety.

In some embodiments, compositions of the present invention, may be administered by any of the methods of administration taught in the copending commonly owned U.S. Provisional Patent Application No. 62/320,864 filed on Apr. 11, 2016, or in U.S. Provisional Application No. 62/466,596 filed Mar. 3, 2017 and the International Publication WO2017/180587, the contents of each of which are incorporated herein by reference in their entirety.

In some embodiments, depending upon the nature of the cells, the cells may be introduced into a host organism e.g. a mammal, in a wide variety of ways including by injection, transfusion, infusion, local instillation or implantation. In some aspects, the cells of the invention may be introduced at the site of the tumor. The number of cells that are employed will depend upon a number of circumstances, the purpose for the introduction, the lifetime of the cells, the protocol to be used, for example, the number of administrations, the ability of the cells to multiply, or the like. The cells may be in a physiologically-acceptable medium.

In some embodiments, the cells of the invention may be administrated in multiple doses to subjects having a disease or condition. The administrations generally effect an improvement in one or more symptoms of cancer or a clinical condition and/or treat or prevent cancer or clinical condition or symptom thereof.

In some embodiments, the compositions for immunotherapy may be administered in vivo. In some embodiments, polypeptides of the present invention comprising biocircuits, effector molecules, SREs, payloads of interest (immunotherapeutic agents) and compositions of the invention may be delivered in vivo to the subject. In vivo delivery of immunotherapeutic agents is well described in the art. For example, methods of delivery of cytokines are described in the E.P. Pat. NO. EP0930892 A1, the contents of which are incorporated herein by reference.

In one embodiment, the payloads of the present invention may be administered in conjunction with inhibitors of SHP-1 and/or SHP-2. The tyrosine-protein phosphatase SHP1 (also known as PTPN6) and SHP2 (also known as PTPN11) are involved in the Programmed Cell Death (PD1) inhibitory signaling pathway. The intracellular domain of PD1 contains an immunoreceptor tyrosine-based inhibitory motif (ITIM) and an immunoreceptor tyrosine-based switch motif (ITSM). ITSM has been shown to recruit SHP-1 and 2. This generates negative costimulatory micro clusters that induce the dephosphorylation of the proximal TCR signaling molecules, thereby resulting in suppression of T cell activation, which can lead to T cell exhaustion. In one embodiment, inhibitors of SHP-1 and 2 may include expressing dominant negative versions of the proteins in T cells, TILs or other cell types to relieve exhaustion. Such mutants can bind to the endogenous, catalytically active proteins, and inhibit their function. In one embodiment, the dominant negative mutant of SHP-1 and/or SHP-2 lack the phosphatase domain required for catalytic activity. In some embodiments, any of the dominant negative SHP-1 mutants taught Bergeron S et al. (2011). Endocrinology. 2011 December; 152(12):4581-8.; Dustin J B et al. (1999) J Immunol. March 1; 162(5):2717-24.; Berchtold S (1998) Mol Endocrinol. April; 12(4):556-67 and Schram et al. (2012) Am J Physiol Heart Circ Physiol. 1; 302(1): H231-43.; may be useful in the invention (the contents of each of which are incorporated by reference in their entirety).

### Routes of Delivery

The pharmaceutical compositions, biocircuits, biocircuit components, effector modules including their SREs (e.g., DDs), payloads (i.e. immunotherapeutic agents), vectors and cells of the present invention may be administered by any route to achieve a therapeutically effective outcome.

These include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intra-arterial (into an artery), intramuscular (into a muscle), intracranial (into the heart), intraosseous infusion (into the bone marrow), intrathecal (into the spinal canal), intraperitoneal, (infusion or injection into the peritoneum), intrasinal infusion, intravitreal, (through the eye), intravenous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

### VII. Definitions

At various places in the present specification, features or functions of the compositions of the present disclosure are disclosed in groups or in ranges. It is specifically intended that the present disclosure include each and every individual sub combination of the members of such groups and ranges. The following is a non-limiting list of term definitions.

Activity: As used herein, the term “activity” refers to the condition in which things are happening or being done. Compositions of the invention may have activity and this activity may involve one or more biological events. In some embodiments, biological events may include cell signaling events. In some embodiments, biological events may include cell signaling events associated protein interactions with one or more corresponding proteins, receptors, small molecules or any of the biocircuit components described herein.

Adoptive cell therapy (ACT): The terms “Adoptive cell therapy” or “Adoptive cell transfer”, as used herein, refer to a cell therapy involving in the transfer of cells into a patient, wherein cells may have originated from the patient, or from another individual, and are engineered (altered) before being transferred back into the patient. The therapeutic cells may be derived from the immune system, such as Immune effector cells: CD4+ T cell; CD8+ T cell, Natural Killer cell (NK cell); and B cells and tumor infiltrating lymphocytes (TILs) derived from the resected tumors. Most commonly transferred cells are autologous anti-tumor T cells after ex vivo expansion or manipulation. For example, autologous peripheral blood lymphocytes can be genetically engineered to recognize specific tumor antigens by expressing T-cell receptors (TCR) or chimeric antigen receptor (CAR).

Agent: As used herein, the term “agent” refers to a biological, pharmaceutical, or chemical compound. Non-limiting examples include simple or complex organic or inorganic molecule, a peptide, a protein, an oligonucleotide, an antibody, an antibody derivative, antibody fragment, a receptor, and soluble factor.

Agonist: the term “agonist” as used herein, refers to a compound that, in combination with a receptor, can produce a cellular response. An agonist may be a ligand that directly binds to the receptor. Alternatively, an agonist may combine with a receptor indirectly by, for example, (a) forming a complex with another molecule that directly binds to the receptor, or (b) otherwise resulting in the modification of another compound so that the other compound directly binds to the receptor. An agonist may be referred to as an agonist of a particular receptor or family of receptors, e.g., agonist of a co-stimulatory receptor.

Antagonist: the term “antagonist” as used herein refers to any agent that inhibits or reduces the biological activity of the target(s) it binds.

Antigen: the term “antigen” as used herein is defined as a molecule that provokes an immune response when it is introduced into a subject or produced by a subject such as tumor antigens which arise by the cancer development itself. This immune response may involve either antibody production, or the activation of specific immunologically-competent cells such as cytotoxic T lymphocytes and T helper cells, or both. An antigen can be derived from organisms, subunits of proteins/antigens, killed or inactivated whole cells or lysates. In the context of the invention, the terms “antigens of interest” or “desired antigens” refers to those proteins and/or other biomolecules provided herein that are immunospecifically bound or interact with antibodies of the present invention and/or fragments, mutants, variants, and/or alterations thereof described herein. In some embodiments, antigens of interest may comprise any of the polypeptides or payloads or proteins described herein, or fragments or portions thereof.

Approximately: As used herein, the term “approximately” or “about,” as applied to one or more values of interest, refers to a value that is similar to a stated reference value. In certain embodiments, the term “approximately” or “about” refers to a range of values that fall within 25, 20, 19, 18, 17, 16, 15, 14, 13, 12, 11, 10, 9, 8, 7, 6, 5, 4, 3, 2, 1, or less in either direction (greater than or less than) of the stated reference value unless otherwise stated or otherwise evident from the context (except where such number would exceed 100 of a possible value).

Associated with: As used herein, the terms “associated with,” “conjugated,” “linked,” “attached,” and “tethered,” when used with respect to two or more moieties, mean that the moieties are physically associated or connected with one another, either directly or via one or more additional moieties that serve as linking agents, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Autologous: the term “autologous” as used herein is meant to refer to any material derived from the same individual to which it is later to be re-introduced into the individual.

Barcode: the term “barcode” as used herein refers to polynucleotide or amino acid sequence that distinguishes one polynucleotide or amino acid from another.

Cancer: the term “cancer” as used herein refers a broad group of various diseases characterized by the uncontrolled growth of abnormal cells in the body. Unregulated cell division and growth results in the formation of malignant tumors that invade neighboring tissues ultimately metastasize to distant parts of the body through the lymphatic system or bloodstream.

Composition: As used herein, the term “composition” refers to a biological compound, a pharmaceutical compound, an immunotherapeutic agent, SRE, an effector molecule, or chemical compound. Non-limiting examples include simple or complex organic or inorganic molecule, a peptide, a protein, an oligonucleotide, a polynucleotide, an antibody, an antibody derivative, antibody fragment, a receptor, a soluble factor, biocircuit systems, effector modules, stimulus response elements (SREs) and immunotherapeutic agents, polynucleotides encoding the same, vectors and cells (e.g. T-cells) containing the polypeptides and/or polynucleotides.

Co-stimulatory molecule: As used herein, in accordance with its meaning in immune T cell activation, refers to a group of immune cell surface receptor/ligands which engage between T cells and APCs and generate a stimulatory signal in T cells which combines with the stimulatory signal in T cells that results from T cell receptor (TCR) recognition of antigen/MHC complex (pMHC) on APCs

Cytokines: the term “cytokines”, as used herein, refers to a family of small soluble factors with pleiotropic functions that are produced by many cell types that can influence and regulate the function of the immune system.

Delivery: the term “delivery” as used herein refers to the act or manner of delivering a compound, substance, entity, moiety, cargo or payload. A “delivery agent” refers to any agent which facilitates, at least in part, the in vivo delivery of one or more substances (including, but not limited to a compound and/or compositions of the present invention) to a cell, subject or other biological system cells.

Destabilized: As used herein, the term “destable,” “destabilize,” “destabilizing region” or “destabilizing domain” means a region or molecule that is less stable than a starting, reference, wild-type or native form of the same region or molecule.

Engineered: As used herein, embodiments of the invention are “engineered” when they are designed to have a feature or property, whether structural or chemical, that varies from a starting point, wild type or native molecule.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

Feature: As used herein, a “feature” refers to a characteristic, a property, or a distinctive element.

Formulation: As used herein, a “formulation” includes at least a compound and/or composition of the present invention and a delivery agent.

Fragment: A “fragment,” as used herein, refers to a portion. For example, fragments of proteins may comprise polypeptides obtained by digesting full-length protein. In some embodiments, a fragment of a protein includes at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250 or more amino acids. In some embodiments, fragments of an antibody include portions of an antibody.

Functional: As used herein, a “functional” biological molecule is a biological entity with a structure and in a form in which it exhibits a property and/or activity by which it is characterized.

Immune cells: the term “an immune cell”, as used herein, refers to any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells and natural killer (NK) cells). Exemplary immune system cells include a CD4+ T cell, a CD8+ T cell, a CD4− CD8− double negative T cell, a Tγδ cell, a Tαβ cell, a regulatory T cell, a natural killer cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

Immunotherapy: the term “immunotherapy” as used herein, refers to a type of treatment of a disease by the induction or restoration of the reactivity of the immune system towards the disease.

Immunotherapeutic agent: the term “immunotherapeutic agent” as used herein, refers to the treatment of disease by the induction or restoration of the reactivity of the immune system towards the disease with a biological, pharmaceutical, or chemical compound.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Linker: As used herein, a linker refers to a moiety that connects two or more domains, moieties or entities. In one embodiment, a linker may comprise 10 or more atoms. In a further embodiment, a linker may comprise a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. In some embodiments, a linker may comprise one or more nucleic acids comprising one or more nucleotides. In some embodiments, the linker may comprise an amino acid, peptide, polypeptide or protein. In some embodiments, a moiety bound by a linker may include, but is not limited to an atom, a chemical group, a nucleoside, a nucleotide, a nucleobase, a sugar, a nucleic acid, an amino acid, a peptide, a polypeptide, a protein, a protein complex, a payload (e.g., a therapeutic agent). or a marker (including, but not limited to a chemical, fluorescent, radioactive or bioluminescent marker). The linker can be used for any useful purpose, such as to form multimers or conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers, Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bonds include an amido bond which may be cleaved for example by the use of tris(2-carboxyethyl) phosphine (TCEP), or other reducing agents, and/or photolysis, as well as an ester bond which may be cleaved for example by acidic or basic hydrolysis.

Checkpoint/factor: As used herein, a checkpoint factor is any moiety or molecule whose function acts at the junction of a process. For example, a checkpoint protein, ligand or receptor may function to stall or accelerate the cell cycle.

Metabolite: Metabolites are the intermediate products of metabolic reactions catalyzed by enzymes that naturally occur within cells. This term is usually used to describe small molecules, fragments of larger biomolecules or processed products.

Modified: As used herein, the term “modified” refers to a changed state or structure of a molecule or entity as compared with a parent or reference molecule or entity. Molecules may be modified in many ways including chemically, structurally, and functionally. In some embodiments, compounds and/or compositions of the present invention are modified by the introduction of non-natural amino acids.

Mutation: As used herein, the term “mutation” refers to a change and/or alteration. In some embodiments, mutations may be changes and/or alterations to proteins (including peptides and polypeptides) and/or nucleic acids (including polynucleic acids). In some embodiments, mutations comprise changes and/or alterations to a protein and/or nucleic acid sequence. Such changes and/or alterations may comprise the addition, substitution and or deletion of one or more amino acids (in the case of proteins and/or peptides) and/or nucleotides (in the case of nucleic acids and or polynucleic acids e.g., polynucleotides). In some embodiments, wherein mutations comprise the addition and/or substitution of amino acids and/or nucleotides, such additions and/or substitutions may comprise 1 or more amino acid and/or nucleotide residues and may include modified amino acids and/or nucleotides. The resulting construct, molecule or sequence of a mutation, change or alteration may be referred to herein as a mutant.

Neoantigen: the term “neoantigen”, as used herein, refers to a tumor antigen that is present in tumor cells but not normal cells and do not induce deletion of their cognate antigen specific T cells in thymus (i.e., central tolerance). These tumor neoantigens may provide a “foreign” signal, similar to pathogens, to induce an effective immune response needed for cancer immunotherapy. A neoantigen may be restricted to a specific tumor. A neoantigen be a peptide/protein with a missense mutation (missense neoantigen), or a new peptide with long, completely novel stretches of amino acids from novel open reading frames (neoORFs). The neoORFs can be generated in some tumors by out-of-frame insertions or deletions (due to defects in DNA mismatch repair causing microsatellite instability), gene-fusion, read-through mutations in stop codons, or translation of improperly spliced RNA (e.g., Saeterdal et al., Proc Natl Acad Sci USA, 2001, 98: 13255-13260).

Off-target: As used herein, “off target” refers to any unintended effect on any one or more target, gene, cellular transcript, cell, and/or tissue.

Operably linked: As used herein, the phrase “operably linked” refers to a functional connection between two or more molecules, constructs, transcripts, entities, moieties or the like.

Payload or payload of interest (POI): the terms “payload” and “payload of interest (POI)”, as used herein, are used interchangeable. A payload of interest (POI) refers to any protein or compound whose function is to be altered. In the context of the present invention, the POI is a component in the immune system, including both innate and adaptive immune systems. Payloads of interest may be a protein, a fusion construct encoding a fusion protein, or non-coding gene, or variant and fragment thereof. Payload of interest may, when amino acid based, may be referred to as a protein of interest.

Pharmaceutically acceptable excipients: the term “pharmaceutically acceptable excipient,” as used herein, refers to any ingredient other than active agents (e.g., as described herein) present in pharmaceutical compositions and having the properties of being substantially nontoxic and non-inflammatory in subjects. In some embodiments, pharmaceutically acceptable excipients are vehicles capable of suspending and/or dissolving active agents. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: Pharmaceutically acceptable salts of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Pharmaceutically acceptable salts include the conventional non-toxic salts, for example, from non-toxic inorganic or organic acids. In some embodiments, a pharmaceutically acceptable salt is prepared from a parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety. Pharmaceutically acceptable solvate: The term “pharmaceutically acceptable solvate,” as used herein, refers to a crystalline form of a compound wherein molecules of a suitable solvent are incorporated in the crystal lattice. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N, N′-dimethylformamide (DMF), N, N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.” In some embodiments, the solvent incorporated into a solvate is of a type or at a level that is physiologically tolerable to an organism to which the solvate is administered (e.g., in a unit dosage form of a pharmaceutical composition).

Stable: As used herein “stable” refers to a compound or entity that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable. In some embodiments, stability is measured relative to an absolute value. In some embodiments, stability is measured relative to a secondary status or state or to a reference compound or entity.

Standard CAR: As used herein, the term “standard CAR” refers to the standard design of a chimeric antigen receptor. The components of a CAR fusion protein including the extracellular scFv fragment, transmembrane domain and one or more intracellular domains are linearly constructed as a single fusion protein.

Stimulus response element (SRE): the term “stimulus response element (SRE), as used herein, is a component of an effector module which is joined, attached, linked to or associated with one or more payloads of the effector module and in some instances, is responsible for the responsive nature of the effector module to one or more stimuli. As used herein, the “responsive” nature of an SRE to a stimulus may be characterized by a covalent or non-covalent interaction, a direct or indirect association or a structural or chemical reaction to the stimulus. Further, the response of any SRE to a stimulus may be a matter of degree or kind. The response may be a partial response. The response may be a reversible response. The response may ultimately lead to a regulated signal or output. Such output signal may be of a relative nature to the stimulus, e.g., producing a modulatory effect of between 1 and 100 or a factored increase or decrease such as 2-fold, 3-fold, 4-fold, 5-fold, 10-fold or more. One non-limiting example of an SRE is a destabilizing domain (DD).

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Tandem: As used herein, the term “tandem” refers to a pattern of arrangement wherein two or more entities are arranged adjacent one another or act in conjunction. In some embodiments, the entity may be a nucleic acid or an amino acid. In one embodiment, the entity may be a payload. In one aspect, the payload may be an immunotherapeutic agent.

T cell: A T cell is an immune cell that produces T cell receptors (TCRs). T cells can be naïve (not exposed to antigen; increased expression of CD62L, CCR7, CD28, CD3, CD127, and CD45RA, and decreased expression of CD45RO as compared to T_{EM}), memory T cells (T_{M}) (antigen-experienced and long-lived), and effector cells (antigen-experienced, cytotoxic). T_{M} can be further divided into subsets of central memory T cells (T_{CM}, increased expression of CD62L, CCR7, CD28, CD127, CD45RO, and CD95, and decreased expression of CD54RA as compared to naïve T cell and effector memory T cells (T_{EM}, decreased expression of CD62L, CCR7, CD28, CD45RA, and increased expression of CD127 as compared to naïve T cells or T_{CM}). Effector T cells (T_{E}) refers to antigen-experienced CD8+ cytotoxic T lymphocytes that have decreased expression of CD62L, CCR7, CD28, and are positive for granzyme and perforin as compared to T_{CM}. Other exemplary T cells include regulatory T cells, such as CD4+CD25+(Foxp3+) regulatory T cells and Treg17 cells, as well as Tr1, Th3, CD8+CD28−, and Qa-1 restricted T cells.

T cell receptor: T cell receptor (TCR) refers to an immunoglobulin superfamily member having a variable antigen binding domain, a constant domain, a transmembrane region, and a short cytoplasmic tail, which is capable of specifically binding to an antigen peptide bound to a MHC receptor. A TCR can be found on the surface of a cell or in soluble form and generally is comprised of a heterodimer having α and β chains (also known as TCRα and TCRβ, respectively), or γ and δ chains (also known as TCRγ and TCRδ, respectively). The extracellular portion of TCR chains (e.g., α-chain, (3-chain) contains two immunoglobulin domains, a variable domain (e.g., α-chain variable domain or V_{α}, β-chain variable domain or V_{β}) at the N terminus, and one constant domain (e.g., α-chain constant domain or C_{α} and β-chain constant domain or C_{β}) adjacent to the cell membrane. Similar to immunoglobulin, the variable domains contain complementary determining regions (CDRs) separated by framework regions (FRs). A TCR is usually associated with the CD3ξ complex to form a TCR complex. As used herein, the term “TCR complex” refers to a complex formed by the association of CD3 with TCR. For example, a TCR complex can be composed of a CD3γ chain, a CD3δ chain, two CD3ε chains, a homodimer of CD3ξ chains, a TCRα chain, and a TCRβ chain. Alternatively, a TCR complex can be composed of a CD3γ chain, a CD3δ chain, two CD3ε chains, a homodimer of CD3ξ chains, a TCRγ chain, and a TCRδ chain. A “component of a TCR complex,” as used herein, refers to a TCR chain (i.e., TCRα, TCRβ, TCRγ or TCRδ), a CD3 chain (i.e., CD3γ, CD3δ, CD3ε or CD3ξ, or a complex formed by two or more TCR chains or CD3ξ chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Treatment or treating: As used herein, the terms “treatment” or “treating” denote an approach for obtaining a beneficial or desired result including and preferably a beneficial or desired clinical result. Such beneficial or desired clinical results include, but are not limited to, one or more of the following: reducing the proliferation of (or destroying) cancerous cells or other diseased, reducing metastasis of cancerous cells found in cancers, shrinking the size of the tumor, decreasing symptoms resulting from the disease, increasing the quality of life of those suffering from the disease, decreasing the dose of other medications required to treat the disease, delaying the progression of the disease, and/or prolonging survival of individuals.

Tune: As used herein, the term “tune” means to adjust, balance or adapt one thing in response to a stimulus or toward a particular outcome. In one non-limiting example, the SREs and/or DDs of the present invention adjust, balance or adapt the function or structure of compositions to which they are appended, attached or associated with in response to particular stimuli and/or environments.

### Equivalents and Scope

Those skilled in the art will recognize or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention. The present invention is further illustrated by the following nonlimiting examples.

### EXAMPLES

### Example 1. TMP Regulation of pELNS Based DD IL12 Constructs

HEK293T cells were transiently transfected for 48 hours with DNA from constructs OT-001444 (pELNS, p40ss-FlexiIL12-hDHFR(I17V)), OT-001445 (pELNS, p40ss-FlexiIL12-Furin hDHFR (I17V)), OT-001446 (pELNS, p40ss-FlexiIL12-hDHFR (Y1220) or OT-001447 (pELNS, p40ss-FlexiIL12-Furin-hDHFR(Y122I)). Vehicle control or 50 μM TMP (+T) was added during the last 24 hours of culture. IL12p40 secreted in the cell supernatants was analyzed in an MSD assay. TMP regulated IL12-hDHFR in HEK293T cells. 100 μM overnight incubation with TMP also increased secretion of all IL12-hDHFR constructs in T cells. IL12 expression was 5.2-fold greater for the OT-001444 construct with TMP, 2-fold for OT-001445, 3.6-fold for OT-001446 and 1.6-fold for OT-001447.

### Example 2. Trimethoprim Dependent IL15-IL15Ra Regulation

HCT-116 cells stably expressing the OT-001111 (OT-IL15-009) cassette were incubated with 0 to 250 μM TMP ligand (or DMSO) for 24 hours. Membrane bound IL15-IL15Ra levels were assessed using flow cytometry detection of IL15Ra. The mean fluorescent intensities (MFI) obtained with each dose, are shown in Table 10.

TABLE 10
TMP dose response

| Dose | | |
|---|---|---|
| (μM) | TMP | DMSO |
| 250 | 1576 | 250 |
| 50 | 1344 | 255 |
| 10 | 1204 | 262 |
| 2 | 1104 | 241 |
| 0.4 | 1001 | 233 |
| 0.08 | 769 | 222 |
| 0.016 | 666 | 219 |
| 0.0032 | 469 | 227 |
| 0.00064 | 304 | 227 |
| 0 | 262 | 238 |

Higher concentrations of TMP resulted in higher expression of IL15Ra. As little as 0.0032 μM of TMP resulted in MFI levels above DMSO controls.

Regulation of IL15-IL15Ra by TMP was also tested in T cells. Constitutive and ecDHFR regulated mbIL15 constructs were inserted into lentiviral transfer vector downstream of EF1a promoter. CD3/CD28 activated primary human T-cells were transduced with OT-001422 OT-IL15-071) or with OT-001471 OT-IL15-073) lentivirus. Cells were incubated with vehicle (DMSO) or 50 μM Trimethoprim for 24 hrs. Following the incubation, IL15 receptor alpha levels were quantified using FACS. TMP dependent IL15Ra expression was observed with the DD regulated construct (OT-001471; OT-IL15-073), which presented two peaks in the FACS plot, while the DMSO control presented only one peak to the left. As expected, IL15Ra levels were also detected with the OT-001422 OT-IL15-071) which expresses IL15-IL15Ra constitutively, which presented two peaks in the FACS plot, when compared to untransduced cells, which only presented one peak. Thus, trimethoprim dose dependently regulates IL15-IL15Ra in primary human T cells.

### Example 3. Effect of Promoter on FKBP Regulated DD-IL12 Constructs

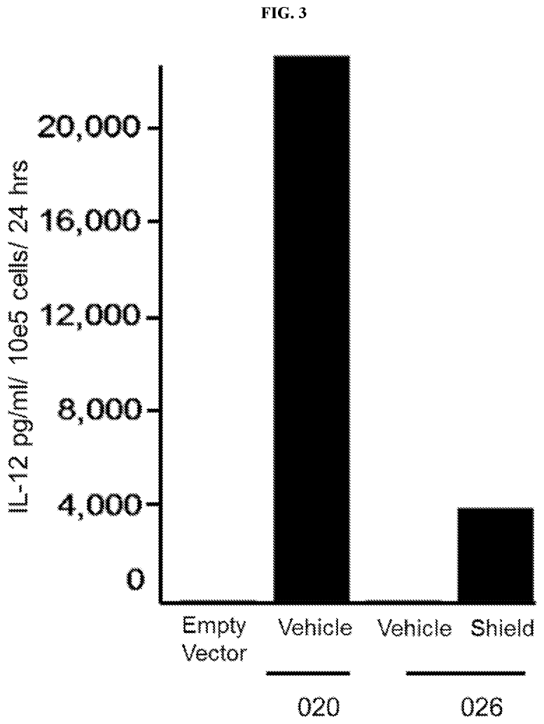

Human T cells were activated with CD3/CD28 Dynabeads (Life Technologies) for 1 day prior to transduction with lentivirus carrying the transgene related to constructs OT-IL12-020 and OT-IL12-026. Seven days after the transduction, cells were treated with 1 μM Shield-1 for 24 hrs. Flexi IL12 was quantified by MSD assay for IL12p70. As shown in FIG. 3, construct OT-IL12-026 (annotated as 026 in FIG. 3) showed IL12 production only upon treatment with Shield-1 at levels of about 4,000 pg/ml. As expected, the positive control construct, OT-IL12-020 (annotated as 020 in FIG. 3) showed constitute IL12 expression in the absence of ligand and the empty vector control did not show any IL12 expression. These data show that shield-1 regulates Flexi IL12 production driven by the EF1a promoter in T-cells.

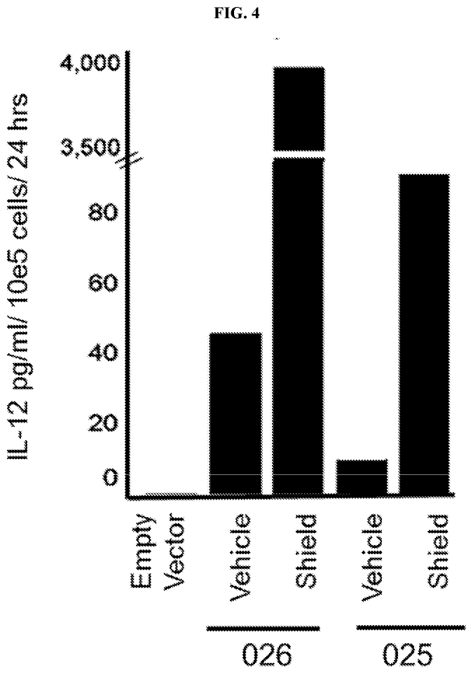

The effect of the PGK promoter on IL12 regulation was compared to the EF1a promoter in FKBP regulated constructs. T cells were transduced with lentivirus carrying transgene related to constructs OT-IL12-025 and OT-IL12-026. T cells were cultured similar to the experiments related to FIG. 3. The IL12 regulation achieved with the PGK construct, OT-IL12-025 was lower in the presence of Shield 1 when compared to the levels obtained with the EF1a promoter driven construct OT-IL12-026 as shown in FIG. 4, where the constructs are denoted as 025 and 026 respectively. In the absence of ligand, lower levels of IL12 (˜10 pg/ml) with the PGK promoter construct. Taken together, these data show that Shield-1 regulates Flexi IL12 production from EF1a or PGK promoter in T-cells, and the PGK promoter drives lower levels of IL12 than the EF1a promoter.

### Example 4. IL12 Regulation in Constructs Co-Expressing CAR

CD19 CAR and IL12 payloads were formatted into tandem expression cassettes where the two payloads were separated by an internal ribosome entry site (IRES)/or P2A (porcine teschovirus-1 2A) site. An FKBP DD was added to the C-terminal of some of the constructs to test the ability of biocircuits of the present disclosure to tune IL12 expression in the constructs.

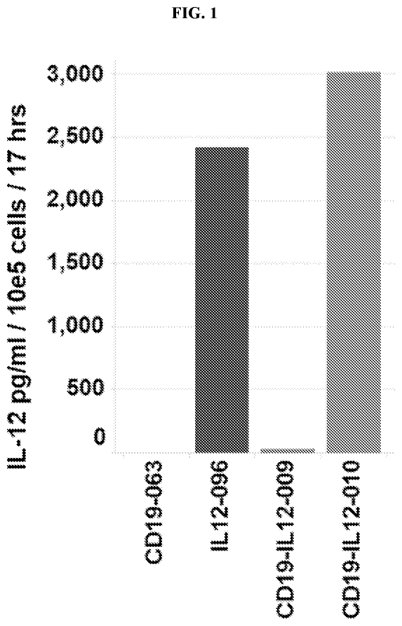

Primary human T cells were transduced with virus related to OT-001356 OT-CD19-IL12-009), OT-001357 OT-CD19-IL12-010), OT-001386 OT-CD19-IL12-011) and OT-001387 OT-CD19-IL12-013). As a control, construct OT-001442 OT-IL12-096) that only expresses IL12 constitutively and OT-001407 (OT-CD19-063), which only expresses CD19 CAR constitutively were also included in the experiment. Seven days after transduction, cells were washed and plated in fresh media. After 24 hours of incubation, Flexi IL12 levels in the supernatant were quantified using MSD for IL12 p70 and the results were calculated as IL12 pg/ml per 1 million cells at 17 hours. As shown in FIG. 1 the placement of an IRES between the CD19 CAR and the IL12 in OT-001356 (OT-CD19-IL12-009), reduced the expression of IL12 from the tandem expression construct. The levels of IL12 observed with the placement of the P2A between the CD19 CAR and the IL12 in the OT-001357 (OT-CD19-IL12-010) resulted in IL12 levels comparable to OT-001442 (OT-IL12-096) construct, which expresses IL12 constitutively. As expected, the OT-001407 (OT-CD19-063) construct did not show any expression of IL12.

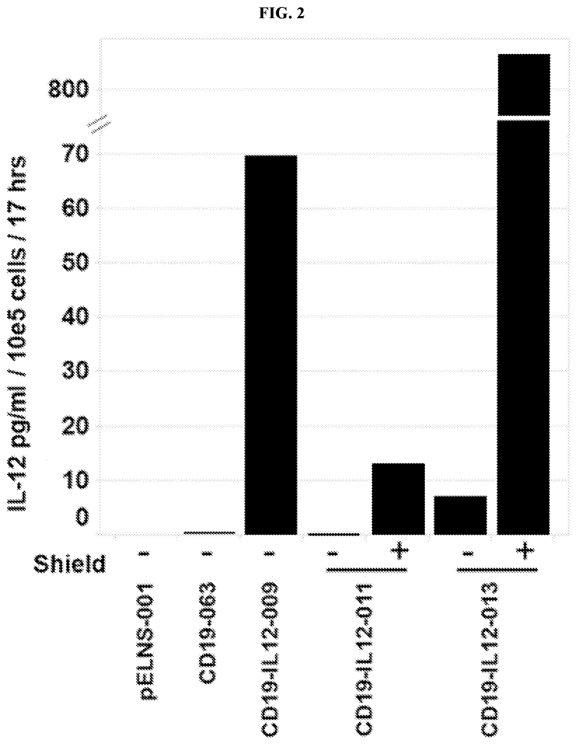

To test the ability of biocircuits described herein to regulate the expression of IL12, OT-001386 OT-CD19-IL12-011) and OT-001387 OT-CD19-IL12-013) constructs were treated with Shield1 or vehicle control for 17 hours. The experiments were performed in T cells in a manner similar to those described for FIG. 1. The IL12 levels obtained were compared to the IL12 levels obtained with OT-001356 (OT-CD19-IL12-009), which expresses IL12 constitutively; OT-001407 (OT-CD19-063), which expresses CD19 CAR constitutively; and the pELNS empty vector (referred to as pELNS-001). As shown in FIG. 2, Shield-1 regulates IL12 production in both OT-001386 (OT-CD19-IL12-011) and OT-001387 (OT-CD19-IL12-013), wherein the payloads were separated by P2A and IRES respectively. Ligand dependent expression of IL23 was higher with OT-001387 (OT-CD19-IL12-013)—about 800 pg/ml and about 10-20 pg/ml with OT-001386 (OT-CD19-IL12-011).

The use of the P2A resulted in lower levels of expression of IL12, both in the presence and absence of Shield 1. Taken together, these data show that IL12 levels can be tuned using biocircuits described herein by the use of P2A sites and/or SREs.

### Example 5. Ligand Dependent Regulation of CD19 CAR-IL12 Tandem Constructs in 293T Cells

pELDS vector based tandem CD19 CAR-IL12 constructs i.e., OT-001386, OT-001387 were transfected into HEK293T cells for 24 hours. Cells were then treated with Shield 1 for 48 hours. The surface expression of CD19 CAR was measured by FACS using CD19 Fc and IL12 was measured using the p40 MSD assay. The expression of the regulatable tandem constructs was compared to constitutively expressed tandem constructs, OT-001356 and OT-001357; the constitutively expressed monocistronic constructs, OT-001407; and negative controls including the empty vector (pELDS) and the parental HEK293T cells. IL12 levels were induced by Shield-1 treatment in OT-001386 and OT-001387. The induction of IL12 was greater in OT-001387, however, the basal levels of IL12 in the absence of ligand was much lower in OT-001386. As expected, the constitutive tandem constructs showed high expression of IL12 and the negative controls did not show IL12 expression.

The percentage of cells with CD19 CAR surface expression obtained for each group is shown in Table 11.

TABLE 11
Percentage CAR positive cells

| | | % CAR |
|---|---|---|
| Construct | Shield 1 | expression |
| OT-001386 | + | 30.7 |
| | − | 33.2 |
| OT-001387 | + | 34.7 |
| | − | 35.7 |
| OT-001356 | − | 33.3 |
| OT-001357 | − | 36.1 |
| OT-001407 | − | 36.8 |
| Vector | − | 0.75 |

As expected, no increase in CAR expression was observed with the addition of ligand to the tandem CD19-IL12 construct expressing cells, since the CAR is not appended to DD.

Tandem CD19 CAR-IL12 constructs, where IL12 is regulated by hDHFR DDs were tested in HEK293T cells. Cells were plated at 1 million cells per well and transiently transfected with 1 μg of DNA/well. At 24 hours, the media was changed and replaced with media containing 50 μM TMP. After 48 hours, the supernatants were collected and utilized for the IL12 p40 MSD assay. Regulation of IL12 in the tandem constructs was compared to constitutively expressed tandem construct, OT-001356, regulated IL12 monocistronic constructs OT-001444, OT-001446 as well the constitutively expressed monocistronic IL12 construct, OT-001442.

OT-001613 construct showed three-fold upregulation in IL12 levels in the ligand treated samples as compared to the untreated control, OT-001614 and OT-001616 showed 1.3-fold and 2-fold upregulation in IL12 in ligand treated samples as compared to the untreated. All DHFR based tandem CD19 CAR-IL12 (Table 29) constructs showed regulation of IL12 in the presence of TMP. The basal expression of IL12 observed with the DHFR regulated tandem constructs was much lower when compared to the corresponding monocistronic DHFR regulated IL12 constructs.

TABLE 12
IL12 levels

| | IL12p40 pg/mL | | | |
|---|---|---|---|---|
| Construct | Ligand | | Vehicle | |
| OT-001442 | — | — | 4693321 | 4613343 |
| OT-001444 | 390606.3 | 381446.7 | 196977.6 | 196000 |
| OT-001446 | 1124950 | 1136322 | 215323.9 | 199243.6 |
| OT-001356 | — | — | 127453.7 | 125010.8 |
| OT-001612 | 16911.73 | 17115.21 | 2483.863 | 2201.019 |
| OT-001615 | 51692.83 | 53613.26 | 10066.25 | 9930.337 |
| OT-001618 | 158125.8 | 162826.1 | 16453.51 | 16807.98 |

Additional hDHFR and ecDHFR based tandem CD19 CAR-IL12 constructs were also tested and ligand dependent regulation was observed in all instances as shown in Table 13.

TABLE 13
Regulatable DHFR construct

| | Fold | |
|---|---|---|
| | induction | % CAR |
| Construct | in IL12 | expression |
| OT-001620 | 3 | 31 |
| OT-001621 | 3 | 38 |
| OT-001617 | 40 | 20 |
| OT-001622 | 12 | 26 |

### Example 6. Ligand Dependent Regulation of CD19 CAR-IL12 Tandem Constructs in the Presence of Antigen

The regulation of IL12 in CD19 CAR IL12 constructs in the presence of the CD19 antigen was measured to assess IL12 regulation in the presence of antigen—an environment likely encountered by the T cells in vivo. ecDHFR and hDHFR constructs were transduced into T cells as previously described. T cells were then co-cultured with K562 cells engineered to ectopically express CD19 or with parental K562 cells that have little to no expression of CD19. Each group was further sub divided into a TMP group, in which the cells were dosed with 50 μM TMP or the and a control group where the cells were left untreated. The cell surface expression of CAR was measured using CD19-Fc and the IL12 levels were measured using MSD assay. The results are shown in Table 31. The fold induction as represented in Table 24 is with reference to the IL12 levels in the absence of ligand.

TABLE 14
IL12 induction in the presence of antigen

| | Fold induction in IL12 | | |
|---|---|---|---|
| | Parental- | CD19 | % CAR |
| Construct | K562 | K562 | expression |
| OT-001619 | 5 | 4 | 22.9 |
| OT-001620 | 5 | 4 | 31 |
| OT-001621 | 10 | 7 | 38 |
| OT-001617 | 30 | 40 | 20% |
| OT-001622 | 30 | 40 | 26% |

As shown in Table 14, IL12 regulation was observed both in the presence and absence of antigen. The ecDHFR constructs showed even higher induction of IL12 in the presence of antigen as compared to the absence of the antigen. The presence of antigen was however required for the interferon gamma and IL2 induction. CAR expressing T cells that produce >20-50 pg/mL levels of IL12 also demonstrate higher IFN gamma than IL12 negative CAR-T cells. CAR expression was also found to positively correlate with IFN gamma and IL2 production.

### Example 7. Anti-Tumor Activity of Tandem CD19-IL12 CARs

The study was designed to determine how IL12 increased the rejection of Nalm-6 tumors by CD19 CAR expressing T cells and whether the presence of the antigen affects IL12 production by CAR T cells in tumor bearing and/or non-tumor bearing mice.

To test in vitro activity, T cells were transduced with one of the following constructs OT-001407, OT-001356, OT-001357, OT-001442 or empty vector, pELDS. The in vitro activity of the T cells expressing these constructs was measured by co culturing them with antigen positive, CD19 expressing K562 cells or antigen negative parental K562 cells. As a control T cells that were not co-cultured with any K562 cells were also used. On day 7 after co culture, CAR-IL12 tandem constructs produced more IFN gamma levels than OT-001407 only or OT-001442 constructs. Secreted IL2 levels were highest in OT-001407 transduced T cells, followed by the CAR-IL12 tandem constructs. In contrast, the highest level of secreted IL12 was observed in OT-001442, followed by OT-001357 and then OT-001356. Thus, IL12 expression does not completely correlate with IFN gamma levels, indicating that an interaction between CD19 CAR expression and IL12 expression may be responsible for the synergistic effect on the IFN gamma expression.

To test in vivo activity, experiments were performed in NSG mice. Nalm6 cells were transfected with Redifect Red-Fluc-GFP (Perkin Elmer) under selection using Puromycin at 2 μg/ml for approximately 2 months in order to generate a line that stably expresses the luciferase reporter; thereafter named Nalm6-Luc. Ten days before tumor implantation, cells were thawed and cultured in puromycin containing media. On the day of the injection (day 0), cells were counted, resuspended in PBS and injected into NSG mice via tail vein injection at 1 million per injection. On day 6, mice were imaged for bioluminescent intensity and sorted into groups based on their tumor size ensure that all groups had approximately the same sized tumors. T cells were injected on day 7 at either 0.1 or 1 million cells per injection. The T cells were transduced with one of the following constructs OT-001356 (IRES), and OT-001357 (P2A) prior to the injection. To assess antitumor activity of the tandem constructs to their corresponding monocistronic constructs, OT-001407 and OT-001442 were also included. Additional controls included cells transduced with the empty vector, pELDS and untransduced cells. Mice that were alive at day 68 (which included mice in the 0.1e⁶ CD19 CAR-IL12 constructs groups) were re-challenged with similar doses of T cells. Tumors were monitored in mice using bioluminescent imaging using which the mean of the Total Flux (photons/second) was measured as an indicator of tumor burden (Table 32). In Table 15, UTD indicates untreated group and pELDS is the empty vector control group.

TABLE 15
Total Flux in tumor bearing mice

| | | | OT-001442 | | OT-001407 | | OT-001356 | | OT-001357 | |
|---|---|---|---|---|---|---|---|---|---|---|
| Day | UTD | pELDS | 0.1e⁶ | 1.0e⁶ | 0.1e⁶ | 1.0e⁶ | 0.1e⁶ | 1.0e⁶ | 0.1e⁶ | 1.0e⁶ |
| 6 | 1.50E+06 | 1.48E+06 | 1.43E+06 | 1.42E+06 | 1.46E+06 | 1.51E+06 | 1.57E+06 | 1.38E+06 | 1.67E+06 | 1.92E+06 |
| 10 | 1.56E+07 | 1.94E+07 | 9.79E+06 | 1.31E+07 | 7.80E+06 | 3.87E+06 | 2.17E+07 | 1.06E+07 | 2.64E+07 | 3.45E+07 |
| 14 | 5.20E+08 | 4.39E+08 | 5.35E+08 | 4.64E+08 | 1.08E+08 | 1.06E+07 | 7.10E+08 | 2.49E+08 | 4.75E+08 | 1.16E+08 |
| 18 | 2.49E+09 | 2.51E+09 | 3.18E+09 | 3.04E+09 | 2.85E+08 | 1.41E+07 | 2.63E+09 | 3.04E+08 | 2.21E+09 | 2.05E+07 |
| 21 | 4.93E+09 | 3.15E+09 | 5.00E+09 | 4.28E+09 | 5.50E+08 | 3.55E+07 | 4.43E+09 | 1.64E+07 | 1.99E+09 | 1.17E+06 |
| 24 | 7.91E+09 | 5.90E+09 | 1.57E+10 | 1.86E+10 | 1.00E+09 | 1.03E+08 | 4.62E+09 | 8.21E+05 | 1.80E+09 | 9.36E+05 |
| 27 | — | — | — | — | — | 3.92E+08 | 1.35E+10 | 1.14E+06 | 8.47E+08 | 8.92E+05 |
| 31 | — | — | — | — | — | 1.48E+09 | — | 7.17E+05 | 1.61E+08 | 9.75E+05 |
| 34 | — | — | — | — | — | 4.19E+09 | — | 1.41E+06 | 9.44E+07 | 1.00E+06 |
| 38 | — | — | — | — | — | 3.40E+09 | — | 1.06E+06 | 1.49E+08 | 1.07E+06 |
| 41 | — | — | — | — | — | 5.91E+09 | — | 1.56E+06 | 2.28E+08 | 1.58E+06 |
| 45 | — | — | — | — | — | 2.02E+09 | — | 1.18E+06 | 3.15E+08 | 1.48E+06 |
| 48 | — | — | — | — | — | 2.10E+09 | — | 2.03E+06 | 3.65E+08 | 1.33E+06 |
| 52 | — | — | — | — | — | 3.87E+09 | — | 2.10E+06 | 7.62E+08 | 1.14E+06 |
| 55 | — | — | — | — | — | 5.76E+09 | — | 8.07E+05 | 4.63E+08 | 6.59E+05 |
| 60 | — | — | — | — | — | — | — | 5.44E+05 | 3.95E+08 | 6.57E+05 |
| 63 | — | — | — | — | — | — | — | 5.82E+05 | 4.78E+08 | 4.41E+05 |
| 67 | — | — | — | — | — | — | — | 7.32E+05 | 4.72E+08 | 6.77E+05 |
| 70 | — | — | — | — | — | — | — | 6.31E+05 | — | 6.89E+05 |
| 73 | — | — | — | — | — | — | — | 5.64E+05 | — | 6.03E+05 |
| 76 | — | — | — | — | — | — | — | 7.15E+05 | — | 6.26E+05 |
| 80 | — | — | — | — | — | — | — | 7.56E+05 | — | 9.39E+05 |
| 83 | — | — | — | — | — | — | — | 7.73E+05 | — | 6.86E+05 |

As shown in Table 15, constitutive IL12 expression in CD19 positive CART cells (i.e. the CD19 CAR IL12 tandem constructs) caused a robust anti-tumor effect at the 1e⁶ dose. OT-001357 (P2A) displayed superior killing compared to OT-001356 (IRES) at the 0.1e⁶ (lower dose). Percent survival was also measured for each group during the course of the experiment and the following numbers were obtained (i) UTD: 0% survival at day 28 (ii) pELDS: 0% survival at day 29 (iii) 0.1e⁶ OT-001442:0% survival at day 24 (iv) 1e⁶ OT-001442:0% survival at day 24 (v) 0.1e⁶ OT-001407:0% survival at day 30 (vi) 1.0e⁶ OT-001407:0% survival at day 60 (vii) 0.1e⁶ OT-001356:0% survival at day 30 (viii) 1.0e⁶ OT-001356:87.5% survival at day 83 (ix) 0.1e⁶ OT-001357:0% survival by day 72 (x) 1.0e⁶ OT-001357:62.5% survival at day 83. Thus, the co-delivery of IL12 with CD19 positive CAR T cells using the tandem constructs resulted in a significant improvement in survival, particularly at the 1.0e⁶ T cell dose.

Weekly serial whole blood and plasma collections were made to assess T cell populations and cytokine production. MSD assays were carried out to measure the levels of IFN gamma and IL12. The IL12 levels begin to rise after the T cell injection with the 1e⁶ OT-001357, and the IL12 levels obtained with 1e⁶ OT-001356 was ten-fold less than the former. Despite these differences in the IL12 levels, comparable levels of IFN gamma were obtained with both constructs, with the 1e⁶ OT-001356 construct producing higher IFN gamma than the 1e⁶ OT-001357. IFN gamma levels were also detectable with the 0.1e⁶ injections of the two constructs. In all constructs, the levels of IFN gamma continued to increase over time, even when the IL12 levels began to decrease.

### Example 8. In Vivo Characterization of T Cell Phenotypes in Constitutive IL12 CD19 Constructs

IL12-transduced T cells have a Th1-skewed phenotype in vitro, i.e. produce IFN gamma). To assess phenotypic effects of IL12 in vivo, GFP positive (IL12 negative) cells were generated and co-transferred with CD19 CAR-IL12 transduced cells or CD19 alone or IL12 alone cells. Nalm 6 tumor bearing NSG mice were injected with 8 million GFP positive cells, in addition to CD19 CAR-IL12 cells or their corresponding monocistronic constructs. The groups utilized in this study included, 1e⁶ empty vector expressing T cells plus 8e⁶ GFP positive cells, 1e⁶ OT-001407 plus 8e⁶ GFP positive cells, 0.1 e⁶ OT-001356 plus 8e⁶ GFP positive cells, 1e⁶ OT-001356 plus 8e⁶ GFP positive cells, 0.1e⁶ OT-001357 plus 8e⁶ GFP positive cells, 1e⁶ OT-001357 plus 8e⁶ GFP positive cells, and 1e⁶ OT-001442 plus 8e⁶ GFP positive cells.

Animals were bled on day 3, day 5, day 7 and euthanized at day 12. Blood, spleen, and bone marrow were collected for analysis of T cell number and CD8 frequency. Phenotypic T cell markers such as Granzyme, Tbet, pSTAT4, CD25, and ICOS were analyzed in the GFP positive cells using FACS. At day 3, 5 and 7, the GFP positive cells co-transferred with CD19 CAR-IL12 constructs showed an increase in Granzyme B and Tbet. By day 12, post transfer, the levels of Granzyme B, Tbet, pSTAT4, CD25 and ICOS levels increased in the blood of the mice injected with the CD19 CAR-IL12 tandem constructs. ANOVA test for significance was p<0.0001 for all markers. Concomitant measurement of plasma IL12 showed that the plasma IL12 levels correlated with dose of the tandem CAR-IL12 constructs. Further, even a level of IL12 as low as 200 pg/ml (EC 50 of IL12 in plasma is ˜100 pg/ml) was sufficient to induce phenotypic changes as measured by the markers described above and by the increase in IFN gamma levels.

At day 12 after T cell transfer, the frequency of human T cells in the blood increased over time and increased to a greater level in groups with higher levels of IL12, including groups that were dosed with CD19 CAR-IL12 constructs. The frequency of human T cells that were GFP positive dropped in groups that received CD19-CAR either in tandem constructs or as a monocistronic construct, suggesting that CAR expressing T cells were expanding more than other T cells. The largest reduction in GFP positive cell frequency was in the groups that received the high dose of CAR positive cells with IL12 namely the 1e⁶ OT-001356 (IRES) injected group. Among the human cells, the percentage of CD8 positive cells increased over time in mice expressing IL12 either in monocistronic constructs or in tandem with CD19 CAR.

### Example 9. Anti-Tumor Activity of Tandem CD19-IL15-IL15Ra CARs

The purpose of the study was to determine if membrane bound IL15-IL15Ra, when present in a tandem construct, could enhance the activity of CD19 CARs to reduce tumor growth. Nalm-Luc cells were generated as described before. Ten days before tumor implantation, cells were thawed and cultured in puromycin containing media. On the day of the injection (day 0), cells were counted, resuspended in PBS and injected into NSG mice via tail vein injection at 1 million per injection. On day 6, mice were imaged for bioluminescent intensity and sorted into groups to ensure that all the groups have the same average tumor burden. Different doses of T cells were injected on day 7 at 0.13 or 0.38 or 1.13 million cells per injection. The cells were transduced with OT-001458(P2A) construct. To assess antitumor activity of the tandem construct to its corresponding monocistronic constructs, T cells expressing OT-001407 and OT-001422 individually or OT-001407 alone were also included. Additionally, untransduced cells were included as a negative control. Tumors were monitored in mice using bioluminescent imaging using which the mean of the Total Flux (photons/second) was measured as an indicator of tumor burden. As shown in Table 16, cohorts of mice injected with different doses of the tandem construct showed a reduction in the total flux measured during the course of the experiment. The least value of total flux was obtained for the highest dose (1.13 million T cells) of OT-001458(P2A) construct, followed by the lowest does of 0.13 million T cells. The 0.13 million T cell dose initially showed a reduction in photon flux till about day 30, but after day 30 displayed an upward trend in photon flux indicating relapse of the Nalm6 tumors. About 80% of the animals in the 1.13 million OT-001458(P2A) construct group survived at day 50, while the 0.13 million and 0.38 million dose groups of OT-001458(P2A) construct showed 0% survival at day 50. All other groups tested including untransduced T cell group, the OT-001407 CAR group and the group comprising T cells co-transduced with OT-001407 and OT-001422, showed 0% survival by day 35. In Table 16, Utd refers to untransduced.

TABLE 16
Total Flux in tumor bearing mice

| | | Utd + | | | | | | | | | |
|---|---|---|---|---|---|---|---|---|---|---|---|
| | | OT- | | | | | | | OT-001407 + | | |
| | Utd | 001422 | OT-001407 | | | OT-001458 | | | OT-001422 | | |
| Days | — | — | 0.13E+06 | 0.38E+06 | 1.13E+06 | 0.13E+06 | 0.38E+06 | 1.13E+06 | 0.13E+06 | 0.38E+06 | 1.13E+06 |
| 6 | 1.00E+06 | 1.00E+06 | 1.01E+06 | 1.00E+06 | 1.01E+06 | 1.04E+06 | 1.04E+06 | 1.03E+06 | 1.02E+06 | 1.03E+06 | 1.07E+06 |
| 9 | 2.72E+06 | 4.55E+06 | 4.40E+06 | 3.11E+06 | 5.18E+06 | 4.44E+06 | 4.17E+06 | 4.60E+06 | 4.71E+06 | 9.28E+06 | 8.11E+06 |
| 13 | 7.1E+07 | 9.40E+07 | 6.21E+07 | 5.88E+07 | 5.10E+07 | 5.13E+07 | 2.64E+07 | 4.34E+06 | 9.70E+07 | 1.08E+08 | 8.61E+07 |
| 16 | 4.47E+08 | 4.66E+08 | 4.73E+08 | 2.13E+08 | 1.96E+08 | 2.93E+08 | 5.22E+07 | 6.08E+06 | 4.25E+08 | 5.84E+08 | 2.82E+08 |
| 20 | 2.78E+09 | 2.80E+09 | 2.13E+09 | 1.16E+09 | 1.10E+09 | 7.81E+08 | 7.58E+07 | 2.24E+06 | 2.78E+09 | 2.06E+09 | 1.16E+09 |
| 23 | 6.58E+09 | 4.50E+09 | 3.47E+09 | 2.20E+09 | 2.07E+09 | 1.75E+09 | 6.89E+07 | 7.06E+05 | 5.23E+09 | 3.14E+09 | 1.60E+09 |
| 27 | — | — | 7.65E+09 | 5.75E+09 | 6.08E+09 | 1.04E+09 | 5.45E+07 | 1.14E+06 | — | 1.19E+10 | 4.39E+09 |
| 30 | — | — | 2.99E+10 | 1.58E+10 | 1.62E+10 | 6.29E+09 | 8.11E+07 | 1.35E+06 | — | 3.19E+10 | 8.28E+09 |
| 34 | — | — | — | — | — | 3.77E+08 | 1.13E+08 | 1.09E+06 | — | — | — |
| 37 | — | — | — | — | — | 1.77E+08 | 6.77E+08 | 1.22E+06 | — | — | — |
| 40 | — | — | — | — | — | 2.00E+08 | 1.50E+09 | 1.12E+06 | — | — | — |
| 44 | — | — | — | — | — | 5.81E+08 | 3.72E+09 | 2.37E+06 | — | — | — |
| 48 | — | — | — | — | — | 7.97E+08 | 3.30E+09 | 1.62E+06 | — | — | — |
| 50 | — | — | — | — | — | 1.43E+09 | 5.84E+09 | 2.57E+06 | — | — | — |
| 55 | — | — | — | — | — | — | — | 1.30E+07 | — | — | — |
| 58 | — | — | — | — | — | — | — | 3.18E+07 | — | — | — |
| 62 | — | — | — | — | — | — | — | 6.12E+07 | — | — | — |
| 64 | — | — | — | — | — | — | — | 4.66E+07 | — | — | — |

### Example 10. CD19 CAR-IL12 Tandem Construct Expression and Function in the Presence of Antigen

Cytokine production CD19 CAR IL12 constructs in the response to antigen was measured. On day 0, primary human T cells were stimulated with Dynabeads (T-expander CD3/CD28) at a 3:1 bead: cell ratio in media containing 10% fetal bovine serum (FBS). The next day, T cells were transduced with lentivirus produced with constructs OT-001407, or OT-001356. On day 2, the cells were diluted 1:2 with fresh 10% FBS media. On day 6, the cells were counted for equal cell number plating, and media was replaced. Transduction efficiency was analyzed on day 7 by flow cytometry using CD19-Fc to detect surface CAR expression. Cytokines that had accumulated in the overnight culture supernatants (from 100,000 cells per 200 uL media) were measured using human IL12p70 (and/or human interferon-gamma) MSD V-plex assay kits (Meso Scale Discovery). IL12 p70 expression on day 7 of expansion was approximately 200 pg/ml with OT-001356, whereas little to no expression was observed with OT-001407.

T cells were co-cultured with K562 cells ectopically expressing CD19 antigen. In the presence of CD19 antigen increased IL12 production by 2-fold in OT-001356 expressing T cells. Interferon gamma levels increased 2.5 fold in OT-001356 compared to OT-001407 expressing T cells suggesting that IL12 leads to increased Interferon gamma production in CAR-T cells. A 3 fold reduction in IL2 levels in OT-001356 expressing T cells compared to OT-001407 expressing T cells was also observed.

Independent populations of T cells were transduced with varying dilutions of virus corresponding to OT-001356, OT-001407, OT-001992, or OT-001386 and co-cultured with K562 parental or CD19 expressing K562 cells. T cells were treated with 1 μM Shield-1 for 17 hours following which, IL12 (p70) levels and CD19 CAR expression were measured. OT-001386 OT-CD19-IL12-011) showed ligand dependent regulation of IL12 at all virus dilutions tested (2 and 10 μL). When co-cultured with parental K562 cells, approximately 10-15 fold induction in IL12 levels compared to vehicle control was observed. When co-cultured with CD19 expressing K562 cells, a 25 fold induction in IL12 expression was observed with the 2 μL virus dilution and a 17 fold induction in IL12 expression (compared to the untreated DMSO control) was observed. Little to no expression of IL12 was observed with the OT-001992 under any of the experimental conditions described herein i.e. plus or minus ligand; plus or minus CD19 expressing K562 cells. The percentage of cells expressing CAR in OT-001386 OT-CD19-IL12-011) expressing T cells was approximately 5.5-6.08% and approximately 2.45-3.83 percent in OT-001992 expressing T cells.

### Example 11. DHFR Regulated CD19-CAR-IL12 Constructs

On day 0, primary human T cells were stimulated with Dynabeads (T-expander CD3/CD28) at a 3:1 bead: cell ratio in media containing 10% fetal bovine serum (FBS). The next day, lentivirus produced with construct OT-001356, OT-001407, OT-001612, OT-001615, OT-001618 were added in the presence of reduced serum (5% FBS). On day 2, the cells were diluted 1:2 with fresh 10% FBS media. On day 6, the cells were counted to ensure equal cell number plating, and media replaced. In some instances, cells are treated with 50 μM TMP or treated in the absence or presence of antigen re-stimulation (with human Immunocult soluble CD3/CD28 reagent from StemCell Technologies or with parental K562 cells versus K562 cells stably expressing the CAR antigen CD19 at a E:T ratio of 1:2). On day 7, after overnight incubation, transduction efficiency was analyzed by flow cytometry using CD19-Fc to detect surface CAR expression. Cytokine that had accumulated in the overnight culture supernatants (from 100,000 cells per 200 uL media) were measured using human IL12p70 (and/or human interferon-gamma) MSD V-plex assay kits (Meso Scale Discovery). The results are shown in Table 14. The “fold change with TMP” column indicates the fold change in IL12 levels with TMP treatment when compared to the vehicle control values (pg/ml).

TABLE 14
IL12 and CAR expression

| | IL12 expression | | | | |
|---|---|---|---|---|---|
| | Parental K562 | | CD19-K562 | | |
| | Vehicle | Fold | Vehicle | Fold | % CAR |
| | control | change | control | change | positive |
| Construct ID | (pg/ml) | with TMP | (pg/ml) | with TMP | cells |
| OT-001612 | 1 | 9 | 4 | 4 | 10.4 |
| OT-001615 | 1 | 6 | 3 | 3 | 8.40 |
| OT-001618 | 1 | 5 | 2 | 5 | 7.18 |

As shown in Table 17, ligand dependent regulation of IL12 was observed with all constructs tested. Little to no IL12 expression was observed with OT-001407 and high levels of IL12 were observed with OT-001356 construct.

Similar experiments were performed with human DHFR regulated CD19CAR-IL12 constructs. The results are shown in Table 35. In Table 35 “—” indicates approximate IL12 values. The “fold change with TMP” column indicates the fold change in IL12 levels with TMP treatment when compared to the vehicle control values (pg/ml)

TABLE 18
IL12 and CAR expression

| | IL12 expression | | | | |
|---|---|---|---|---|---|
| | Parental K562 | | CD19-K562 | | |
| | Vehicle | Fold | Vehicle | Fold | % CAR |
| | control | change | control | change | positive |
| Construct ID | (pg/ml) | with TMP | (pg/ml) | with TMP | cells |
| OT-001619 | 90 | 5 | ~275 | 4 | 29 |
| OT-001620 | 130 | 5 | ~300 | 4 | 23.8 |
| OT-001621 | 20 | 10 | 50 | 7 | 9.28 |
| OT-001617 | 0.3 | 30 | 0.6 | 40 | 6.8 |
| OT-001622 | 20 | 70 | 70 | 40 | 21 |

All constructs tested showed ligand and antigen dependent expression of IL12. OT-001621 and OT-001622 were considered for in vivo characterization as they demonstrated strong CAR expression as well tunable expression of IL12 in the presence of TMP and low basal expression of IL12 in the absence of TMP.

Constructs shown in Table 19 were transduced into T cells using methods described herein. T cells were then treated increasing doses of TMP (also shown in Table 19) for 23 hours and human IL12p70 was analyzed by MSD V-plex assay kits (Meso Scale Discovery). The results are shown in Table 19.

TABLE 19
TMP dose response

| TMP Concentration | OT- | OT- | OT- | OT- | OT- | OT- |
|---|---|---|---|---|---|---|
| (μM) | 001407 | 001357 | 001619 | 001620 | 001621 | 001622 |
| 100.0000 | 0.1724 | 1339.48 | 54.118 | 108.718 | 174.47 | 194.33 |
| 50.0000 | 0.1803 | 1456.46 | 45.475 | 93.986 | 147.48 | 191.32 |
| 16.6667 | 0.1529 | 1339.09 | 34.163 | 72.579 | 105.09 | 186.57 |
| 5.5556 | 0.0000 | 1472.32 | 25.482 | 56.034 | 57.867 | 173.62 |
| 1.8519 | 0.1724 | 1525.97 | 25.749 | 51.543 | 44.655 | 144.99 |
| 0.6173 | 0.0000 | 1454.19 | 20.950 | 42.869 | 34.011 | 116.79 |
| 0.2058 | 0.0000 | 1548.31 | 21.650 | 39.186 | 30.550 | 70.402 |
| 0.0686 | 0.0000 | 1460.43 | 21.190 | 39.578 | 28.198 | 41.929 |
| 0.0229 | 0.1529 | 1476.81 | 20.310 | 42.142 | 28.843 | 25.205 |
| 0.0076 | 0.0000 | 1631.58 | 20.283 | 39.103 | 29.831 | 12.5051 |
| 0.0025 | 0.1842 | 1560.01 | 20.820 | 42.375 | 26.997 | 7.7922 |
| 0.0001 | 0.2569 | 1294.83 | 19.955 | 38.719 | 27.232 | 4.7976 |

About a 3-fold increase in IL12 levels were observed with OT-001621 with increasing TMP doses, whereas OT-001622 showed a 6-fold induction over the TMP dose range tested. The EC50 values were calculated as OT-001619 (130 μM); OT-001620 (65 μM); OT-001621 (25 μM); OT-001622 (0.45 μM). These EC50 values are within the reported human exposure ranges for TMP depending on the dose regime selected i.e. 80-160 mg BID (twice a day) PO (oral) dosing: 1-6 μM steady state levels and 150 mg/m2 IV (intravenous) dosing every 8 hours: 17-34 μM maximum peak levels.

### Example 12. In Vivo Anti-Tumor Activity of Tandem CD19-IL12 CAR

The study was designed to determine how a basal expression of IL12 produced by a CD19 targeting CAR (CD19-CAR) T cells is able to: (a) increase expansion of CD19-CAR T cells, (b) increase IFNg production by CD19-CAR T cells in vivo, and (c) enhance anti-tumor efficacy of CAR⁺ T cell dose against CD19+ Nalm6 tumors. To test in vivo anti-tumor activity, experiments were performed in NSG mice. Nalm6 cells were transfected with Redifect Red-Fluc-GFP (Perkin Elmer) and selected using Puromycin for 2 months to generate a cell line that stably expressed the luciferase reporter; hereafter named Nalm6-Luc. Ten days before tumor implantation, Nalm6-Luc cells were thawed and cultured in puromycin-containing media. On day 0, cells were counted, resuspended in PBS and injected into NSG mice via tail vein. On day 6, mice were imaged for bioluminescent intensity (BLI) and sorted into groups based on their tumor size ensure that all groups had the same sized tumors. Human T cells were transduced with one of the following constructs: OT-001356, OT-001407, OT-001442, OT-001617, empty vector. Next, keeping total CARP T cell dose constant at 1 million cells per animal, OT-001407 were mixed with different amounts of OT-001356 transduced T cells (IL12+ cell dose titration, indicated in Table 20. In Table 20, “M” indicates million. In Table 20 and Table 21, “sIL12” indicates soluble IL12 which is recombinant human IL12 also referred to herein as “hIL12.”

TABLE 20
Dose groups of mice infused with T cells

| | | | # CD19car- | |
|---|---|---|---|---|
| | | # CD19- | IRES- | Total |
| | | CAR+ | IL12+ | Dose |
| Group Purpose | Group | OT-001407 | OT-001356 | Cell |
| IL12 titration | 1 | Empty Vector (pELDS-001) | | 3.33M |
| | | cells only (low total cell dose) | | |
| with 1M total CAR+ cells | 2 | 1M | — | |
| | 3 | 0.997M | 3,000 | |
| | 4 | 0.99M | 10,000 | |
| | 5 | 0.97M | 30,000 | |
| | 6 | 0.9M | 100,000 | |
| | 7 | 0.7M | 300,000 | |
| | 8 | — | 1M | |
| Control for phenotype changes in | 9 | 1M CAR-T | — | 3.33M |
| CAR-T during in vitro expansion with | | expanded with | | |
| IL12 | | sIL12 | | |

| | | | |
|---|---|---|---|
| Control for IL12 exposure in vivo | 10 | 1M IL12+ cells without CAR | |
| without CAR | | (OT-001442) | |
| Test construct with lowest basal IL12 | 11 | 1M CD19car-IRES-IL12- | 13.33M |
| for lack of off-state in vivo efficacy | | ecDHFR (OT-001617) | |
| Controls for group 11 | 12 | 1M CD19-CAR+ cells (OT- | |
| | | 001407 (high total cell dose) | |
| | 13 | Empty Vector (pELDS) cells | |
| | | only (high total cell dose) | |

Tumors were monitored in mice using BLI and the mean of the Total Flux (photons/second) was calculated for each group as an indicator of tumor burden (Table 38 and Table 39).

TABLE 21
Total Flux in tumor bearing mice after infusion of mixture of CD19-CAR
expressing human T cells and CD19-CAR-IL12 expressing T cells
Dose titration of OT-001407 plus OT-001356 (Cell # x e6)

| | | 0.997e6 | 0.99e6 | 0.97e6 | 0.97e6 | | 0.7e6 |
|---|---|---|---|---|---|---|---|
| | | OT- | OT- | OT- | OT- | | OT- |
| Days | 1.0e6 OT- | 001407 + | 001407 + | 001407 + | 001407 + | 0.9e6 OT- | 001407 + |
| Post | 001407 | 0.003e6 | 0.01e6 | 0.03e6 | 0.03e6 | 001407 + | 0.3e6 |
| Tumor | (lower | OT- | OT- | OT- | OT- | 0.1e6 OT- | OT- |
| Implant | cell dose) | 001356 | 001356 | 001356 | 001356 | 001356 | 001356 |
| 6 | 2.7E+06 | 2.7E+06 | 2.7E+06 | 2.7E+06 | 2.7E+06 | 2.7E+06 | 2.7E+06 |
| 13 | 1.4E+06 | 9.6E+05 | 9.1E+05 | 2.2E+06 | 2.2E+06 | 1.1E+06 | 1.2E+06 |
| 20 | 8.8E+05 | 6.8E+05 | 6.8E+05 | 8.0E+05 | 8.0E+05 | 6.0E+05 | 6.9E+05 |
| 27 | 2.0E+06 | 6.2E+05 | 6.8E+05 | 7.5E+05 | 7.5E+05 | 7.4E+05 | 7.4E+05 |
| 33 | 1.4E+07 | 6.9E+05 | 7.7E+05 | 7.5E+05 | 7.5E+05 | 6.8E+05 | 7.6E+05 |
| 40 | 4.6E+08 | 7.1E+05 | 7.3E+05 | 8.2E+05 | 8.2E+05 | 7.8E+05 | 7.9E+05 |
| 43 | 4.1E+09 | 6.8E+05 | 8.1E+05 | 6.8E+05 | 6.8E+05 | 7.3E+05 | 7.8E+05 |
| 47 | 8.2E+09 | 7.1E+05 | 6.6E+05 | 6.5E+05 | 6.5E+05 | 6.6E+05 | 6.1E+05 |

TABLE 22
Total Flux in tumor bearing mice after infusion of CD19-CAR, ILI2 or CD19-CAR-IL12
expressing human T cells
Controls (for groups in Table 38)

| | | | | | | | 1.0e6 |
|---|---|---|---|---|---|---|---|
| | | | | | | | OT- |
| Days | pELDS | pELDS- | | 1.0e6 | | | 001407 |
| Post | (lower | (higher | 1.0e6 | OT- | 1.0e6 | 1.0e6 | (higher |
| Tumor | cell | cell | OT- | 001407 + | OT- | OT- | cell |
| Implant | dose) | dose) | 001356 | hIL12 | 001442 | 001617 | dose) |
| 6 | 2.7E+06 | 2.7E+06 | 2.7E+06 | 2.7E+06 | 2.7E+06 | 2.7E+06 | 2.7E+06 |
| 13 | 5.6E+08 | 5.0E+08 | 5.2E+07 | 4.5E+08 | 4.5E+08 | 1.1E+06 | 1.2E+06 |
| 20 | 7.0E+09 | 7.5E+09 | 6.8E+05 | 4.2E+09 | 9.2E+09 | 1.2E+06 | 9.8E+05 |
| 27 | — | — | 7.7E+05 | 1.1E+10 | — | 7.7E+05 | 1.6E+06 |
| 33 | — | — | 7.7E+05 | — | — | 8.3E+05 | 5.6E+06 |
| 40 | — | — | 8.4E+05 | — | — | 8.1E+05 | 2.3E+08 |
| 43 | — | — | 7.3E+05 | — | — | 7.7E+05 | 1.3E+09 |
| 47 | — | — | 7.5E+05 | — | — | 7.2E+05 | 6.1E+08 |

As shown in Table 21 co-delivery of any amount of IL12-CAR⁺ T cells tested (as low as 3000 IL12-CAR+ T cells mixed with 0.979e6 CARP T cells) was sufficient to control tumor growth in vivo. Importantly, tumor regression was also observed in mice infused with T cell transduced with OT-001617 in the absence of ligand treatment, suggesting that the low basal level of IL12 produced by T cell transduced with this vector is sufficient to control tumor burden (Table 39). In contrast, recipients of T cells transduced with either CAR or IL12 monocistronic lentivirus vectors did not control tumor growth (Table 22). Percent survival was also measured for each group during the course of the experiment and the numbers shown in Table 23 were obtained.

TABLE 23
Percent Survival

| | Days Post Tumor Implant | | | | | | | | | | | |
|---|---|---|---|---|---|---|---|---|---|---|---|---|
| Construct | 0 | 20 | 21 | 22 | 23 | 25 | 26 | 27 | 29 | 43 | 44 | 47 |
| pELDS (lower | 100 | 87.5 | 50 | 12.5 | 0 | — | — | — | — | — | — | — |
| cell dose) | | | | | | | | | | | | |
| 1.0e6 OT- | 100 | — | — | — | — | — | — | — | — | 87.5 | 75 | 37.5 |
| 001407 (lower | | | | | | | | | | | | |
| cell dose) | | | | | | | | | | | | |
| 0.997e6 OT- | 100 | — | — | — | — | — | — | — | — | — | — | 100 |
| 001407 + | | | | | | | | | | | | |
| 0.003e6 OT- | | | | | | | | | | | | |
| 001356 | | | | | | | | | | | | |
| 0.99e6 OT- | 100 | — | — | — | — | — | — | — | — | — | — | 100 |
| 001407 + 0.01e6 | | | | | | | | | | | | |
| OT-001356 | | | | | | | | | | | | |
| 0.97e6 OT- | 100 | — | — | — | — | — | — | — | — | — | — | 100 |
| 001407 + 0.03e6 | | | | | | | | | | | | |
| OT-001356 | | | | | | | | | | | | |
| 0.9e6 OT- | 100 | — | — | — | — | — | — | — | — | — | — | 100 |
| 001407+ 0.1e6 | | | | | | | | | | | | |
| OT-001356 | | | | | | | | | | | | |
| 0.7e6 OT- | 100 | 87.5 | — | — | — | — | — | — | — | — | — | 87.5 |
| 001407 + 0.3e6 | | | | | | | | | | | | |
| OT-001356 | | | | | | | | | | | | |
| 1.0e6 T-001356 | 100 | — | — | — | — | — | — | — | — | — | — | 100 |
| 1.0e6 OT- | 100 | — | — | — | — | 75 | 50 | 37.5 | 0 | — | — | — |
| 001407+ hIL12 | | | | | | | | | | | | |
| 1.0e6 OT- | 100 | 62.5 | 12.5 | 0 | — | — | — | — | — | — | — | — |
| 001442 | | | | | | | | | | | | |
| 1.0e6 OT- | 100 | — | — | — | — | — | — | — | — | — | — | 100 |
| 001617 | | | | | | | | | | | | |
| 1.0e6 OT- | 100 | — | — | — | — | — | — | — | — | 87.5 | — | 87.5 |
| 001407 (higher | | | | | | | | | | | | |
| cell dose) | | | | | | | | | | | | |
| pELDS (higher | 100 | 75 | 62.5 | 37.5 | 0 | — | — | — | — | — | — | — |
| cell dose) | | | | | | | | | | | | |

Animals were bled once a week and plasma and T cells were analyzed. OT-001617 led to the expansion of CAR positive T cells in the blood of the mice at days 27 and 34 which was accompanied by an increase in IL12 and IFN gamma levels. OT-001356 resulted in consistent CAR positive T cell expansion at all time points measured namely 13, 20, 27 and 34 days and was accompanied by an increase in IL12 levels. Granzyme B levels were also elevated at most timepoints in all IL12 positive groups. Plasma IL12 also correlated with CAR-T cell numbers, plasma interferon gamma and granzyme B expression.

### Example 13. In Vivo Pharmacokinetics of IL12-ecDHFR Expression after Exposure to Trimethoprim

To evaluate the pharmacokinetics of IL12 production, NSG mice were infused with human T cells transduced with OT-001622. On day 0 (48 hours after T cell infusion), mice were bled to establish a baseline level of IL12 (pre TMP dosing) in the plasma and were then administered 500 m/kg TID (three times a day at 2, 4, 8 hours). Several blood draws were taken over 24 hours for MSD analysis of IL12 in plasma. At 2, 6, 10, and 24 hours post first the initial TMP dose, there was a 50-fold, 25-fold, 20-fold, and 4-fold increase over baseline in plasma levels of IL12 (Table 24). This fold change in plasma IL12 levels was not seen in mice treated with vehicle alone.

TABLE 24
Plasma IL12 levels in NSG mice

| Time | | | | | | | | |
|---|---|---|---|---|---|---|---|---|
| After | | | | | | | | |
| (hours) | | | | | | | | |
| First | Plasma IL12p70 (pg/mL) | | | | | | | |
| Dose | Vehicle | | | | 500 mg/kg Trimethoprim | | | |
| 0 | 0.00 | 0.00 | 1.49 | 0.00 | 0.00 | 0.00 | 0.00 | 0.00 |
| 2 | 1.45 | 1.42 | 1.38 | 0.00 | 81.57 | 65.56 | 62.34 | 70.24 |
| 6 | 3.96 | 1.56 | 1.42 | 1.38 | 56.18 | 49.70 | 49.33 | 54.44 |
| 10 | 3.09 | 0.00 | 1.73 | 0.00 | 56.47 | 53.64 | 46.89 | 47.50 |
| 24 | 1.94 | 1.52 | 1.76 | 0.00 | 6.02 | 13.85 | 4.59 | 4.83 |

### Example 14. In Vivo Regulation of ecDHFR-Regulated IL12 After Exposure to TMP

The study was designed to evaluate how pulsatile dosing of Trimethoprim (TMP) induces expression of ecDHFR-regulated IL12 by T cells transduced with OT-001617. To test this in vivo and to evaluate the effect of regulated IL12 production on in vivo anti-tumor activity, experiments were performed in NSG mice. Nalm6 cells were transfected with Redifect Red-Fluc-GFP (Perkin Elmer) under selection using puromycin for to generate a line that stably expresses the luciferase reporter; thereafter named Nalm6-Luc. Ten days before tumor implantation, Nalm6-Luc cells were thawed and cultured in puromycin-containing media. On day 0, cells were counted, resuspended in PBS and 1e6 cells were injected into NSG mice via tail vein. On day 6, mice were imaged for bioluminescent intensity (BLI) and sorted into groups based on their tumor size ensure that all groups had the same sized tumors. T cells activated with CD3/CD28 Dynabeads, transduced with lentiviral vectors carrying the constructs shown in Table 25, and expanded for 10 days. On day 7, T cells were thawed and transferred into the mice. Eight days following T cell transfer, animals were orally dosed once a day for 6 days with the indicated levels of TMP.

In vitro all constructs utilized in the study showed greater than 5% CAR positive CD3/CD45 double positive T cells, when co-cultured with K562 cells (parental or CD19 expressing). Ligand dependent IL12 expression was observed with OT-001617 expressing T cells treated with 50 μM TMP, whereas OT-001356 constitutively expressed IL12. Tumor bearing mice were infused with modified T cells and then treated with TMP (Q.D. i.e. once a day dosing) on days 15, 16, 17, 18, 19 and 20. The dose groups are shown in Table 25 and the total flux values are shown in Table 26.

TABLE 25
Dose groups of mice infused with T cells

| | CAR T | | |
|---|---|---|---|
| | Cells | Vector | TMP dose |
| Group | (×l0⁶) | (Name) | (mg/kg) |
| 1 | 0 | Empty Vector | 0 |
| 2 | 1 | OT-001407 | 0 (vehicle) |
| 3 | 1 | OT-001407 | 500 |
| 4 | 1 | OT-001356 | 0 (vehicle) |
| 5 | 1 | OT-001356 | 500 |
| 6 | 1 | OT-001617 | 500 |
| 7 | 1 | OT-001617 | 150 |
| 8 | 1 | OT-001617 | 50 |
| 9 | 1 | OT-001617 | 0 (vehicle) |

TABLE 26
Total Flux in tumor bearing mice

| | | OT-001407 | | OT-001356 | | OT-001617 | | | |
|---|---|---|---|---|---|---|---|---|---|
| | Empty | 500 | | 500 | | 500 | 150 | 50 | |
| | (pELDS) | mg/kg | | mg/kg | | mg/kg | mg/kg | mg/kg | |
| Days | Vector | TMP | Vehicle | TMP | Vehicle | TMP | TMP | TMP | Vehicle |
| 6 | 5.4E+06 | 5.3E+06 | 5.3E+06 | 5.3E+06 | 5.2E+06 | 4.0E+06 | 4.0E+06 | 4.0E+06 | 4.1E+06 |
| 14 | 4.8E+08 | 4.9E+06 | 1.4E+07 | 5.7E+06 | 3.9E+06 | 1.2E+06 | 1.1E+06 | 1.8E+06 | 1.3E+06 |
| 21 | 6.4E+09 | 9.4E+06 | 6.3E+07 | 8.8E+05 | 1.0E+06 | 9.1E+05 | 8.7E+05 | 1.0E+06 | 9.3E+05 |

As shown in Table 26, co-expression of IL12 increases CD19 CAR mediated anti-tumor activity in vivo. The basal level of IL12 expressed in the context of the CD19 CAR-IRES-IL12-ecDHFR transduced T cells was sufficient to control tumor burden in mice treated with vehicle alone.

24 hours following the final TMP dose, the number of CAR-T cells per 504 blood was analyzed by flow cytometry (mouse CD45-neg, human CD45+, human CD3+, CD19-Fc+). The number of CAR-T cells in 504 are shown in Table 27 with average values in bold.

TABLE 27
CAR-T cells in the blood

| | OT-001407 | | OT-001356 | | OT-001617 | | | |
|---|---|---|---|---|---|---|---|---|
| Naïve | | 500 | | 500 | 500 | 150 | 50 | |
| (No | | mg/kg | | mg/kg | mg/kg | mg/kg | mg/kg | |
| Tumor) | Vehicle | TMP | Vehicle | TMP | TMP | TMP | TMP | Vehicle |
| 3.42 | 27.35 | 6.84 | 52603.42 | 57181.20 | 605.13 | 203.42 | 95.73 | 68.38 |
| 1.71 | 63.25 | 44.44 | 45603.42 | 53601.71 | 611.97 | 345.30 | 160.68 | 64.96 |
| 1.71 | 32.48 | 3.42 | 33135.04 | 67005.13 | 336.75 | 85.47 | 78.63 | 71.79 |
| 0.00 | 25.64 | 42.74 | 53818.80 | 63287.18 | 326.50 | 153.85 | 157.26 | 131.62 |
| — | — | — | — | — | 526.50 | 208.55 | 47.86 | 23.93 |
| — | — | — | — | — | 210.26 | 427.35 | 100.85 | 20.51 |
| — | — | — | — | — | 642.74 | 832.48 | 49.57 | 23.93 |
| — | — | — | — | — | 423.93 | 389.74 | 78.63 | 247.86 |
| 1.71 | 37.18 | 24.36 | 46290.17 | 60268.80 | 470.09 | 197.01 | 123.08 | 84.19 |

As shown in Table 27, TMP dependent increase in T cell numbers was observed with OT-001617, whereas positive control OT-001356 showed ligand independent expansion of T cells and the negative control OT-001407 did not show any T cell expansion.

Blood samples were also collected prior to, as well as 6 and 24 hours post each dose and analyzed for IL12 levels. Pulsatile expression of IL12 was observed with TMP treatment as shown in Table 28 with peak plasma levels being achieved at 6 hours following first dose.

TABLE 28
IL12 expression in blood

| Time | | | OT- | | OT- | OT- | OT- | OT- | |
|---|---|---|---|---|---|---|---|---|---|
| After | | | 001407 | | 001356 | 001617 | 001617 | 001617 | |
| First | | OT- | 500 | OT- | 500 | 500 | 150 | 50 | OT- |
| Dose | Empty | 001407 | mg/kg | 001356 | mg/kg | mg/kg | mg/kg | mg/kg | 001617 |
| (h) | Vector | Vehicle | TMP | Vehicle | TMP | TMP | TMP | TMP | Vehicle |
| 0 | 0.06 | 0.00 | 0.00 | 1486.49 | 1561.62 | 0.44 | 0.49 | 0.44 | 0.51 |
| 6 | 0.25 | 0.00 | 0.00 | 1327.64 | 1286.87 | 15.23 | 12.91 | 9.03 | 0.50 |
| 24 | 0.46 | 0.00 | 0.00 | 878.70 | 899.92 | 0.79 | 0.57 | 0.47 | 0.53 |
| 144 | 1.00 | 0.09 | 0.00 | 232.84 | 356.07 | 1.04 | 0.86 | 0.91 | 2.14 |
| 150 | 1.30 | 0.68 | 0.00 | 230.88 | 314.10 | 7.72 | 4.41 | 3.46 | 2.21 |
| 168 | 0.97 | 0.12 | 0.07 | 300.24 | 377.69 | 1.21 | 1.01 | 0.86 | 1.63 |

### Example 15. Effect of IRES on IL12 Expression

Human T cells were activated with CD3/CD28 Dynabeads (Life Technologies) for 1 day prior to transduction with lentivirus carrying the transgene related to constructs OT-001405 or OT-001406. Seven days after the transduction, cells were treated with 1 μM Shield-1 for 24 hours or vehicle control. Flexi IL12 was quantified by MSD assay for IL12p70. Constitutive IL12 expression was observed under the control of PGK promoter for OT-001406. Bicistronic construct with CD19 and IL12 (or IL12 FKBP-DD) separated by internal ribosome entry sequence (IRES) OT-001405 showed low basal expression in the absence of ligand and approximately 5-10 pg/ml IL12 levels in the presence of shield 1.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A modified cell comprising:
(a) a first recombinant protein comprising an effector module, said effector module comprising:
(i) a stimulus response element (SRE) linked to at least one recombinant protein selected from: a cytokine, a cytokine-cytokine receptor fusion protein, and a CD19 chimeric antigen receptor (CD19 CAR); and
(ii) the SRE comprises a DD, wherein said DD is derived from a parent protein or a mutant protein having one or more amino acid mutations in the amino acid sequence of the DD compared to said parent protein, wherein the parent protein is selected from the group consisting of:
(i) human DHFR (hDHFR) (SEQ ID NO: 1);
(ii} E. coli DHFR (ecDHFR) (SEQ ID NO: 2); and
(iii) human protein FKBP (SEQ ID NOs: 3 or 1087); and
(b) optionally, a second recombinant protein comprising a CD19 chimeric antigen receptor (CAR).

2. The cell of claim 1, wherein the cytokine comprises IL12, IL15, or combinations thereof.

3. The cell of claim 2, wherein the IL12 is a fusion protein comprising a p40 subunit, a linker, and a p35 subunit.

4. The cell of claim 3, wherein said p40 subunit is a p40 (23-328 of WT) (SEQ ID NO: 563), a p40 (WT) (SEQ ID NO:1091) or a p40 (23-328 of WT) (K217N) (SEQ ID NO: 578).

5. The cell of claim 4, wherein said p40 subunit is p40 (23-328 of WT) (SEQ ID NO: 563).

6. The cell of claim 3, wherein the p35 subunit is a p35 (57-253 of WT) (SEQ ID NO: 564) or p35 (WT) (SEQ ID NO: 1093).

7. The cell of claim 6, wherein the p35 subunit is a p35 (57-253 of WT) (SEQ ID NO: 564).

8. The cell of claim 1, wherein the cytokine-cytokine receptor fusion polypeptide comprises the whole or a portion of SEQ. ID NOs: 616, 632 fused to the whole or a portion of any of SEQ. ID NOs: 632; 855, or 1097 to produce a IL15-1L15 receptor fusion polypeptide.

9. The cell of claim 1, wherein the parent protein is a human DHFR (hDHFR), and the DD comprises one or more mutations selected from the group consisting of: Mdel1, I17A, I17V, Q36F, Q36K, N65F, Y122I, N127Y, and A125F.

10. The cell of claim 1, wherein the parent protein is a human DHFR (hDHFR), and the DD comprises one or more mutations selected from:
a single mutation selected from the group consisting of: Mdel1, I17A, I17V, Q36F, Q36K, N65F, Y122I, and A125F;
a double mutation selected from the group consisting of: (M1del, I17A), (M1del, I17V), and (M1del, Y122I);
a triple mutation selected from the group consisting of: (M1del, Y122I, A125F), (M1del, Q36K, Y122I), (M1del, I17V, Y122I), and (M1del, I17A, Y122I); and
a quadruple or higher mutation selected from the group consisting of: (M1del, Q36F, N65F, Y122I).

11. The cell of claim 10, wherein the DD comprises an hDHFR mutant protein having three mutations (M1del, Y122I, N127Y).

12. The cell of claim 10, wherein the DD comprises an hDHFR mutant protein having three mutations (M1del, I17V, Y122I).

13. The cell of claim 10, wherein the DD comprises an hDHFR mutant protein having two mutations (M1del, I17V).

14. The cell of claim 1, wherein the CD19 CAR is linked to the effector module.

15. The cell of claim 1, wherein the CD19 CAR is not linked to the effector module.

16. The cell of claim 1, wherein the CD19 CAR comprises:
(a) a CD19 binding moiety;
(b) a transmembrane domain;
(c) an intracellular signaling domain; and
(d) optionally, one or more co-stimulatory domains.

17. The cell of claim 16, wherein the CD19 binding moiety is selected from:
(a) a single chain variable fragment (scFv),
(b) an Ig NAR,
(c) a Fab fragment,
(d) a Fab′ fragment,
(e) a F(ab)′2 fragment,
(f) a F(ab)′3 fragment,
(g) an Fv,
(h) a bis-scFv, a (scFv)2,
(i) a minibody,
(j) a diabody,
(k) a triabody,
(l) a tetrabody,
(m) an intrabody,
(n) a disulfide stabilized Fv protein (dsFv),
(o) a unibody,
(p) a nanobody, and
(q) an antigen binding region derived from any one of (a) to (p) that binds to CD19.

18. The cell of claim 17, wherein the CD19 binding moiety is a scFv that specifically binds a CD19 antigen.

19. The cell of claim 18, wherein the scFv is a CD19 scFv comprising an amino acid sequence of SEQ ID NO: 465.

20. The cell of claim 1, wherein the cytokine, cytokine-cytokine receptor fusion protein or CAR component is further linked to at least one of:
(a) a leader sequence;
(b) a signal peptide:
(c) a linker;
(d) a spacer;
(e) a cleavage site;
(f) a tag;
(g) a co-stimulatory domain;
(h) a fluorescence protein; and
(i) a hinge.

21. The cell of claim 16, wherein the intracellular signaling domain of the CD19 CAR is the signaling domain derived from T cell receptor CD3zeta or a cell surface molecule selected from the group consisting of FcR gamma, FcR beta, CD3 gamma, CD3 delta, CD3 epsilon, CD5, CD22, CD79a, CD79b, and CD66d; and the co-stimulatory domain is present and is selected from the group consisting of 4-1BB (CD137), 2B4, HVEM, ICOS, LAG3, DAP10, DAP12, CD27, CD28, OX40 (CD134), CD30, CD40, ICOS (CD278), glucocorticoid-induced tumor necrosis factor receptor (GITR), lymphocyte function-associated antigen-1 (LFA-1), CD2, CD7, LIGHT, NKG2C, and B7-H3.

22. The cell of claim 21, wherein the intracellular signaling domain of the CD19 CAR comprises a T-cell receptor CD3zeta signaling domain comprising the amino acid sequence of SEQ ID NO: 299.

23. The cell of claim 22, wherein the intracellular signaling domain of the CD19 CAR is a T-cell receptor CD3zeta signaling domain comprising the amino acid sequence of SEQ ID NO: 467 and when the co-stimulatory domain is present, the co-stimulatory domain has an amino acid sequence selected from SEQ ID NOs: 233, 228-232, and 234-334.

24. The cell of claim 16, wherein the transmembrane domain is derived from any of the members of the group consisting of:
(a) a molecule selected from the group consisting of CD8α, CD4, CD5, CD8, CD8α, CD9, CD16, CD22, CD33, CD28, CD37, CD45, CD64, CD80, CD86, CD148, DAP 10, EpoRI, GITR, LAG3, ICOS, Her2, OX40 (CD134), 4-1BB (CD137), CD152, CD154, PD-1, or CTLA-4
(b) a transmembrane region of an alpha, beta or zeta chain of a T-cell receptor;
(c) the CD3 epsilon chain of a T-cell receptor; and
(d) an immunoglobulin selected from IgG1, IgD, IgG4, and an IgG4 Fc region.

25. The cell of claim 24, wherein the transmembrane domain comprises an amino acid sequence selected from the group consisting of SEQ ID NOs: 369, 335-368, 370-385 and 697-707.

26. The cell of claim 16, wherein the CAR further comprises a hinge region near the transmembrane domain, said hinge region comprising an amino acid sequence selected from the group consisting of SEQ ID NOs: 400, 386-399, and 401-464.

27. The cell of claim 1, wherein the SRE is responsive to or interacts with at least one stimulus.

28. The cell of claim 27, wherein the stimulus is Trimethoprim (TMP) or Methotrexate (MTX).

29. The cell of claim 1, wherein
(a) the effector module is selected from the group consisting of SEQ ID NOs: 1121, 1123, 1129, 1131, 1133, 1135, 1137, 1139, and 1141; and
(b) the CD19 CAR is selected from the group consisting of SEQ ID NOs: 1120, 1122, 1128, 1130, 1132, 1134, 1136, 1138, and 1140.

30. The cell of claim 1, comprising at least one recombinant protein comprising an amino acid sequence selected from the group consisting of SEQ ID NOs: 1127, 1125, 1126, 1082, 1118, 1119, 1124, and 1127.

31. The cell of claim 1, wherein the cell is a T-cell.

32.-48. (canceled)
